SEQUENCE LISTING (11 sequences)

SEQ ID NO: 1 - protein, 400 aa, Homo sapiens
MLLLVTSLLLCELPHPAFLLIPEKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 2 - protein, 447 aa, Artificial Sequence
  source: /note=“Description of Artificial Sequence Synthetic polypeptide”
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 217 aa, Artificial Sequence
  source: /note=“Description of Artificial Sequence Synthetic polypeptide”
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 120 aa, Artificial Sequence
  source: /note=“Description of Artificial Sequence Synthetic polypeptide”
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSS

SEQ ID NO: 5 - protein, 111 aa, Artificial Sequence
  source: /note=“Description of Artificial Sequence Synthetic polypeptide”
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVL

SEQ ID NO: 6 - protein, 5 aa, Artificial Sequence
  source: /note=“Description of Artificial Sequence Synthetic peptide”
ELSIH

SEQ ID NO: 7 - protein, 17 aa, Artificial Sequence
  source: /note=“Description of Artificial Sequence Synthetic peptide”
GFDPEENEIVYAQRFQG

SEQ ID NO: 8 - protein, 11 aa, Artificial Sequence
  source: /note=“Description of Artificial Sequence Synthetic peptide”
VGSFSPLTLGL

SEQ ID NO: 9 - protein, 14 aa, Artificial Sequence
  source: /note=“Description of Artificial Sequence Synthetic peptide”
TGSGSNIGAPYDVS

SEQ ID NO: 10 - protein, 7 aa, Artificial Sequence
  source: /note=“Description of Artificial Sequence Synthetic peptide”
HNNKRPS

SEQ ID NO: 11 - protein, 11 aa, Artificial Sequence
  source: /note=“Description of Artificial Sequence Synthetic peptide”
ATVEAGLSGSV

(19) United States
(12) Patent Application Publication
(10) Pub. No.: US 2022/0281914 A1
(43) Pub. Date: Sep. 8, 2022

(54) PROTEIN COMPOSITIONS AND METHODS FOR PRODUCING AND USING THE SAME

(71) Applicant: KINIKSA PHARMACEUTICALS, LTD., Hamilton (BM)

(72) Inventors: Mei Jang, Lexington, MA (US); Dave Nichols, Lexington, MA (US); Baochuan Huang, Lexington, MA (US); Shaun Grier, Lexington, MA (US)

(73) Assignee: KINIKSA PHARMACEUTICALS, LTD., Hamilton (BM)

(21) Appl. No.: 17/554,659

(22) Filed: Dec. 17, 2021

Related U.S. Application Data
(60) Provisional application No. 63/127,973, filed on Dec. 18, 2020.

(51) Int. Cl. C07K 1/36; C07K 1/18; C07K 1/22; B01D 15/38; B01D 15/32

(52) U.S. Cl. CPC C07K 1/36; C07K 1/18; C07K 1/22; B01D 15/3809; B01D 15/327

(57) ABSTRACT

The instant invention relates to compositions comprising a protein, e.g., an antibody, or antigen-binding portion thereof, e.g., an anti-GM-CSFRα antibody or antigen binding portion thereof, and methods, e.g., cell culture and/or protein purification methods, for producing such compositions. Methods for using such compositions to treat a disorder, e.g., a GM-CSFRα-associated disorder, are also provided.

### RELATED APPLICATIONS

This application claims the benefit of priority to U.S. Provisional Application No. 63/127,973, filed on Dec. 18, 2020, the entire contents of which are incorporated hereby by reference.

### SEQUENCE LISTING

The instant application contains a Sequence Listing which has been submitted electronically in ASCII format and is hereby incorporated by reference in its entirety. Said ASCII copy, created Mar. 24, 2022, is named 132261_00102_SL.txt and is 13,656 bytes in size.

### BACKGROUND OF THE INVENTION

Antibodies, such as monoclonal antibodies (mAbs), are an important class of therapeutic drugs in the pharmaceutical industry. Antibody therapeutics have been developed for treating many diseases such as cancer, inflammation and autoimmune disorders.

The production of proteins such as monoclonal antibodies for pharmaceutical applications generally involves the use of upstream process technologies (e.g., cell culture) and downstream process technologies (e.g., protein purification). Typically, antibodies are produced as recombinant proteins in mammalian cell cultures to ensure proper folding and post-translational modification. Monoclonal antibodies produced from cell cultures need to be purified from host cell proteins and other impurities in order to be effectively utilized, for example, to improve the safety profile.

Proteins exhibiting varying levels of variants and impurities may be produced through the upstream and downstream process. Such protein variants and impurities include, but are not limited to, product-related substances, e.g., protein aggregates, fragments, or charged species, e.g., acidic or basic species; and/or process-related impurities, e.g., host cell proteins, nucleic acids, and residual media components.

### SUMMARY OF THE INVENTION

The present invention is based on the identification and optimization of upstream and downstream process technologies for protein production, e.g., production of antibodies or antigen-binding portions thereof, resulting in compositions having low levels of variants and/or impurities, e.g., low levels of product-related substances, e.g., product aggregates, fragments or charged species, e.g., acidic or basic species, and/or low levels of process-related impurities, e.g., host cell proteins.

Accordingly, in one aspect, the present invention provides a method of producing a preparation including a protein of interest having a reduced level of half antibody, the method including subjecting a sample including the protein of interest and half antibody to a cation exchange chromatography resin or a mixed mode chromatography resin, thereby producing the preparation including the protein of interest having a reduced level of half antibody.

In another aspect, the present invention provides a method of reducing the level of half antibody in a preparation including a protein of interest, the method including subjecting a sample including the protein of interest and half antibody to a cation exchange chromatography resin or a mixed mode chromatography resin, thereby reducing the level of half antibody in the preparation including the protein of interest.

In some embodiments, the protein of interest is an antibody or antigen-binding portion thereof. In some embodiments, the antibody or antigen-binding portion thereof is an anti-GM-CSFRα antibody or antigen-binding portion thereof. In some embodiments, the anti-GM-CSFRα antibody or antigen-binding portion thereof is mavrilimumab.

In some embodiments, the sample is subject to a cation exchange chromatography resin. In some embodiments, the cation exchange chromatography resin includes a functional group selected from the group consisting of sulfhydryl, sulfonate, sulfate, carboxymethyl, sulfoethyl, sulfopropyl, phosphate and sulfonate. In some embodiments, the cation exchange chromatography resin is selected from the group consisting of POROS™ XS CEX, Capto™ S ImpAct, TOTOPEARL™ GigaGap CM 650M, and TOYOPEAL™ sulfate 650F. In some embodiments, the cation exchange chromatography resin runs in bind-elute mode.

In some embodiments, the sample is subject to a mixed mode chromatography resin. In some embodiments, the mixed mode chromatography resin includes a functional group selected from the group consisting of carboxyl, hydroxyl, N-Benzyl-N-methyl ethanol amine, phenylpropylamine and hexylamine. In some embodiments, the mixed mode chromatography resin is selected from the group consisting of Capt™ MMC ImpRes and Capto™ Adhere ImpRes.

In some embodiments, the preparation includes less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

In some embodiments, the preparation includes less than about 2.8% half antibody.

In some embodiments, the preparation includes about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody.

In some embodiments, the preparation includes about 0.6-1.7% half antibody.

In some embodiments, the level of half antibody in the preparation is reduced by at least about 90%, about 80%, about 70%, about 60%, about 50%, about 40%, about 30%, about 20% or about 10% as compared to the level of half antibody in the sample.

In some embodiments, the methods further include collecting an eluate fraction using an elution buffer.

In some embodiments, the eluate fraction includes less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

In some embodiments, the eluate fraction includes about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18%, or about 1-17% half antibody.

In some embodiments, the eluate fraction is collected from cation exchange chromatography resin. In some embodiments, the eluate fraction collected from cation exchange chromatography resin includes about 0.6-18% half antibody.

In some embodiments, the eluate fraction is collected from mixed mode chromatography resin. In some embodiments, the eluate fraction collected from mixed mode chromatography resin and includes about 1-17% half antibody.

In some embodiments, the level of half antibody in the eluate fraction is reduced by at least about 90%, about 80%, about 70%, about 60%, about 50%, about 40%, about 30%, about 20% or about 10% as compared to the level of half antibody in the sample.

In some embodiments, the elution buffer includes about 1-500 mM, about 10-250 mM, about 10-150 mM, about 10-100 mM, about 20-90 mM, about 30-80 mM, about 40-70 mM, or about 50-60 mM sodium acetate. In some embodiments, wherein the elution buffer includes about 40-60 mM sodium acetate.

In some embodiments, the elution buffer includes about 1-500 mM, about 10-250 mM, about 10-150 mM, about 10-100 mM, about 20-90 mM, about 30-80 mM, about 40-70 mM, or about 50-60 mM sodium chloride. In some embodiments, the elution buffer includes about 40-60 mM sodium chloride.

In some embodiments, the elution buffer includes a pH of about 4-7, about 5-6, about 5-5.5. In some embodiments, the elution buffer includes a pH of about 5-5.5.

In some embodiments, the elution buffer includes about 50 mM sodium acetate, about 55 mM sodium chloride, and a pH of about 5.35.

In some embodiments, the protein of interest is loaded onto the cation exchange chromatography resin or the mixed mode chromatography resin at a level of about 10-100 g/L, about 20-90 g/L, about 30-80 g/L, about 40-70 g/L, or about 50-60 g/L. In some embodiments, the protein of interest is loaded onto the cation exchange chromatography resin or the mixed mode chromatography resin at a level of about 30-60 g/L.

In some embodiments, the level of half antibody is determined by non-reduced CE-SDS (capillary electrophoresis with sodium dodecylsulfate).

In one aspect, the present invention provides a composition including an anti-GM-CSFRα antibody, or antigen-binding portion thereof, including less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

In some embodiments, the composition includes less than about 2.8% half antibody.

In some embodiments, the composition includes about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody. In some embodiments, the composition includes about 0.6-1.7% half antibody.

In one aspect, the present invention provides a composition including an anti-GM-CSFRα antibody, or antigen-binding portion thereof, wherein the composition includes an eluate fraction collected from a cation exchange chromatography resin or a mixed mode chromatography resin, and wherein the eluate fraction includes less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

In some embodiments, the eluate fraction includes about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody.

In some embodiments, the eluate fraction is collected from a cation exchange resin and includes about 0.6-18% half antibody.

In some embodiments, the eluate fraction is collected from a mixed mode resin and includes about 1-17% half antibody.

In another aspect, the present invention provides a composition including an anti-GM-CSFRα antibody, or antigen-binding portion thereof, wherein the composition includes a flow through and/or a wash fraction collected from a cation exchange chromatography resin, and wherein the flow through and/or wash fraction includes less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% half antibody.

In some embodiments, the flow through and/or wash fraction includes less than about 6% half antibody.

In some embodiments, the level of half antibody is determined by non-reduced CE-SDS (capillary electrophoresis with sodium dodecylsulfate).

In some embodiments, the anti-GM-CSFRα antibody or antigen-binding portion thereof is mavrilimumab.

In one aspect, the present invention provides a pharmaceutical composition including any one of the compositions described herein, and a pharmaceutically acceptable carrier.

In one aspect, the present invention provides a method of producing a preparation including an anti-GM-CSFRα antibody, or antigen-binding portion thereof, having a reduced level of acidic species, the method including subjecting a sample including the anti-GM-CSFRα antibody, or antigen-binding portion thereof, and acidic species to an anion exchange chromatography resin or a mixed mode chromatography resin, thereby producing the preparation including the anti-GM-CSFRα antibody, or antigen-binding portion thereof, having a reduced level of acidic species.

In another aspect, the present invention provides a method of reducing the level of acidic species in a preparation including an anti-GM-CSFRα antibody, or antigen-binding portion thereof, the method including subjecting a sample including the anti-GM-CSFRα antibody, or antigen-binding portion thereof, and acidic species to an anion exchange chromatography resin or a mixed mode chromatography resin, thereby reducing the level of acidic species in the preparation including the anti-GM-CSFRα antibody, or antigen-binding portion thereof.

In some embodiments, the anti-GM-CSFRα antibody or antigen-binding portion thereof is mavrilimumab.

In some embodiments, the sample is subject to an anion exchange chromatography resin. In some embodiments, the anion exchange chromatography resin includes a functional group selected from the group consisting of diethylaminoethyl, quaternary aminoethyl and quaternary amine. In some embodiments, the anion exchange chromatography resin is selected from the group consisting of POROS™ XQ AEX and Capto™ Q ImpRes. In some embodiments, the anion exchange chromatography resin runs in bind-elute mode.

In some embodiments, the sample is subject to a mixed mode chromatography resin. In some embodiments, th mixed mode chromatography resin includes a functional group selected from the group consisting of carboxyl, hydroxyl, N-Benzyl-N-methyl ethanol amine, phenylpropylamine and hexylamine. In some embodiments, the mixed mode chromatography resin is Capto™ Adhere ImpRes.

In some embodiments, the preparation includes less than about 40%, about 35%, about 30%, about 25%, about 24%, about 23%, about 22%, about 21%, about 20%, about 19%, about 18%, about 17%, about 16%, about 15%, about 10%, or about 5% acidic species.

In some embodiments, the preparation includes about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species.

In some embodiments, the preparation includes about 11-22% acidic species. In some embodiments, the preparation includes about 11-38% acidic species. In some embodiments, the preparation includes about 9-18% acidic species. In some embodiments, the preparation includes about 11-38% acidic species and less than about 24% basic species. In some embodiments, the preparation includes about 11-38% acidic species and either (i) between about 58-62% main species or (ii) more than about 64% main species.

In some embodiments, the methods further include collecting an eluate fraction using an elution buffer.

In some embodiments, the eluate fraction includes less than about 40%, about 35%, about 30%, about 25%, about 24%, about 23%, about 22%, about 21%, about 20%, about 19%, about 18%, about 17%, about 16%, about 15%, about 10%, or about 5% acidic species.

In some embodiments, the eluate fraction includes about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species.

In some embodiments, the eluate fraction is collected from an anion exchange chromatography resin. In some embodiments, the eluate fraction collected from anion exchange chromatography resin includes about 11-22% acidic species.

In some embodiments, the eluate fraction is collected from a mixed mode chromatography resin. In some embodiments, the eluate fraction collected from mixed mode chromatography resin includes about 12-38% acidic species.

In some embodiments, the level of acidic species in the preparation or the eluate fraction is reduced by at least about 90%, about 80%, about 70%, about 60%, about 50%, about 40%, about 30%, about 20% or about 10% as compared to the level of acidic species in the sample.

In some embodiments, the elution buffer includes about 1-500 mM, about 10-250 mM, about 50-200 mM, about 70-150 mM, about 90-130 mM, or about 100-110 mM sodium chloride. In some embodiments, the elution buffer includes about 100-110 mM sodium chloride.

In some embodiments, the elution buffer includes about 1-500 mM, about 10-250 mM, about 20-150 mM, about 30-100 mM, about 20-90 mM, about 30-80 mM, about 40-70 mM, or about 50-60 mM histidine. In some embodiments, the elution buffer includes about 40-60 mM histidine.

In some embodiments, the elution buffer includes about 1-500 mM, about 10-250 mM, about 10-150 mM, about 10-100 mM, about 20-90 mM, about 30-80 mM, about 40-70 mM, or about 50-60 mM acetate. In some embodiments, the elution buffer includes about 40-60 mM acetate.

In some embodiments, the elution buffer includes about 1-500 mM, about 10-250 mM, about 10-150 mM, about 10-100 mM, about 20-90 mM, about 30-80 mM, about 40-70 mM, or about 50-60 mM Bis-Tris. In some embodiments, the elution buffer includes about 40-60 mM Bis-Tris.

In some embodiments, the elution buffer includes a pH of about 5-7 or about 5.5-6.5. In some embodiments, the elution buffer includes a pH of about 5.5-6.5.

In some embodiments, the elution buffer includes about 50 mM histidine, about 105 mM NaCl, and has a pH of about 6.0.

In some embodiments, the protein of interest is loaded onto the anion exchange chromatography resin or the mixed mode chromatography resin at a level of about 10-100 g/L, about 20-90 g/L, about 30-80 g/L, or about 40-70 g/L. In some embodiments, the protein of interest is loaded onto the anion exchange chromatography resin or the mixed mode chromatography resin at a level of about 50-60 g/L.

In some embodiments, the level of acidic species is determined by ion exchange chromatography.

In some embodiments, prior to subjecting said sample to an anion exchange chromatography resin or a mixed mode chromatography resin, the sample is subjected to a cation exchange chromatography resin or a mixed mode chromatography resin.

In one aspect, the present invention provides a composition including an anti-GM-CSFRα antibody, or antigen-binding portion thereof, wherein the composition includes less than about 40%, about 35%, about 30%, about 25%, about 24%, about 23%, about 22%, about 21%, about 20%, about 19%, about 18%, about 17%, about 16%, about 15%, about 10%, or about 5% acidic species of the antibody.

In another aspect, the present invention provides a composition including an anti-GM-CSFRα antibody, or antigen-binding portion thereof, wherein the composition includes about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species.

In some embodiments, the composition includes about 11-22% acidic species. In some embodiments, the composition includes about 9-18% acidic species.

In one aspect, the present invention provides a composition including anti-GM-CSFRα antibody, or antigen-binding portion thereof, wherein the composition includes less than about 45%, about 40%, about 35%, about 30%, about 25%, about 24%, about 23%, about 22%, about 21%, about 20%, about 19%, about 18%, about 17%, about 16%, about 15%, about 10%, or about 5% basic species of the antibody. In some embodiments, the composition includes less than about 24% basic species.

In another aspect, the present invention provides a composition including an anti-GM-CSFRα antibody, or antigen-binding portion thereof, wherein the composition includes about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 17-26%, about 9-29%, about 9-41%, or about 16-41% basic species.

In some embodiments, the composition includes about 16-41% basic species.

In one aspect, the present invention provides a composition including anti-GM-CSFRα antibody, or antigen-binding portion thereof, wherein the composition includes more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species of the antibody.

In some embodiments, the composition includes more than about 64% main species.

In another aspect, the present invention provides a composition including anti-GM-CSFRα antibody, or antigen-binding portion thereof, wherein the composition includes about 40-99%, about 45-99%, about 50-99%, about 55-99%, about 50-90%, about 55-90%, about 50-80%, about 55-80%, about 50-70%, about 55-70%, about 50-65%, about 46-67%, about 55-65%, about 58-62%, about 58-63%, about 58-67%, about 53-61%, or about 46-66% main species.

In some embodiments, the composition includes about 46-67% main species.

In some embodiments, the composition includes about 58-62% main species.

In some embodiments, the composition includes about 11-38% acidic species and less than about 24% basic species. In some embodiments, the composition includes about 11-38% acidic species and more than about 64% main species. In some embodiments, the composition includes about 11-38% acidic species and about 58-62% main species. In some embodiments, the composition includes about 9-41% basic species and about 9-18% acidic species. In some embodiments, the composition includes about 9-41% basic species and more than about 64% main species. In some embodiments, the composition includes about 16-41% basic species and about 58-62% main species. In some embodiments, the composition includes about 46-67% main species and about 9-18% acidic species. In some embodiments, the composition includes about 46-67% main species and less than 24% basic species.

In one aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes less than about 40%, about 35%, about 30%, about 25%, about 24%, about 23%, about 22%, about 21%, about 20%, about 19%, about 18%, about 17%, about 16%, about 15%, about 10%, or about 5% acidic species.

In another aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species.

In some embodiments, the eluate fraction includes about 11-38% acidic species. In some embodiments, the eluate fraction is collected from anion exchange chromatography resin and includes about 11-22% acidic species. In some embodiments, the eluate fraction is collected from mixed mode chromatography resin and includes about 12-38% acidic species.

In one aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes less than about 45%, about 40%, about 35%, about 30%, about 25%, about 24%, about 23%, about 22%, about 21%, about 20%, about 19%, about 18%, about 17%, about 16%, about 15%, about 10%, or about 5% basic species.

In another aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 17-26%, about 9-29%, about 9-41%, or about 16-41% basic species.

In some embodiments, the eluate fraction includes about 9-41% basic species.

In some embodiments, the eluate fraction is collected from mixed mode chromatography resin and includes about 9-29% basic species. In some embodiments, the eluate fraction is collected from anion exchange chromatography resin and includes about 16-41% basic species.

In one aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes more than about 40%, about 45%, about 50%, about 55%, about 60%, about 63%, about 64%, about 65%, about 66%, about 70%, about 75%, about 80%, about 85%, about 90%, about 95% or about 99% main species.

In another aspect, the present invention provides a composition including an anti-GM-CSFRα antibody, or antigen-binding portion thereof, wherein the composition includes an eluate fraction collected from an anion exchange chromatography resin or a mixed mode chromatography resin, and wherein the eluate fraction includes about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 58-63%, about 58-67%, about 46-67%, about 53-61%, or about 46-66% main species.

In some embodiments, the eluate fraction is collected from mixed mode chromatography resin and includes about 53-61% main species.

In some embodiments, the eluate fraction is collected from anion exchange chromatography resin and includes about 46-66% main species.

In some embodiments of any of the foregoing aspects, the anti-GM-CSFRα antibody or antigen-binding portion thereof is mavrilimumab.

In some embodiments, the level of acidic species, the level of main species or the level of basic species is determined by ion exchange chromatography.

In one aspect, the present invention provides a pharmaceutical composition including any one of the compositions described herein, and a pharmaceutically acceptable carrier.

In one aspect, the present invention provides a method of producing a preparation including a protein of interest having a reduced level of high molecular weight aggregates and/or host cell proteins, the method including subjecting a sample including the protein of interest, high molecular weight aggregates and/or host cell proteins (HCP) to a chromatography resin, wherein the chromatography resin is selected from a group consisting of a cation exchange chromatography resin, an anion exchange chromatography resin and a mixed mode chromatography resin, thereby producing the preparation including the protein of interest having a reduced level of high molecular weight aggregates and/or host cell proteins.

In another aspect, the present invention provides a method of reducing the level of high molecular weight aggregates and/or host cell proteins (HCP) in a preparation including a protein of interest, the method including subjecting a sample including the protein of interest and half antibody to a chromatography resin, wherein the chromatography resin is selected from a group consisting of a cation exchange chromatography resin, an anion exchange chromatography resin and a mixed mode chromatography resin, thereby reducing the level of high molecular weight aggregates and/or host cell proteins in the preparation including the protein of interest.

In some embodiments, the protein of interest is an antibody or antigen-binding portion thereof. In some embodiments, the antibody or antigen-binding portion thereof is an anti-GM-CSFRα antibody or antigen-binding portion thereof. In some embodiments, the anti-GM-CSFRα antibody or antigen-binding portion thereof is mavrilimumab.

In some embodiments, the chromatography resin is a cation exchange chromatography resin.

In some embodiments, the cation exchange chromatography resin includes a functional group selected from the group consisting of sulpfhydryl, sulfonate, sulfate, carboxymethyl, sulfoethyl, sulfopropyl, phosphate and sulfonate. In some embodiments, the cation exchange chromatography resin is selected from the group consisting of POROS™ XS CEX, Capto™ S ImpAct, TOTOPEARL™ GigaGap CM 650M, and TOYOPEAL™ sulfate 650F. In some embodiments, the cation exchange chromatography resin runs in bind-elute mode.

In some embodiments, the chromatography resin is an anion exchange chromatography resin. In some embodiments, the anion exchange chromatography resin includes a functional group selected from the group consisting of diethylaminoethyl, quaternary aminoethyl and quaternary amine. In some embodiments, the anion exchange chromatography resin is selected from the group consisting of POROS™ XQ AEX and Capto™ Q ImpRes. In some embodiments, the anion exchange chromatography resin runs in bind-elute mode.

In some embodiments, the chromatography resin is a mixed mode chromatography resin. In some embodiments, the mixed mode chromatography resin includes a functional group selected from the group consisting of carboxyl, hydroxyl, N-Benzyl-N-methyl ethanol amine, phenylpropylamine and hexylamine. In some embodiments, the mixed mode chromatography resin is selected from the group consisting of Capt™ MMC ImpRes and Capto™ Adhere ImpRes.

In some embodiments, the preparation includes less than about 10%, about 9%, about 8%, about 7%, about 6%, about 5%, about 4%, about 3%, about 1%, about 0.9%, about 0.8%, about 0.7%, about 0.6%, about 0.5%, about 0.4%, about 0.3%, about 0.2%, or about 0.1% high molecular weight aggregates. In some embodiments, the preparation includes less than 0.5% high molecular weight aggregates.

In some embodiments, the preparation includes about 0.01-10%, about 0.01-5%, about 0.01-1%, about 0.04-0.2%, about 0.1-0.4%, about 0.04-0.4%, about 0.04-0.8%, about 0.5-0.8%, about 1-10%, about 2-10%, about 3-10%, or about 4-10% high molecular weight aggregates. In some embodiments, the preparation includes about 0.04-0.8% high molecular weight aggregates.

In some embodiments, the level of high molecular weight aggregates in the preparation is reduced by at least about 90%, about 80%, about 70%, about 60%, about 50%, about 40%, about 30%, about 20% or about 10% as compared to the level of high molecular weight aggregates in the sample.

In some embodiments, the preparation includes less than about 10, about 9, about 8, about 7, about 6, about 5, about 4, about 3, about 2, about 1, about 0.9, about 0.8, about 0.7, about 0.6 or about 0.5 ppm HCP.

In some embodiments, the preparation includes about 0.1-10, about 1-10, about 2-10, about 3-10, about 4-10, about 1-5, about 5-10, about 0.1-2, about 0.1-3, about 2-8 or about 0.1-8 ppm HCP.

In some embodiments, the preparation includes about 0.1-2 ppm HCP.

In some embodiments, the level of HCP in the preparation is reduced by at least about 90%, about 80%, about 70%, about 60%, about 50%, about 40%, about 30%, about 20% or about 10% as compared to the level of HCP in the sample.

In some embodiments, the preparation includes more than about 90%, about 91%, about 92%, about 93%, about 94%, about 95%, about 96%, about 97%, about 98%, about 99%, about 99.1% or about 99.5% of monomer of the protein of interest.

In some embodiments, the preparation includes more than about 99.1% of monomer of the protein of interest.

In some embodiments, the preparation includes about 90-99.9%, about 90-95%, about 95-99.9%, about 99-99.9%, about 99.1-99.9%, about 98-99%, about 98-99.9%, or about 98.5-99.5% of monomer of the protein of interest.

In some embodiments, the preparation includes about 98-99% monomer of the protein of interest. In some embodiments, the preparation includes about 98-99.9% monomer of the protein of interest.

In some embodiments, the preparation includes less than 10%, about 9%, about 8%, about 7%, about 6%, about 5%, about 4%, about 3%, about 2%, about 1%, about 0.5%, about 0.4%, about 0.3%, about 0.2% or about 0.1% fragments of protein of interest. In some embodiments, the preparation includes less than about 0.4% or less than 0.3% fragments of protein of interest.

In some embodiments, the preparation includes about 0.1-10%, about 0.1-5%, about 0.1-3%, about 0.1-2%, about 0.6-1.5%, about 0.5-1.5%, about 0.5-1.1%, about 0.4-0.8% or about 0.4-1.1% fragments of protein of interest. In some embodiments, the preparation includes about 0.6-1.5% fragments of protein of interest. In some embodiments, the preparation includes about 0.5-1.5% fragments of protein of interest.

In some embodiments, the method further includes collecting an eluate fraction using an elution buffer.

In some embodiments, the eluate fraction includes less than about 10%, about 9%, about 8%, about 7%, about 6%, about 5%, about 4%, about 3%, about 1%, about 0.9%, about 0.8%, about 0.7%, about 0.6%, about 0.5%, about 0.4%, about 0.3%, about 0.2%, or about 0.1% high molecular weight aggregates.

In some embodiments, the eluate fraction includes about 0.01-10%, about 0.01-5%, about 0.01-1%, about 0.04-0.8%, about 0.04-0.2%, about 0.1-0.4%, about 0.04-0.4%, about 0.5-0.8%, about 0.1-6%, about 1-10%, about 2-10%, about 3-10%, or about 4-10% high molecular weight aggregates. In some embodiments, the eluate fraction includes about 0.04-0.4% high molecular weight aggregates.

In some embodiments, the eluate fraction is collected from a cation exchange resin and includes about 0.01-10%, about 0.01 to 5%, about 0.01 to 1%, about 0.04-0.2%, about 0.04-0.8%, about 0.1-0.4%, about 0.04-0.4%, about 0.5-0.7%, about 0.1-6%, about 1-10%, about 2-10%, about 3-10%, or about 4-10% high molecular weight aggregates.

In some embodiments, the eluate fraction is collected from a cation exchange resin and includes about 0.1-0.4% high molecular weight aggregates.

In some embodiments, the eluate fraction is collected from anion exchange chromatography resin and includes about 0.01-10%, about 0.01-5%, about 0.0-1%, about 0.04-0.2%, about 0.04-0.8%, about 0.1-0.4%, about 0.04-0.8%, about 0.5-0.8%, about 1-10%, about 2-10%, about 3-10%, or about 4-10% high molecular weight aggregates.

In some embodiments, the eluate fraction is collected from an anion exchange resin and includes about 0.04-0.2% high molecular weight aggregates.

In some embodiments, the level of high molecular weight aggregates in the eluate fraction is reduced by at least about 90%, about 80%, about 70%, about 60%, about 50%, about 40%, about 30%, about 20% or about 10% as compared to the level of high molecular weight aggregates in the sample.

In some embodiments, the eluate fraction includes less than about 10, about 9, about 8, about 7, about 6, about 5, about 4, about 3, about 2, about 1, about 0.9, about 0.8, about 0.7, about 0.6 or about 0.5 ppm HCP.

In some embodiments, the eluate fraction includes about 0.1-10, about 1-10, about 2-10, about 3-10, about 4-10, about 1-5, about 5-10, about 0.1-2, about 0.1-3, about 2-8 ppm, or about 0.1-8 ppm HCP. In some embodiments, the eluate fraction includes about 0.1-8 ppm HCP.

In some embodiments, the eluate fraction is collected from anion exchange chromatography resin and includes about 0.1-2 ppm HCP.

In some embodiments, the eluate fraction is collected from cation exchange chromatography resin and includes about 2-8 ppm HCP.

In some embodiments, the level of HCP in the eluate fraction is reduced by at least about 90%, about 80%, about 70%, about 60%, about 50%, about 40%, about 30%, about 20% or about 10% as compared to the level of HCP in the sample.

In some embodiments, the eluate fraction includes more than about 90%, about 91%, about 92%, about 93%, about 94%, about 95%, about 96%, about 97%, about 98%, about 99%, about 99.1% or about 99.5% of monomer of the protein of interest.

In some embodiments, the eluate fraction includes about 90-99.9%, about 90-95%, about 94-99.9%, about 95-99.9%, about 99-99.9%, about 99.1-99.9%, about 98-99%, about 98-99.9%, or about 98.5-99.5% of monomer of the protein of interest.

In some embodiments, the eluate fraction includes about 98-99.9% of monomer of the protein of interest. In some embodiments, the eluate fraction includes about 98.5-99.5% of monomer of the protein of interest.

In some embodiments, the eluate fraction includes less than about 10%, about 9%, about 8%, about 7%, about 6%, about 5%, about 4%, about 3%, about 2%, about 1%, about 0.5%, about 0.4%, about 0.3%, about 0.2% or about 0.1% fragments of protein of interest.

In some embodiments, the eluate fraction includes about 0.1-10%, about 0.1-5%, about 0.1-3%, about 0.1-2%, about 0.1-1.1%, about 0.1-0.8%, about 0.5-1.5%, about 0.6-1.5%, about 0.5-1.1%, about 0.4-0.8% or about 0.4-1.1% fragments of protein of interest.

In some embodiments, the eluate fraction includes about 0.1-1.1% of fragments of protein of interest. In some embodiments, the eluate fraction includes about 0.1-0.8% of fragments of protein of interest. In some embodiments, the eluate fraction includes about 0.4-1.1% of fragments of protein of interest.

In some embodiments, the eluate fraction is collected from cation exchange chromatography and includes about 0.1-0.8% or about 0.4-0.8% of fragments of protein of interest. In some embodiments, the eluate fraction is collected from anion exchange chromatography resin and includes about 0.1-1.1% or about 0.5-1.1% of fragments of protein of interest.

In some embodiments, the level of high molecular weight aggregates, the level of fragments of protein of interest or the level of monomer of protein of interest are determined by size exclusion chromatography.

In some embodiments, the level of HCP is determined by ELISA.

In one aspect, the present invention provides a composition including an anti-GM-CSFRα antibody, or antigen-binding portion thereof, wherein the composition includes less than about 10%, about 9%, about 8%, about 7%, about 6%, about 5%, about 4%, about 3%, about 1%, about 0.9%, about 0.8%, about 0.7%, about 0.6%, about 0.5%, about 0.4%, about 0.3%, about 0.2%, or about 0.1% high molecular weight aggregates.

In some embodiments, the composition includes less than about 0.5% high molecular weight aggregates.

In another aspect, the present invention provides a composition including an anti-GM-CSFRα antibody, or antigen-binding portion thereof, wherein the composition includes about 0.01-10%, about 0.01-5%, about 0.01-1%, about 0.04-0.2%, about 0.1-0.4%, about 0.04-0.4%, about 0.04-0.8%, about 0.5-0.8%, about 1-10%, about 2-10%, about 3-10%, or about 4-10% high molecular weight aggregates.

In some embodiments, the composition includes about 0.04-0.8% of high molecular weight aggregates.

In one aspect, the present invention provides a composition including an anti-GM-CSFRα antibody, or antigen-binding portion thereof, wherein the composition includes less than about 10, about 9, about 8, about 7, about 6, about 5, about 4, about 3, about 2, about 1, about 0.9, about 0.8, about 0.7, about 0.6 or about 0.5 ppm host cell proteins.

In another aspect, the present invention provides a composition including an anti-GM-CSFRα antibody, or antigen-binding portion thereof, wherein the composition includes about 0.1-10, about 1-10, about 2-10, about 3-10, about 4-10, about 1-5, about 5-10, about 0.1-2, about 0.1-3, about 2-8 ppm or about 0.1-8 ppm HCP. In some embodiments, the composition includes about 0.1-2 ppm HCP.

In one aspect, the present invention provides a composition including an anti-GM-CSFRα antibody, wherein the composition includes more than about 90%, about 91%, about 92%, about 93%, about 94%, about 95%, about 96%, about 97%, about 98%, about 99%, about 99.1% or about 99.5% of the antibody monomer.

In some embodiments, the composition includes more than 99.1% of the antibody monomer.

In another aspect, the present invention provides a composition including an anti-GM-CSFRα antibody, wherein the composition includes about 90-99.9%, about 90-95%, about 95-99.9%, about 99-99.9%, about 99.1-99.9%, about 98-99%, about 98-99.9%, or about 98.5-99.5% of the antibody monomer.

In some embodiments, the composition includes about 98-99% of the antibody monomer. In some embodiments, the composition includes about 98-99.9% of the antibody monomer.

In one aspect, the present invention provides a composition including an anti-GM-CSFRα antibody, wherein the composition includes less than about 10%, about 9%, about 8%, about 7%, about 6%, about 5%, about 4%, about 3%, about 2%, about 1%, about 0.5%, about 0.4%, about 0.3%, about 0.2% or about 0.1% antibody fragments.

In some embodiments, the composition includes less than about 0.4% of antibody fragments.

In another aspect, the present invention provides a composition including an anti-GM-CSFRα antibody, wherein the composition includes about 0.1-10%, about 0.1-5%, about 0.1-3%, about 0.1-2%, about 0.6-1.5%, about 0.5-1.5%, about 0.5-1.1%, about 0.4-0.8% or about 0.4-1.1% antibody fragments.

In some embodiments, the composition includes about 0.5-1.5% of antibody fragments. In some embodiments, the composition includes about 0.6-1.5% of antibody fragments.

In one aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes less than about 10%, about 9%, about 8%, about 7%, about 6%, about 5%, about 4%, about 3%, about 1%, about 0.9%, about 0.8%, about 0.7%, about 0.6%, about 0.5%, about 0.4%, about 0.3%, about 0.2%, or about 0.1% high molecular weight aggregates.

In another aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes about 0.1-10%, about 0.01-5%, about 0.01-1%, about 0.04-0.8%, about 0.04-0.2%, about 0.1-0.4%, about 0.5-0.8%, about 0.04-0.4%, about 0.1-6%, about 1-10%, about 2-10%, about 3-10%, or about 4-10% high molecular weight aggregates.

In some embodiments, the eluate fraction includes about 0.04-0.4% of high molecular weight aggregates. In some embodiments, the eluate fraction is collected from an anion exchange chromatography resin and includes about 0.04-0.2% of high molecular weight aggregates. In some embodiments, the eluate fraction is collected from cation exchange chromatography resin and includes about 0.1-0.4% of high molecular weight aggregates.

In one aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes less than about 10, about 9, about 8, about 7, about 6, about 5, about 4, about 3, about 2, about 1, about 0.9, about 0.8, about 0.7, about 0.6 or about 0.5 ppm host cell proteins.

In another aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes about 0.1-10, about 1-10, about 2-10, about 3-10, about 4-10, about 1-5, about 5-10, about 0.1-2, about 0.1-3, about 2-8 ppm or about 0.1-8 ppm HCP.

In some embodiments, the eluate fraction includes about 0.1-8 ppm HCP.

In some embodiments, the eluate fraction is collected from anion exchange chromatography resin and includes about 0.1-2 ppm HCP. In some embodiments, the eluate fraction is collected from cation exchange chromatography resin and includes about 2-8 ppm HCP.

In one aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes more than about 90%, about 91%, about 92%, about 93%, about 94%, about 95%, about 96%, about 97%, about 98%, about 99%, about 99.1% or about 99.5% of the antibody monomer.

In another aspect, the present invention provides a composition including an anti-GM-CSFRα antibody, or antigen-binding portion thereof, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes about 90-99.9%, about 90-95%, about 94-99.9%, about 95-99.9%, about 99-99.9%, about 99.1-99.9%, about 98-99%, about 98-99.9%, or about 98.5-99.5%, of the antibody monomer.

In some embodiments, the eluate fraction includes about 98-99.9% of the antibody monomer. In some embodiments, the eluate fraction includes about 98.5-99.5% of the antibody.

In one aspect, the present invention provides a composition including an anti-GM-CSFRα antibody, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes less than 10%, about 9%, about 8%, about 7%, about 6%, about 5%, about 4%, about 3%, about 2%, about 1%, about 0.5%, about 0.4%, about 0.3%, about 0.2% or about 0.1% antibody fragments.

In another aspect, the present invention provides a composition including an anti-GM-CSFRα antibody, wherein the composition includes an eluate fraction collected from a chromatography resin, wherein the chromatography resin is selected from a group consisting of a cation exchange chromatography resin, an anion exchange chromatography resin and a mixed mode chromatography resin, and wherein the eluate fraction includes about 0.1-10%, about 0.1-5%, about 0.1-3%, about 0.1-2%, about 0.1-1.1%, about 0.1-0.8%, about 0.6-1.5%, about 0.5-1.5%, about 0.5-1.1%, about 0.4-0.8% or about 0.4-1.1% antibody fragments.

In some embodiments, the eluate fraction includes about 0.4-1.1% antibody fragments. In some embodiments, the eluate fraction is collected from anion exchange chromatography resin and includes about 0.1-1.1% or about 0.5-1.1% antibody fragments.

In some embodiments, the eluate fraction is collected from cation exchange chromatography resin and includes about 0.1-0.8% or about 0.4-0.8% antibody fragments.

In some embodiments, the level of high molecular weight aggregates, the level of antibody monomer and/or the level of antibody fragments is determined by size exclusion chromatography.

In some embodiments, the level of HCP is determined by ELISA.

In some embodiments, the anti-GM-CSFRα antibody or antigen-binding portion thereof is mavrilimumab.

In one aspect, the present invention provides a method for preparing a preparation including a protein of interest having a reduced level of acidic species from a cell culture, the method including incubating the cell culture in a bioreactor; maintaining the pH of the cell culture at a pH of about 6-7.5; thereby preparing a preparation including the protein of interest with a reduced level of acidic species.

In another aspect, the present invention provides a method for reducing the level of acidic species of a protein of interest in a cell culture, the method including incubating the cell culture in a bioreactor; maintaining the pH of the cell culture at a pH of about 6-7.5; thereby reducing the level of acidic species of the protein of interest.

In one aspect, the present invention provides a method for increasing production yield of a protein of interest from a cell culture, the method including incubating the cell culture in a bioreactor; maintaining the pH of the cell culture at a pH of about 6-7.5; thereby increasing the production yield of the protein of interest.

In some embodiments, the protein of interest is an antibody or antigen-binding portion thereof. In some embodiments, the antibody or antigen-binding portion thereof is an anti-GM-CSFRα antibody or antigen-binding portion thereof. In some embodiments, the anti-GM-CSFRα antibody or antigen-binding portion thereof is mavrilimumab.

In some embodiments, the pH of the cell culture is maintained at a pH of about 6-7.5, about 6.5-7.5, about 6-7, about 6.5-7, or about 6.7-7. In some embodiments, the pH of the cell culture is maintained at a pH of about 6.5-7.

In some embodiments, the pH of the cell culture is decreased by about 0.01-0.4, about 0.02-0.4, about 0.05-0.3, about 0.1-0.4, about 0.1-0.3, about 0.1-0.2, or about 0.1-0.2 during Day 2-Day 8 of the incubation period, but maintains at a pH between about 6.5-7.

In some embodiments, the pH of the cell culture is maintained by one or more steps selected from the group consisting of (a) increasing the level of CO₂ in the cell culture; (b) maintaining the level of lactate in the cell culture at about 0.1-5 g/L; (c) increasing lactate production in the cell culture; and (d) increasing the level of cell culture supplement during the incubation period.

In some embodiments, the level of CO₂ in the cell culture is increased by at least about 0.1%, about 0.5%, about 1%, about 2%, about 3%, about 4%, about 5%, about 6%, about 7%, about 8%, about 9% or about 10%. In some embodiments, the level of CO₂ in the cell culture is increased by about 0.1-5%, about 0.2-6%, about 0.3-7%, about 0.4-8%, or about 0.5-10%.

In some embodiments, the level of lactate in the cell culture is maintained at about 0.1-5 g/L, 0.1-4 g/L, 0.1-3 g/L, 0.1-2 g/L, 0.2-2 g/L, about 0.3-2 g/L, about 0.4-2 g/L, about 0.5-2 g/L, about 0.6-2 g/L, about 0.7-2 g/L, about 0.8-2 g/L, about 0.9-2 g/L, about 0.1-1.9, about 0.2-1.8, about 0.3-1.7, about 0.4-1.6, about 0.5-1.5 g/L, about 0.6-1.4, about 0.7-1.3, about 0.8-1.2, or about 0.9-1.1.

In some embodiments, the cell culture supplement includes one or more supplements.

In some embodiments, the level of cell culture supplement is increased by about 0.1%-20%, about 0.1%-10%, about 0.1%-5%, about 0.5%-20%, about 0.5%-10%, or about 1%-10% during the incubation period.

In some embodiments, the cell culture supplement at a level of about 0.1-3% is added to the cell culture during Day 2-Day 8 of the incubation period, and the level of the cell culture supplement is increased by about 50% or greater of the initial level during Day 4-Day 10 of the incubation period.

In some embodiments, increasing the cell culture supplement results in an increase in lactate production, an increase in osmolality, an increase in cell viability, and/or a decrease in pH of the cell culture.

In some embodiments, the cell culture is maintained at a temperature of about 35-37° C.

In one aspect, the present invention provides a method for preparing a preparation including a protein of interest having a reduced level of acidic species from a cell culture, the method including incubating the cell culture in a bioreactor; and one of more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 0.1-5 g/L; (d) increasing lactate production in the cell culture; (e) increasing the level of CO₂ in the cell culture; and/or (f) decreasing the pH of the cell culture, thereby preparing a preparation including the protein of interest with a reduced level of acidic species.

In another aspect, the present invention provides a method for reducing the level of acidic species of a protein of interest in a cell culture, the method including incubating the cell culture in a bioreactor; and one or more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 0.1-5 g/L; (d) increasing lactate production in the cell culture; (e) increasing the level of CO₂ in the cell culture; and/or (f) decreasing the pH of the cell culture, thereby reducing the level of acidic species of the protein of interest.

In another aspect, the present invention provides a method for increasing production yield of a protein of interest from a cell culture, the method including incubating the cell culture in a bioreactor; and one or more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 0.1-5 g/L; (d) increasing lactate production in the cell culture; (e) increasing the level of CO₂ in the cell culture; and/or (f) decreasing the pH of the cell culture, thereby increasing the production yield of the protein of interest.

In some embodiments, the protein of interest is an antibody or antigen-binding portion thereof. In some embodiments, the antibody or antigen-binding portion thereof is an anti-GM-CSFRα antibody or antigen-binding portion thereof. In some embodiments, the anti-GM-CSFRα antibody or antigen-binding portion thereof is mavrilimumab.

### BRIEF DESCRIPTION OF THE DRAWING

FIG. 1 depicts an overview of an exemplary purification process.

### DETAILED DESCRIPTION OF THE INVENTION

The present invention is based on the identification and optimization of upstream and downstream process technologies for protein production, e.g., production of antibodies or antigen-binding portions thereof, for example, an anti-GM-CSFRα antibody such as mavrilimumab, resulting in the production of compositions comprising proteins having low levels of variants and/or impurities, e.g., low levels of product-related substances, e.g., product aggregates, fragments or charged species, e.g., acidic or basic species, and/or low levels of process-related impurities, e.g., host cell proteins.

By modulating conditions during upstream protein production, such as the process parameters of cell culture, e.g., the pH, CO₂ or lactate levels of the cell culture or the level of cell culture feeds and/or supplements, and/or by optimizing the downstream purification process, e.g., the chromatography steps, the inventors of the present invention have successfully generated compositions comprising a protein of interest, e.g., an antibody or antigen binding portion thereof, e.g., an anti-GM-CSFRα antibody such as mavrilimumab or antigen binding portion thereof, having a reduced level of variants and/or impurities, such as a reduced level of acidic species or basic species, a reduced level of aggregates or fragments, a reduced level of half antibody and/or a reduced level of host cell proteins. Compositions with such a low level of variants and/or impurities are highly desirable since the resulting protein product would provide therapeutic benefits with higher potency, higher efficacy, or better stability without undesired effect. For example, compositions with lower aggregates, half antibodies or fragments, and/or higher levels of antibody monomer, will exhibit higher potency and efficacy, at least in part, by maximizing the level of active antibody present in the composition.

The following description is presented to enable a person of ordinary skill in the art to make and use the various embodiments. Descriptions of specific methods, compositions, techniques, and applications are provided only as examples. Various modifications to the examples described herein will be readily apparent to those of ordinary skill in the art, and the general principles described herein may be applied to other examples and applications without departing from the spirit and scope of the various embodiments. Thus, the various embodiments are not intended to be limited to the examples described herein and shown, but are to be accorded the scope consistent with the claims

Unless defined otherwise, all technical and scientific terms used herein have the same meaning as is commonly understood by one of ordinary skill in the art to which this application belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference. The headings provided herein are for convenience only and do not limit the application in any way. All patents, applications, published applications and other publications referred to herein are incorporated by reference in their entirety.

### I. Definition

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means+±10%. In certain embodiments, about means±5%. When about is present before a series of numbers or a range, it is understood that “about” can modify each of the numbers in the series or range.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein that includes modifications, such as deletions, additions, and substitutions (generally conservative in nature), to a native sequence, as long as the protein maintains the desired activity. These modifications may be deliberate, as through site-directed mutagenesis, or may be accidental, such as through mutations of hosts that produce the proteins or errors due to PCR amplification.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term antibody also includes chimeric antibodies, humanized antibodies, and antibodies of various species such as mouse, human, cynomolgus monkey, llama, camel, etc. The term also includes multivalent antibodies such as bivalent or tetravalent antibodies. A multivalent antibody includes, e.g., a single polypeptide chain comprising multiple antigen binding (CDR-containing) domains, as well as two or more polypeptide chains, each containing one or more antigen binding domains, such two or more polypeptide chains being associated with one another, e.g., through a hinge region capable of forming disulfide bond(s) or any other covalent or noncovalent interaction.

The term “antigen-binding portion” of an antibody (or “antibody portion”) includes fragments of an antibody, e.g., one or more antigen-binding domains, that retain the ability to specifically bind to an antigen (e.g., in the case of mavrilimumab, granulocyte/macrophage colony stimulating factor receptor alpha subunit (GM-CSFRα)). It has been shown that the antigen-binding function of an antibody can be performed by fragments of a full-length antibody. Examples of binding fragments encompassed within the term “antigen-binding portion” of an antibody include molecules comprising at least CDR1, CDR2, and CDR3 of a single domain antibody (sdAb), wherein the molecule is capable of binding to an antigen. The term antibody-binding portion also refers to molecules comprising at least CDR1, CDR2, and CDR3 of a heavy chain and CDR1, CDR2, and CDR3 of a light chain, wherein the molecule is capable of binding to an antigen. The term antibody-binding portion also includes fragments that are capable of binding an antigen, such as (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fab′ fragment, which can be formed by the reduction of F(ab′)2 fragment; (iv) a Fc fragment comprising the CH2 and CH3 region and part of the hinge region held together by one or more disulfides and noncovalent interactions; (v) a Fd fragment comprising the VH and CH1 domains; (vi) a Fv fragment comprising the VL and VH domains of a single arm of an antibody, (vii) a reduced IgG or half IgG; and (viii) a dAb fragment (Ward et al., (1989) Nature 341:544-546, the entire teaching of which is incorporated herein by reference), which comprises a VH domain. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The term “half antibody” or “reduced IgG”, as used herein, refers to one immunoglobulin heavy chain which is associated to one immunoglobulin light chain with a molecular weight of 75 kDa. It is the product of selectively reducing just the hinge-region disulfide bonds within an antibody molecule.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody”, as used herein, refers to an antibody that is substantially free of other antibodies having different antigenic specificities (e.g., an isolated antibody that specifically binds GM-CSFRα is substantially free of antibodies that specifically bind antigens other than GM-CSFRα).

An isolated antibody that specifically binds GM-CSFRα may, however, have cross-reactivity to other antigens, such as GM-CSFRα molecules from other species. Moreover, an isolated antibody may be substantially free of other cellular material and/or chemicals. A suitable anti-GM-CSFRα antibody is mavrilimumab.

The terms “Kabat numbering” “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “product”, as used herein refers to a protein of interest, which may be present in the context of a sample comprising one or more variants and/or impurities, e.g., product-related substances, e.g., product aggregates, fragments or charged species, e.g., acidic or basic species, and/or process-related impurities, e.g., host cell proteins. In certain embodiments, the product, i.e., the protein of interest, is an antibody or antigen binding fragment thereof.

The terms “product-related substances” or “product-related variants” refer to any variants of the product, for example, charged species, aggregates, half-antibodies, fragments or any other protein product species derived from alternative post-translational modifications. Removal of product-related substances, e.g. protein aggregates, fragments or charged species, e.g., acidic species or basic species, from the resulting protein product, e.g., an antibody or antigen-binding portion thereof, are desirable such that the resulting protein product would provide therapeutic benefits with higher potency, higher efficacy, or better stability without undesired effect.

The term “fragments” as used herein refers to any truncated protein species from the protein of interest due to disruption of one or more bonds along the peptide backbone of a protein of interest, or dissociation of enzymatic and/or chemical modifications. For instance, antibody fragments include, but not limited to, Fab, F(ab′)2, Fab′, Fc, Fv, scFv, Fd, dAb, half antibody, or other compositions that contain a portion of the antibody molecule.

The terms “aggregates” or “high molecular weight aggregates” or “high molecular weight impurities”, as used herein, refer to the oligomerization of two or more individual molecules of protein of interest, including but not limiting to, protein dimers, trimers, tetramers, oligomers and other high molecular weight species.

The terms “charge variants” or “charged species”, as used herein, refer to the full complement of product with different charges. In certain embodiments, such variants can include product aggregates and/or product fragments, to the extent that such aggregation and/or fragmentation results in a product with charge variations as seen in an analytical technique used for that purpose. In certain embodiments, such variants refer to products with different modifications that give rise to charge heterogeneity. In monoclonal antibody preparations, charged variants, e.g., acidic species, or basic species, can be detected by charged based separation techniques such as isoelectric focusing (IEF) gel electrophoresis, capillary isoelectric focusing (cIEF) gel electrophoresis, cation exchange chromatography (CEX) and anion exchange chromatography (AEX).

As used herein, the term “acidic species” refers to the variants of a protein, e.g., an antibody or antigen-binding portion thereof, which are characterized by an overall acidic charge. Acidic species are variants with lower apparent pI when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX.

Acidic species of an antibody may include charge variants, structure variants, and/or fragmentation variants. Exemplary charge variants include, but are not limited to, deamidation variants, afucosylation variants, methylglyoxal variants, glycation variants, and citric acid variants. Exemplary structure variants include, but are not limited to, glycosylation variants and acetonation variants. Exemplary fragmentation variants include any truncated protein species from the protein of interest due to dissociation of peptide chain, enzymatic and/or chemical modifications, including, but not limited to, Fc and Fab fragments, fragments missing a Fab, fragments missing a heavy chain variable domain, C-terminal truncation variants, variants with excision of N-terminal Asp in the light chain, and variants having N-terminal truncation of the light chain. Other acidic species variants also include variants containing unpaired disulfides, host cell proteins, and host nucleic acids, chromatographic materials, and media components.

The acidic species may be the result of product preparation (referred to herein as “preparation-derived acidic species”), or the result of storage (referred to herein as “storage-derived acidic species”). Preparation-derived acidic species are acidic species that are formed during the preparation (upstream and/or downstream processing) of the protein, e.g., the antibody or antigen-binding portion thereof. For example, preparation-derived acidic species can be formed during cell culture (“cell culture-derived acidic species”). Storage-derived acidic species are acidic species that may or may not be present in the population of proteins directly after preparation, but are formed or generated while the sample is being stored. The type and amount of storage-derived acidic species can vary based on the formulation of the sample. Formation of storage-derived acidic species can be partially or completely inhibited when the preparation is stored under particular conditions. For example, an aqueous formulation can be stored at a particular temperature to partially or completely inhibit acidic species formation. For example, formation or storage-derived acidic species can be partially inhibited in an aqueous formulation stored at between about 2° C. and 8° C., and completely inhibited when stored at −80° C. In addition, a low acidic species composition can be lyophilized or freeze-dried to partially or completely inhibit the formation of storage-derived acidic species.

The term “basic species”, as used herein, refers to the variants of a protein, e.g., an antibody or antigen-binding portion thereof, which are characterized by an overall basic charge. Basic species are variants with higher apparent pI when antibodies are analyzed using IEF based methods. When analyzed by chromatography-based methods, basic species are the variants that elute later than the main peak from CEX or earlier than the main peak from AEX.

Basic species of an antibody may include charge variants, structure variants, and/or fragmentation variants. Exemplary modifications that result in generation of basic species include, but are not limited to, C-terminal lysine, N-terminal glutamine, isomerization of aspartate, succinimide, methionine oxidation, amidation, incomplete disulfide bonds, incomplete removal of leader sequence, mutation from serine to arginine, aglycosylation, fragments or aggregates. In some embodiments, the basic species refers to an antibody or antigen binding portion thereof comprising a heavy chain having one or two C-terminal lysines.

The term “main species” as used herein, refers to the form of a protein, e.g., an antibody or antigen binding portion thereof, that elutes as the major peak on chromatograms, i.e., the majority species detected during fractionation of charged variants of a protein. For example, in particular embodiments, the “main species” refers to an anti-GM-CSFRα antibody. In a particular embodiment, the main species refers to mavrilimumab.

The term “process-related impurity,” as used herein, refers to impurities that are present in a composition comprising a protein but are not derived from the protein itself. Process-related impurities include, but are not limited to, host cell proteins (HCPs), host cell nucleic acids, e.g., DNA or RNA, chromatographic materials, and media components. Removal of process-related impurities, such host cell proteins, from the resulting protein product, e.g., an antibody or antigen-binding portion thereof, are desirable such that the resulting protein product would provide therapeutic benefits with higher potency, higher efficacy, or better stability without undesired effect.

The term “host cell proteins” (HCPs), as used herein, is intended to refer to non-target protein-related, proteinaceous impurities derived from host cells.

As used herein, the terms “granulocyte/macrophage colony stimulating factor receptor alpha subunit (GM-CSFRα)” or “GM-CSFRα” or “GM-CSFR alpha” refer to the alpha chain of the receptor for granulocyte macrophage colony stimulating factor (GM-CSF). GM-CSFRα is also known as colony stimulating factor 2 receptor subunit alpha; GMR; CD116; CSF2R; SMDP4; CDw116; CSF2RX; CSF2RY; GMCSFR; CSF2RAX; CSF2RAY; alphaGMR; GMR-alpha; GMCSFR-alpha; and GM-CSF-R-alpha.

GM-CSFR is a member of a highly conserved cytokine receptor super family GM-CSFR comprises two subunits which result in different affinities for GM-CSF observed on some hematopoietic cells. The first subunit is commonly referred to as the alpha subunit, and is an 85 Kd protein which can bind GM-CSF by itself with low affinity. Multiple other isoforms of the GM-CSFRα chain, some membrane-bound and some soluble, have been described, however the al isoform appears to be the predominant form expressed on the cell surface of neutrophils and macrophages (Crosier et al., Br J Haematol. 98:540-548 (1997)). The extracellular portion of GM-CSFRα is highly glycosylated. The receptor has a second subunit, the f3 chain, which does not bind to GM-CSF by itself. Rather, it binds GM-CSF when associated with the alpha chain. GM-CSF normally binds to the extracellular domain of the mature GM-CSF receptor alpha chain. This binding can be inhibited by anti-GM-CSFRα antibodies, e.g., mavrilimumab.

The term “GM-CSFRα” includes human GM-CSFRα, the amino acid sequence of which may be found in for example, GenBank Accession No. NP_006131.2 (SEQ ID NO:1). The term “GM-CSFRα” also includes cynomolgus GM-CSFRα, mouse GM-CSFRα, and rat GM-CSFRα. The term “GM-CSFRα” includes a wild type, a variant or an isoform of GM-CSFRα protein or a fragment or domain thereof. In certain embodiments, The GM-CSFRα protein may be coupled to a signal peptide sequence, and/or a protein tag.

As used herein, the term “mavrilimumab” refers to a human IgG4 monoclonal antibody designed to modulate macrophage activation, differentiation, and survival by targeting GM-CSFRα (see PCT Publication No. WO2007/110631, the entire contents of which, including the sequences described therein, are incorporated herein by reference). Mavrilimumab comprises a heavy chain comprising the sequence set forth as SEQ ID NO:2, and a light chain comprising the sequence set forth as SEQ ID NO:3. The heavy chain variable region of mavrilimumab comprises the sequence set forth as SEQ ID NO:4, and the light chain variable region of mavrilimumab comprises the sequence set forth as SEQ ID NO:5. The heavy chain variable region of mavrilimumab comprises a CDR1 having the sequence set forth as SEQ ID NO:6, a CDR2 having the sequence set forth as SEQ ID NO:7, and a CDR3 having the sequence set forth as SEQ ID NO:8. The light chain variable region of mavrilimumab comprises a CDR1 having the sequence set forth as SEQ ID NO:9, a CDR2 having the sequence set forth as SEQ ID NO:10 and a CDR3 having the sequence set forth as SEQ ID NO:11.

As used herein, the term “GM-CSFRα-associated disease or disorder” is intended to include diseases and other disorders in which the presence of GM-CSFRα in a subject suffering from the disorder has been shown to be or is suspected of being either responsible for the pathophysiology of the disorder or a factor that contributes to a worsening of the disorder. Accordingly, a GM-CSFRα-associated disorder is a disorder in which inhibition of GM-CSFRα activity is expected to alleviate the symptoms and/or progression of the disorder. Since GM-CSF binds specifically to GM-CSFRα, pathological and/or symptomatic effects of GM-CSF can also be countered by inhibiting binding of GM-CSF to GM-CSFRα and, thereof, any disease or disorder associated with GM-CSF is also encompassed within the definition of “a GM-CSFRα-associated disorder”, as used herein. Thereof, “a GM-CSFRα-associated disorder” may be evidenced, for example, by an increase in the concentration of GM-CSFRα and/or GM-CSF in a biological fluid of a subject suffering from the disorder (e.g., an increase in the concentration of GM-CSFRα and/or GM-CSF in serum, plasma, synovial fluid, etc. of the subject), which can be detected, for example, using an anti-GM-CSFRα antibody or an anti-GM-CSF antibody.

There are numerous examples of GM-CSFRα-associated diseases or disorders. In one embodiment, the GM-CSFRα-associated disease or disorder is an autoimmune disorder. In one embodiment, the autoimmune disorder is selected from the group consisting of rheumatoid arthritis, juvenile idiopathic arthritis, rheumatoid spondylitis, ankylosing spondylitis, psoriasis, osteoarthritis, gouty arthritis, an allergy, multiple sclerosis, psoriatic arthritis, autoimmune diabetes, autoimmune uveitis, nephrotic syndrome, juvenile rheumatoid arthritis, Crohn's disease, ulcerative colitis, active axial spondyloarthritis (active axSpA) and non-radiographic axial spondyloarthritis (nr-axSpA). In a particular embodiment, the GM-CSFRα-associated disease or disorder is rheumatoid arthritis. In another embodiment, the GM-CSFRα-associated disease or disorder is giant cell arteritis (GCA). In another embodiment, the GM-CSFRα-associated disease or disorder is coronavirus disease 2019 (COVID-19). The use of GM-CSFRα antibodies and antibody portions obtained using methods of the invention for the treatment of specific disorders is discussed in further detail below.

As used herein, the term “upstream process technology,” in the context of protein, e.g., antibody, preparation, refers to activities involving the production and collection of proteins (e.g. antibodies) from cells (e.g., during production of protein of interest from cell cultivation). As used herein, the term “cell culture” refers to methods for generating and maintaining a population of host cells capable of producing a recombinant protein of interest, as well as the methods and techniques for optimizing the production and collection of the protein of interest. For example, once an expression vector has been incorporated into an appropriate host, the host can be maintained under conditions suitable for expression of the relevant nucleotide coding sequences, and the collection and purification of the desired recombinant protein.

When using the cell culture techniques of the instant invention, the protein of interest can be produced intracellularly, in the periplasmic space, or directly secreted into the medium. In embodiments where the protein of interest is produced intracellularly, the particulate debris, either host cells or lysed cells (e.g., resulting from homogenization) can be removed by a variety of means, including but not limited to, centrifugation or ultrafiltration. Where the protein of interest is secreted into the medium, supernatants from such expression systems can be first concentrated using a commercially available protein concentration filter.

As used herein, the term “downstream process technology” refers to one or more techniques used after the upstream process technologies to purify the protein of interest, e.g., antibody. For example, downstream process technology includes purification of the protein product using, for example, affinity chromatography, including Protein A affinity chromatography, ion exchange chromatography, such as anion or cation exchange chromatography, hydrophobic interaction chromatography, mixed-mode or multi-modal chromatography or displacement chromatography.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “recombinant protein” refers to a protein produced as the result of the transcription and translation of a gene carried on a recombinant expression vector that has been introduced into a host cell. In certain embodiments, the recombinant protein is an antibody, e.g., a chimeric, humanized, or fully human antibody. In certain embodiments the recombinant protein is an antibody of an isotype selected from group consisting of: IgG (e.g., IgG1, IgG2, IgG3, IgG4), IgM, IgA1, IgA2, IgD, or IgE. In certain embodiments the antibody molecule is a full-length antibody (e.g., an IgG1 or IgG4 immunoglobulin) or alternatively the antibody can be a fragment (e.g., an Fc fragment or a Fab fragment).

### II. Compositions of the Invention

The present invention provides compositions comprising a protein of interest, e.g., an antibody or antigen-binding portion thereof, for example, an anti-GM-CSFRα antibody such as mavrilimumab. The composition comprises a protein of interest having a reduced level of variants and/or impurities, e.g., product-related substances, e.g., protein aggregates, half antibodies, fragments or charged species, e.g., acidic species or basic species, and/or process-related impurities, e.g., host cell proteins.

In some embodiments, the composition comprises a protein of interest, e.g., an antibody or antigen-binding portion thereof, for example, an anti-GM-CSFRα antibody such as mavrilimumab, having a reduced level of half antibody, wherein the composition comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the composition comprises less than about 20% half antibody. In some embodiments, the composition comprises less than about 18%. In some embodiments, the composition comprises less than about 2.8% half antibody. In some embodiments, the composition comprises less than about 1.7%.

In some embodiments, the composition comprises about 0.1-25%, 0.1-20%, about 0.1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one or more of the preceding. In some embodiments, the composition comprises about 0.6-1.7% half antibody.

In some embodiments, the composition comprising the protein of interest, e.g., an antibody or antigen-binding portion thereof, for example, an anti-GM-CSFRα antibody such as mavrilimumab, comprises less than about 2.8% half antibody and at least one of a) about 11-38% acidic species, b) about 9-41% basic species, c) about 46-67% main species, d) about 0.04-0.8% aggregates of protein of interest, e) about 98-99.9% monomer of protein of interest, f) about 0.4-1.5% fragments of protein of interest, or g) about 0.1-8 ppm host cell protein.

In some embodiments, the composition comprises a protein of interest, e.g., an antibody or antigen-binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises less than about 40% acidic species. In some embodiments, the composition comprises less than about 20% acidic species.

In some embodiments, the composition comprising the protein of interest, e.g., an antibody or antigen-binding portion thereof, for example, an anti-GM-CSFRα antibody such as mavrilimumab, comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding. In some embodiments, the composition comprises about 12-20% acidic species. In some embodiments, the composition comprises about 11-22% acidic species. In some embodiments, the composition comprises about 18-40% acidic species. In other embodiments, the composition about 9-18%, 10-17%, or 11-16% acidic species.

In some embodiments, the composition comprising the protein of interest, e.g., an antibody or antigen-binding portion thereof, for example, an anti-GM-CSFRα antibody such as mavrilimumab, comprises about 9-18% acidic species, and at least one of a) about 0.6-18% half antibody, b) about 9-41% basic species, c) about 46-67% main species, d) about 0.04-0.8% aggregates of protein of interest, e) about 98-99.9% monomer of protein of interest, f) about 0.4-1.5% fragments of protein of interest, or g) about 0.1-8 ppm host cell protein.

In particular embodiments, the composition comprising the protein of interest, e.g., an antibody or antigen-binding portion thereof, for example, an anti-GM-CSFRα antibody such as mavrilimumab, comprises about 9-18% acidic species and about 9-41% basic species. In another particular embodiment, the composition comprising the protein of interest, e.g., an antibody or antigen-binding portion thereof, for example, an anti-GM-CSFRα antibody such as mavrilimumab, comprises about 9-18% acidic species and about 46-67% main species.

In some embodiments, the composition comprising the protein of interest, e.g., an antibody or antigen-binding portion thereof, for example, an anti-GM-CSFRα antibody such as mavrilimumab, comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% basic species, and ranges within one or more of the preceding. In some embodiments, the composition comprises less than 45% basic species. In some embodiments, the composition comprises less than 24% basic species. In some embodiments, the composition comprises less than 23% basic species. In some embodiments, the composition comprises less than 20% basic species.

In some embodiments, the composition comprising the protein of interest, e.g., an antibody or antigen-binding portion thereof, for example, an anti-GM-CSFRα antibody such as mavrilimumab, comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding. In some embodiments, the composition comprises about 17-26% basic species. In some embodiments, the composition comprises about 16-41% basic species.

In some embodiments, the composition comprising the protein of interest, e.g., an antibody or antigen-binding portion thereof, for example, an anti-GM-CSFRα antibody such as mavrilimumab, comprises less than about 24% basic species, and at least one of a) about 0.6-18% half antibody, b) about 11-38% acidic species, c) about 46-67% main species, d) about 0.04-0.8% aggregates of protein of interest, e) about 98-99.9% monomer of protein of interest, f) about 0.4-1.5% fragments of protein of interest, or g) about 0.1-8 ppm host cell protein.

In a particular embodiment, the composition comprising the protein of interest, e.g., an antibody or antigen-binding portion thereof, for example, an anti-GM-CSFRα antibody such as mavrilimumab, comprises less than about 24% basic species and about 11-38% acidic species. In another particular embodiment, the composition comprising the protein of interest, e.g., an antibody or antigen-binding portion thereof, for example, an anti-GM-CSFRα antibody such as mavrilimumab, comprises less than about 24% basic species and about 46-67% main species.

In some embodiments, the composition comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, e.g., anti-GM-CSFRα antibody such as mavrilimumab, and ranges within one or more of the preceding. In a particular embodiment, the composition comprises more than 64% main species. In another embodiment, the composition comprises more than 65% main species. In some embodiments, the composition comprises more than 40% main species.

In some embodiments, the composition comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-63%, about 58-62%, about 59-61%, about 58-67%, about 53-61%, about 46-67%, or about 46-66% main species, e.g., anti-GM-CSFRα antibody such as mavrilimumab, and ranges within one or more of the preceding. In some embodiments, the composition comprises about 58-67% main species. In some embodiments, the composition comprises about 46-67% main species.

In some embodiments, the composition comprising the protein of interest, e.g., an antibody or antigen-binding portion thereof, for example, an anti-GM-CSFRα antibody such as mavrilimumab, comprises greater than about 64% main species, and at least one of a) about 0.6-18% half antibody, b) about 11-38% acidic species, c) about 9-41% basic species, d) about 0.04-0.8% aggregates of protein of interest, e) about 98-99.9% monomer of protein of interest, f) about 0.4-1.5% fragments of protein of interest, or g) about 0.1-8 ppm host cell protein.

In some embodiments, the composition comprising the protein of interest, e.g., an antibody or antigen-binding portion thereof, for example, an anti-GM-CSFRα antibody such as mavrilimumab, comprises about 58-62% main species, and at least one of a) about 0.6-18% half antibody, b) about 11-38% acidic species, c) about 9-41% basic species, d) about 0.04-0.8% aggregates of protein of interest, e) about 98-99.9% monomer of protein of interest, f) about 0.4-1.5% fragments of protein of interest, or g) about 0.1-8 ppm host cell protein.

In a particular embodiment, the composition comprising the protein of interest, e.g., an antibody or antigen-binding portion thereof, for example, an anti-GM-CSFRα antibody such as mavrilimumab, comprises greater than about 64% main species and about 11-38% acidic species. In another particular embodiment, the composition comprising the protein of interest, e.g., an antibody or antigen-binding portion thereof, for example, an anti-GM-CSFRα antibody such as mavrilimumab, comprises greater than about 64% main species and about 9-41% basic species.

In yet another embodiment, the composition comprising the protein of interest, e.g., an antibody or antigen-binding portion thereof, for example, an anti-GM-CSFRα antibody such as mavrilimumab, comprises about 58-62% main species and about 11-38% acidic species. In yet a further embodiment, the composition comprising the protein of interest, e.g., an antibody or antigen-binding portion thereof, for example, an anti-GM-CSFRα antibody such as mavrilimumab, comprises about 58-62% main species and about 9-41% basic species.

In some embodiments, the composition comprising the protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the composition comprises less than about 10% high molecular weight aggregates. In some embodiments, the composition comprises less than about 0.5% high molecular weight aggregates. In some embodiments, the composition comprises less than about 0.4% high molecular weight aggregates. In further embodiments, the composition comprises less than about 0.3% high molecular weight aggregates.

In some embodiments, the composition comprising the protein of interest, e.g., an antibody or antigen-binding portion thereof, for example, an anti-GM-CSFRα antibody such as mavrilimumab, comprises about 0.01-10%, about 0.01-5%, about 0.01-1%, about 0.01-0.4%, about 0.04-0.2%, about 0.1-0.4%, about 0.04-0.4%, about 0.04-0.8%, about 0.5-0.8%, about 1-10%, about 2-10%, about 3-10%, or about 4-10% high molecular weight aggregates, and ranges within one or more of the preceding. In some embodiments, the composition comprises about 0.5-0.8% high molecular weight aggregates. In some embodiments, the composition comprises about 0.01-0.4% high molecular weight aggregates. In some embodiments, the composition comprises about 0.04-0.8% high molecular weight aggregates.

In some embodiments, the composition comprising the protein of interest, e.g., an antibody or antigen-binding portion thereof, for example, an anti-GM-CSFRα antibody such as mavrilimumab, comprises less than about 0.5% aggregates of protein of interest, and at least one of a) about 0.6-18% half antibody, b) about 11-38% acidic species, c) about 9-41% basic species, d) about 46-67% main species, e) about 98-99.9% monomer of protein of interest, f) about 0.4-1.5% fragments of protein of interest, and g) about 0.1-8 ppm host cell protein.

In some embodiments, the composition comprising the protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1% about 0.5%, about 0.4%, about 0.3% protein fragments, and ranges within one or more of the preceding. In some embodiments, the composition comprises less than about 10% of fragments of protein of interest. In some embodiments, the composition comprises less than 0.4% fragments of protein of interest. In some embodiments, the composition comprises less than 0.3% fragments of protein of interest.

In some embodiments, the composition comprising the protein of interest, e.g., an antibody or antigen-binding portion thereof, for example, an anti-GM-CSFRα antibody such as mavrilimumab, comprises about 0.1-10%, about 0.1-5%, about 0.1-3%, about 0.1-2%, about 0.5-1.5%, about 0.6-1.5%, about 0.5-1.1%, about 0.4-0.8% or about 0.4-1.1% protein fragments, and ranges within one or more of the preceding. In some embodiments, the composition comprises about 0.5-1.5% protein fragments. In some embodiments, the composition comprises about 0.6-1.5% protein fragments.

In some embodiments, the composition comprising the protein of interest, e.g., an antibody or antigen-binding portion thereof, for example, an anti-GM-CSFRα antibody such as mavrilimumab, comprises less than about 0.4% fragments of protein of interest, and at least one of a) about 0.6-18% half antibody, b) about 11-38% acidic species, c) about 9-41% basic species, d) about 46-67% main species, e) about 0.04-0.8% aggregates of protein of interest, f) about 98-99.9% monomer of protein of interest, or g) about 0.1-8 ppm host cell protein.

In some embodiments, the composition comprising the protein of interest, e.g., an antibody or antigen-binding portion thereof, for example, an anti-GM-CSFRα antibody such as mavrilimumab, comprises more than about 90%, about 91%, about 92%, about 93%, about 94%, about 95%, about 96%, about 97%, about 98%, about 99%, about 99.1% or about 99.5% of monomer of the protein of interest, e.g., antibody monomer. In some embodiments, the composition comprises more than about 90% of monomer, e.g., antibody monomer. In some embodiments, the composition comprises more than about 99.1% of monomer, e.g., antibody monomer.

In some embodiments, the composition comprises about 90-99.9%, about 90-95%, about 95-99.9%, about 99-99.9%, about 99.1-99.9%, about 98-99%, about 98-99.9%, or about 98.5-99.5% of monomer of the protein of interest. In some embodiments, the composition comprises about 98-99% monomer. In some embodiments, the composition comprises about 98-99.9% monomer.

In some embodiments, the composition comprising the protein of interest, e.g., an antibody or antigen-binding portion thereof, for example, an anti-GM-CSFRα antibody such as mavrilimumab, comprises greater than about 99.1% monomer of protein of interest, and at least one of a) about 0.6-18% half antibody, b) about 11-38% acidic species, c) about 9-41% basic species, d) about 46-67% main species, e) about 0.04-0.8% aggregates of protein of interest, f) about 0.4-1.5% fragments of protein of interest, or g) about 0.1-8 ppm host cell protein.

In some embodiments, the composition comprising the protein of interest, e.g., an antibody or antigen-binding portion thereof, for example, an anti-GM-CSFRα antibody such as mavrilimumab, comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the composition comprises less than about 10 ppm HCP.

In some embodiments, the composition comprising the protein of interest, e.g., an antibody or antigen-binding portion thereof, for example, an anti-GM-CSFRα antibody such as mavrilimumab, comprises about 0.1-10, about 1-10, about 2-10, about 3-10, about 4-10, about 1-5, about 5-10, about 1-3, about 0.1-2, about 0.1-3, about 2-8, or about 0.1-8 ppm HCP, and ranges within one or more of the preceding. In some embodiments, the composition comprises about 0.1-2 HCP.

The protein in the compositions of the invention comprises an antibody or antigen binding portion thereof. For example, the antibody, or antigen binding portion thereof may be an anti-GM-CSFRα antibody, or antigen binding portion thereof, such as mavrilimumab, or an antigen binding portion thereof. In one aspect of this embodiment, the antibody, or antigen binding portion thereof, comprises a heavy chain comprising the sequence set forth as SEQ ID NO:2, and a light chain comprising the sequence set forth as SEQ ID NO:3. In one aspect of this embodiment, the antibody, or antigen binding portion thereof, comprises a heavy chain variable region comprising the sequence set forth as SEQ ID NO:4, and a light chain variable region comprising the sequence set forth as SEQ ID NO:5. In another aspect of this embodiment, the antibody, or antigen binding portion thereof, comprises a heavy chain variable region comprising a CDR1 having the sequence set forth as SEQ ID NO:6, a CDR2 having the sequence set forth as SEQ ID NO:7, and a CDR3 having the sequence set forth as SEQ ID NO:8. In another aspect of this embodiment, the antibody, or antigen binding portion thereof, comprises a light chain variable region comprising a CDR1 having the sequence set forth as SEQ ID NO:9, a CDR2 having the sequence set forth as SEQ ID NO:10 and a CDR3 having the sequence set forth as SEQ ID NO:11.

In some embodiments, the compositions of the invention comprise an eluate fraction comprising an anti-GM-CSFRα antibody, or antigen-binding portion thereof, wherein the eluate fraction is collected from a chromatography resin selected from a group consisting of a cation exchange chromatography resin, an anion exchange chromatography resin and a mixed mode chromatography resin.

In some embodiments, the eluate fraction comprising the protein of interest, e.g., an antibody or antigen-binding portion thereof (for example, an anti-GM-CSFRα antibody such as mavrilimumab), for example, collected following a cation exchange or mixed mode chromatography step, comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the eluate fraction comprises less than 20% half antibody. In some embodiments, the eluate fraction comprises less than 18% half antibody.

In some embodiments, the eluate fraction comprising the protein of interest, e.g., an antibody or antigen-binding portion thereof (for example, an anti-GM-CSFRα antibody such as mavrilimumab), for example, collected following a cation exchange or mixed mode chromatography step, comprises about 0.1-25%, 0.1-20%, about 0.1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one of more of the preceding. In some embodiments, the eluate fraction comprises about 0.6-18% or about 1-17% half antibody. In some embodiments, the eluate fraction is collected from a cation exchange chromatography resin and comprises about 0.6-18% half antibody. In some embodiments, the eluate fraction is collected from a mixed mode chromatography resin and comprises about 1-17% half antibody.

In some embodiments, the eluate fraction comprising the protein of interest, e.g., an antibody or antigen-binding portion thereof (for example, an anti-GM-CSFRα antibody such as mavrilimumab), for example, collected following a cation exchange, anion exchange, or mixed mode chromatography step, comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the eluate fraction comprises less than about 40% acidic species.

In some embodiments, the eluate fraction comprising the protein of interest, e.g., an antibody or antigen-binding portion thereof (for example, an anti-GM-CSFRα antibody such as mavrilimumab), for example, collected following a cation exchange, anion exchange or mixed mode chromatography step, comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding. In some embodiments, the eluate fraction comprises about 14-28%, about 11-22%, about 11-38%, or about 12-38% acidic species.

In some embodiments, the eluate fraction is collected from a cation exchange chromatography resin and comprises about 14-28% acidic species. In some embodiments, the eluate fraction is collected from an anion exchange chromatography resin and comprises about 11-22% acidic species. In some embodiments, the eluate fraction is collected from a mixed mode chromatography resin and comprises about 12-38% acidic species.

In some embodiments, the eluate fraction comprising the protein of interest, e.g., an antibody or antigen-binding portion thereof (for example, an anti-GM-CSFRα antibody such as mavrilimumab), for example, collected following a cation exchange, anion exchange or mixed mode chromatography step, comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% basic species, and ranges within one or more of the preceding. In some embodiments, the eluate fraction comprises less than about 45% basic species.

In some embodiments, the eluate fraction comprising the protein of interest, e.g., an antibody or antigen-binding portion thereof (for example, an anti-GM-CSFRα antibody such as mavrilimumab), for example, collected following a cation exchange, anion exchange or mixed mode chromatography step, comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding. In some embodiments, the eluate fraction comprises about 15-25%, about 9-29%, about 9-41%, or about 16-41% basic species. In some embodiments, the eluate fraction is collected from a cation exchange chromatography resin and comprises about 15-25% basic species. In some embodiments, the eluate fraction is collected from an anion exchange chromatography resin and comprises about 16-41% basic species. In some embodiments, the eluate fraction is collected from a mixed mode chromatography resin and comprises about 9-29% basic species.

In some embodiments, the eluate fraction comprising an anti-GM-CSFRα antibody, or antigen-binding portion thereof, such as mavrilimumab, for example, collected following a cation exchange, anion exchange or mixed mode chromatography step, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the eluate fraction comprises more than 40% main species.

In some embodiments, the eluate fraction comprising an anti-GM-CSFRα antibody, or antigen-binding portion thereof, such as mavrilimumab, for example, collected following a cation exchange, anion exchange or mixed mode chromatography step, comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 59-61%, about 58-63%, about 58-67%, about 53-61%, about 46-67%, or about 46-66% main species, and ranges within one or more of the preceding.

In some embodiments, the eluate fraction comprising an anti-GM-CSFRα antibody, or antigen-binding portion thereof, such as mavrilimumab, for example, collected following a cation exchange, anion exchange or mixed mode chromatography step, comprises about 55-65%, about 53-61%, or about 46-66% main species. In some embodiments, the eluate fraction is collected from a cation exchange chromatography resin and comprises about 55-65% main species. In some embodiments, the eluate fraction is collected from an anion exchange chromatography resin and comprises about 46-66% main species. In some embodiments, the eluate fraction is collected from a mixed mode chromatography resin and comprises about 53-61% main species.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the eluate fraction comprises less than about 10% high molecular weight aggregates.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises about 0.01-10%, about 0.01 to 5%, about 0.01 to 1%, about 0.01-0.4%, about 0.04-0.2%, about 0.1-0.4%, about 0.04-0.4%, about 0.04-0.8%, about 0.5-0.8%, about 1-10%, about 2-10%, about 3-10%, or about 4-10% high molecular weight aggregates, and ranges within one or more of the preceding. In some embodiments, the eluate fraction comprises about 0.04-0.2%, about 0.1-0.4%, about 0.04-0.4% high molecular weight aggregates. In some embodiments, the eluate fraction is collected from a cation exchange chromatography resin and comprises about 0.1-0.4% high molecular weight aggregates. In some embodiments, the eluate fraction is collected from an anion exchange chromatography resin and comprises about 0.04-0.2% high molecular weight aggregates.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4% or about 0.3% protein fragments, e.g., antibody fragments, and ranges within one or more of the preceding. In some embodiments, the eluate fraction comprises less than about 10% of fragments of protein of interest.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises about 0.1-10%, about 0.1-5%, about 0.1-3%, about 0.1-2%, about 0.5-1.5%, about 0.5-1.1%, about 0.6-1.5%, about 0.4-0.8% or about 0.4-1.1% protein fragments, and ranges within one or more of the preceding. In some embodiments, the eluate fraction comprises about 0.5-1.1%, about 0.4-0.8% or about 0.4-1.1% protein fragments. In some embodiments, the eluate fraction is collected from a cation exchange chromatography resin and comprises about 0.4-0.8% protein fragments. In some embodiments, the eluate fraction is collected from an anion exchange chromatography resin and comprises about 0.5-1.1% protein fragments.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises more than about 90%, about 91%, about 92%, about 93%, about 94%, about 95%, about 96%, about 97%, about 98%, about 99%, about 99.1% or about 99.5% of monomer of the protein of interest, e.g., antibody monomer. In some embodiments, the eluate fraction comprises more than about 90% monomer, e.g., antibody monomer.

In some embodiment, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises about 90-99.9%, about 90-95%, about 95-99.9%, about 99-99.9%, about 99.1-99.9%, about 98-99%, about 98-99.9%, or about 98.5-99.5% of monomer of the protein of interest, e.g., antibody monomer. In some embodiments, the eluate fraction comprises about 98-99.9% or about 98.5-99.5% of monomer, e.g., antibody monomer. In some embodiments, the eluate fraction is collected from a cation exchange chromatography resin and comprises about 98-99.9% or about 98.5-99.5% of monomer, e.g., antibody monomer. In some embodiments, the eluate fraction is collected from an anion exchange chromatography resin and comprises about 98-99.9% or about 98.5-99.5% of monomer, e.g., antibody monomer.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the eluate fraction comprises less than 10 ppm HCP.

In some embodiments, the eluate fraction comprising the protein of interest, e.g., an antibody or antigen-binding portion thereof (for example, an anti-GM-CSFRα antibody such as mavrilimumab), for example, collected following a cation exchange or anion exchange step, comprises about 0.1-10, about 1-10, about 2-10, about 3-10, about 4-10, about 1-5, about 5-10, about 1-3, about 0.1-2, about 0.1-3, about 2-8 or about 0.1-8 ppm HCP, and ranges within one or more of the preceding. In some embodiments, the eluate fraction comprises about 2-8 ppm or about 0.1-2 ppm HCP. In some embodiments, the eluate fraction is collected from a cation exchange chromatography resin and comprises about 2-8 ppm HCP. In some embodiments, the eluate fraction is collected from an anion exchange chromatography resin and comprises about 0.1-2 ppm HCP.

In some embodiments, the compositions of the invention comprise a clarified harvest from cell culture comprising protein of interest, e.g., an antibody or antigen-binding portion thereof, for example, an anti-GM-CSFRα antibody such as mavrilimumab. As used herein, the term “clarified harvest” or “clarified cell culture harvest” refers to a harvest from which the cells have been separated from the growing medium. The clarification process may be performed by centrifugation, microfiltration, depth filtration or filtration through membranes with different pore sizes, or a combination thereof, to remove solids, or it may include the use of chemical additives or solid materials bearing chemically interactive surfaces to extract particular classes of soluble contaminants from the harvest.

In some embodiments, the clarified harvest comprising the protein of interest, e.g., an antibody or antigen-binding portion thereof (for example, an anti-GM-CSFRα antibody such as mavrilimumab), comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the clarified harvest comprises less than 25% half antibody. In some embodiments, the clarified harvest comprises less than 20% half antibody.

In some embodiments, the clarified harvest comprising the protein of interest, e.g., an antibody or antigen-binding portion thereof (for example, an anti-GM-CSFRα antibody such as mavrilimumab), comprises about 0.1-25%, 0.1-20%, about 0.1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one of more of the preceding. In some embodiments, the clarified harvest comprises about 0.1-25% half antibody.

In some embodiments, the clarified harvest comprising the protein of interest, e.g., an antibody or antigen-binding portion thereof (for example, an anti-GM-CSFRα antibody such as mavrilimumab), comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the clarified harvest comprises less than about 40% acidic species.

In some embodiments, the clarified harvest comprising the protein of interest, e.g., an antibody or antigen-binding portion thereof (for example, an anti-GM-CSFRα antibody such as mavrilimumab), comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding. In some embodiments, the clarified harvest comprises about 1-40% acidic species.

In some embodiments, the clarified harvest comprising the protein of interest, e.g., an antibody or antigen-binding portion thereof (for example, an anti-GM-CSFRα antibody such as mavrilimumab), comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% basic species, and ranges within one or more of the preceding. In some embodiments, the clarified harvest comprises less than about 45% basic species.

In some embodiments, the clarified harvest comprising the protein of interest, e.g., an antibody or antigen-binding portion thereof (for example, an anti-GM-CSFRα antibody such as mavrilimumab), comprises about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding. In some embodiments, the clarified harvest comprises about 1-40% basic species.

In some embodiments, the clarified harvest comprising an anti-GM-CSFRα antibody, or antigen-binding portion thereof, such as mavrilimumab, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the clarified harvest comprises more than 50% main species.

In some embodiments, the clarified harvest comprising an anti-GM-CSFRα antibody, or antigen-binding portion thereof, such as mavrilimumab, comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 59-61%, about 58-63%, about 58-67%, about 53-61%, about 46-67%, or about 46-66% main species, and ranges within one or more of the preceding. In some embodiments, the clarified harvest comprises about 40-99% main species.

In some embodiments, the clarified harvest comprising the protein of interest, e.g., an antibody or antigen-binding portion thereof (for example, an anti-GM-CSFRα antibody such as mavrilimumab), comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the clarified harvest comprises less than about 10% high molecular weight aggregates.

In some embodiments, the clarified harvest comprising the protein of interest, e.g., an antibody or antigen-binding portion thereof (for example, an anti-GM-CSFRα antibody such as mavrilimumab), comprises about 0.01-10%, about 0.01 to 5%, about 0.01 to 1%, about 0.01-0.4%, about 0.04-0.2%, about 0.1-0.4%, about 0.04-0.8%, about 0.04-1%, about 0.5-0.8%, about 1-10%, about 2-10%, about 3-10%, or about 4-10% high molecular weight aggregates, and ranges within one or more of the preceding. In some embodiments, the clarified harvest comprises about 0.01-10% high molecular weight aggregates.

In some embodiments, the clarified harvest comprising the protein of interest, e.g., an antibody or antigen-binding portion thereof (for example, an anti-GM-CSFRα antibody such as mavrilimumab), comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4% or about 0.3% protein fragments, e.g., antibody fragments, and ranges within one or more of the preceding. In some embodiments, the clarified harvest comprises less than about 10% of fragments of protein of interest.

In some embodiments, the clarified harvest comprising the protein of interest, e.g., an antibody or antigen-binding portion thereof (for example, an anti-GM-CSFRα antibody such as mavrilimumab), comprises about 0.1-10%, about 0.1-5%, about 0.1-3%, about 0.1-2%, about 0.4-1.5%, about 0.5-1.5%, about 0.5-1.1%, about 0.6-1.5%, about 0.4-0.8% or about 0.4-1.1% protein fragments, and ranges within one or more of the preceding. In some embodiments, the clarified harvest comprises about 0.1-10% protein fragments.

In some embodiments, the clarified harvest comprising the protein of interest, e.g., an antibody or antigen-binding portion thereof (for example, an anti-GM-CSFRα antibody such as mavrilimumab), comprises more than about 90%, about 91%, about 92%, about 93%, about 94%, about 95%, about 96%, about 97%, about 98%, about 99%, about 99.1% or about 99.5% of monomer of the protein of interest, e.g., antibody monomer. In some embodiments, the clarified harvest comprises more than about 90% monomer of the protein of interest, e.g., antibody monomer.

In some embodiment, the clarified harvest comprising the protein of interest, e.g., an antibody or antigen-binding portion thereof (for example, an anti-GM-CSFRα antibody such as mavrilimumab), comprises about 90-99.9%, about 90-95%, about 95-99.9%, about 99-99.9%, about 99.1-99.9%, about 98-99%, about 98-99.9%, or about 98.5-99.5% of monomer of the protein of interest, e.g., antibody monomer. In some embodiments, the clarified harvest comprises about 90-99.9% of monomer of the protein of interest, e.g., antibody monomer.

In some embodiments, the clarified harvest comprising the protein of interest, e.g., an antibody or antigen-binding portion thereof (for example, an anti-GM-CSFRα antibody such as mavrilimumab), comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the clarified harvest comprises less than 10 ppm HCP.

In some embodiments, the clarified harvest comprising the protein of interest, e.g., an antibody or antigen-binding portion thereof (for example, an anti-GM-CSFRα antibody such as mavrilimumab), comprises about 0.1-10, about 1-10, about 2-10, about 3-10, about 4-10, about 1-5, about 5-10, about 1-3, about 0.1-2, about 0.1-3, about 2-8 or about 0.1-8 ppm HCP, and ranges within one or more of the preceding. In some embodiments, the clarified harvest comprises about 0.1-10 ppm HCP.

In certain aspects of the invention, the protein of interest is an antibody, or an antigen binding portion thereof. The antibody, or antigen binding portion thereof, that can be used in the compositions of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495.

Somatic cell hybridization procedures can be used. In principle, other techniques for producing monoclonal antibody can be employed as well, including viral or oncogenic transformation of B lymphocytes.

One exemplary animal system for preparing hybridomas is the murine system. Hybridoma production is a very well-established procedure Immunization protocols and techniques for isolation of immunized splenocytes for fusion are known in the art. Fusion partners (e.g., murine myeloma cells) and fusion procedures are also known.

An antibody used in the compositions of the invention can be a human, a chimeric, or a humanized antibody. Chimeric or humanized antibodies used in the compositions of the invention can be prepared based on the sequence of a non-human monoclonal antibody prepared as described above.

DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies to be used in the compositions of the invention are human monoclonal antibodies. Such human monoclonal antibodies can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen). The antibodies, or antigen-binding portions thereof, used in the compositions of the invention can also be produced using the methods described in U.S. Pat. No. 6,090,382, the entire contents of which is expressly incorporated herein by reference.

Moreover, alternative transchromosomic animal systems expressing human immunoglobulin genes are available in the art and can be used to raise antibodies of the disclosure. For example, mice carrying both a human heavy chain transchromosome and a human light chain transchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise antibodies of this disclosure.

Recombinant human antibodies to be used in the compositions of the invention can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibody Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies to be used in the compositions of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In certain embodiments, the human antibodies to be used in the compositions of the invention are anti-GM-CSFRα antibodies and antibody portions thereof, anti-GM-CSFRα-related antibodies and antibody portions, and human antibodies and antibody portions with equivalent properties to anti-GM-CSFRα antibodies, such as high affinity binding to GM-CSFRα with low dissociation kinetics and high neutralizing capacity. In one embodiment, an anti-GM-CSFRα antibody to be used in the compositions of the invention binds to the same epitope on GM-CSFRα as mavrilimumab. In another embodiment, an anti-GM-CSFRα antibody to be used in the compositions of the invention competitively inhibits binding of mavrilimumab to GM-CSFRα under physiological conditions. In one embodiment, the compositions of the invention comprise mavrilimumab, or an antigen binding portion thereof.

Antibodies or antigen binding portion thereof to be used in the compositions of the invention can be altered, wherein the constant region of the antibody is modified to reduce at least one constant region-mediated biological effector function relative to an unmodified antibody. To modify an antibody of the invention such that it exhibits reduced binding to the Fc receptor, the immunoglobulin constant region can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### III. Preparation of Compositions Using Upstream Process Technologies

The compositions of the present invention comprising a protein, e.g., an antibody, or antigen binding portion thereof, for example, an anti-GM-CSFRα antibody such as mavrilimumab, having a reduced level of variants and/or impurities, e.g., a reduced level of product-related substances, e.g., protein aggregates, fragments, e.g., half antibody, or charged species, e.g., acidic or basic species; and/or a reduced level of process-related impurities, e.g., host cell proteins, can be produced by modulating conditions during upstream protein production, such as cell culture. In certain embodiments, the compositions of the present invention include, but are not limited to, compositions comprising an anti-GM-CSFRα antibody or antigen-binding portion thereof such as mavrilimumab having a reduced level of variants and/or impurities, e.g., a reduced level of product-related substance, e.g., protein aggregates, fragments, e.g., half antibody, or charged species, e.g., acidic species or basic species; and/or a reduced level of process-related impurities, e.g., host cell protein. Such variants and/or impurities-reduced compositions address the need for improved product characteristics, including, but not limited to, product stability, product safety and product efficacy.

The present invention provides methods for producing a preparation comprising a protein of interest, e.g., an antibody, or antigen binding portion thereof, for example, an anti-GM-CSFRα antibody such as mavrilimumab, having a reduced level of variants and/or impurities, e.g., a reduced level of product-related substance, e.g., protein aggregates, fragments, e.g., half antibody, or charged species, e.g., acidic species or basic species; and/or a reduced level of process-related impurities, e.g., host cell protein, from a cell culture by modulating conditions for cell culture, e.g., by modulating the pH level, the CO₂ level, the level of cell culture supplement, and/or the level of lactate or lactate production from the cell culture.

The present invention also provides methods for reducing the level of variants and/or impurities, e.g., the level of product-related substance, e.g., protein aggregates, fragments, e.g., half antibody, or charged species, e.g., acidic species or basic species; and/or the level of process-related impurities, e.g., host cell protein, of a protein of interest, e.g., an antibody or antigen-binding portion thereof, for example, an anti-GM-CSFRα antibody such as mavrilimumab, in a cell culture, by modulating conditions for cell culture, e.g., by modulating the pH level, the CO₂ level, the level of cell culture supplement, and/or the level of lactate or lactate production from the cell culture.

Furthermore, the present invention provides methods for increasing production yield of a protein of interest, e.g., an antibody or antigen-binding portion thereof, for example, an anti-GM-CSFRαantibody such as mavrilimumab, from a cell culture, by modulating conditions for cell culture, e.g., by modulating the pH level, the CO₂ level, the level of cell culture supplement, and/or the level of lactate or lactate production from the cell culture.

In some embodiments, the present invention provides a method for preparing a preparation comprising a protein of interest, e.g., an antibody, or antigen binding portion thereof, for example, an anti-GM-CSFRα antibody such as mavrilimumab, having a reduced level of acidic species from a cell culture by incubating the cell culture in a bioreactor and maintaining the pH of the cell culture at a pH of about 6-7.5, e.g., about 6-7, about 6.1-7, about 6.2-7, about 6.3-7, about 6.4-7, about 6.5-7, about 6.5-7.5, about 6.5-7.4, about 6.5-7.3, about 6.5-7.2, about 6.5-7.1, about 6.6-7, about 6.7-7, about 6.75-6.95, thereby preparing a preparation comprising the protein of interest with a reduced level of acidic species.

In other embodiments, the present invention provides a method for preparing a preparation comprising a protein of interest, e.g., an antibody, or antigen binding portion thereof, for example, an anti-GM-CSFRα antibody such as mavrilimumab, having a reduced level of acidic species from a cell culture, by incubating the cell culture in a bioreactor; and one of more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 1 g/L; (d) increasing lactate production in the cell culture; (e) increasing the level of CO2 in the cell culture; and/or (f) decreasing the pH of the cell culture, thereby preparing a preparation comprising the protein of interest with a reduced level of acidic species.

In another embodiment, the present invention provides a method for reducing the level of acidic species of a protein of interest, e.g., an antibody, or antigen binding portion thereof, for example, an anti-GM-CSFRα antibody such as mavrilimumab, in a cell culture, by incubating the cell culture in a bioreactor; and one or more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 1 g/L; (d) increasing lactate production in the cell culture; (e) increasing the level of CO2 in the cell culture; and/or (f) decreasing the pH of the cell culture, thereby reducing the level of acidic species of the protein of interest.

In a further embodiment, the present invention provides a method for increasing production yield of a protein of interest, e.g., an antibody, or antigen binding portion thereof, for example, an anti-GM-CSFRα antibody such as mavrilimumab, from a cell culture, by incubating the cell culture in a bioreactor; and one or more steps selected from the group consisting of (a) maintaining the pH of the cell culture at a pH of about 6-7.5, about 6-7 or about 6.5-7; (b) increasing the level of cell culture supplement during the incubation period; (c) maintaining the level of lactate in the cell culture at about 1 g/L; (d) increasing lactate production in the cell culture; (e) increasing the level of CO2 in the cell culture; and/or (f) decreasing the pH of the cell culture, thereby increasing the production yield of the protein of interest.

The upstream process technologies may be used alone or in combination with the downstream process technologies described in Section IV, below, and as described in Example 1.

In one embodiment, one or more of the upstream process technologies described herein produce a composition of the present invention comprising a protein, e.g., an antibody, or antigen binding portion thereof, for example, an anti-GM-CSFRα antibody such as mavrilimumab, having a reduced level of variants and/or impurities, e.g., a reduced level of product-related substances, e.g., protein aggregates, fragments, e.g., half antibody, or charged species, e.g., acidic or basic species; and/or a reduced level of process-related impurities, e.g., host cell proteins, as described herein.

Some embodiments of the invention comprise culturing host cells to express a protein of interest under conditions that limit the amount of product-related substances e.g., acidic species, that are expressed by the cells. Some embodiments of the invention comprise culturing host cells under conditions that limit the conversion of the product to acidic species variants.

In certain embodiments, the composition comprising a protein of interest, e.g., an antibody, or antigen binding portion thereof, for example, an anti-GM-CSFRα antibody such as mavrilimumab, having a reduced level of acidic species is produced by culturing host cells in a culture wherein the process parameters, such as pH or carbon dioxide (CO₂) levels of the cell culture, are modulated to decrease the amount of acidic species produced by the host cells and/or reduce the conversion of the product to the acidic species variants. In other embodiments, the composition comprising a protein of interest, e.g., an antibody, or antigen binding portion thereof, for example, an anti-GM-CSFRα antibody such as mavrilimumab, having a reduced level of acidic species is produced by culturing host cells in a culture wherein the levels of lactate and/or cell culture supplements are modulated to decrease the amount of acidic species produced by the host cells and/or reduce the conversion of the product to the acidic species variants.

In one embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest in a bioreactor, and maintaining the pH of the cell culture at a pH of about 6-7.5.

In another embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest in a bioreactor, and increasing the level of cell culture supplement during the incubation period.

In still another embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest in a bioreactor, and maintaining the level of lactate in the cell culture at about 1 g/L.

In yet another embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest in a bioreactor, and increasing lactate production in the cell culture.

In still another embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest in a bioreactor, and increasing the level of CO₂ in the cell culture.

In another embodiment, the composition comprising a protein of interest, e.g., an antibody, or antigen binding portion thereof, for example, an anti-GM-CSFRα antibody such as mavrilimumab, having a reduced level of acidic species is produced by incubating cells expressing the protein of interest, in a bioreactor, and decreasing the pH of the cell culture.

In another embodiment, one or more of the above supplements and modifications can be combined and used during cell culture of the protein, e.g., antibody, composition.

To express a protein of interest to be used in the compositions of the invention, DNAs encoding the protein, such as DNAs encoding partial or full-length light and heavy chains of an antibody, are inserted into one or more expression vector such that the genes are operatively linked to transcriptional and translational control sequences. (See, e.g., U.S. Pat. No. 6,090,382, the entire teaching of which is incorporated herein by reference.) In this context, the term “operatively linked” is intended to mean that a gene encoding the protein of interest is ligated into a vector such that transcriptional and translational control sequences within the vector serve their intended function of regulating the transcription and translation of the gene. The expression vector and expression control sequences are chosen to be compatible with the expression host cell used. In certain embodiments, the protein of interest will comprise multiple polypeptides, such as the heavy and light chains of an antibody. Thus, in certain embodiments, genes encoding multiple polypeptides, such as antibody light chain genes and antibody heavy chain genes, can be inserted into a separate vector or, more typically, the genes are inserted into the same expression vector. Genes are inserted into expression vectors by standard methods (e.g., ligation of complementary restriction sites on the gene fragment and vector, or blunt end ligation if no restriction sites are present). Prior to insertion of the gene or genes, the expression vector may already carry additional polypeptide sequences, such as, but not limited to, antibody constant region sequences. For example, one approach to converting the anti-GM-CSFRα antibody or anti-GM-CSFRα antibody-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the protein from a host cell. The gene can be cloned into the vector such that the signal peptide is linked in-frame to the amino terminus of the gene. The signal peptide can be an immunoglobulin signal peptide or a heterologous signal peptide (i.e., a signal peptide from a non-immunoglobulin protein).

In addition to protein coding genes, a recombinant expression vector can carry one or more regulatory sequence that controls the expression of the protein coding genes in a host cell. The term “regulatory sequence” is intended to include promoters, enhancers and other expression control elements (e.g., polyadenylation signals) that control the transcription or translation of the protein coding genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

A recombinant expression vector may also carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody, or antibody portion, e.g., an anti-GM-CSFRα antibody such as mavrilimumab, to be used in the compositions of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of protein, for example, the light and heavy chains of an antibody, the expression vector(s) encoding the protein is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the proteins of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active protein. Prokaryotic expression of protein genes has been reported to be ineffective for production of high yields of active protein (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated proteins, for example, glycosylated antibodies, are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Mammalian cells can be used for expression and production of the recombinant protein used in the compositions of the invention, however other eukaryotic cell types can also be employed in the context of the instant invention. See, e.g., Winnacker, From Genes to Clones, VCH Publishers, N.Y., N.Y. (1987). Suitable mammalian host cells for expressing recombinant proteins according to the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding protein genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for protein production and cultured in conventional nutrient media modified as appropriate for inducing promoters, selecting transformants, or amplifying the genes encoding the desired sequences.

The host cells used to produce a protein may be cultured in a variety of media. Commercially available media such as Ham's F10™ (Sigma), Minimal Essential Medium™ (MEM), (Sigma), RPMI-1640 (Sigma), and Dulbecco's Modified Eagle's Medium™ (DMEM), (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact proteins, for example, antibodies, including Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody. Recombinant DNA technology may also be used to remove some or all of the DNA encoding either or both of the light and heavy chains that is not necessary for binding to an antigen. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the target antibody, depending on the specificity of the antibody of the invention, by crosslinking an antibody of the invention to a second antibody by standard chemical crosslinking methods.

In a suitable system for recombinant expression of a protein, for example, an antibody, or antigen-binding portion thereof, a recombinant expression vector encoding the protein, for example, both an antibody heavy chain and an antibody light chain, is introduced into dhfr-CHO cells by calcium phosphate-mediated transfection. Within the recombinant expression vector, the protein gene(s) are each operatively linked to CMV enhancer/AdMLP promoter regulatory elements to drive high levels of transcription of the gene(s). The recombinant expression vector also carries a DHFR gene, which allows for selection of CHO cells that have been transfected with the vector using methotrexate selection/amplification. The selected transformant host cells are cultured to allow for expression of the protein, for example, the antibody heavy and light chains, and intact protein, for example, an antibody, is recovered from the culture medium. Standard molecular biology techniques are used to prepare the recombinant expression vector, transfect the host cells, select for transformants, culture the host cells and recover the protein from the culture medium.

When using recombinant techniques, the protein, for example, antibodies or antigen binding fragments thereof, can be produced intracellularly, in the periplasmic space, or directly secreted into the medium. For antibodies made intracellularly, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter. Where the antibody is secreted into the medium, the recombinant host cells can also be separated from the cell culture medium, e.g., by tangential flow filtration. Antibodies can be further recovered from the culture medium using the antibody purification methods of the invention.

### Controlling pH Level to Modulate Acidic Species

In certain embodiments, the pH level of the cell culture is controlled (e.g., increased or decreased) in order to produce a composition of the invention comprising a protein of interest, e.g., an antibody or antigen-binding portion thereof, for example, an anti-GM-CSFRα antibody such as mavrilimumab, having a desired level of variants and/or impurities, e.g., a reduced level of acidic species. For example, the pH may be decreased in order to achieve the desired pH, e.g., between about 6-7.5. Alternatively, the pH may be maintained at the desired pH, e.g., between about 6-7.5.

In certain embodiments, the pH of the cell culture is decreased to or maintained at about 6, 6.05, 6.1, 6.15, 6.2, 6.25, 6.3, 6.35, 6.4, 6.45, 6.5, 6.55, 6.6, 6.65, 6.7, 6.75, 6.8, 6.85, 6.9, 6.95, 7, 7.05, 7.1, 7.15, 7.2, 7.25, 7.3, 7.35, 7.4, 7.45 or 7.5. In some embodiments, the pH of the cell culture is maintained at about 6-7.5, e.g., about 6-7, about 6.1-7, about 6.2-7, about 6.3-7, about 6.4-7, about 6.5-7, about 6.5-7.5, about 6.5-7.4, about 6.5-7.3, about 6.5-7.2, about 6.5-7.1, about 6.6-7, about 6.7-7, about 6.75-6.9 or about 6.75-6.95.

The pH is maintained in such a manner as to produce a composition comprising a protein of interest, e.g., an antibody or antigen-binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding acidic species, and ranges within one or more of the preceding.

In some embodiments, the composition comprises a clarified harvest from cell culture, wherein the clarified harvest comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the clarified harvest comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding acidic species, and ranges within one or more of the preceding.

In certain embodiments, the pH is maintained in such a manner as to reduce the amount of acidic species in a protein or antibody composition by about 1%, 2%, 3%, 4%, 5%, 6%, 7%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, and ranges within one or more of the preceding.

In certain embodiments, pH is either increased or decreased in order to increase or decrease the amount of acidic species and/or the rate at which such acidic species form. In some embodiments, the pH of the cell culture, initially at between about 6.5-7.0, is decreased by between about 0.01-0.5, about 0.02-0.4, about 0.05-0.3, or about 0.1-0.5, about 0.1-0.4, about 0.1-0.3, or about 0.1-0.2. For example, but not by way of limitation, as detailed in Example 1 below, a reduction in pH from about 6.9 to about 6.75 can be employed to decrease the acidic species during cell culture and the rate of acidic species formation in the context of a clarified harvest.

In certain embodiments, the pH of the cell culture is modulated at different time points during incubation. For example, but not limited to, increasing or decreasing the pH of the cell culture may occur at an earlier time point, e.g., at Day 1, 2, 3, 4, 5, 6, 7, 8, 9, or 10, during the incubation period, or a later time point, e.g., at the third last day, the second last day, or the last day of the incubation period. The cell culture pH may be changed, e.g., increased or decreased, multiple times throughout the incubation period, e.g., once, twice, or three times during the incubation period, in order to achieve a desirable level of acidic species in the composition.

In some embodiments, the pH of the cell culture, initially at about 6.5-7.0, is decreased by about 0.01-0.5, about 0.02-0.4, about 0.05-0.3, about 0.1-0.5, about 0.1-0.4, about 0.1-0.3, or about 0.1-0.2 during Day 2-Day 8, e.g., Day 2, 3, 4, 5, 6, 7, 8, of the incubation period. In some embodiments, the pH of the cell culture, initially at about 6.5-7.0, is decreased by between about 0.01-0.5, about 0.02-0.4, about 0.05-0.3, or about 0.1-0.5, about 0.1-0.4, about 0.1-0.3, or about 0.1-0.2 on Day 4 or Day 5 of the incubation period.

In some embodiments, the pH shift occurs on Day 4 of the incubation period, wherein the pH of the cell culture is reduced from 6.9 to 6.75. In other embodiments, the pH shift occurs on Day 5 of the incubation period, wherein the pH of the cell culture is reduced from 6.9 to 6.75. In another embodiment, the pH shift occurs on Day 4 of the incubation period, wherein the pH of the cell culture is dropped from 6.9 to 6.65. In yet another embodiment, the pH shift occurs on Day 5 of the incubation period, wherein the pH of the cell culture is dropped from 6.9 to 6.65. In various embodiments, the decrease in pH is achieved by increasing the CO₂ level of the cell culture, or by increasing the lactate level of the cell culture (for example, by increasing the amount of cell culture media feed added to the cell culture at day 3, day 4, day 5 or day 6), or a combination thereof. In certain embodiments, a composition of the invention having a reduced level of variants and/or impurities, e.g., a reduced level of acidic species, can be produced from cell culture by maintaining the pH of the cell culture expressing the protein of interest as described herein along with choice of suitable temperature or temperature shift strategies, for example, but not limited to, increase or decrease process temperature of operation, temperature shift to a lower temperature or a higher temperature, or a temperature shift at an earlier culture time point. These culture conditions can be used in various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, and any other configuration appropriate for optimal growth and productivity of the desired cell line

These methods of modulating pH and/or temperature may also be used in combination with methods of modulating CO₂ levels, methods of modulating the lactate level in cell culture or lactate production from the cell culture, or supplementation of culture media with additives such as one or more cell culture supplements (media feeds), or combinations thereof, as described below, to maintain or achieve a desired level of acidic species or to reduce the formation of acidic species during cell culture.

### Adjusting CO₂ Level to Modulate Acidic Species

In certain embodiments, the CO₂ level of the cell culture is modulated (e.g., increased or decreased) in order to produce a composition of the invention comprising a protein of interest, e.g., an antibody or antigen-binding portion thereof, for example, an anti-GM-CSFRα antibody such as mavrilimumab, having a reduced level of variants and/or impurities, e.g., a reduced level of acidic species (see the Examples Section, below). Such adjustments include increasing the CO₂ level of the cell culture and/or maintaining the CO₂ level of the cell culture at about 1-20%. A high level of CO₂ is generally considered as an undesirable condition for cell culture. However, the inventors of the present invention have unexpectedly found that by increasing the level of CO₂ in the cell culture in the methods of the present invention, which in turn results in a decrease in cell culture pH, a significant reduction of the level of acidic species in the protein product, e.g., an antibody, or antigen binding portion thereof, for example, an anti-GM-CSFRα antibody such as mavrilimumab, is achieved.

In certain embodiments, the CO₂ level of the cell culture is maintained at about 0.1%, 0.5%, 1%, 2%, 3%, 4%, 5%, 6%, 7%, 9%, 10%, 11%, 12%, 13%, 14%, 15%, 16%, 17%, 18%, 19%, or 20%. In certain embodiments, the CO₂ level of the cell culture is maintained at about 0.1-10%, 0.1-0.5%, 0.1-5%, 1-10%, 2-9%, 3-8%, 4-7%, 5-8%, 4-9%, or 1-5% and ranges within one or more of the preceding.

In certain embodiments, pCO₂ level of the cell culture is maintained at about 200 mmHg, about 190 mmHg, about 180 mmHg, about 170 mmHg, about 160 mmHg, about 150 mmHg, 140 mmHg, 130 mmHg, 120 mmHg, 110 mmHg, 100 mmHg, 90 mmHg, 85 mmHg, 80 mmHg, 75 mmHg, 70 mmHg, 65 mmHg, 60 mmHg, 55 mmHg, 50 mmHg, 45 mmHg, 40 mmHg, 35 mmHg, 30 mmHg, 25 mmHg, 20 mmHg, 15 mmHg, 10 mmHg or 5 mmHg. In some embodiments, the pCO₂ level of the cell culture is maintained at about 5-200 mmHg, about 10-190 mmHg, about 15-180 mmHg, about 20-170 mmHg, about 25-160 mmHg, about 30-150 mmHg, about 35-140 mmHg, about 40-130 mmHg, about 45-120 mmHg, about 50-110 mmHg, about 5-100 mmHg, about 10-110 mmHg, about 20-120 mmHg, about 30-130 mmHg, about 40-140 mmHg, about 50-150 mmHg, about 60-160 mmHg, about 70-170 mmHg, about 80-180 mmHg, about 90-190 mmHg or about 100-200 mmHg.

The CO₂ level of the cell culture is maintained in such a manner as to produce a composition comprising a protein of interest, e.g., an antibody, or antigen binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding.

In some embodiments, the composition comprises a clarified harvest from cell culture, wherein the clarified harvest comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the clarified harvest comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding acidic species, and ranges within one or more of the preceding.

In certain embodiments, the CO₂ level of the cell culture is maintained in such a manner as to reduce the amount of acidic species in a protein or antibody composition by about 1%, 2%, 3%, 4%, 5%, 6%, 7%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, and ranges within one or more of the preceding.

In certain embodiments, the CO₂ level of the cell culture is either increased or decreased in order to increase or decrease the amount of acidic species and/or the rate at which such acidic species form. In some embodiments, the percent CO₂ level of the cell culture is increased by about 0.1, 0.2, 0.3, 0.4, 0.5, 0.6, 0.7, 0.8, 0.9, 1.0, 1.5, 2, 2.5, 3, 3.5, 4, 4.5, 5, 6, 7, 8, 9, or 10 in order to decrease the amount of acidic species and/or the rate at which such acidic species form. For example, the percent CO₂ level of the cell culture is increased by 0.5 from 3% to 3.5% in order to decrease the amount or rate of acidic species.

In certain embodiments, the pH is either increased or decreased by increasing or decreasing the CO₂ level of the cell culture in order to increase or decrease the amount of acidic species and/or the rate at which such acidic species form. In some embodiments, the CO₂ level of the cell culture is increased in order to decrease the pH of the cell culture, initially at between about 6.5-7.0, by between about 0.01-0.5, about 0.02-0.4, about 0.05-0.3, or about 0.1-0.5, about 0.1-0.4, about 0.1-0.3, or about 0.1-0.2. In another embodiment the CO₂ level of the cell culture is increased to reduce the pH from about 6.9 to about 6.75, wherein the amount of acidic species produced during cell culture and the rate of acidic species formation, in the context of a clarified harvest, is decreased.

In certain embodiments, the CO₂ level of the cell culture is modulated at different time points during incubation. For example, but not limited to, increasing or decreasing the CO₂ level of the cell culture may occur at an earlier time point, e.g., at Day 1, 2, 3, 4, 5, 6, 7, 8, 9, or 10, during the incubation or production period, or a later time point, e.g., at the third last day, the second last day, or the last day of the incubation or production period. The cell culture CO₂ level may be changed, e.g., increased or decreased, multiple times throughout the incubation or production period, e.g., once, twice, or three times during the incubation period, in order to achieve a desirable level of acidic species in the composition.

In certain embodiments, a composition of the invention having a reduced level of variants and/or impurities, e.g., a reduced level of acidic species, can be produced from cell culture by maintaining the CO₂ level of the cell culture expressing the protein of interest as described herein along with choice of suitable pH or pH shift strategies, and/or suitable temperature or temperature shift strategies, as described above. These culture conditions can be used in various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, and any other configuration appropriate for optimal growth and productivity of the desired cell line

These methods of modulating CO₂ level may also be used in combination with methods of modulating pH and/or temperature, methods of modulating the lactate level in cell culture or lactate production from the cell culture, or supplementation of culture media with additives such as one or more cell culture supplements, or combinations thereof, as described herein, to maintain or achieve a desired level of acidic species or to reduce the formation of acidic species during cell culture.

### Adjusting Lactate Level to Modulate Acidic Species

In certain embodiments, the lactate level of the cell culture is modulated (e.g., increased or decreased) in order to produce a composition of the invention comprising a protein of interest, e.g., an antibody or antigen-binding portion thereof, for example, an anti-GM-CSFRα antibody such as mavrilimumab, having a reduced level of variants and/or impurities, e.g., a reduced level of acidic species (see the Examples Section, below). Such adjustments include increasing the lactate level in the cell culture and/or maintaining the lactate level of the cell culture at a certain level, e.g., about 0.1-5 g/L. A high level of lactate and/or a lower pH is generally considered undesirable for cell culture. However, the inventors of the present invention have unexpectedly found that by increasing the level of lactate in the cell culture in the methods of the present invention, which in turn results in a decrease in cell culture pH, a significant reduction of the level of acidic species in the protein product, e.g., an antibody, or antigen binding portion thereof, for example, an anti-GM-CSFRα antibody such as mavrilimumab, is achieved.

In certain embodiments, the lactate level of the cell culture is maintained at about 0.1 g/L, 0.2 g/L, 0.3 g/L, 0.4 g/L, 0.5 g/L, 0.6 g/L, 0.7 g/L, 0.8 g/L, 0.9 g/L, 1 g/L, 1.1 g/L, 1.2 g/L, 1.3 g/L, 1.4 g/L, 1.5 g/L, 1.6 g/L, 1.7 g/L, 1.8 g/L, 1.9 g/L, 2 g/L, 2.1 g/L, 2.2 g/L, 2.3 g/L, 2.4 g/L, 2.5 g/L, 2.6 g/L, 2.7 g/L, 2.8 g/L, 2.9 g/L, 3 g/L, 4 g/L or 5 g/L. In certain embodiments, the lactate level of the cell culture is maintained at about 0.1-5 g/L, at about 0.1-4 g/L, at about 0.1-3 g/L, about 0.1-2 g/L, about 0.2-2 g/L, about 0.3-2 g/L, about 0.4-2 g/L, about 0.5-2 g/L, about 0.6-2 g/L, about 0.7-2 g/L, about 0.8-2 g/L, about 0.9-2 g/L, about 0.1-1.9, about 0.2-1.8, about 0.3-1.7, about 0.4-1.6, about 0.5-1.5 g/L, about 0.6-1.4, about 0.7-1.3, about 0.8-1.2, or about 0.9-1.1. In certain embodiments, the lactate level of the cell culture is maintained at about 0.1-2 g/L.

In some embodiments, the lactate level of the cell culture is modulated by supplementing the cell culture with additional cell culture feeds or supplements, thereby increasing lactate production by cells within the culture.

The lactate level is maintained in such a manner as to produce a composition comprising a protein of interest, e.g., an antibody or antigen-binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding.

In some embodiments, the composition comprises a clarified harvest from cell culture, wherein the clarified harvest comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the clarified harvest comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding acidic species, and ranges within one or more of the preceding.

In certain embodiments, the lactate level is maintained in such a manner as to reduce the amount of acidic species in a protein or antibody composition by about 1%, 2%, 3%, 4%, 5%, 6%, 7%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, and ranges within one or more of the preceding.

In certain embodiments, pH is either increased or decreased by increasing or decreasing the lactate level of the cell culture in order to increase or decrease the amount of acidic species and/or the rate at which such acidic species form. In some embodiments, the lactate level of the cell culture is increased in order to decrease the pH of the cell culture, initially at between about 6.5-7.0, by between about 0.01-0.5, about 0.02-0.4, about 0.05-0.3, or about 0.1-0.5, about 0.1-0.4, about 0.1-0.3, or about 0.1-0.2. In another embodiment, the lactate level of the cell culture is increased to reduce the pH from about 6.9 to about 6.75, wherein the amount of acidic species produced during cell culture and the rate of acidic species formation, in the context of a clarified harvest, is decreased.

In certain embodiments, the level of lactate in cell culture is either increased or decreased in order to increase or decrease the amount of acidic species and/or the rate at which such acidic species form.

In certain embodiments, the lactate level of the cell culture is modulated at different time points during incubation. For example, but not limited to, increasing or decreasing the lactate level of the cell culture may occur at an earlier time point, e.g., at Day 1, 2, 3, 4, 5, 6, 7, 8, 9, or 10, during the incubation or production period, or a later time point, e.g., at the third last day, the second last day, or the last day of the incubation or production period. The cell culture lactate level may be changed, e.g., increased or decreased, multiple times throughout the incubation or production period, e.g., once, twice, or three times during the incubation period, in order to achieve a desirable level of acidic species in the composition.

In certain embodiments, a composition of the invention having a reduced level of variants and/or impurities, e.g., a reduced level of acidic species, can be produced from cell culture by increasing the lactate level and/or maintaining the lactate level of the cell culture expressing the protein of interest as described herein along with choice of suitable pH, CO₂, temperature or temperature shift strategies, as described above. These culture conditions can be used in various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, and any other configuration appropriate for optimal growth and productivity of the desired cell line.

These methods of modulating the lactate level in cell culture or lactate production from the cell culture may also be used in combination with methods of modulating pH, temperature and/or CO₂ level, or supplementation of culture media with additives such as one or more cell culture supplements, or combinations thereof, as described herein, to maintain or achieve a desired level of acidic species or to reduce the formation of acidic species during cell culture.

### Adjusting Cell Culture Supplements to Modulate Acidic Species

In certain embodiments, one or more cell culture supplements (media feeds) can be added in order to produce a composition of the invention comprising a protein of interest, e.g., an antibody or antigen-binding portion thereof, for example, an anti-GM-CSFRα antibody such as mavrilimumab, having a reduced level of variants and/or impurities, e.g., a reduced level of acidic species (see the Examples Section, below).

The cell culture supplements are intended to enhance cell culture performance and increase yields of recombinant proteins from cell cultures. Any known cell culture supplements are suitable for the methods of the present invention. In some embodiments, the one or more cell culture supplements are chemically defined and animal-derived component-free, and are optimized for high-yield protein production in fed-batch processes. In some embodiments, the one or more cell culture supplements do not contain any growth factors (such as insulin), peptides, hydrolysates, phenol red, or 2-merCapto™ ethanol, ensuring batch-to-batch consistency and increased cell culture process efficiency. In some embodiments, the one or more cell culture supplements have a pH close to neutral and contains amino acids, vitamins, salts, trace elements, and glucose. In other embodiments, the one or more cell culture supplements have an alkaline pH and are a concentrated solution of amino acids.

In some embodiments, the one or more culture supplements comprise HyClone™ Cell Boost™ 7a (Cytiva Life Sciences, Amersham, UK). In other embodiments, the one or more cell culture supplements comprise HyClone™ Cell Boost™ 7 b (Cytiva Life Sciences, Amersham, UK). The recommended ratio of Cell Boost 7a to 7b is about 10 to 1 (v/v).

The total amount of cell culture supplements added and the specific feeding regime are adjusted according to the nutritional requirements of each specific cell culture. In certain embodiments, one or more cell culture supplements is added to the cell culture at a volume of about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5%, 6%, 7%, 8%, 9%, or 10% of the initial culture volume.

The cell culture supplement is added to the cell culture in such a manner as to produce a composition comprising a protein of interest, e.g., an antibody or antigen-binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding.

In some embodiments, the composition comprises a clarified harvest from cell culture, wherein the clarified harvest comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the clarified harvest comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 11-19%, about 11-16%, about 12-20%, about 12-38%, about 13-19%, about 15-30%, about 14-28%, or about 18-40% acidic species, and ranges within one or more of the preceding acidic species, and ranges within one or more of the preceding.

In certain embodiments, the cell culture supplements is added to the cell culture in such a manner as to reduce the amount of acidic species in a protein or antibody composition by about 1%, 2%, 3%, 4%, 5%, 6%, 7%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, and ranges within one or more of the preceding.

In certain embodiments, the level of cell culture supplements in cell culture is either increased or decreased in order to increase or decrease the amount of acidic species and/or the rate at which such acidic species form. In some embodiments, a cell culture supplement at a level of about 0.1-3% is added to the cell culture, and the level of the cell culture supplement is further increased, e.g., by about 50% or greater of the initial level, in order to decrease the amount of acidic species and/or the rate at which such acidic species form. For example, but not by way of limitation, as detailed in Example 1 below, an increase in cell culture supplement level from 2% to about 3% or from 0.2% to about 0.3% can be employed to decrease the acidic species during cell culture and the rate of acidic species formation in the context of a clarified harvest.

In certain embodiments, the cell culture supplement level of the cell culture is modulated at different time points during incubation. For example, addition of cell culture supplement may occur at an earlier time point, e.g., at Day 1, 2, 3, 4, 5, 6, 7, 8, 9, or 10, during the incubation or production period, or a later time point, e.g., at the third to last day, the second to last day, or the last day of the incubation or production period. The amount of cell culture supplement being added to the cell culture may be changed, e.g., increased or decreased, multiple times throughout the incubation period, e.g., once, twice, or three times during the incubation or production period, in order to achieve a desirable level of acidic species in the composition.

In some embodiments, a cell culture supplement is at a level of about 0.1-3% is added to the cell culture during Day 2-Day 8, e.g., Day 2, 3, 4, 5, 6, 7, or 8, of the incubation or production period, and the level of the cell culture supplement is further increased, e.g., by more than about 20%, about 30%, about 40%, about 50%, about 60%, about 70%, about 80%, about 90%, about 100%, about 150%, or about 200% of the initial level, during Day 4-Day 10, e.g., Day 4, 5, 6, 7, 8, 9 or 10, of the incubation or production period. In some embodiments, a cell culture supplement at a level of about 0.1-3% is added to the cell culture on Day 2 or Day 3 of the incubation or production period, and the level of the cell culture supplement is further increased, e.g., by about 50% or more of the initial level, on Day 5 or Day 6 of the incubation or production period.

In some embodiments, a cell culture supplement is added to the culture media on Day 2 or Day 3 at about 2% of initial volume, and added again on Day 5 or Day 6 at about 3% of initial volume. In other embodiments, a cell culture supplement is added to the culture media on Day 2 or Day 3 at about 0.2% of initial volume, and added again on Day 5 or Day 6 at about 0.3% of initial volume. In some embodiments, a cell culture supplement is added to the culture media on Day 2 or Day 3 at about 2% of initial volume, added again on Day 5 or Day 6 at about 3% of initial volume, and on Day 9 at about 2% of initial volume. In some embodiments, a cell culture supplement is added to the culture media on Day 2 or Day 3 at about 0.2% of initial volume, added again on Day 5 or Day 6 at about 0.3% of initial volume, and on Day 9 at about 0.2% of initial volume. In some embodiments, a first cell culture supplement is added to the culture media on Day 2 or Day 3 at about 2% of initial volume and added again on Day 5 or Day 6 at about 3% of initial volume, and a second cell culture supplement is added to the culture media on Day 2 or Day 3 at about 0.2% of initial volume and added again on Day 5 or Day 6 at about 0.3% of initial volume. In one embodiment, a first cell culture supplement is added to the culture media on Day 3 at about 2% of initial volume and added again on Day 5 at about 3% of initial volume, and a second cell culture supplement is added to the culture media on Day 3 at about 0.2% of initial volume and added again on Day 5 at about 0.3% of initial volume. In another embodiment, a first cell culture supplement is added to the culture media on Day 3 at about 2% of initial volume and added again on Day 6 at about 3% of initial volume, and a second cell culture supplement is added to the culture media on Day 3 at about 0.2% of initial volume and added again on Day 6 at about 0.3% of initial volume. In some embodiments, the first cell supplement is a cell culture media feed, such as Cell Boost 7a, and the second supplement is a cell culture media feed, such as Cell Boost 7b.

In some embodiments, addition or one or more cell culture supplement results in an increase in lactate production, an increase in osmolality, an increase in cell viability, and/or a decrease in pH of the cell culture.

Addition of one or more supplements may be based on measured amount of acidic species. The resulting media can be used in various cultivation methods including, but not limited to, batch, fed-batch, chemostat and perfusion, and with various cell culture equipment including, but not limited to, shake flasks with or without suitable agitation, spinner flasks, stirred bioreactors, airlift bioreactors, membrane bioreactors, reactors with cells retained on a solid support or immobilized/entrapped as in microporous beads, and any other configuration appropriate for optimal growth and productivity of the desired cell line. In addition, the harvest criterion for these cultures may be chosen, for example, based on choice of harvest viability or culture duration, to further optimize a certain targeted acidic species profile.

These methods of adjusting cell culture supplements may also be used in combination with methods of modulating pH, temperature and/or CO₂ level, methods of modulating lactate level in cell culture or lactate production from cell culture, or combinations thereof, as described herein, to maintain or achieve a desired level of acidic species or to reduce the formation of acidic species during cell culture.

### IV. Preparation of Compositions Using Downstream Process Technologies

The invention provides methods and compositions for producing a preparation comprising a protein of interest, e.g., an antibody or antigen-binding portion thereof, for example, an anti-GM-CSFRα antibody such as mavrilimumab, having a reduced level of variants and/or impurities, e.g., a reduced level of product-related substances, e.g., protein aggregates, fragments, e.g., half antibody, or charged species, e.g., acidic species or basic species; and/or a reduced level of process-related impurities, e.g., host cell protein. In certain embodiments, the compositions of the present invention include, but are not limited to, compositions comprising an anti-GM-CSFRα antibody or antigen-binding portion thereof, such as marvilimumab, having a reduced level of variants and/or impurities, e.g., a reduced level of product-related substance, e.g., protein aggregates, fragments, e.g., half antibody, or charged species, e.g., acidic species or basic species; and/or a reduced level of process-related impurities, e.g., host cell protein. Such variants and/or impurities-reduced compositions address the need for improved product characteristics, including, but not limited to, product stability, product safety and product efficacy.

In certain embodiments, the present invention is directed to a method for producing a preparation comprising a protein of interest, e.g., an antibody or antigen-binding portion thereof, for example, an anti-GM-CSFRα antibody such as mavrilimumab, having a reduced level of variants and/or impurities, e.g., a reduced level of product-related substance, e.g., protein aggregates, fragments (for example, half antibody), or charged species (for example, acidic species or basic species); and/or a reduced level of process-related impurities, e.g., host cell protein, by subjecting a sample comprising the protein of interest and variants and/or impurities to a chromatography resin, such as a cation exchange (CEX) chromatography resin, an anion exchange (AEX) chromatography resin, and/or a mixed-mode (MM) chromatography resin.

In some embodiments, the present invention provides a method for producing a preparation comprising a protein of interest, e.g., an antibody or antigen-binding portion thereof, for example, an anti-GM-CSFRα antibody such as mavrilimumab, having a reduced level of half antibody, by subjecting a sample comprising the protein of interest and half antibody to a cation exchange chromatography resin, and/or a mixed-mode chromatography resin.

In some embodiments, the present invention provides a method reducing the level of half antibody in a preparation comprising a protein of interest, e.g., an antibody or antigen-binding portion thereof, for example, an anti-GM-CSFRα antibody such as mavrilimumab, by subjecting a sample comprising the protein of interest and half antibody to a cation exchange chromatography resin, and/or a mixed-mode chromatography resin.

In some embodiments, the present invention provides a method of producing a preparation comprising an anti-GM-CSFRα antibody, or antigen-binding portion thereof, such as mavrilimumab, having a reduced level of acidic species, by subjecting a sample comprising the anti-GM-CSFRα antibody, or antigen-binding portion thereof, and acidic species to an anion exchange chromatography resin or a mixed mode chromatography resin.

In some embodiments, the present invention provides a method of reducing the level of acidic species in a preparation comprising an anti-GM-CSFRα antibody, or antigen-binding portion thereof, such as mavrilimumab, by subjecting a sample comprising the anti-GM-CSFRα antibody, or antigen-binding portion thereof, and acidic species to an anion exchange chromatography resin or a mixed mode chromatography resin.

In some embodiments, the present invention provides a method of producing a preparation comprising a protein of interest, such as an antibody or antigen-binding portion thereof, such as mavrilimumab, having a reduced level of high molecular weight aggregates and/or host cell proteins, by subjecting a sample comprising the protein of interest, high molecular weight aggregates and/or host cell proteins (HCP) to a chromatography resin, such as a cation exchange chromatography resin, an anion exchange chromatography resin and/or a mixed mode chromatography resin.

In some embodiments, the present invention provides a method of reducing the level of high molecular weight aggregates and/or host cell proteins in a preparation comprising a protein of interest, e.g., an antibody or antigen-binding portion thereof, for example, an anti-GM-CSFRα antibody such as mavrilimumab, by subjecting a sample comprising the protein of interest, high molecular weight aggregates and/or host cell proteins (HCP) to a chromatography resin, such as a cation exchange chromatography resin, an anion exchange chromatography resin and/or a mixed mode chromatography resin.

In certain embodiments, the compositions of the present invention may be produced using the downstream process technologies (e.g., purification), as described herein, following cell culture of a protein. The downstream process technologies may be used alone or in combination with the upstream process technologies described in Section III, above, and as described in Examples 2 and 3.

In one embodiment, the downstream process technologies described herein, alone or in combination with one or more upstream process technology, produce a composition comprises a protein of interest, e.g., an antibody or antigen-binding portion thereof, for example, an anti-GM-CSFRα antibody such as mavrilimumab, having a reduced level of variants and/or impurities, e.g., a reduced level of product-related substances, e.g., protein aggregates, fragments (for example, half antibody), or charged species (for example, acidic species or basic species); and/or a reduced level of process-related impurities, e.g., host cell protein.

In some embodiments, the method results in a composition comprising a protein of interest, e.g., an antibody or antigen-binding portion thereof, for example, an anti-GM-CSFRα antibody such as mavrilimumab, having a reduced level of half antibody, wherein the composition comprises less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the composition comprises about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one or more of the preceding.

In some embodiments, the methods result in a composition comprising a protein of interest, e.g., an antibody or antigen-binding portion thereof, for example, an anti-GM-CSFRα antibody such as mavrilimumab, having a reduced level of acidic species, wherein the composition comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the composition comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding.

In some embodiments, the method results in a composition comprising a protein of interest, e.g., an antibody or antigen-binding portion thereof, for example, an anti-GM-CSFRα antibody such as mavrilimumab, having a reduced level of basic species, wherein the composition comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% basic species, and ranges within one or more of the preceding. In some embodiments, the composition comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding.

In some embodiments, the method results in a composition comprising a protein of interest, e.g., an antibody or antigen-binding portion thereof, for example, an anti-GM-CSFRα antibody such as mavrilimumab, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the composition comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 58-63%, about 58-67%, about 53-61%, about 46-67%, or about 46-66% main species, and ranges within one or more of the preceding.

In some embodiments, method results in a composition comprises a protein of interest, e.g., an antibody or antigen-binding portion thereof, for example, an anti-GM-CSFRα antibody such as mavrilimumab, having a reduced level of high molecular weight aggregates, wherein the composition comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the composition comprises about 0.01-10%, about 0.01 to 5%, about 0.01 to 1%, about 0.04-0.2%, about 0.1-0.4%, about 0.04-0.4%, about 0.04-0.8%, about 0.5-0.8%, about 1-10%, about 2-10%, about 3-10%, or about 4-10% high molecular weight aggregates, and ranges within one or more of the preceding.

In some embodiments, the method results in a composition comprises a protein of interest, e.g., an antibody or antigen-binding portion thereof, for example, an anti-GM-CSFRα antibody such as mavrilimumab, having a reduced level of protein fragments, wherein the composition comprises less than about 10%, about 9%, about 8%, about 7%, about 6%, about 5%, about 4%, about 3%, about 2%, about 1%, about 0.5%, about 0.4%, about 0.3%, about 0.2% or about 0.1% protein fragments, and ranges within one or more of the preceding. In some embodiments, the composition comprises about 0.1-10%, about 0.1-5%, about 0.1-3%, about 0.1-2%, about 0.5-1.5%, about 0.5-1.1%, about 0.4-0.8% or about 0.4-1.1% protein fragments, and ranges within one or more of the preceding.

In some embodiments, the method results in a composition comprising the protein of interest, e.g., an antibody or antigen-binding portion thereof, for example, an anti-GM-CSFRα antibody such as mavrilimumab, which comprises more than about 90%, about 91%, about 92%, about 93%, about 94%, about 95%, about 96%, about 97%, about 98%, about 99%, about 99.1% or about 99.5% of monomer of the protein of interest, and ranges within one or more of the preceding. In some embodiments, the composition comprises more than about 90% of monomer. In some embodiments, the composition comprises more than about 99.1% of monomer. In some embodiments, the composition comprises about 90-99.9%, about 90-95%, about 95-99.9%, about 99-99.9%, about 99.1-99.9%, about 98-99%, about 98-99.9%, or about 98.5-99.5% of monomer of the protein of interest. In some embodiments, the composition comprises about 98-99% monomer, and ranges within one or more of the preceding. In some embodiments, the composition comprises about 98-99.9% monomer.

In some embodiments, the method results in a composition comprises a protein of interest, e.g., an antibody or antigen-binding portion thereof, for example, an anti-GM-CSFRα antibody such as mavrilimumab, having a reduced level of host cell proteins, wherein the composition comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the composition comprises about 0.1-10 ppm, about 1-10 ppm, about 2-10 ppm, about 3-10 ppm, about 4-10 ppm, about 1-5 ppm, about 5-10 ppm, about 1-3 ppm, about 0.1-2 ppm, about 0.1-3 ppm, about 2-8, or about 0.1-8 ppm HCP, and ranges within one or more of the preceding.

In certain embodiments, the downstream process technologies described herein, alone or in combination with one or more upstream process technology, reduces the level of variants and/or impurities, e.g., product-related substances, e.g., protein aggregates, fragments, e.g., half antibody, or charged species, e.g., acidic species or basic species; and/or process-related impurities, e.g., host cell protein, in a protein or antibody composition, by about 1%, 2%, 3%, 4%, 5%, 6%, 7%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, and ranges within one or more of the preceding.

Protein Purification

Following upstream production of a protein of interest, e.g., an antibody or antigen-binding portion thereof, for example, an anti-GM-CSFRα antibody such as mavrilimumab, downstream process technologies can be used to purify the protein. For example, but not by way of limitation, once a clarified solution or mixture comprising the protein of interest, for example, an antibody or antigen binding fragment thereof, has been obtained, separation of the protein of interest from the protein variants and/or impurities, e.g., product-related substance, e.g., protein aggregates, fragments, e.g., half antibody, or charged species, e.g., acidic species or basic species; and/or process-related impurities, e.g., host cell proteins, can be effected using a combination of different purification techniques, including, but not limited to, ion exchange separation steps, mixed mode separation steps, affinity separation steps, and hydrophobic interaction separation steps singularly or in combination. The separation steps separate mixtures of proteins on the basis of their charge, degree of hydrophobicity, or size, or any combination thereof, depending on the particular form of separation, including chromatographic separation. In one aspect of the invention, separation is performed using chromatography, including cationic, anionic, and hydrophobic interaction. Several different chromatography resins are available for each of these techniques, allowing accurate tailoring of the purification scheme to the particular protein involved. Each of the separation methods result in the protein traversing at different rates through a column, to achieve a physical separation that increases as they pass further through the column, or adhere selectively to the separation medium. The proteins are then differentially eluted by different elution buffers. In some cases, the protein of interest is separated from variants and/or impurities when the variants and/or impurities preferentially adhere to the column's resin and the protein of interest does not, i.e., the protein of interest is present in the flow through fraction, while in other cases the protein of interest will adhere to the column's resin, while the variants and/or impurities are extruded from the column's resin during a wash cycle.

In certain embodiments, a composition of the present invention comprising a protein of interest, e.g., an antibody or antigen-binding portion thereof, for example, an anti-GM-CSFRα antibody such as mavrilimumab, is produced using chromatographic separation to identify the particular conditions, e.g., salt concentration, pH, buffer, temperature, load amount and conditions, washing conditions, and elution condition, sufficient to elicit the desired fractionation profiles, e.g., fractionation of product-related substances, e.g., protein aggregates, fragments, e.g., half antibody, or charged species, e.g., acidic species or basic species; and/or process-related impurities, e.g., host cell proteins, of a sample comprising the protein of interest and at least one such variants and/or impurities. In certain embodiments, the method further comprises pooling the resulting fractions comprising the desired compositions.

Primary Recovery and Virus Inactivation

In certain embodiments, the initial steps of the purification methods of the present invention involve the clarification and primary recovery of the protein of interest, e.g., an antibody or antigen-binding portion thereof, for example, an anti-GM-CSFRα antibody such as mavrilimumab, from a sample matrix. In certain embodiments, the primary recovery will include one or more centrifugation steps to separate the protein of interest=from the cells and cell debris. Centrifugation of the sample can be performed at, for example, but not by way of limitation, 7,000 g to approximately 12,750 g. In the context of large scale purification, such centrifugation can occur on-line with a flow rate set to achieve, for example, but not by way of limitation, a turbidity level of 150 NTU in the resulting supernatant. Such supernatant can then be collected for further purification, or in-line filtered through one or more depth filters for further clarification of the sample.

In certain embodiments, the primary recovery will include the use of one or more depth filtration steps to clarify the sample matrix and thereby aid in purifying the antibodies of interest in the present invention. In other embodiments, the primary recovery will include the use of one or more depth filtration steps post centrifugation to further clarify the sample matrix. Non-limiting examples of depth filters that can be used in the context of the instant invention include the Millistak+ X0HC, F0HC, D0HC, A1HC, B1HC depth filters (EMD Millipore), Cuno™ model 30/60ZA, 60/90 ZA, VR05, VR07, delipid depth filters (3M Corp.). A 0.2 μm filter such as Sartorius's 0.45/0.2 μm Sartopore™ bi-layer or Millipore's Express SHR or SHC filter cartridges typically follows the depth filters.

In certain embodiments, the primary recovery process can also be a point at which to reduce or inactivate viruses that can be present in the sample matrix. For example, any one or more of a variety of methods of viral reduction/inactivation can be used during or after the primary recovery phase of purification including heat inactivation (pasteurization), pH inactivation, buffer/detergent treatment, UV and γ-ray irradiation and the addition of certain chemical inactivating agents such as β-propiolactone or e.g., copper phenanthroline as described in U.S. Pat. No. 4,534,972. In certain embodiments of the present invention, the sample matrix is exposed to detergent viral inactivation during or after the primary recovery phase. In other embodiments, the sample matrix may be exposed to low pH inactivation during or after the primary recovery phase.

In those embodiments where viral reduction/inactivation is employed, the sample mixture can be adjusted, as needed, for further purification steps. For example, following low pH viral inactivation, the pH of the sample mixture is typically adjusted to a more neutral pH, e.g., from about 4.5 to about 8.5, from about 5 to about 8, from about 5.5 to about 7.5, or from about 6 to about 7, prior to continuing the purification process. Additionally, the mixture may be diluted with water for injection (WFI) to obtain a desired conductivity.

Affinity Chromatography

It is advantageous to subject a sample produced by the techniques of the instant invention to affinity chromatography to further purify the protein of interest, e.g., an antibody or antigen-binding portion thereof, for example, an anti-GM-CSFRα antibody such as mavrilimumab, away from variants and/or impurities. In certain embodiments the chromatographic material is capable of selectively or specifically binding to the protein of interest (“capture”). Non-limiting examples of such chromatographic material include: Protein A, Protein G, chromatographic material comprising, for example, an antigen bound by an antibody of interest, and chromatographic material comprising an Fc binding protein. In specific embodiments, the affinity chromatography step involves subjecting the primary recovery sample to a column comprising a suitable Protein A resin. In certain embodiments, Protein A resin is useful for affinity purification and isolation of a variety of antibody isotypes, particularly IgG1, IgG2, and IgG4. Protein A is a bacterial cell wall protein that binds to mammalian IgGs primarily through their Fc regions. In its native state, Protein A has five IgG binding domains as well as other domains of unknown function.

There are several commercial sources for Protein A resin. Suitable resins include, but not limited to, MabSelect SuRe LX, MabSelect SuRe™, MabSelect, MabSelect Xtra, rProtein A Sepharose from GE Healthcare, ProSep HC, ProSep Ultra, and ProSep Ultra Plus from EMD Millipore, MapCapture from Life Technologies.

The Protein A column can be equilibrated with a suitable buffer prior to sample loading. Following the loading of the column, the column can be washed one or multiple times using a suitable set of buffers. The Protein A column can then be eluted using an appropriate elution buffer. For example, glycine-HCL or citric acid can be used as an elution buffer. The eluate can be monitored using techniques well known to those skilled in the art. The eluate fractions of interest can be collected and then prepared for further processing.

The Protein A eluate may subject to a viral inactivation step either by detergent or low pH, provided this step is not performed prior to the Protein A capture operation. A proper detergent concentration or pH and time can be selected to obtain desired viral inactivation results. After viral inactivation, the Protein A eluate is usually pH and/or conductivity adjusted for subsequent purification steps.

The Protein A eluate may be subjected to filtration through a depth filter to remove turbidity and/or various impurities from the antibody of interest prior to additional chromatographic polishing steps. Examples of depth filters include, but not limited to, Millistak+X0HC, F0HC, D0HC, A1HC, and B1HC Pod filters (EMD Millipore), or Zeta Plus 30ZA/60ZA, 60ZA/90ZA, delipid, VR07, and VR05 filters (3M). The Protein A eluate pool may need to be conditioned to proper pH and conductivity to obtain desired impurity removal and product recovery from the depth filtration step.

The invention is not limited to capture of the protein of interest using Protein A chromatography. A non-Protein A chromatography capture step can also be carried out. For example, cation exchange capture and non-chromatographic methods, such as aqueous two phase extraction or precipitation, or other methods known in the art, can be used.

Cation Exchange Chromatography

The compositions of the present invention comprising a protein of interest, e.g., an antibody or antigen-binding portion thereof, for example, an anti-GM-CSFRα antibody such as mavrilimumab, can be produced by subjecting a sample comprising a protein of interest, to a cation exchange (CEX) separation step. In certain embodiments, the CEX step occurs after the above-described affinity chromatography, e.g., Protein A affinity, step.

The use of a cationic exchange material versus an anionic exchange material, such as those anionic exchange materials discussed in detail herein, is based on the local charges of the protein of interest in a given solution. Thereof, it is within the scope of this invention to employ a cationic exchange step prior to the use of an anionic exchange step, or an anionic exchange step prior to the use of a cationic exchange step. Furthermore, it is within the scope of this invention to employ only a cationic exchange step, only an anionic exchange step, or any serial combination of the two (including serial combinations of one or both ion exchange steps with the other chromatographic separation technologies described herein).

In performing the separation, the initial protein mixture can be contacted with the cation exchange material by using any of a variety of techniques, e.g., using a batch purification technique or a chromatographic technique, as described above in connection with Protein A.

For example, in the context of batch purification, cation exchange material is prepared in, or equilibrated to, the desired starting buffer. Upon preparation, or equilibration, a slurry of the cation exchange material is obtained. In some embodiments, the protein of interest, e.g., antibody, solution is contacted with the CEX resin to allow for protein adsorption to the resin. The solution comprising the variants and/or impurities may not bind to the CEX resin. Alternatively, in other embodiments, the solution comprising the variants and/or impurities may bind tighter to the CEX resin than the protein of interest. The resin can be subjected to one or more washing steps and/or elution steps. Alternatively, the variants and/or impurities may bind to the resin, while the protein of interest does not.

A packed cation-exchange chromatography column, cation-exchange membrane device, cation-exchange monolithic device, or depth filter media can be operated either in bind-elute mode, flow-through mode, or a hybrid mode wherein the product exhibits binding to the chromatographic material, yet can be washed from the column using a buffer that is the same or substantially similar to the loading buffer. In the bind-elute mode, the column or the membrane device is first conditioned with a buffer with appropriate ionic strength and pH under conditions where certain proteins will be immobilized on the resin based matrix. For example, in certain embodiments, during the feed load, the protein of interest will be adsorbed to the resin due to electrostatic attraction. After washing the column or the membrane device with the equilibration buffer or another buffer with different pH and/or conductivity, the product recovery is achieved by increasing the ionic strength (i.e., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution). In the flow-through mode, the column or the membrane device is operated at selected pH and conductivity such that the protein of interest does not bind to the resin or the membrane while the acidic species will either be retained on the column or will have a distinct elution profile as compared to the protein of interest. In the context of this hybrid strategy, acidic species will bind to the chromatographic material (or Flow Through) in a manner distinct from the protein of interest, e.g., while the protein of interest and certain aggregates and/or fragments of the protein of interest may bind the chromatographic material, washes that preferentially remove the protein of interest can be applied. The column is then regenerated before next use.

In certain embodiments, in the context of chromatographic separation, a chromatographic apparatus, commonly cylindrical in shape, is employed to contain the chromatographic support material (e.g., CEX resin) prepared in an appropriate buffer solution. The chromatographic apparatus, if cylindrical, can have a diameter of about 5 mm to about 2 meters, and a height of 5 cm to 50 cm, and in certain embodiments, particularly for large scale processing, a height of ≤30 cm is employed. Once the chromatographic material is added to the chromatographic apparatus, a sample containing the protein of interest, e.g., an antibody, is contacted to the chromatographic resin to induce separation. Any portion of the solution that does not bind to the chromatographic resin, e.g., which may comprise the protein of interest or the variants and/or impurities, e.g., product-related substances, e.g., protein aggregates, fragments, e.g., half antibody, charged variants, e.g. acidic or basic species, and/or process-related impurities, e.g., host cell proteins, depending on the CEX resin being employed, is separated from the chromatographic resin by washing the resin and collecting fractions. The chromatographic resin can be subjected to one or more wash steps. If desired, the chromatographic resin can then be contacted with a solution designed to desorb or elute any components of the solution that have bound to the chromatographic resin.

In certain embodiments, a wash step can be performed in the context of CEX chromatography using conditions similar to the load conditions or alternatively by changing the pH and/or the ionic strength/conductivity of the wash buffer in a step wise or linear gradient manner. The resulting flow through and wash fractions can be analyzed and appropriate fractions pooled to achieve the desired reduction in variants and/impurities.

In certain embodiments, the aqueous salt solution used as both the loading and wash buffer has a pH that is lower than the isoelectric point (pI) of the protein of interest. In certain embodiments, the pH of the loading and wash buffer is about 0 to 5 units lower than the pI of the protein. In certain embodiments, the pH of the loading and wash buffer is about 1 to 2 units lower than the pI of the protein. In certain embodiments, the pH of the loading and wash buffer is about 1 to 1.5 units lower than the pI of the protein.

In certain embodiments, the aqueous salt solution used as the elution buffer has a pH that is higher than the isoelectric point (pI) of the protein of interest. In certain embodiments, the pH of the elution buffer is about 0 to 5 units higher than the pI of the protein. In certain embodiments, the pH of the elution buffer is about 1 to 2 units higher than the pI of the protein. In certain embodiments, the pH of the elution buffer is about 1 to 1.5 units higher than the pI of the protein.

In certain embodiments, the pH of the loading, wash or elution buffer is about 3.5-10.5, about 4-10, about 4.5-9.5, about 5-9, abut 5.5-8.6, about 6-8, about 6.5-7.5, about 6-7, about 5-8, about 4-7, about 5-7, about 5-6, about 5-5.5. In certain embodiments, the pH of the loading, wash or elution buffer is about 4.9, 5, 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.5, 9, 9.5, or 10.

Buffer systems suitable for use in the CEX methods include, but are not limited to tris formate, tris acetate, ammonium sulfate, sodium acetate, sodium chloride, and sodium sulfate. In certain embodiments, the conductivity and pH of the buffer is adjusted by increasing or decreasing the concentration of cationic or anionic agents. In certain non-limiting embodiments, the cationic agent is selected from the group consisting of sodium, Tris, tromethalmine, ammonium, arginine, histidine, or combinations thereof. In certain non-limiting embodiments, the anionic agent is selected from the group consisting of formate, acetate, citrate, chloride anion, sulphate, phosphate or combinations thereof.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 130 mM, 120 mM, 110 mM, 100 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM sodium acetate. In some embodiments, the elution buffer comprises about 1-500 mM, about 10-250 mM, about 10-150 mM, about 10-100 mM, about 20-90 mM, about 30-80 mM, about 40-70 mM, or about 50-60 mM, about 40-60 mM sodium acetate. In some embodiments, the elution buffer comprises about 40-60 mM sodium acetate.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 130 mM, 125 mM, 120 mM, 110 mM, 100 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM sodium chloride. In some embodiments, the elution buffer comprises about 1-500 mM, about 10-250 mM, about 10-150 mM, about 10-100 mM, about 20-90 mM, about 30-80 mM, about 40-70 mM, about 50-60 mM, about 50-250 mM, about 50-150 mM or about 40-60 mM sodium chloride. In some embodiments, the elution buffer comprises about 40-60 mM sodium chloride.

In some embodiments, the elution buffer comprises about 50 mM sodium acetate, about 55 mM sodium chloride, and a pH of about 5.35.

Any cation exchange chromatography resins known in the art are suitable for the preparation of the composition of the present invention. Exemplary CEX resins include, but are not limited to, sulfhydryl (XS), sulfonate (S), sulfate, carboxymethyl (CM), sulfoethyl (SE), sulfopropyl (SP), phosphate (P) and sulfonate (S). In certain embodiments, the resin employed for a CEX separation is POROS™ XS. POROS™ XS is a strong cation exchanger of a support matrix of cross-linked poly(styrene-divinylbenzene) with a sulfopropyl functionality. In certain embodiments, the resin employed for a CEX separation is Capto™ S Impact.Capto™ ™ S Impact is a cation exchanger of a high-flow agarose matrix with a sulfonate group and a neutral pyrrolidone. In certain embodiments, the resin employed for a CEX separation is Toyopearl™ sulfate 650. Toyopearl™ sulfate 650 is a cation exchange resin of polymethacrylate beads with a proprietary sulfate containing ligand. In certain embodiments, the resin employed for a CEX separation is Toyopearl™ GigaGap CM 650M. Toyopearl™ CM GigaGap 650M is a cation exchange resin composed of polymethacrylate beads that have been chemically modified to provide a higher number of cationic binding sites and functionalized with carboxymethyl groups. Additional CEX resins include, but are not limited to, Capto™ SP ImpRes, CM™ Ceramic HyperD grade F, Eshmuno™ S, Nuvia™ C Prime, Nuvia™ S, Poros™ HS; Poros™ HQ, Toyopearl™ GigaCap S 650M, Toyopearl™ MX Trp 650M. It is noted that CEX chromatography can be used with MM resins, described herein.

In certain embodiments, the protein of interest, e.g., an antibody or antigen-binding portion thereof, for example, an anti-GM-CSFRα antibody such as mavrilimumab, is loaded onto the cation exchange chromatography resin at a level of about 10-100 g/L, about 20-90 g/L, about 30-80 g/L, about 30-60 g/L, about 40-70 g/L, or about 50-60 g/L. In certain embodiments, the protein of interest is loaded onto the cation exchange chromatography resin at a level of about 30-60 g/L.

In certain embodiments, the methods of the instant invention can be used to selectively remove, significantly reduce, or essentially remove all of variants and/or impurities, e.g., product-related substances, e.g., protein aggregates, fragments, e.g., half antibody, charged variants, e.g. acidic or basic species, and/or process-related impurities, e.g., host cell proteins, from the protein of interest, wherein the variants and/or impurities, e.g., product-related substances, e.g., protein aggregates, fragments, e.g., half antibody, charged variants, e.g. acidic or basic species, and/or process-related impurities, e.g., host cell proteins, are collected in the Flow Through and wash fractions, and the protein of interest is enriched in the elution fraction, thereby producing protein compositions that have a reduced level of variants and/or impurities.

In certain embodiments, the eluate fractions comprising a protein of interest, e.g., an antibody or antigen-binding portion thereof, for example, an anti-GM-CSFRα antibody such as mavrilimumab, collected from the CEX chromatography step comprise a reduced level of variants and/or impurities, e.g., product-related substances, e.g., protein aggregates, fragments, e.g., half antibody, charged variants, e.g. acidic or basic species, and/or process-related impurities, e.g., host cell proteins.

In some embodiments, the eluate fraction comprising a protein of interest, e.g., an antibody or antigen-binding portion thereof, for example, an anti-GM-CSFRα antibody such as mavrilimumab, comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the eluate fraction comprises about 0.1-25%, about 0.1-20%, about 1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one of more of the preceding.

In some embodiments, the eluate fraction comprising a protein of interest, e.g., an antibody or antigen-binding portion thereof, for example, an anti-GM-CSFRα antibody such as mavrilimumab, comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the eluate fraction comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% basic species, and ranges within one or more of the preceding. In some embodiments, the eluate fraction comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the eluate fraction comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 59-61%, about 58-63%, about 58-67%, about 53-61%, about 46-67%, or about 46-66% main species, e.g., anti-GM-CSFRα antibody such as mavrilimumab, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the eluate fraction comprises about 0.01-10%, about 0.01 to 5%, about 0.01 to 1%, about 0.01-0.4%, about 0.04-0.2%, about 0.1-0.4%, about 0.04-0.4%, about 0.04-0.8%, about 0.5-0.8%, about 0.1-6%, about 1-10%, about 2-10%, about 3-10%, or about 4-10% high molecular weight aggregates, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.5%, about 0.4%, or about 0.3% protein fragments, e.g., antibody fragments, and ranges within one or more of the preceding. In some embodiments, the eluate fraction comprises about 0.1-10%, about 0.1-5%, about 0.1-3%, about 0.1-2%, about 0.1-1.1%, about 0.1-0.8%, about 0.6-1.5%, about 0.5-1.5%, about 0.5-1.1%, about 0.4-0.8% or about 0.4-1.1% protein fragments, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 90%, about 91%, about 92%, about 93%, about 94%, about 95%, about 96%, about 97%, about 98%, about 99%, about 99.1% or about 99.5% of monomer of the protein of interest, e.g., antibody monomer, and ranges within one or more of the preceding. In some embodiments, the eluate fraction comprises about 90-99.9%, about 90-95%, about 94-99.9%, about 95-99.9%, about 99-99.9%, about 99.1-99.9%, about 98-99%, about 98-99.9%, or about 98.5-99.5% of monomer of the protein of interest, e.g., antibody monomer.

In some embodiments, the eluate fraction comprising a protein of interest, e.g., an antibody or antigen-binding portion thereof, for example, an anti-GM-CSFRα antibody such as mavrilimumab, comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the eluate fraction comprises about 0.1-10, about 1-10, about 2-10, about 3-10, about 4-10, about 1-5, about 5-10, about 1-3, about 0.1-2, about 0.1-3, about 2-8, or about 0.1-8 ppm HCP, and ranges within one or more of the preceding.

In certain embodiments, the loading, pH, conductivity of the CEX chromatography step, as well as elution pH and/or conductivity, can be modified to achieve a desired distribution of variants and/or impurities away from the protein of interest, e.g., the antibody or antigen binding portion thereof, for example, an anti-GM-CSFRα antibody such as mavrilimumab,

In certain embodiments, a CEX chromatographic separation can be performed and combinations of fractions can be pooled to achieve a combination of desired process-related impurity and/or product-relates substance levels, in addition to, or in place of merely modulating charge variant concentration.

In certain embodiments, spectroscopy methods such as UV, NIR, FTIR, Fluorescence, Raman may be used to monitor levels of variants and/or impurities, e.g., product-related substances, e.g., charge variants, aggregates, fragments of the protein of interest, and/or process-related impurities, e.g., host cell proteins, in an on-line, at-line or in-line mode, which can then be used to control the level of variants and/or impurities in the pooled material collected from the CEX effluent. In certain embodiments, on-line, at-line or in-line monitoring methods can be used either on the effluent line of the chromatography step or in the collection vessel, to enable achievement of the desired product quality/recovery. In certain embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed and the target product quality can be achieved. In certain embodiments, such measurements can be combined with in-line dilution methods such that ion concentration/conductivity of the load/wash can be controlled by feedback and hence facilitate product quality control.

In certain embodiments, a combination of CEX and AEX and/or MM methods can be used to prepare compositions of the invention comprising a protein of interest, e.g., an antibody or antigen-binding portion thereof, for example, an anti-GM-CSFRα antibody such as mavrilimumab, including certain embodiments where one technology is used in a complementary/supplementary manner with another technology. In some embodiments, such a combination can be performed such that certain sub-species are removed predominantly by one technology, such that the combination provides the desired final composition/product quality. In some embodiments, such combinations include the use of additional chromatography, filtration, nanofiltration, ultrafiltration/diafiltration (UF/DF) steps so as to achieve the desired product quality.

Anion Exchange Chromatography

In certain embodiments, the compositions comprising a protein of interest, e.g., an antibody or antigen-binding portion thereof, for example, an anti-GM-CSFRα antibody such as mavrilimumab, are produced by subjecting the sample comprising a protein of interest to an anion exchange separation step. In some embodiments, the anion exchange step occurs after the above-described affinity chromatography, e.g., Protein A affinity, step. In some embodiments, the anion exchange step occurs after the cation exchange step. In certain embodiments, the anion exchange step occurs before the cation exchange step.

The use of an anionic exchange material versus a cationic exchange material, such as those cation exchange materials discussed in detail above, is based on the local charges of the protein of interest in a given solution. Thereof, it is within the scope of this invention to employ an anionic exchange step prior to the use of a cationic exchange step, or a cationic exchange step prior to the use of an anionic exchange step. Furthermore, it is within the scope of this invention to employ only an anionic exchange step, only an cationic exchange step, or any serial combination of the two (including serial combinations of one or both ion exchange steps with the other chromatographic separation technologies described herein).

In performing the separation, the initial protein composition can be contacted with the anion exchange material by using any of a variety of techniques, e.g., using a batch purification technique or a chromatographic technique, as described above.

In certain embodiments, the aqueous salt solution used as both the loading and wash buffer has a pH at or near the isoelectric point (pI) of the protein of interest. In certain embodiments, the pH of the loading and wash buffer is about 0 to 2 units higher or lower than the pI of the protein of interest. In certain embodiments, the pH of the loading and wash buffer is about 0 to 0.5 units higher or lower than the pI of the protein of interest. In certain embodiments, the pH of the loading and wash buffer is at the pI of the protein of interest.

In certain embodiments, the pH of the loading, wash or elution buffer is about 5.9-6.1, 3.5-10.5, about 4-10, about 4.5-9.5, about 5-9, abut 5.5-8.6, about 6-8, about 6.5-7.5, about 6-7, about 5-8, about 4-7, about 5-7, about 5-6, about 5-5.5. In certain embodiments, the pH of the loading, wash or elution buffer is about 5, 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.5, 9, 9.5, or 10.

Buffer systems suitable for use in the AEX methods include, but are not limited to pyridine, piperazine, L-histidine, Bis-tris, Bis-tris propane, imidazole, N-Ethylmorpholine, TEA (triethanolamine), Tris, Morpholine, N-Methyldiethanolamine, AMPD (2-amino-2-methyl-1,3-propanediol), diethanolamine, ethanolamine, AMP (2-amino-2-methyl-1-propaol), piperazine, 1,3-Diaminopropane, piperidine. In certain embodiments, the conductivity and pH of the buffer is adjusted by increasing or decreasing the concentration of cationic or anionic agents. In certain non-limiting embodiments, the cationic agent is selected from the group consisting of sodium, Tris, tromethalmine, ammonium, arginine, histidine, or combinations thereof. In certain non-limiting embodiments, the anionic agent is selected from the group consisting of formate, acetate, citrate, chloride anion, sulphate, phosphate or combinations thereof.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 130 mM, 120 mM, 110 mM, 100 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM sodium acetate. In some embodiments, the elution buffer comprises about 1-500 mM, about 10-250 mM, about 10-150 mM, about 10-100 mM, about 20-90 mM, about 30-80 mM, about 40-70 mM, or about 50-60 mM, about 40-60 mM sodium acetate. In some embodiments, the elution buffer comprises about 40-60 mM sodium acetate.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 135 mM, 130 mM, 125 mM, 120 mM, 115 mM, 110 mM, 105 mM, 100 mM, 95 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM sodium chloride. In some embodiments, the elution buffer comprises about 1-500 mM, about 10-250 mM, about 10-150 mM, about 10-100 mM, about 20-90 mM, about 30-80 mM, about 40-70 mM, about 50-250 mM, about 50-170 mM, about 50-60 mM, or about 40-60 mM sodium chloride. In some embodiments, the elution buffer comprises about 40-60 mM sodium chloride.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 130 mM, 120 mM, 110 mM, 100 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM histidine. In some embodiments, the elution buffer comprises about 1-500 mM, about 10-250 mM, about 10-150 mM, about 10-100 mM, about 20-90 mM, about 30-80 mM, about 40-70 mM, or about 50-60 mM, about 40-60 mM histidine. In some embodiments, the elution buffer comprises about 40-60 mM histidine.

In some embodiments, the elution buffer comprises about 500 mM, 490 mM, 480 mM, 470 mM, 460 mM, 450 mM, 440 mM, 430 mM, 420 mM, 410 mM, 400 mM, 390 mM, 380 mM, 370 mM, 360 mM, 350 mM, 340 mM, 330 mM, 320 mM, 310 mM, 300 mM, 290 mM, 280 mM, 270 mM, 260 mM, 250 mM, 240 mM, 230 mM, 220 mM, 210 mM, 200 mM, 190 mM, 180 mM, 170 mM, 160 mM, 150 mM, 140 mM, 130 mM, 120 mM, 110 mM, 100 mM, 90 mM, 85 mM, 80 mM, 75 mM, 70 mM, 65 mM, 60 mM, 55 mM, 50 mM, 45 mM, 40 mM, 35 mM, 30 mM, 25 mM, 20 mM, 15 mM, 10 mM or 5 mM Bis-Tris. In some embodiments, the elution buffer comprises about 1-500 mM, about 10-250 mM, about 10-150 mM, about 10-100 mM, about 20-90 mM, about 30-80 mM, about 40-70 mM, about 50-60 mM, or about 40-60 mM Bis-Tris. In some embodiments, the elution buffer comprises about 40-60 mM Bis-Tris.

In some embodiments, the elution buffer comprises about 50 mM histidine, about 105 mM sodium chloride, and a pH of about 6.

A packed anion-exchange chromatography column, anion-exchange membrane device, anion-exchange monolithic device, or depth filter media can be operated either in bind-elute mode, flow-through mode, or a hybrid mode wherein the product exhibits binding to the chromatographic material, yet can be washed from the column using a buffer that is the same or substantially similar to the loading buffer. In the bind-elute mode, the column or the membrane device is first conditioned with a buffer with appropriate ionic strength and pH under conditions where certain proteins will be immobilized on the resin based matrix. For example, in certain embodiments, during the feed load, the protein of interest will be adsorbed to the resin due to electrostatic attraction. After washing the column or the membrane device with the equilibration buffer or another buffer with different pH and/or conductivity, the product recovery is achieved by increasing the ionic strength (i.e., conductivity) of the elution buffer to compete with the solute for the charged sites of the anion exchange matrix. Changing the pH and thereby altering the charge of the solute is another way to achieve elution of the solute. The change in conductivity or pH may be gradual (gradient elution) or stepwise (step elution). In the flow-through mode, the column or the membrane device is operated at selected pH and conductivity such that the protein of interest does not bind to the resin or the membrane while the acidic species will either be retained on the column or will have a distinct elution profile as compared to the protein of interest. In the context of this hybrid strategy, acidic species will bind to the chromatographic material (or Flow Through) in a manner distinct from the protein of interest, e.g., while the protein of interest and certain aggregates and/or fragments of the protein of interest may bind the chromatographic material, washes that preferentially remove the protein of interest can be applied. The column is then regenerated before next use.

Any anion exchange chromatography resins known in the art are suitable for the preparation of the composition of the present invention. Non-limiting examples of AEX resin include diethylaminoethyl (DEAE), quaternary aminoethyl (QAE) and quaternary amine (Q) groups. In certain embodiments, the resin employed for an AEX separation is POROS™ XQ. POROS™ XQ is a strong anion exchanger of a support matrix of cross-linked poly(styrene-divinylbenzene) functionalized with quaternary amines. In certain embodiments, the resin employed for an AEX separation is Capto™ Q ImRes. Capto™ Q™ ImRes is a strong anion exchanger of high-flow agarose resin functionalized with quaternary amines Additional non-limiting examples include: POROS™ 50PI, POROS™ 50HQ, Capto™ DEAE, Toyopearl™ QAE-550, Toyopearl™ DEAE-650, Toyopearl™ GigaCap Q-650, Fractogel® EMD TMAE Hicap, Sartobind STIC® PA nano, Sartobind Q nano; CUNO™ BioCap and X0HC.

In certain embodiments, the protein of interest, e.g., an antibody or antigen-binding portion thereof, for example, an anti-GM-CSFRα antibody such as mavrilimumab, is loaded onto the anion exchange chromatography resin at a level of about 10-100 g/L, about 20-90 g/L, about 30-80 g/L, about 40-70 g/L, or about 50-60 g/L. In certain embodiments, the protein of interest is loaded onto the anion exchange chromatography resin at a level of about 50-60 g/L.

In certain embodiments, the methods of the instant invention can be used to selectively remove, significantly reduce, or essentially remove all of variants and/or impurities, e.g., product-related substances, e.g., protein aggregates, fragments, e.g., half antibody, charged variants, e.g. acidic or basic species, and/or process-related impurities, e.g., host cell proteins, from the protein of interest, e.g., an antibody or antigen-binding portion thereof, for example, an anti-GM-CSFRα antibody such as mavrilimumab, wherein the variants and/or impurities, e.g., product-related substances, e.g., protein aggregates, fragments, e.g., half antibody, charged variants, e.g. acidic or basic species, and/or process-related impurities, e.g., host cell proteins, are collected in the Flow Through and wash fractions, and the protein of interest is enriched in the elution fraction, thereby producing protein compositions that have a reduced level of variants and/or impurities.

In certain embodiments, the eluate fractions comprising a protein of interest, e.g., an antibody or antigen-binding portion thereof, for example, an anti-GM-CSFRα antibody such as mavrilimumab, collected from the AEX chromatography step comprise a reduced level of variants and/or impurities, e.g., product-related substances, e.g., protein aggregates, fragments, e.g., half antibody, charged variants, e.g. acidic or basic species, and/or process-related impurities, e.g., host cell proteins. In some embodiments,

In some embodiments, the eluate fraction comprising a protein of interest, e.g., an antibody or antigen-binding portion thereof, for example, an anti-GM-CSFRα antibody such as mavrilimumab, comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the eluate fraction comprises about 0.1-25%, about 0.1-20%, about 1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one of more of the preceding.

In some embodiments, the eluate fraction comprising a protein of interest, e.g., an antibody or antigen-binding portion thereof, for example, an anti-GM-CSFRα antibody such as mavrilimumab, comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the eluate fraction comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% basic species, and ranges within one or more of the preceding. In some embodiments, the eluate fraction comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the eluate fraction comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 59-61%, about 58-63%, about 58-67%, about 53-61%, about 46-67%, or about 46-66% main species, e.g., anti-GM-CSFRα antibody such as mavrilimumab, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the eluate fraction comprises about 0.01-10%, about 0.01 to 5%, about 0.01 to 1%, about 0.01-0.4%, about 0.04-0.2%, about 0.1-0.4%, about 0.04-0.4%, about 0.04-0.8%, about 0.5-0.8%, about 0.1-6%, about 1-10%, about 2-10%, about 3-10%, or about 4-10% high molecular weight aggregates, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.5%, about 0.4%, or about 0.3% protein fragments, e.g., antibody fragments, and ranges within one or more of the preceding. In some embodiments, the eluate fraction comprises about 0.1-10%, about 0.1-5%, about 0.1-3%, about 0.1-2%, about 0.1-1.1%, about 0.1-0.8%, about 0.6-1.5%, about 0.5-1.5%, about 0.5-1.1%, about 0.4-0.8% or about 0.4-1.1% protein fragments, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 90%, about 91%, about 92%, about 93%, about 94%, about 95%, about 96%, about 97%, about 98%, about 99%, about 99.1% or about 99.5% of monomer of the protein of interest, e.g, antibody monomer, and ranges within one or more of the preceding. In some embodiments, the eluate fraction comprises about 90-99.9%, about 90-95%, about 94-99.9%, about 95-99.9%, about 99-99.9%, about 99.1-99.9%, about 98-99%, about 98-99.9%, or about 98.5-99.5% of monomer of the protein of interest, e.g., antibody monomer.

In some embodiments, the eluate fraction comprising a protein of interest, e.g., an antibody or antigen-binding portion thereof, for example, an anti-GM-CSFRα antibody such as mavrilimumab, comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the preceding. In some embodiments, the eluate fraction comprises about 0.1-10, about 1-10, about 2-10, about 3-10, about 4-10, about 1-5, about 5-10, about 1-3, about 0.1-2, about 0.1-3, about 2-8, or about 0.1-8 ppm HCP, and ranges within one or more of the preceding.

In certain embodiments, the loading, pH, conductivity of the AEX chromatography step, as well as elution pH and/or conductivity, can be modified to achieve a desired distribution of variants and/or impurities away from the protein of interest, e.g., the antibody or antigen binding portion thereof, for example, an anti-GM-CSFRα antibody such as mavrilimumab.

In certain embodiments, an AEX chromatographic separation can be performed and combinations of fractions can be pooled to achieve a combination of desired process-related impurity and/or product-relates substance levels, in addition to, or in place of merely modulating charge variant concentration.

In certain embodiments, spectroscopy methods such as UV, NIR, FTIR, Fluorescence, Raman may be used to monitor levels of variants and/or impurities, e.g., product-related substances, e.g., charge variants, aggregates, fragments of the protein of interest, and/or process-related impurities, e.g., host cell proteins, in an on-line, at-line or in-line mode, which can then be used to control the level of variants and/or impurities in the pooled material collected from the AEX effluent. In certain embodiments, on-line, at-line or in-line monitoring methods can be used either on the effluent line of the chromatography step or in the collection vessel, to enable achievement of the desired product quality/recovery. In certain embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed and the target product quality can be achieved. In certain embodiments, such measurements can be combined with in-line dilution methods such that ion concentration/conductivity of the load/wash can be controlled by feedback and hence facilitate product quality control.

In certain embodiments, a combination of CEX and AEX and/or MM methods can be used to prepare compositions of the invention comprising a protein of interest, e.g., an antibody or antigen-binding portion thereof, for example, an anti-GM-CSFRα antibody such as mavrilimumab, including certain embodiments where one technology is used in a complementary/supplementary manner with another technology. In some embodiments, such a combination can be performed such that certain sub-species are removed predominantly by one technology, such that the combination provides the desired final composition/product quality. In some embodiments, such combinations include the use of additional chromatography, filtration, nanofiltration, ultrafiltration/diafiltration (UF/DF) steps so as to achieve the desired product quality.

Mixed Mode Chromatography

Mixed mode (“MM”) chromatography column, membrane device, monolithic device, or depth filter media may also be used to prepare the compositions of the invention comprising a protein of interest, e.g., an antibody or antigen-binding portion thereof, for example, an anti-GM-CSFRα antibody such as mavrilimumab. Mixed mode chromatography, also referred to herein as “multimodal chromatography”, is a chromatographic strategy that utilizes a support comprising a ligand that is capable of providing at least two different, and in certain embodiments co-operative, sites that interact with the substance to be bound. In certain embodiments, one of these sites gives an attractive type of charge-charge interaction between the ligand and the substance of interest and the other site provides for electron acceptor-donor interaction and/or hydrophobic and/or hydrophilic interactions. Electron donor-acceptor interactions include interactions such as hydrogen-bonding, π-π, cation-π, charge transfer, dipole-dipole, induced dipole etc.

Any mixed mode chromatography resins known in the art are suitable for the preparation of the composition of the present invention comprising a protein of interest, e.g., an antibody or antigen-binding portion thereof, for example, an anti-GM-CSFRα antibody such as mavrilimumab. In certain embodiments, the resin employed for a mixed mode separation is Capto™ MMC ImpRes. Capto™ MMC ImpRes is a weak cation exchanger with multimodal functionality with a base matrix of high-flow agarose with carboxylic and hydroxyl ligands. In certain embodiments, the resin employed for a mixed mode separation is Capto™ Adhere ImpRes and/or Capto™ Adhere. Capto™ Adhere ImpRes and Capto™ Adhere are strong anion exchangers with multimodal functionality. The base matrix is a highly cross-linked agarose with a ligand (N-Benzyl-N-methyl ethanol amine) that exhibits many functionalities for interaction, such as ionic interaction, hydrogen bonding and hydrophobic interaction. In certain embodiments, the resin employed for a mixed mode separation is selected from PPA-HyperCel and HEA-HyperCel. The base matrices of PPA-HyperCel and HEA-HyperCel are high porosity cross-linked cellulose. Their ligands are Phenylpropylamine and Hexylamine, respectively. Phenylpropylamine and Hexylamine offer different selectivity and hydrophobicity options for protein separations. Additional mixed mode chromatographic supports include, but are not limited to, Nuvia™ C Prime, Toyo Pearl™ MX Trp 650M, and Eshmuno® HCX.

In certain embodiments, the mixed mode chromatography resin is comprised of ligands coupled to an organic or inorganic support, sometimes denoted a base matrix, directly or via a spacer. The support may be in the form of particles, such as essentially spherical particles, a monolith, filter, membrane, surface, capillaries, etc. In certain embodiments, the support is prepared from a native polymer, such as cross-linked carbohydrate material, such as agarose, agar, cellulose, dextran, chitosan, konjac, carrageenan, gellan, alginate etc. To obtain high adsorption capacities, the support can be porous, and ligands are then coupled to the external surfaces as well as to the pore surfaces. Such native polymer supports can be prepared according to standard methods, such as inverse suspension gelation (S Hjerten: Biochim Biophys Acta 79(2), 393-398 (1964). Alternatively, the support can be prepared from a synthetic polymer, such as cross-linked synthetic polymers, e.g., styrene or styrene derivatives, divinylbenzene, acrylamides, acrylate esters, methacrylate esters, vinyl esters, vinyl amides etc. Such synthetic polymers can be produced according to standard methods, see e.g., “Styrene based polymer supports developed by suspension polymerization” (R Arshady: Chimica e L'Industria 70(9), 70-75 (1988)). Porous native or synthetic polymer supports are also available from commercial sources, such as Amersham Biosciences, Uppsala, Sweden.

In certain embodiments, the protein of interest, e.g., an antibody or antigen-binding portion thereof, for example, an anti-GM-CSFRα antibody such as mavrilimumab, is loaded onto the mixed mode chromatography resin at a level of about 10-100 g/L, about 20-90 g/L, about 30-60 g/L, about 30-80 g/L, about 40-70 g/L, or about 50-60 g/L. In certain embodiments, the protein of interest is loaded onto the mixed mode chromatography resin at a level of about 50-60 g/L.

In certain embodiments, the methods of the instant invention can be used to selectively remove, significantly reduce, or essentially remove all of variants and/or impurities, e.g., product-related substances, e.g., protein aggregates, fragments, e.g., half antibody, charged variants, e.g. acidic or basic species, and/or process-related impurities, e.g., host cell proteins, from the protein of interest, e.g., an antibody or antigen-binding portion thereof, for example, an anti-GM-CSFRα antibody such as mavrilimumab, wherein the variants and/or impurities, e.g., product-related substances, e.g., protein aggregates, fragments, e.g., half antibody, charged variants, e.g. acidic or basic species, and/or process-related impurities, e.g., host cell proteins, are collected in the Flow Through and wash fractions, and the protein of interest is enriched in the elution fraction, thereby producing protein compositions that have a reduced level of variants and/or impurities.

In certain embodiments, the eluate fractions comprising a protein of interest, e.g., an antibody or antigen-binding portion thereof, for example, an anti-GM-CSFRα antibody such as mavrilimumab, collected from the mixed mode chromatography step comprise a reduced level of variants and/or impurities, e.g., product-related substances, e.g., protein aggregates, fragments, e.g., half antibody, charged variants, e.g. acidic or basic species, and/or process-related impurities, e.g., host cell proteins. In some embodiments,

In some embodiments, the eluate fraction comprising a protein of interest, e.g., an antibody or antigen-binding portion thereof, for example, an anti-GM-CSFRα antibody such as mavrilimumab, comprises less than about 25%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3.9%, about 3.8%, about 3.7%, about 3.6%, about 3.5%, about 3.4%, about 3.3%, about 3.2%, about 3.1%, about 3%, about 2.9%, about 2.8%, about 2.7%, about 2.6%, about 2.5%, about 2.4%, about 2.3%, about 2.2%, about 2.1%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% half antibody, and ranges within one or more of the preceding. In some embodiments, the eluate fraction comprises about 0.1-25%, about 0.1-20%, about 1-18%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody, and ranges within one of more of the preceding.

In some embodiments, the eluate fraction comprising a protein of interest, e.g., an antibody or antigen-binding portion thereof, for example, an anti-GM-CSFRα antibody such as mavrilimumab, comprises less than about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% acidic species, and ranges within one or more of the preceding. In some embodiments, the eluate fraction comprises about 1-40%, about 1-35%, about 1-30%, about 1-28%, about 1-25%, about 2-20%, about 3-15%, about 5-25%, about 5-28%, about 5-30%, about 10-28%, about 10-30%, about 10-40%, about 9-18%, about 11-22%, about 11-38%, about 12-20%, about 12-38%, about 15-30%, about 14-28%, or about 18-40% acidic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 3%, about 2%, or about 1% basic species, and ranges within one or more of the preceding. In some embodiments, the eluate fraction comprises about 1-45%, about 1-40%, about 1-35%, about 1-25%, about 5-35%, about 10-35%, about 15-35%, about 1-30%, about 1-25%, about 1-24%, about 5-25%, about 5-30%, about 5-45%, about 10-25%, about 10-30%, about 10-40%, about 15-25%, about 15-30%, about 15-35%, about 15-25%, about 16-31%, about 17-26%, about 9-29%, about 9-41%, or about 16-41% basic species, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 40%, about 45%, about 50%, about 55%, about 60%, about 61%, about 62%, about 63%, about 64%, about 65%, about 66%, about 67%, about 68%, about 69%, about 70%, about 75%, about 80%, about 85%, about 90%, about 95% or about 99% main species, and ranges within one or more of the preceding. In some embodiments, the eluate fraction comprises about 40-99%, about 45-99%, about 50-99%, about 55-99%, about 50-90%, about 55-90%, about 50-80%, about 55-80%, about 50-70%, about 55-70%, about 50-65%, about 55-65%, about 58-62%, about 59-61%, about 58-63%, about 58-67%, about 53-61%, about 46-67%, or about 46-66% main species, e.g., anti-GM-CSFRα antibody such as mavrilimumab, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, or about 0.1% high molecular weight aggregates, and ranges within one or more of the preceding. In some embodiments, the eluate fraction comprises about 0.01-10%, about 0.01 to 5%, about 0.01 to 1%, about 0.01-0.4%, about 0.04-0.2%, about 0.1-0.4%, about 0.04-0.4%, about 0.04-0.8%, about 0.5-0.8%, about 0.1-6%, about 1-10%, about 2-10%, about 3-10%, or about 4-10% high molecular weight aggregates, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises less than about 10%, about 9%, about 8%, about 7%, about 6%, about 5%, about 4%, about 3%, about 2%, about 1%, about 0.5%, about 0.4%, or about 0.3% protein fragments, e.g., antibody fragments, and ranges within one or more of the preceding. In some embodiments, the eluate fraction comprises about 0.1-10%, about 0.1-5%, about 0.1-3%, about 0.1-2%, about 0.1-1.1%, about 0.1-0.8%, about 0.6-1.5%, about 0.5-1.5%, about 0.5-1.1%, about 0.4-0.8% or about 0.4-1.1% protein fragments, and ranges within one or more of the preceding.

In some embodiments, the eluate fraction comprising a protein of interest, e.g., an antibody or antigen-binding portion thereof, for example, an anti-GM-CSFRα antibody such as mavrilimumab, comprises more than about 90%, about 91%, about 92%, about 93%, about 94%, about 95%, about 96%, about 97%, about 98%, about 99%, about 99.1% or about 99.5% of monomer of the protein of interest, e.g., antibody monomer, and ranges within one or more of the preceding. In some embodiments, the eluate fraction comprises about 90-99.9%, about 90-95%, about 94-99.9%, about 95-99.9%, about 99-99.9%, about 99.1-99.9%, about 98-99%, about 98-99.9%, or about 98.5-99.5% of monomer of the protein of interest, e.g., antibody monomer.

In some embodiments, the eluate fraction comprising a protein of interest, e.g., an antibody or antigen-binding portion thereof, for example, an anti-GM-CSFRα antibody such as mavrilimumab, comprises less than about 10, about 9, about 8, about 7, about 6, about 5, about 4, about 3, about 2, about 1, about 0.9, about 0.8, about 0.7, about 0.6 or about 0.5 ppm host cell proteins, and ranges within one or more of the proceeding. In some embodiments, the eluate fraction comprises about 0.1-10, about 1-10, about 2-10, about 3-10, about 4-10, about 1-5, about 5-10, about 1-3, about 0.1-2, about 0.1-3, about 2-8, or about 0.1-8 ppm HCP, and ranges within one or more of the proceeding.

In certain embodiments, the loading, pH, conductivity of the mixed mode chromatography step, as well as elution pH and/or conductivity, can be modified to achieve a desired distribution of variants and/or impurities away from the protein of interest, e.g., the antibody or antigen binding portion thereof, for example, an anti-GM-CSFRα antibody such as mavrilimumab.

In certain embodiments, a mixed mode chromatographic separation can be performed and combinations of fractions can be pooled to achieve a combination of desired process-related impurity and/or product-relates substance levels, in addition to, or in place of merely modulating charge variant concentration.

In certain embodiments, spectroscopy methods such as UV, NIR, FTIR, Fluorescence, Raman may be used to monitor levels of variants and/or impurities, e.g., product-related substances, e.g., charge variants, aggregates, fragments of the protein of interest, and/or process-related impurities, e.g., host cell proteins, in an on-line, at-line or in-line mode, which can then be used to control the level of variants and/or impurities in the pooled material collected from the mixed mode effluent. In certain embodiments, on-line, at-line or in-line monitoring methods can be used either on the effluent line of the chromatography step or in the collection vessel, to enable achievement of the desired product quality/recovery. In certain embodiments, the UV signal can be used as a surrogate to achieve an appropriate product quality/recovery, wherein the UV signal can be processed appropriately, including, but not limited to, such processing techniques as integration, differentiation, moving average, such that normal process variability can be addressed and the target product quality can be achieved. In certain embodiments, such measurements can be combined with in-line dilution methods such that ion concentration/conductivity of the load/wash can be controlled by feedback and hence facilitate product quality control.

In certain embodiments, a combination of CEX and AEX and/or mixed mode methods can be used to prepare compositions of the invention comprising a protein of interest, e.g., an antibody or antigen-binding portion thereof, for example, an anti-GM-CSFRα antibody such as mavrilimumab, including certain embodiments where one technology is used in a complementary/supplementary manner with another technology. In some embodiments, such a combination can be performed such that certain sub-species are removed predominantly by one technology, such that the combination provides the desired final composition/product quality. In some embodiments, such combinations include the use of additional chromatography, filtration, nanofiltration, ultrafiltration/diafiltration (UF/DF) steps so as to achieve the desired product quality.

Viral Filtration/Nanofiltration

Certain embodiments of the present invention employ nanofiltration steps to reduce the viral load and concentrate the protein of interest, e.g., an antibody, or antigen binding portion thereof, for example, an anti-GM-CSFRα antibody such as mavrilimumab. Following the intermediate/final polishing chromatography step, the eluate pool may be subjected to a nanofiltration step. In an embodiment, the nanofiltration step is accomplished via one or more nanofilters or viral filters. In a particular embodiment, the nanofiltration step may be accomplished via a filter train comprised of a prefilter and a nanofilter or viral filters. The filters may be any known in the art to be useful for this purpose and may include, for example, EMD Millipore Viresolve VPro, Viresolve NFP, Viresolve NFR, Pellicon or Millipak filters, Sartorius Vivaspin, ViroStart CPV, or Sartopore filters, Pall Ultipor DVD, DV50, DV20 filtres, or Planova 15N, 20N, and 35N virus removal filters from Asashi Kasei Pharma. In certain embodiments, the nanofiltration filter has a mean pore size of between about 15 nm and about 200 nm. In a specific embodiment, the nanofilter may have a mean pore size of between about 15 nm and about 72 nm, or between about 19 nm and about 35 nm, or of at or about 15 nm, 19 nm, 35 nm, or 72 nm. One of skill in the art will understand that the selection of types and numbers of filters will be dependent on the volume of sample being processed and the desired filtration performance

Ultrafiltration/Diafiltration

Certain embodiments of the present invention employ ultrafiltration and diafiltration steps to further concentrate and formulate the protein of interest, e.g., an antibody, or antigen binding portion thereof, for example, an anti-GM-CSFRα antibody such as mavrilimumab. The nanofiltration step may be followed by ultrafiltration and diafiltration to achieve the targeted drug substance concentration and buffer condition before formulation.

Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample buffer through the filter membrane pores while proteins, such as antibodies, are retained above the membrane surface.

Diafiltration is a method of using membrane filters to remove and exchange salts, sugars, and non-aqueous solvents, to separate free from bound species, to remove low molecular-weight species, and/or to cause the rapid change of ionic and/or pH environments. Microsolutes are removed most efficiently by adding solvent to the solution being diafiltered at a rate approximately equal to the permeate flow rate. This washes away microspecies from the solution at a constant volume, effectively purifying the retained protein of interest. In certain embodiments of the present invention, a diafiltration step is employed to exchange the various buffers used in connection with the instant invention, optionally prior to further chromatography or other purification steps, as well as to remove impurities from the protein preparations.

One of ordinary skill in the art can select appropriate membrane filter device for the UF/DF operation. Examples of membrane cassettes suitable for the present invention include, but not limited to, Pellicon 2 or Pellicon 3 cassettes with 10 kD, 30 kD or 50 kD membranes from EMD Millipore, Kvick 10 kD, 30 kD or 50 kD membrane cassettes from GE Healthcare, and Centramate or Centrasette 10 kD, 30 kD or 50 kD cassettes from Pall Corporation.

Upon completion of the diafiltration step, the protein concentration of the solution can be adjusted to with the diafiltration buffer to a final concentration of between about 5% and about 20% (w/v), or between about 10% and about 20% (w/v), or between about 15% and about 20% (w/v), or between about 18% and about 20% (w/v), or to a final concentration of about 5%, or 6%, 7%, 8%, 9%, 10%, 11%, 12%, 13%, 14%, 15%, 16%, 17%, 18%, 19%, or 20% for formulation.

In some embodiments, the formulated solution can be further sterilized by first filtering through a membrane filter with an absolute pore size of 0.2 micron or less with or without pre-filter. Then the solution is aseptically dispensed into final containers for proper sealing, with samples taken for testing.

Exemplary Purification Strategies

In certain embodiments, primary recovery can proceed by sequentially employing pH reduction, centrifugation, and filtration steps to remove cells and cell debris (including HCPs) from the production bioreactor harvest. In certain embodiments, the present invention is directed to subjecting a sample mixture from said sample to one or more affinity (e.g., protein A), AEX, CEX, and/or MM purification steps. Certain embodiments of the present invention may include further purification steps, which can be performed prior to, during, or following the affinity and/or ion exchange chromatography steps. Examples of additional purification procedures include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, and dialysis.

In certain embodiments the unbound Flow Through and wash fractions can be further fractionated and a combination of fractions providing a target product purity can be pooled.

In certain embodiments the protein concentration can be adjusted to achieve a differential partitioning behavior between the antibody product and the variants and/or impurities, e.g., the product-related substances and/or the process-related impurities, such that the purity and/or yield can be further improved. In certain embodiments the loading can be performed at different protein concentrations during the loading operation to improve the product quality/yield of any particular purification step. In certain embodiments the column temperature can be independently varied to improve the separation efficiency and/or yield of any particular purification step.

In certain embodiments, the loading, washing and/or elution buffer matrices can be different or composed of mixtures of chemicals, while achieving similar “resin interaction” behavior such that the above novel separation can be effected. For example, but not by way of limitation, the loading and washing buffers can be different, in terms of ionic strength or pH, while remaining substantially similar in function in terms of the washout of the product achieved during the wash step.

In certain embodiments, the loading, washing and/or eluting steps can be controlled by in-line, at-line or off-line measurement of the variants and/or impurities levels, e.g., the product related substance levels and/or the process-related impurity levels, either in the column effluent, or the collected pool or both, so as to achieve the target product quality and/or yield. In certain embodiments, the loading concentration can be dynamically controlled by in-line or batch or continuous dilutions with buffers or other solutions to achieve the partitioning necessary to improve the separation efficiency and/or yield.

### V. Methods of Assaying Sample Purity

Assaying Charged Variants

The levels of charged variants, e.g., acidic or basic species, in the chromatographic samples produced using the techniques described herein may be analyzed by any charged based separation techniques known in the art. For example, charged variants, e.g., acidic species, or basic species, can be detected by charged based separation techniques such as isoelectric focusing (IEF) gel electrophoresis, capillary isoelectric focusing (cIEF) gel electrophoresis, cation exchange chromatography (CEX) and anion exchange chromatography (AEX).

Acidic species are variants with lower apparent pI and basic species are variants with higher apparent pI when antibodies are analyzed using IEF based methods. When analyzed by chromatography-based methods, acidic species and basic species are defined based on their retention times relative to the main peak. Acidic species are the variants that elute earlier than the main peak from CEX or later then than the main peak from AEX, basic species are the variants that elute later than the main peak from CEX or earlier than the main peak from AEX.

In certain embodiments, the charged variants are assayed by an ion exchange chromatography step. In some embodiments, quantitation is based on the relative area percent of detected peaks.

Assaying Size Variants

In certain embodiments, the levels of aggregates, monomer, fragments, and half antibody in the chromatographic samples produced using the techniques described herein are analyzed. In certain embodiments, the aggregates, monomer, and fragments are measured using a size exclusion chromatographic (SEC) method for each molecule. In certain embodiments, quantification is based on the relative area of detected peaks. In some embodiments, the level of half antibody is measured using non-reduced capillary electrophoresis sodium dodecyl sulfate (CE-SDS).

Any additional technique, such as mass spectroscopy, can also be used for assaying size variants.

Assaying Host Cell Protein

The present invention also provides methods for determining the residual levels of host cell protein (HCP) concentration in the compositions of the invention. As described above, HCPs are desirably excluded from the final target substance product. Exemplary HCPs include proteins originating from the source of the antibody production. Failure to identify and sufficiently remove HCPs from the target antibody may lead to reduced efficacy and/or adverse reactions in a subject.

As used herein, the term “HCP ELISA” refers to an ELISA where the second antibody used in the assay is specific to the HCPs produced from cells used to generate the antibody of interest. The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with antibody DNA. In an exemplary embodiment, the second antibody is produced using HCPs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody is added, and binds to the HCPs within the sample. In certain embodiments the first and second antibodies are polyclonal antibodies.

In certain aspects the first and second antibodies are blends of polyclonal antibodies raised against HCPs. The amount of HCP contained in the sample is determined using the appropriate test based on the label of the second antibody.

HCP ELISA may be used for determining the level of HCPs in an antibody composition, such as an eluate or flow-through obtained using the process described above. The present invention also provides a composition comprising an antibody, wherein the composition has no detectable level of HCPs as determined by an HCP Enzyme Linked Immunosorbent Assay (“ELISA”).

### VI. Methods of Treatment Using the Compositions of the Invention

The compositions of the invention comprising a protein of interest, e.g., an antibody or antigen-binding portion thereof, for example, an anti-GM-CSFRα antibody such as mavrilimumab, may be used to treat any disorder in a subject for which the therapeutic protein comprised in the composition is appropriate for treating.

A “disorder” is any condition that would benefit from treatment with the protein. This includes chronic and acute disorders or diseases including those pathological conditions which predispose the subject to the disorder in question. In the case of an anti-GM-CSFRα antibody, or antigen binding portion thereof, such as mavrilimumab, a therapeutically effective amount of the composition may be administered to treat a GM-CSFRα-associated disorder.

A GM-CSFRα-associated disorder includes a disorder in which inhibition of GM-CSFRα activity is expected to alleviate the symptoms and/or progression of the disorder. Since GM-CSF binds specifically to GM-CSFRα, pathological and/or symptomatic effects of GM-CSF can also be countered by inhibiting binding of GM-CSF to GM-CSFRα. Thereof, a GM-CSFRα-associated disorder may be evidenced, for example, by an increase in the concentration of GM-CSFRα and/or GM-CSF in a biological fluid of a subject suffering from the disorder (e.g., an increase in the concentration of GM-CSFRα and/or GM-CSF in serum, plasma, synovial fluid, etc. of the subject).

The compositions of the invention comprising a protein of interest, e.g., an antibody or antigen-binding portion thereof, for example, an anti-GM-CSFRα antibody such as mavrilimumab, can be used in treating any GM-CSFRα associated diseases or disorders known in the art including, but not limited to, autoimmune, inflammatory and/or respiratory conditions, diseases and disorders. In one embodiment, a GM-CSFRα-associated disorder includes autoimmune diseases (including rheumatoid arthritis, rheumatoid spondylitis, osteoarthritis and gouty arthritis, allergy, multiple sclerosis, autoimmune diabetes, autoimmune uveitis, nephrotic syndrome, multisystem autoimmune diseases, lupus (including systemic lupus, lupus nephritis and lupus cerebritis), Crohn's disease and autoimmune hearing loss), active axial spondyloarthritis (active axSpA) and non-radiographic axial spondyloarthritis (nr-axSpA), infectious diseases (including malaria, meningitis, acquired immune deficiency syndrome (AIDS), influenza and cachexia secondary to infection), sepsis (including septic shock, endotoxic shock, gram negative sepsis and toxic shock syndrome), allograft rejection and graft versus host disease, malignancy, myeloid leukemia, pulmonary disorders (including adult respiratory distress syndrome (ARDS), shock lung, chronic pulmonary inflammatory disease, pulmonary sarcoidosis, pulmonary fibrosis, silicosis, idiopathic interstitial lung disease and chronic obstructive airway disorders (COPD), such as asthma), intestinal disorders (including inflammatory bowel disorders, idiopathic inflammatory bowel disease, Crohn's disease and Crohn's disease-related disorders (including fistulas in the bladder, vagina, and skin; bowel obstructions; abscesses; nutritional deficiencies; complications from corticosteroid use; inflammation of the joints; erythem nodosum; pyoderma gangrenosum; lesions of the eye, Crohn's related arthralgias, fistulizing Crohn's indeterminant colitis and pouchitis), cardiac disorders (including ischemia of the heart, heart insufficiency, restenosis, congestive heart failure, coronary artery disease, angina pectoris, myocardial infarction, cardiovascular tissue damage caused by cardiac arrest, cardiovascular tissue damage caused by cardiac bypass, cardiogenic shock, and hypertension, atherosclerosis, cardiomyopathy, coronary artery spasm, coronary artery disease, valvular disease, arrhythmias, and cardiomyopathies), spondyloarthropathies (including ankylosing spondylitis, psoriatic arthritis/spondylitis, enteropathic arthritis, reactive arthritis or Reiter's syndrome, and undifferentiated spondyloarthropathies), metabolic disorders (including obesity and diabetes, including type 1 diabetes mellitus, type 2 diabetes mellitus, diabetic neuropathy, peripheral neuropathy, diabetic retinopathy, diabetic ulcerations, retinopathy ulcerations and diabetic macrovasculopathy), anemia, pain (including acute and chronic pains, such as neuropathic pain and post-operative pain, chronic lower back pain, cluster headaches, herpes neuralgia, phantom limb pain, central pain, dental pain, opioid-resistant pain, visceral pain, surgical pain, bone injury pain, pain during labor and delivery, pain resulting from burns, including sunburn, post partum pain, migraine, angina pain, and genitourinary tract-related pain including cystitis), hepatic disorders (including hepatitis, alcoholic hepatitis, viral hepatitis, alcoholic cirrhosis, al antitypsin deficiency, autoimmune cirrhosis, cryptogenic cirrhosis, fulminant hepatitis, hepatitis B and C, and steatohepatitis, cystic fibrosis, primary biliary cirrhosis, sclerosing cholangitis and biliary obstruction), skin and nail disorders (including psoriasis (including chronic plaque psoriasis, guttate psoriasis, inverse psoriasis, pustular psoriasis and other psoriasis disorders), pemphigus vulgaris, scleroderma, atopic dermatitis (eczema), sarcoidosis, erythema nodosum, hidradenitis suppurative, lichen planus, Sweet's syndrome, scleroderma and vitiligo), vasculitides (including Behcet's disease), and other disorders, such as juvenile rheumatoid arthritis (JRA), endometriosis, prostatitis, choroidal neovascularization, sciatica, Sjogren's syndrome, uveitis, wet macular degeneration, osteoporosis and osteoarthritis.

In one embodiment, the GM-CSFRα-associated disease or disorder is rheumatoid arthritis. In some embodiments, the GM-CSFRα-associated disease or disorder is giant cell arteritis (GCA) or acute repiratory distress syndrome (ARDS), or cytokine release syndrome (CRS). In another embodiment, the GM-CSFRα-associated disease or disorder is coronavirus disease 2019 (COVID-19).

As used herein, the term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “treatment” or “treat” refers to both therapeutic treatment and prophylactic or preventative measures. Those in need of treatment include those already with the disorder, as well as those in which the disorder is to be prevented.

In one embodiment, the invention provides a method of administering a composition comprising an anti-GM-CSFRα antibody, or antigen binding portion thereof, to a subject such that GM-CSFRα activity is inhibited or a GM-CSFRα-associated disorder is treated. In one embodiment, the GM-CSFRα is human GM-CSFRα and the subject is a human subject. In one embodiment, the anti-GM-CSFRα antibody is mavrilimumab.

The composition can be administered by a variety of methods known in the art. Exemplary routes/modes of administration include intravenous, intramuscular, intranasal, oral, topical or subcutaneous delivery. As will be appreciated by the skilled artisan, the route and/or mode of administration will vary depending upon the desired results.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In certain embodiments it is especially advantageous to formulate parenteral compositions in dosage unit form for ease of administration and uniformity of dosage. Dosage unit form as used herein refers to physically discrete units suited as unitary dosages for the mammalian subjects to be treated; each unit comprising a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of a composition of the invention is about 0.01-30 mg/kg, 0.1-20 mg/kg, 1-10 mg/kg, 2-8 mg/kg, or 5-15 mg/kg. With respect to compositions comprising an anti-GM-CSFRα antibody, or antigen-binding portion thereof, such as mavrilimumab, an exemplary dose is about 30, 50, 100, or 150 mg every other week.

In some embodiments, in particular for treatment of rheumatoid arthritis, an exemplary dose includes a single intravenous dose of up to 10 mg/kg. In some embodiments, an exemplary dose includes repeat subcutaneous doses of up to 150 mg biweekly for up to 3 years.

In some embodiments, in particular for treatment of COVID-19, an exemplary dose includes a single intravenous dose of about 6 mg/kg or about 10 mg/kg.

In some embodiments, in particular for treatment of giant cell arteritis, an exemplary dose includes a subcutaneous dose of about 150 mg biweekly for 26 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### VII. Pharmaceutical Formulations Containing the Compositions of the Invention

The present invention further provides preparations and formulations comprising the compositions of the invention. It should be understood that the compositions comprising a protein of interest, e.g., an antibody and antigen binding portion thereof, described herein, may be formulated or prepared as described below. In one embodiment, the antibody is an anti-GM-CSFRα antibody, or antigen-binding portion thereof. In another embodiment, the anti-GM-CSFRα antibody is mavrilimumab.

In certain embodiments, the compositions of the invention may be formulated with a pharmaceutically acceptable carrier as pharmaceutical (therapeutic) compositions, and may be administered by a variety of methods known in the art. As will be appreciated by the skilled artisan, the route and/or mode of administration will vary depending upon the desired results.

The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the antibodies of the present invention, and with each other, in a manner such that there is no interaction which would substantially impair the desired pharmaceutical efficacy.

The compositions of the invention are present in a form known in the art and acceptable for therapeutic uses. In one embodiment, a formulation of the compositions of the invention is a liquid formulation. In another embodiment, a formulation of the compositions of the invention is a lyophilized formulation. In a further embodiment, a formulation of the compositions of the invention is a reconstituted liquid formulation. In one embodiment, a formulation of the compositions of the invention is a stable liquid formulation. In one embodiment, a liquid formulation of the compositions of the invention is an aqueous formulation. In another embodiment, the liquid formulation is non-aqueous. In a specific embodiment, a liquid formulation of the compositions of the invention is an aqueous formulation wherein the aqueous carrier is distilled water.

The compositions of the invention can be formulated for particular routes of administration, such as oral, nasal, pulmonary, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. By way of example, in certain embodiments, the antibodies (including antibody fragments) are formulated for intravenous administration. In certain other embodiments, the antibodies (including antibody fragments) are formulated for local delivery to the cardiovascular system, for example, via catheter, stent, wire, intramyocardial delivery, intrapericardial delivery, or intraendocardial delivery. In a particular embodiment, the composition comprises an anti-GM-CSFRα antibody such as mavrilimumab and is formulated for subcutaneous administration.

Formulations of the compositions of the invention which are suitable for topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required (U.S. Pat. Nos. 7,378,110; 7,258,873; 7,135,180; 7,923,029; and US Publication No. 20040042972).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Actual dosage levels of the active ingredients in the pharmaceutical compositions of the compositions of the invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The present invention is further illustrated by the following examples which should not be construed as limiting in any way.

### EXAMPLES

### Example 1: Upstream Process for Production of an Anti-GM-CSFRα Antibody

This Example provides the details of the upstream process for production of an anti-GM-CSFRα antibody, mavrilimumab. The development work consisted of a series of 10 L bioreactor studies and a 200 L demonstration run (Process 7 200 L Non-GMP Lot 1). The goal of the first 10 L experiment (Study No. 1) was to establish the mavrilimumab upstream process and to evaluate feed percentage. During this initial experiment, one bioreactor (R1) was run under control conditions and the other bioreactor (R2) was run with an increase in feed percentage from day 6 to day 14 to compensate for the increase in cell density. The increase in feed percentage in bioreactor 2 led to an increase in lactate production which correlated to lower pH trend compared to the control. Subsequently, the product quality results showed that the lower pH resulted in a more desirable charge profile.

To confirm the results obtained from the first experiment, the conditions for bioreactors 1 and 2 were repeated in the second study (Study No. 2) with the addition of a pH shift in both bioreactors from 6.90 to 6.75 on day 4. The results from bioreactors 3 and 4 indicate that both the increase in feed percentage on day 6 and decrease in pH setpoint on day 4 are beneficial to the charged species of the antibody.

The third experiment, Study No. 3, consisted of four 10 L bioreactors. The control, bioreactor 5, matched the conditions of bioreactor 4 from the previous run (increase in feed percentage from day 6 to day 14 and pH shift to 6.75 on day 4). Bioreactor 6 maintained the same feeding scheme as the control and the pH was shifted to 6.65 rather than 6.75 on day 4. Bioreactor 7 maintained the same pH shift as the control and feeding was initiated on day 2, increased on day 6 and decreased on day 9. Finally, bioreactor 8 was run under the same conditions as the control with the addition of a temperature shift from 36 to 32° C. on day 9. The conditions tested in bioreactors 6-8 resulted in either poor culture performance and/or undesirable product quality; thereof, the control conditions (bioreactor 4, 5) were chosen for scale-up to the 200 L demonstration run.

Three additional 10 L bioreactors were run concurrently with the 200 L demonstration run in Study No. 4. Bioreactor 9 was a direct satellite to the 200 L bioreactor and bioreactor 10 was used to evaluate the cell generation number at time of inoculation. Bioreactors 9 and 10 ran comparably to the 200 L demonstration run and produced material with similar product quality.

Detailed results from Studies Nos. 1-4 are shown in Tables 22-26.

ACRONYMS & DEFINITIONS

| Term | Definition |
|---|---|
| CDMO | Contract Development and Manufacturing Organization |
| kDa | Kilodalton |
| VCD | Viable Cell Density |
| DO | Dissolved Oxygen |
| MS | Microsparger |
| NR CE-SDS | Non-reduced capillary electrophoresis sodium dodecyl |
| | sulfate |
| IEC | Ion Exchange Chromatography |
| SEC | Size-exclusion Chromatography |

The bioreactor parameters used for all 10 L runs and the 200 L demonstration run are captured in Table 1-1.

TABLE 1-1
Bioreactor parameters

| | Set Point | | Set Point | | |
|---|---|---|---|---|---|
| Parameter | 10 L | | Non-GMP 200 L | | Units |
| Seeding Density | 0.8 | | 0.8 | | ×10⁶ c/mL |
| Initial Culture | 7.0 | | 147.0 | | L |
| Volume | | | | | |
| Final Culture Volume | 10 | | 200.0 | | L |
| Media Feed 7a | Experimental | | Day 3-5: 2% | | % Initial |
| Percentage | Condition | | Day 6-14: 3%* | | Culture Volume |
| Media Feed 7b | Experimental | | Day 3-5: 0.2% | | % Initial |
| Percentage | Condition | | Day 6-14: 0.3%* | | Culture Volume |
| Temperature | Experimental | | 36 | | ° C. |
| | Condition | | | | |
| Agitation | 120 | | 130 | | rpm |
| P/V at final volume | 58 | | 27 | | W/m³ |
| DO | 30 | | 30 | | % |
| DO Control Strategy | DO Input | O₂ | DO Input | O₂ | N/A |
| | (%) | (slpm) | (%) | (slpm) | |
| | 0 | 0 | 0 | 0 | |
| | 100 | 0.5 | 20 | 1.0 | |

| | |
|---|---|
| 40 | 2.0 |
| 100 | 10 |

| | | | |
|---|---|---|---|
| pH | Experimental | 6.90 ± 0.15 (D 0-D 3) | N/A |
| | Condition | 6.75 ± 0.10 (D 4-D 15) | |
| Air Overlay | 0.10 | 2.0 | slpm |
| Background Air | 0.05 | 1.0-5.0 | slpm |
| CO2 Sparge | 0-0.5 | 0-5.0 | slpm |
| Sparger (air and CO2) | 1 mm Drilled Hole | 1 mm Drilled Hole | N/A |
| Sparger (O2) | 20 um Sintered | 20 um Sintered | N/A |

| | |
|---|---|
| Harvest Criteria | DO set point will be increased and maintained |
| | at 80% until culture is below the DO probe |
| | Bioreactor will be cooled to 8° C. +/− 2° C. within 4 hr |
| | Maintain agitation rate until harvest volume is half |
| | of final volume, then decrease agitation rate to 60 rpm |
| | Final oxygen and air set-points will be set |
| | on manual when culture dips below DO probe |

*During optimization of the cell culture process, it was determined that the increase in media feed percentage could occur one day earlier, on day 5 rather than day 6, to mitigate unwanted lactate consumption and achieve the desired product quality.

The glucose feeding strategy used for all 10 L runs and the 200 L demonstration run are captured in Table 1-2.

TABLE 1-2
Glucose and antifoam feeding scheme

| Feed | Feeding Scheme and Strategy |
|---|---|
| 50% | Day 1-7: Target final glucose concentration of 4 g/L if pre- |
| Glucose | feed glucose concentration is less than or equal to 3 g/L. |
| (w/w) | Day 8-14: Target final glucose concentration of 5 g/L if pre- |
| | feed glucose concentration is less than or equal to 5 g/L. |

The clarification parameters for 10 L and 200 L processing are captured in Table 1-3.

TABLE 1-3
Clarification parameters

| | Set Point | Set Point | | |
|---|---|---|---|---|
| Parameter | 10 L | 200 L | Units | Notes |
| Harvest Initiation Day | 15 | 15 | Day | NA |
| Aeration During | 0.1/0.05 | 2.5 | slpm | Headsweep air/ |
| Harvest | | | | Background air |
| XDR DO Control Set | 80 | 30 | % | Worst-case DO |
| Point & Range for | | | | used for 200 L |
| Harvest | | | | |
| XDR Temperature Set | 8-12 | 36 | ° C. | Worst-case temp |
| Point & Range for | | | | used for 200 L |
| Harvest | | | | |

### Results and Discussion

### Study No. 1—Bioreactor 1 (R1) and Bioreactors 2 (R2)

### Experimental Conditions

The experimental conditions for Study No. 1 are captured in Table 1-4. The goal of this experiment was the establish the mavrilimumab upstream process and to evaluate feed percentage.

TABLE 1-4
Experimental conditions for Study No. 1

| Parameter | Brx-1 | | Brx-2 | |
|---|---|---|---|---|
| Media Feed 7a Percentage | 2% | (D 3-D 14) | 2% | (D 3-D 5) |
| (% initial Vol) | | | 3% | (D 6-D 14) |
| Media Feed 7b Percentage | 0.2% | (D 3-D 14) | 0.2% | (D 3-D 5) |

| | | |
|---|---|---|
| (% initial Vol) | 0.3% | (D 6-D 14) |

### Production Data

Bioreactor 1 achieved a higher viable cell density (VCD) than bioreactor 2 and both had a higher peak VCD compared to the bioreactor, 4×10 L R2. The viability of bioreactor 2 was in line with 4×10 L R2 while bioreactor 1 maintained a viability of >90% through day 15.

The discrepancy in final viability between bioreactors 1 and 2 can be attributed to an increase in lactate observed for bioreactor 2 from day 8-15. The increase in lactate resulted in a lower pH profile for bioreactor 2 compared to both bioreactor 1 and 4×10 L R2. Higher osmolality was also observed for bioreactor 2 which can be attributed to increased feed % as well as increased base addition to compensate for lower pH. CO₂ and all other metabolites were comparable between the runs. All additional data for the production bioreactors in Study No. 1 can be found in Table 1-22.

### Titer

Despite lower final viability, bioreactor 2 yielded slightly higher titer than bioreactor 1 and the day 15 value was nearly identical to 4×10 L R2. The final titer results from this experiment are in line with what was expected of the phase 3 conditions.

### Product Quality

Samples were prepared for TECAN purification and product quality analysis. Samples from days 13 and 15 were submitted for NR CE-SDS, SEC and Glycan. In addition to the day 13 and 15 samples, CEX load material from the downstream scale-down model (SDM) was submitted for NR CE-SDS and IEC. An increase in half-antibody from day 13 to day 15 was observed for both conditions (Error! Reference source not found.). However, the half-antibody levels in the CEX load material of the SDM are lower than the day 13 and 15 values for both reactors. The difference is believed to be due to an increased hold-time at 2-8° C. for the SDM material compared to the day 13 and 15 samples. The reduction in half-antibody over time is consistent with previous hold data. Previous studies were performed externally at CDMO to evaluate cell culture conditions that impact half-antibody; however, these studies were unsuccessful at identifying parameters that modulate this attribute. The SEC results, as shown in Table 1-7, show a slight decrease in monomer from day 13 to 15 and as expected, the fragment and aggregate levels are higher which has been processed through the entire purification process. The glycan results are stable over time. The IEC results can be seen in Error! Reference source not found. An increase in acidic peak was observed for both bioreactors. However, bioreactor 2 had approximately 8% lower acidic species compared to bioreactor 1 and it was hypothesized that this was a result of the lower cell culture pH throughout the run.

TABLE 1-5
Study No. 1 NR CE-SDS

| Bioreactor ID | Day | Half-Antibody (%) | IgG (%) |
|---|---|---|---|
| R1 | 13.0 | 8.7 | 90.3 |
| R1 | 15.0 | 9.3 | 86.7 |
| R1 | SDM CEX Load | 7.39 | 89.2 |
| R2 | 13.0 | 11.8 | 87.6 |
| R2 | 15.0 | 14.3 | 85.7 |
| R2 | SDM CEX Load | 6.2 | 90.8 |

TABLE 1-6
Study No. 1 IEC

| | | Acidic | Main | Basic | | | | | | | |
|---|---|---|---|---|---|---|---|---|---|---|---|
| Brx | | Peak | Peak | Peak | Acidic | Acidic | Acidic | Basic | Basic | Basic | Basic |
| ID | Day | (%) | (%) | (%) | 3 (%) | 2 (%) | 1 (%) | 1 (%) | 2 (%) | 3 (%) | 4 (%) |
| R1 | SDM | 27 | 49.14 | 23.9 | 4.75 | 5.36 | 16.86 | 8.5 | 10.39 | 2.31 | 2.7 |
| | CEX | | | | | | | | | | |
| | Load | | | | | | | | | | |
| R2 | SDM | 18.9 | 58.29 | 22.8 | 2.09 | 4.72 | 12.11 | 9.44 | 9.35 | 2.63 | 1.37 |
| | CEX | | | | | | | | | | |
| | Load | | | | | | | | | | |

TABLE 1-7
Study No. 1 SEC

| Bioreactor | | Monomer | Aggregate | Fragment |
|---|---|---|---|---|
| ID | Day | (%) | (%) | (%) |
| R1 | 13 | 96.7 | 1.6 | 1.7 |
| R1 | 15 | 95.3 | 2 | 1.7 |
| R2 | 13 | 96.9 | 1.9 | 1.2 |
| R2 | 15 | 96.2 | 2 | 1.8 |

### Study No. 2—Bioreactor 3 (R3) and Bioreactor 4 (R4)

At the conclusion of Study No. 2, it was hypothesized that lower culture pH resulted in lower levels of acidic species for bioreactor 2. To test this hypothesis, the conditions of bioreactors 1 and 2 were repeated in bioreactors 3 and 4 with the implementation of a pH shift on day 4. The experimental conditions can be found in Table 1-8.

### Experimental Conditions

TABLE 1-8
Study No. 2 experimental conditions

| Parameter | Brx-3 | Brx-4 |
|---|---|---|
| 7a Feed | 2% (D 3-D 14) | 2% (D 3-D 5) 3% |
| Percentage | | (D 6-D 14) |
| (% initial Vol) | | |
| 7b Feed | 0.2% (D 3-D 14) | 0.2%(D 3-D 5), 0.3% |
| Percentage | | (D 6-D 14) |
| (% initial Vol) | | |
| pH | 6.90 ± 0.15(D 0-D 3) to | 6.90 ± 0.15(D 0-D 3) to |
| | 6.75 ± 0.10 (D 4-D 15) | 6.75 ± 0.10 (D 4-D 15) |

### Production Data

Bioreactor 3 grew comparably to bioreactor 1, and bioreactor 4 grew comparably to bioreactor 2. This indicates that the pH shift did not negatively impact cell growth. Both bioreactors maintained a viability of >90% through day 15, in line with bioreactor 1. The higher viability observed for bioreactor 4 compared to bioreactor 2 can be attributed to the lower level of lactate produced during this run. The cellular metabolism was likely regulated by the pH shift. The off-line pH profile shows that lower pH was achieved through the implementation of the pH shift for bioreactor 3 compared to bioreactor 1. However, the pH was maintained at the top of the dead band, 6.85, due to the metabolic conversion from lactate production to consumption during the later days of culture. The pH level for bioreactor 4 was more desirable. Following the pH shift on day 4, the level remained at 6.7-6.75 through day 15. This was achievable due to the increase in lactate during the later days in culture. From this experiment it was concluded that the increase in feed percentage on day 6 causes osmolality to rise which subsequently prevents the cells from undergoing the metabolic flux from lactate production to consumption. The CO₂ and all other metabolites were comparable between the runs. All additional data for the production bioreactors in Study No. 2 can be found in Table 1-23.

### Titer

Bioreactor 3 produced more than reactors 1, 2 and 4 which all had comparable day 15 results. The increase in titer observed for bioreactor 3 may be attributed to the increase in cell growth coupled with low pH condition.

### Product Quality

Time course-samples were purified using the HiTrap Pro A method and submitted for IEC analysis. Samples purified through the downstream SDM pro A method were submitted for NR CE-SDS, SEC and glycan. The day 15 results for SEC and glycan were as expected. Half-antibody levels for bioreactor 3 and 4 remained higher, but within the historical data set for small-scale runs. Bioreactor 3 and 4 had lower half-antibody levels compared to bioreactor 1 and 2 on day 15.

The IEC results confirm that lower culture pH results in lower acidic species. Samples from bioreactors 3 and 4 were run and shown in Table 1-10. The data indicate that both feed percentage (used to induce lactate production) and pH set point are important in maintaining a charge profile.

TABLE 1-9
Study No. 2 NR CE-SDS

| Bioreactor ID | Day | Half-Antibody (%) | IgG (%) |
|---|---|---|---|
| R3 | SDM ProA Eluate | 7.33 | 91.2 |
| R4 | SDM ProA Eluate | 9.73 | 88.5 |

TABLE 1-10
Study No. 2 IEC

| | | Acidic | Main | Basic | | | | | | | | |
|---|---|---|---|---|---|---|---|---|---|---|---|---|
| Bioreactor | | Peak | Peak | Peak | Acidic | Acidic | Acidic | Basic | Basic | Basic | Basic | Basic |
| ID | Day | (%) | (%) | (%) | 3 (%) | 2 (%) | 1 (%) | 1 (%) | 2 (%) | 3 (%) | 4 (%) | 5 (%) |
| R3 | 15 | 19.3 | 60.2 | 20.5 | 1.31 | 4.24 | 13.8 | 9.51 | 9.37 | 1.2 | 0.41 | — |
| R4 | 13 | 13.4 | 55.1 | 31.5 | 1.16 | 3.11 | 9.12 | 10.09 | 14.46 | 2.8 | 2.8 | 1.38 |
| R4 | 14 | 15.3 | 53.4 | 31.3 | 1.85 | 3.85 | 9.62 | 9.94 | 13.72 | 2.14 | 5.51 | — |
| R4 | 15 | 17.4 | 60.2 | 22.4 | 1.34 | 3.46 | 12.6 | 10.22 | 10.4 | 1.17 | 0.63 | — |

TABLE 1-11
Study No. 2 SEC

| Bioreactor | | Monomer | Aggregate | Fragment |
|---|---|---|---|---|
| ID | Day | (%) | (%) | (%) |
| R3 | SDM ProA Eluate | 98.1 | 0.74 | 1.14 |
| R4 | SDM ProA Eluate | 97.8 | 0.76 | 1.42 |

### Study No. 3—Bioreactor 5 (R5), Bioreactor 6 (R6), Bioreactor 7 (R7) and Bioreactor 8 (R8)

### Experimental Conditions

The first goal of this experiment was to confirm that increased feed coupled with a pH shift would reproducibly result in the desired cellular metabolism to keep pH at a low enough level to positively impact acidic species. Thus, bioreactor 5 was run under the same conditions as bioreactor 4. A pH shift to 6.65 rather than 6.75 was implemented for bioreactor 6 to evaluate whether further improvement could be made to the charge profile. Bioreactor 7 was utilized to evaluate the feeding scheme. Feeding commenced one day earlier on day 2, it was increased on day 6 and then subsequently decreased on day 9. Finally, an additional attempt to modulate the charge profile was made by implementing a temperature shift to 32° C. on day 9 for bioreactor 8. Details of the four bioreactors in this study have been captured in Table 1-12.

TABLE 1-12
Study No. 3 Experimental Conditions

| Parameter | Brx-5 | Brx-6 | Brx-7 | Brx-8 |
|---|---|---|---|---|
| 7a Feed Percentage | 2% (D 3-D 5) 3% | 2% (D 3-D 5) 3% | 2% (D 2-D 5), 3% | 2% (D 3-D 5) 3% |
| (% initial Vol) | (D 6-D 14) | (D 6-D 14) | (D 6-D 8), 2% | (D 6-D 14) |
| | | | (D 9-D 14) | |
| 7b Feed Percentage | 0.2%(D 3-D 5), | 0.2%(D 3-D 5), 0.3% | 0.2%(D 2-D 5), 0.3% | 0.2%(D 3-D 5), 0.3% |
| (% initial Vol) | 0.3% (D 6-D 14) | (D 6-D 14) | (D 6-D 8), 0.2% | (D 6-D 14) |
| | | | (D 9-D 14) | |
| Temperature (° C.) | 36 | | | 36(D 0-D 8), 32 |
| | | | | (D 9-D 15) |
| pH | 6.90 ± 0.15(D 0-D 3) to | 6.90 ± 0.15(D 0-D 3) to | 6.90 ± 0.15(D 0-D 3) to | 6.90 ± 0.15(D 0-D 3) to |
| | 6.75 ± 0.10 (D 4-D 15) | 6.65 ± 0.10 (D 4-D 15) | 6.75 ± 0.10 (D 4-D 15) | 6.75 ± 0.10 (D 4-D 15) |

### Production Data

The control condition, bioreactor 5, was comparable to the previous controls, bioreactors 2 and 4. This run confirmed the ability to control at a low pH through feeding and a pH set point shift. Bioreactors 6 and 7 both grew to a much lower peak cell density than the control and exhibited lower final viability. The overall culture performance for these two bioreactors was suboptimal relative to the control and demonstrated that the changes made to the pH set point and feeding scheme were ineffective. Bioreactor 8, with the temperature shift, performed comparably to the control with the exception of pH which was slightly higher from days 9-15. All additional data for the production bioreactors in Study No. 3 can be found in Table 1-24 and Table 1-25.

### Titer

As expected, the control condition produced more than bioreactors 6 and 7 which both exhibited poor cell growth and viability. Bioreactor 8 also had low productivity which can be attributed to the temperature shift to 32° C.

### Product Quality

Samples were purified using the HiTrap Pro A method and submitted for product quality analysis. The day 15 clarified harvest (CH) product quality results from this experiment are shown in Table 1-13 through Table 1-15. In respect to NR CE-SDS, SEC and Glycan all four bioreactors performed similarly. Bioreactors 6 and 7 had slightly higher G0 glycoforms than the other two reactors which is inconsequential since the culture performance was poor. The IEC data indicate that temperature had a large impact on acidic species; however, the conversion was not to main, but to basic which resulted in a relatively higher basic species level for R8. Based on culture performance, productivity and product quality, a recommendation was made to move the control condition forward to the 200 L demonstration run.

TABLE 1-13
Study No. 3 NR CE-SDS

| Bioreactor ID | Day | Half Antibody (%) | IgG (%) |
|---|---|---|---|
| R5 | 15 CH | 9.56 | 88.86 |
| R6 | 15 CH | 10.36 | 87.45 |
| R7 | 15 CH | 10.48 | 87.21 |
| R8 | 15 CH | 9.11 | 88.86 |

TABLE 1-14
Study No. 3 IEC

| | | IEC | IEC | IEC | IEC | IEC | IEC | IEC | IEC | IEC | IEC |
|---|---|---|---|---|---|---|---|---|---|---|---|
| | | Acidic | Main | Basic | Acidic | Acidic | Acidic | Basic | Basic | Basic | Basic |
| Bioreactor | | Peak | Peak | Peak | 3 | 2 | 1 | 1 | 2 | 3 | 4 |
| ID | Day | (%) | (%) | (%) | (%) | (%) | (%) | (%) | (%) | (%) | (%) |
| R5 | 15 CH | 14.7 | 58.14 | 27.2 | 1.47 | 3.42 | 9.82 | 8.75 | 13.59 | 2.62 | 2.19 |
| R6 | 15 CH | 16.7 | 51.93 | 31.4 | 1.01 | 4.2 | 11.48 | 12.71 | 12.28 | 4.2 | 2.18 |
| R7 | 15 CH | 14.6 | 52.54 | 32.9 | 1.1 | 3.12 | 10.38 | 13.15 | 13.33 | 4.22 | 2.16 |
| R8 | 15 CH | 11.6 | 50.66 | 37.7 | 0.81 | 2.52 | 8.26 | 11.57 | 17.0 | 3.72 | 5.45 |

TABLE 1-15
Study No. 3 SEC

| Bioreactor | | Monomer | Aggregate | Fragment |
|---|---|---|---|---|
| ID | Day | (%) | (%) | (%) |
| R5 | 15 CH | 98.4 | 0.57 | 1.02 |
| R6 | 15 CH | 98.4 | 0.42 | 1.15 |
| R7 | 15 CH | 97.7 | 1.09 | 1.2 |
| R8 | 15 CH | 98.4 | 0.56 | 1.09 |

### Study No. 4—Bioreactor 9 (R9), Bioreactor 10 (R10), and 200 L

### 10 L Experimental Conditions

Two 10 L bioreactors were run concurrently with the 200 L demonstration run, the conditions of each can be found in Table. Bioreactor 9 was a satellite to the 200 L run. Following inoculation, 7.0 L of culture was drained from the 200 L reactor and transferred to a 10 L XDR. All additions and feeds for the satellite were aliquoted from the 200 L run. Bioreactor 10 was inoculated with cells at a lower generation to evaluate the impact of cell age on process performance and product quality.

TABLE 1-16
Study No. 4 Experimental Conditions

| Brx-9 | Brx-10 |
|---|---|
| Satellite to the 200 L | Production bioreactor inoculated with |
| bioreactor | lower generation cells |

### 200 L Production Bioreactor Parameters

The final conditions for the 200 L run can be found in Table which match parameters used in bioreactor 4 and 5.

TABLE 1-17
200 L Production Bioreactor Parameters

| Parameter | 200 L |
|---|---|
| 7a Feed Percentage | 2% (D 3-D 5) 3% (D 6-D 14) |
| (% initial Vol) | |
| 7b Feed Percentage | 0.2%(D 3-D 5), 0.3% (D 6-D 14) |
| (% initial Vol) | |
| Temperature (° C.) | 36 ± 1 |
| pH | 6.90 ± 0.15(D 0-D 3) to |
| | 6.75 ± 0.10 (D 4-D 15) |

### Production Data

The VCD for the 200 L reactor outperformed the satellite and all other 10 L controls. The increase in cell growth is likely due to bioreactor geometry and scales. The satellite and low generation bioreactor grew comparably to the previous control conditions indicating that the scale down model is robust and cell age had no impact on cell growth. Viability was comparable between the 200 L run and the 10 L bioreactors. The lactate profile for the 200 L bioreactor differed from the satellite run and 10 L controls. It peaked at a lower concentration and began consuming lactate on day 5. In response to the lactate consumption observed in the 200 L bioreactor, a decision was made to add an additional 1%/0.1% bolus of both feed media to the 200 L bioreactor and 10 L satellite. The timing and volume of all additions to the 200 L bioreactor can be found in Table 1-. The additional bolus was effective in reverting the 200 L cellular metabolism to lactate production. However, a dramatic spike in lactate was observed from day 9 to 10. At the time of the lactate spike, a discrepancy was observed between the controlling and ancillary DO probes. The controlling probe was reading approximately 30% while the ancillary probe was reading approximately 3%. Considering the lactate spike, it was determined that the ancillary probe was reading correctly, and the controlling probe had drifted causing a low oxygen environment in the bioreactor. Upon switching the probes, the lactate decreased. The pH in the 200 L reactor trended on the higher side of the dead band with the exception of day 9-10 where it was impacted by the spike in lactate. The difference in pH profiles is due to the lower lactate production in the 200 L reactor. As expected, the CO₂ level in the 200 L bioreactor was slightly higher than the 10 L's. Osmolality was consistent among scales. The oscillations seen in the 200 L osmolality data are due to pre-feed and post-feed samples being taken on the same day. All additional production data in Study No. 4 can be found in Table 1-26.

This data demonstrates that, in respect to culture performance, the 10 L scale-down model is representative of the 200 L bioreactor with the exception of a slight offset in cell density. Since feed percentage is purposely used to influence cellular metabolism, the increased VCD at large scale likely impacted the lactate production. To mitigate unwanted lactate consumption, the increase in feed percentage should occur one day earlier on day 5 rather than day 6.

TABLE 1-18
200 L Bioreactor Additions

| | | | | Cell | Cell | | |
|---|---|---|---|---|---|---|---|
| | Glucose | Glucose | Glucose | Boost 7a | Boost 7b | Base | Antifoam |
| | Addition | Addition | Consumption | Addition | Addition | addition | addition |
| Day | (g/L) | (L) | (g/L/day) | (L) | (mL) | (mL) | (mL) |
| 0.0 | 0.000 | 0 | 0 | 0 | 0 | 0 | 0 |
| 1.0 | 0.000 | 0 | 0.60 | 0 | 0 | 0 | 0 |
| 2.0 | 0.000 | 0 | 0.82 | 0 | 0 | 0 | 0 |
| 3.0 | 1.540 | 0 | 1.14 | 2.794 | 280 | 0 | 5 |
| 4.0 | 1.540 | 0 | 1.16 | 2.8 | 280 | 0 | 5 |
| 5.0 | 1.540 | 0 | 1.38 | 2.82 | 280 | 0 | 5 |
| 6.0 | 2.310 | 0 | 1.86 | 4.21 | 420 | 0 | 0 |
| 6.8 | 4.220 | 0.588 | 2.63 | 4.21 | 420 | 0 | 5 |
| 8.0 | 3.920 | 0.512 | 4.05 | 4.22 | 420 | 0 | 10 |
| 9.0 | 6.110 | 0.996 | 4.53 | 4.26 | 420 | 0 | 5 |
| 9.1 | 0 | 0 | — | 1.406 | 140 | 0 | 5 |
| 10.0 | 3.560 | 0.427 | 5.11 | 4.2 | 420 | 0 | 5 |
| 10.1 | 0 | 0 | 1.62 | 0 | 0 | 12.6 | 5 |
| 11.0 | 3.830 | 0.533 | 3.72 | 4.208 | 420 | 0 | 10 |
| 12.0 | 4.730 | 0.872 | 4.93 | 4.2 | 420 | 0 | 10 |
| 13.0 | 4.54 | 0.826 | 4.38 | 4.2 | 420 | 0 | 10 |
| 14.0 | 4.34 | 0.773 | 4.33 | 4.2 | 420 | 0 | 10 |
| 15.0 | 4.19 | 0 | 4.33 | 0 | 0 | 0 | 0 |

### Titer

The increased cell density observed for the 200 L run resulted in an increase in titer compared to the 10 L satellite and previous controls. Reactor 10 had slightly lower titer than the satellite run however, the day 15 value was in line with previous control runs and within expectations for process variation.

### Product Quality

Samples for both the 200 L run and 10 L bioreactors were purified using the HiTrap Pro A method and submitted for product quality analysis. Pro A eluate from the downstream pilot scale run was also submitted for the 200 L reactor. Product quality results for bioreactor 9, the satellite, and bioreactor 10, the low generation condition, were comparable which indicates that cell age does not impact product quality. Half-antibody was slightly lower in the 200 L reactor material and decreased from day 12 to 15, with a slight increase observed in the clarified harvest, Table 1-19. This increase can be attributed to the worst-case harvest conditions. The charge heterogeneity for the 200 L reactor was comparable to the 10 L satellite and low generation bioreactor despite the higher pH profile, Table. The acidic and basic species for the 200 L run were in line with the 10 L model. Aggregate and fragment levels by SEC were marginally higher for the 200 L run compared to the 10 L model, Table 1-21. The day 15 glycan results for the 200 L run were in line with the 10 L model.

TABLE 1-19
Study No. 4 NR CE-SDS

| Bioreactor ID | Day | Half Antibody (%) | IgG (%) |
|---|---|---|---|
| R9 | 12 | 10.19 | 88.02 |
| R9 | 13 | 10.29 | 87.54 |
| R9 | 14 | 10.17 | 87.55 |
| R9 | 15 | 10.49 | 87.13 |
| R9 | 15 CH | 9.78 | 87.87 |
| R10 | 12 | 10.25 | 87.71 |
| R10 | 13 | 10.2 | 87.67 |
| R10 | 14 | 10.49 | 86.98 |
| R10 | 15 | 10.74 | 86.62 |
| 200 L R1 | 12 | 8.64 | 89.69 |
| 200 L R1 | 13 | 8.62 | 89.47 |
| 200 L R1 | 14 | 8.21 | 89.99 |
| 200 L R1 | 15 | 7.71 | 90.15 |
| 200 L R1 | 15 CH | 8.37 | 90.04 |
| 200 L R1 | Pro A Eluate | 6.86 | 90.97 |

TABLE 1-20
Study No. 4 IEC

| | | IEC | IEC | IEC | IEC | IEC | IEC | IEC | IEC | IEC | IEC |
|---|---|---|---|---|---|---|---|---|---|---|---|
| | | Acidic | Main | Basic | Acidic | Acidic | Acidic | Basic | Basic | Basic | Basic |
| Bioreactor | | Peak | Peak | Peak | 3 | 2 | 1 | 1 | 2 | 3 | 4 |
| ID | Day | (%) | (%) | (%) | (%) | (%) | (%) | (%) | (%) | (%) | (%) |
| R9 | 12 | 11 | 56.04 | 33 | 0.54 | 2.34 | 8.14 | 10.18 | 15.72 | 3.26 | 3.78 |
| R9 | 13 | 12 | 58.06 | 29.9 | 1.15 | 3.07 | 7.82 | 7.3 | 15.99 | 2.84 | 3.78 |
| R9 | 14 | 13.4 | 56.09 | 30.5 | 1.32 | 3.08 | 9.03 | 9.99 | 13.69 | 3.49 | 3.32 |
| R9 | 15 | 14.5 | 58.25 | 27.3 | 1.11 | 3.75 | 9.64 | 8.93 | 12.53 | 3.28 | 2.52 |
| R9 | 15 CH | 14.6 | 58.59 | 26.8 | 1.95 | 2.83 | 9.83 | 9.47 | 12.05 | 3.2 | 2.08 |
| R10 | 12 | 11 | 55.6 | 33.4 | 0.74 | 2.6 | 7.64 | 9.56 | 16.66 | 3.18 | 4.01 |
| R10 | 13 | 12.5 | 54.13 | 33.4 | 1.18 | 2.76 | 8.58 | 9.85 | 15.76 | 3.28 | 4.47 |
| R10 | 14 | 14.5 | 53.01 | 32.5 | 1.45 | 3.33 | 9.75 | 10.64 | 14.78 | 3.38 | 3.67 |
| R10 | 15 | 15.4 | 53.29 | 31.4 | 1.66 | 3.61 | 10.08 | 11.25 | 13.34 | 3.72 | 3.06 |
| 200 L R1 | 12 | 11.9 | 57.89 | 30.2 | 0.99 | 3.45 | 7.44 | 8.57 | 15 | 2.89 | 3.78 |
| 200 L R1 | 13 | 12.3 | 58.51 | 29.2 | 1.25 | 3.55 | 7.5 | 8.6 | 13.78 | 3.24 | 3.57 |
| 200 L R1 | 14 | 15.2 | 54.36 | 30.5 | 1.5 | 3.25 | 10.43 | 10.18 | 13.87 | 2.9 | 3.51 |
| 200 L R1 | 15 | 16.1 | 56.57 | 27.4 | 1.46 | 4.1 | 10.53 | 8.97 | 11.94 | 4.02 | 2.43 |
| 200 L R1 | 15 CH | 15.5 | 59.08 | 25.5 | 1.69 | 5.26 | 8.52 | 7.35 | 12.19 | 3.39 | 2.51 |
| 200 L R1 | Pro A Eluate | 16.4 | 57.29 | 26.3 | 1.49 | 3.62 | 11.25 | 9.09 | 11.43 | 3.59 | 2.22 |

TABLE 1-21
Study No. 4 SEC

| | | Monomer | Aggregate | Fragment |
|---|---|---|---|---|
| Bioreactor ID | Day | (%) | (%) | (%) |
| R9 | 12 | 98 | 0.94 | 1.06 |
| R9 | 13 | 98.31 | 0.61 | 1.08 |
| R9 | 14 | 98.06 | 0.62 | 1.32 |
| R9 | 15 | 98.26 | 0.61 | 1.14 |
| R9 | 15 CH | 98.38 | 0.48 | 1.14 |
| R10 | 12 | 98.37 | 0.52 | 1.11 |
| R10 | 13 | 98.19 | 0.57 | 1.23 |
| R10 | 14 | 98.34 | 0.55 | 1.1 |
| R10 | 15 | 98.15 | 0.58 | 1.27 |
| 200 L R1 | 12 | 98.31 | 0.39 | 1.29 |
| 200 L R1 | 13 | 97.84 | 0.76 | 1.4 |
| 200 L R1 | 14 | 97.78 | 0.54 | 1.68 |
| 200 L R1 | 15 | 97.87 | 0.63 | 1.50 |
| 200 L R1 | 15 CH | 97.5 | 0.94 | 1.56 |
| 200 L R1 | Pro A Eluate | 98.6 | 0.8 | 0.65 |

### Conclusion

This example demonstrates that the upstream process for mavrilimumab production is robust and scalable. The 10 L small scale model is reproducible and predictive of the 200 L bioreactor. The harvest procedure proved to be scalable with high throughput and high yield. By increasing feed and shifting pH of the cell culture, desired product quality was achieved.

The operating parameters that were chosen to move forward to the confirmation run are identical to those used during this demonstration run and are detailed in Table 1-1: Bioreactor parameters.

TABLE 1-22a
Production Data
Study No. 1

| BRX | | VCD | Viability | pH | | | Glutamine | Glutamate | Glucose | Lactate | Ammonium |
|---|---|---|---|---|---|---|---|---|---|---|---|
| ID | Day | (e6 vc/mL) | (%) | offline | pCO2 | pO2 | (g/L) | (g/L) | (g/L) | (g/L) | (g/L) |
| R1 | 0.0 | 0.87 | 97.80 | 7.07 | 60.4 | 101.5 | N/A | 3.47 | 5.6 | 0.55 | 3.27 |
| R1 | 1.0 | 1.61 | 97.90 | 7.11 | 44.5 | 91.60 | N/A | 3.64 | 5 | 0.9 | 3.54 |
| R1 | 2.0 | 2.67 | 97.50 | 7.04 | 23.8 | 81.30 | N/A | 3.83 | 4.14 | 1.51 | 4.1 |
| R1 | 3.0 | 3.96 | 98.30 | 6.85 | 16.4 | 68.90 | N/A | 3.56 | 2.9 | 1.86 | 4.35 |
| R1 | 4.0 | 6.49 | 97.90 | 6.77 | 25.7 | 0.00 | N/A | 5.16 | 4.84 | 2.02 | 4.61 |
| R1 | 5.0 | 9.98 | 98.30 | 6.80 | 36.2 | 0.00 | 0.67 | 5.72 | 5.08 | 1.95 | 4.31 |
| R1 | 6.0 | 12.20 | 98.00 | 6.80 | 51.2 | 33.00 | 0.42 | 7.43 | 4.93 | 1.94 | 3.77 |
| R1 | 7.0 | 13.60 | 98.00 | 6.83 | 59.2 | 28.50 | N/A | N/A | 4.16 | 1.94 | 3.26 |
| R1 | 8.0 | 17.40 | 97.70 | 6.84 | 70.8 | 13.40 | 1.22 | 5.18 | 2.64 | 1.91 | 2.79 |
| R1 | 9.0 | 20.60 | 97.80 | 6.89 | 73.6 | 86.50 | 1.03 | 4.39 | 3.28 | 1.97 | 2.57 |
| R1 | 10.0 | 21.50 | 95.90 | 6.88 | 84.9 | 6.20 | N/A | N/A | 2.74 | 1.8 | 2.66 |
| R1 | 11.0 | 19.60 | 95.70 | 7.01 | 68.2 | 8.60 | 0.67 | 0.34 | 2.03 | 1.77 | 1.59 |
| R1 | 12.0 | 22.55 | 95.00 | 7.05 | 64.8 | 11.8 | N/A | N/A | 2.89 | 1.61 | 1.76 |
| R1 | 13.0 | 22.10 | 93.80 | 7.08 | 58.9 | 0.00 | 0.78 | 0.28 | 2.74 | 1.47 | 2.25 |
| R1 | 14.0 | 24.75 | 93.50 | 7.08 | 51.8 | 16.3 | N/A | N/A | 2.75 | 1.36 | 2.52 |
| R1 | 15.0 | 22.15 | 91.25 | 6.96 | 48.3 | 24.8 | N/A | N/A | 3.19 | 1.52 | 3.38 |
| R2 | 0.0 | 0.976 | 97.8 | 7.035 | 64.5 | 103.4 | | 3.4 | 5.66 | 0.54 | 3.15 |
| R2 | 1.0 | 1.36 | 97.3 | 7.068 | 50.6 | 96.9 | | 3.51 | 5.14 | 0.88 | 3.56 |
| R2 | 2.0 | 2.52 | 98.8 | 7.031 | 32.8 | 97.3 | | 3.64 | 4.19 | 1.34 | 3.96 |
| R2 | 3.0 | 4.17 | 98.4 | 6.865 | 18.9 | 69.9 | | 3.56 | 2.98 | 1.79 | 4.23 |
| R2 | 4.0 | 6.46 | 95.5 | 6.803 | 29 | 71.8 | | 4.83 | 4.4 | 1.95 | 4.37 |
| R2 | 5.0 | 8.12 | 98.1 | 6.824 | 39.7 | 58.7 | 0.28 | 6.09 | 4.59 | 1.93 | 4.06 |
| R2 | 6.0 | 10.2 | 97.9 | 6.82 | 54.7 | 28 | 0.57 | 6.24 | 4.16 | 1.85 | 3.4 |
| R2 | 7.0 | 14.1 | 97.6 | 6.823 | 73.3 | 40.8 | 0.82 | 6.94 | 4.04 | 1.84 | 3.28 |
| R2 | 8.0 | 15.1 | 97 | 6.846 | 64.2 | 23.6 | 1.25 | 6.09 | 2.73 | 2 | 3 |
| R2 | 9.0 | 19.3 | 97.3 | 6.859 | 67 | 66.4 | 1.05 | 5.55 | 3.5 | 2.06 | 2.81 |
| R2 | 10.0 | 20.1 | 96.1 | 6.844 | 73.1 | 23.3 | N/A | N/A | 1.73 | 2.17 | 2.63 |
| R2 | 11.0 | 18.3 | 94.5 | 6.821 | 72.6 | 9.2 | 0.87 | 3.01 | 2.7 | 2.32 | 2.52 |
| R2 | 12.0 | 20.25 | 92.8 | 6.803 | 63.1 | 40.1 | 0.81 | 2.48 | 3.12 | 2.58 | 2.75 |
| R2 | 13.0 | 20.9 | 91.6 | 6.73 | 67.1 | 4.9 | 0.91 | 2.36 | 3.54 | 2.81 | 3.45 |
| R2 | 14.0 | 21.4 | 88.95 | 6.692 | 60.1 | 37.8 | N/A | N/A | 3.56 | 3.13 | 4.68 |
| R2 | 15.0 | 18.8 | 84.25 | 6.673 | 57.1 | 53.4 | 1.38 | 3.59 | 3.95 | 4.32 | 5.84 |

TABLE 1-22b
Production Data
Study No. 1

| | | | | | | Glucose | Feed A | Feed B | Base | Antifoam |
|---|---|---|---|---|---|---|---|---|---|---|
| BRX | | Na+ | K+ | Osmolality | Titer | Addition | Addition | Addition | addition | addition |
| ID | Day | (mM) | (mM) | (mOsm/kg) | (g/L) | (mL) | (mL) | (mL) | (mL) | (mL) |
| R1 | 0.0 | 119.3 | 10.77 | 315 | N/A | 0.0 | 0 | 0 | 0.0 | 0.0 |
| R1 | 1.0 | 120.5 | 10.61 | 309 | N/A | 0.0 | 0 | 0 | 0.0 | 0.0 |
| R1 | 2.0 | 121.2 | 10.65 | 303 | N/A | 0.0 | 0 | 0 | 0.0 | 0.0 |
| R1 | 3.0 | 118 | 10.02 | 296 | N/A | 17.0 | 152.0 | 15.0 | 0.0 | 0.0 |
| R1 | 4.0 | 123.5 | 10.98 | 323 | N/A | 0.0 | 150.0 | 15.0 | 0.0 | 0.0 |
| R1 | 5.0 | 124.8 | 11.15 | 337 | N/A | 0.0 | 150.0 | 15.0 | 0.0 | 0.0 |
| R1 | 6.0 | 126.1 | 11.46 | 346 | N/A | 0.0 | 150.0 | 15.0 | 0.0 | 0.0 |
| R1 | 7.0 | 128.9 | 11.66 | 346 | N/A | 0.0 | 150.0 | 15.0 | 0.0 | 0.0 |
| R1 | 8.0 | 132.8 | 11.7 | 341 | N/A | 40.0 | 150.0 | 15.0 | 0.0 | 0.0 |
| R1 | 9.0 | 132.8 | 11.53 | 345 | N/A | 29.0 | 150.0 | 15.0 | 0.0 | 0.6 |
| R1 | 10.0 | 137.3 | 11.63 | 341 | N/A | 41.1 | 150.7 | 15.0 | 0.0 | 0.0 |
| R1 | 11.0 | 137.6 | 11.41 | 334 | 4.7 | 53.0 | 150.0 | 15.0 | 0.0 | 0.5 |
| R1 | 12.0 | 140.9 | 12.22 | 348 | 0 | 39.0 | 151.0 | 15.0 | 0.0 | 1.0 |
| R1 | 13.0 | 146 | 13.18 | 353 | 5.71 | 42.0 | 150.0 | 15.0 | 0.0 | 0.5 |
| R1 | 14.0 | 145.7 | 13.73 | 359 | 0 | 42.7 | 150.0 | 15.0 | 0.0 | 0.5 |
| R1 | 15.0 | 142.4 | 14.13 | 373 | 6.48 | 0 | 0 | 0 | 0 | 0 |
| R2 | 0.0 | 120.3 | 10.81 | 314 | N/A | 0 | 0 | 0 | 0 | 0.0 |
| R2 | 1.0 | 120.1 | 10.69 | 312 | N/A | 0 | 0 | 0 | 0 | 7.068 |
| R2 | 2.0 | 118.8 | 10.41 | 303 | N/A | 0.00 | 0 | 0 | 0 | 0 |
| R2 | 3.0 | 113.8 | 9.82 | 296 | N/A | 14.00 | 141 | 14 | 0 | 0 |
| R2 | 4.0 | 119.9 | 10.72 | 317 | N/A | 0.00 | 141.3 | 14 | 0 | 0 |
| R2 | 5.0 | 123 | 10.88 | 326 | N/A | 0.00 | 140 | 14 | 0 | 0 |
| R2 | 6.0 | 124.5 | 11.01 | 333 | N/A | 0.00 | 210 | 21 | 0 | 0 |
| R2 | 7.0 | 130.8 | 11.78 | 346 | N/A | 0.00 | 210 | 21 | 0 | 0 |
| R2 | 8.0 | 132.6 | 11.79 | 348 | N/A | 35.70 | 210 | 21 | 0 | 0 |
| R2 | 9.0 | 136.8 | 11.95 | 354 | N/A | 0.00 | 211 | 21 | 0 | 0.5 |
| R2 | 10.0 | 140 | 12.49 | 350 | N/A | 55.10 | 211.3 | 21 | 0 | 0 |
| R2 | 11.0 | 140.1 | 12.51 | 360 | 4.55 | 40.00 | 210 | 21 | 0 | 0.5 |
| R2 | 12.0 | 140.3 | 13.25 | 368 | N/A | 33.00 | 210 | 21 | 0 | 0.8 |
| R2 | 13.0 | 144.8 | 14.55 | 385 | 6.15 | 26.00 | 210 | 21 | 0 | 0.6 |
| R2 | 14.0 | 146.2 | 15.35 | 411 | N/A | 26.70 | 211 | 21 | 14 | 0.7 |
| R2 | 15.0 | 158.8 | 17.38 | 446 | 6.83 | 0.00 | 0 | 0 | 0 | 0 |

TABLE 1-23a
Production Data
Study No. 2

| BRX | | VCD | Viability | pH | | | Glutamine | Glutamate | Glucose | Lactate | Ammonium |
|---|---|---|---|---|---|---|---|---|---|---|---|
| ID | Day | (e6 vc/mL) | (%) | offline | pCO2 | pO2 | (g/L) | (g/L) | (g/L) | (g/L) | (g/L) |
| R3 | 0.0 | 0.777 | 98.2 | 7.094 | 43.3 | 107.3 | N/A | 3.05 | 5.71 | 0.93 | 3.09 |
| R3 | 1.0 | 1.275 | 96.5 | 7.096 | 30.3 | 68.7 | N/A | 3.1 | 5.24 | 1.04 | 3.4 |
| R3 | 2.0 | 2.31 | 98.9 | 7.016 | 11.9 | 30.7 | N/A | 3.27 | 4.46 | 1.69 | 4.03 |
| R3 | 3.0 | 3.685 | 97.5 | 6.745 | 11 | 52.4 | N/A | 3.38 | 3.56 | 2.14 | 4.64 |
| R3 | 4.0 | 5.46 | 97.6 | 6.698 | 16 | 49.6 | N/A | 6.59 | 4.4 | 1.82 | 4.96 |
| R3 | 5.0 | 6.67 | 98.2 | 6.763 | 24.4 | 51.5 | 3.54 | 7.5 | 4.99 | 2.02 | 5.19 |
| R3 | 6.0 | 9.4 | 98.4 | 6.77 | 30.7 | 44.4 | 6.35 | 9.73 | 5.09 | 2.04 | 5 |
| R3 | 7.0 | 12.5 | 98.3 | 6.736 | 44.9 | 34.9 | 0 | 8.76 | 4.59 | 1.97 | 4.4 |
| R3 | 8.0 | 14.85 | 97.7 | 6.818 | 48.9 | 42.6 | 0 | 9.82 | 3.7 | 1.95 | 3.94 |
| R3 | 9.0 | 17.35 | 98.4 | 6.837 | 54.8 | 42.5 | 0 | 9.02 | 3.28 | 1.97 | 3.37 |
| R3 | 10.0 | 20.85 | 97.4 | 6.837 | 63.4 | 1.5 | 0.25 | 5.79 | 2.66 | 1.96 | 2.93 |
| R3 | 11.0 | 22.35 | 97.2 | 6.894 | 71.8 | 9.7 | 0.17 | 3.73 | 2.24 | 1.67 | 2.62 |
| R3 | 12.0 | 23.65 | 96.1 | 6.916 | 107 | 32.5 | 0.35 | 0.87 | 2.08 | 1.23 | 2.67 |
| R3 | 13.0 | 25.75 | 95.4 | 6.879 | 107.9 | 46.4 | 0.39 | 0 | 2.27 | 1.38 | 3.62 |
| R3 | 14.0 | 26.65 | 94.1 | 6.89 | 82.9 | 51.2 | 0.44 | 0 | 1.77 | 1.54 | 4.28 |
| R3 | 15.0 | 25.1 | 93.9 | 6.863 | 89.8 | 40 | 0.35 | 0.41 | 2.5 | 1.38 | 4.17 |
| R4 | 0.0 | 0.805 | 97.6 | 7.079 | 46 | 127.1 | N/A | 3.09 | 5.81 | 0.98 | 3.17 |
| R4 | 1.0 | 1.28 | 98.2 | 7.08 | 32.9 | 70.9 | N/A | 3.1 | 5.18 | 1.26 | 3.37 |
| R4 | 2.0 | 2.19 | 98.7 | 7.06 | 12.3 | 93.1 | N/A | 3.26 | 4.37 | 1.63 | 3.96 |
| R4 | 3.0 | 3.84 | 97.2 | 6.775 | 9.9 | 53.3 | N/A | 3.28 | 3.4 | 2.07 | 4.49 |
| R4 | 4.0 | 5.535 | 98.3 | 6.742 | 18.8 | 76.1 | N/A | 6.05 | 4.17 | 1.84 | 5.03 |
| R4 | 5.0 | 6.98 | 98 | 6.686 | 26.9 | 69.2 | N/A | 7.44 | 4.47 | 1.91 | 4.96 |
| R4 | 6.0 | 9.48 | 98.3 | 6.754 | 33.2 | 35.2 | N/A | 8.97 | 4.47 | 1.95 | 4.69 |
| R4 | 7.0 | 11.9 | 97.6 | 6.758 | 42.5 | 41.5 | 0.09 | 8.33 | 4.6 | 1.99 | 4.45 |
| R4 | 8.0 | 14.7 | 97.6 | 6.772 | 57.9 | 50.9 | N/A | 9.88 | 4.23 | 1.97 | 4.32 |
| R4 | 9.0 | 17.35 | 98.2 | 6.776 | 72.1 | 50.7 | 0.12 | 8.85 | 3.93 | 2.02 | 4.19 |
| R4 | 10.0 | 20.9 | 97 | 6.808 | 70.4 | 49.3 | 0.3 | 8.12 | 3.38 | 2.06 | 3.98 |
| R4 | 11.0 | 20.33333333 | 96.4 | 6.822 | 71.3 | 46.2 | 0.4 | 7.11 | 2.85 | 2.14 | 3.77 |
| R4 | 12.0 | 21.55 | 95.2 | 6.814 | 73.5 | 36.3 | 1.06 | 5.32 | 2.54 | 2.37 | 3.67 |
| R4 | 13.0 | 21.13333333 | 94.1 | 6.797 | 68 | 58.7 | 0.64 | 5.6 | 3.28 | 2.52 | 3.89 |
| R4 | 14.0 | 21.7 | 93 | 6.773 | 60.3 | 59.4 | 0.61 | 5.73 | 3.16 | 2.72 | 4.53 |
| R4 | 15.0 | 21.6 | 90.9 | 6.718 | 62.4 | 65.2 | 1.4 | 5.21 | 3.65 | 2.92 | 5.4 |

TABLE 1-23b
Production Data
Study No. 2

| | | | | | | Glucose | Feed A | Feed B | Base | Antifoam |
|---|---|---|---|---|---|---|---|---|---|---|
| BRX | | Na+ | K+ | Osmolality | Titer | Addition | Addition | Addition | addition | addition |
| ID | Day | (mM) | (mM) | (mOsm/kg) | (g/L) | (mL) | (mL) | (mL) | (mL) | (mL) |
| R3 | 0.0 | 115.8 | 10.42 | 305 | N/A | 0.0 | 0 | 0 | 0.0 | 0.0 |
| R3 | 1.0 | 115.4 | 10.34 | 309 | N/A | 0.0 | 0 | 0 | 0.0 | 0.0 |
| R3 | 2.0 | 119.2 | 10.37 | 299 | N/A | 0.0 | 0 | 0 | 0.0 | 0.0 |
| R3 | 3.0 | 118.3 | 10.22 | 305 | N/A | 0.0 | 142.0 | 14.0 | 0.0 | 0.0 |
| R3 | 4.0 | 116.7 | 10.57 | 317 | N/A | 0.0 | 140.1 | 15.0 | 0.0 | 0.0 |
| R3 | 5.0 | 124.2 | 11.51 | 335 | N/A | 0.0 | 140.0 | 15.0 | 0.0 | 0.0 |
| R3 | 6.0 | 125.6 | 11.81 | 346 | N/A | 0.0 | 140.0 | 15.0 | 0.0 | 0.0 |
| R3 | 7.0 | 126 | 11.79 | 351 | N/A | 0.0 | 140.0 | 14.0 | 0.0 | 0.0 |
| R3 | 8.0 | 129.6 | 12.03 | 352 | N/A | 20.1 | 140.2 | 14.0 | 0.0 | 0.0 |
| R3 | 9.0 | 133 | 11.95 | 348 | N/A | 26.8 | 140.0 | 12.0 | 0.0 | 0.0 |
| R3 | 10.0 | 137.9 | 11.94 | 345 | N/A | 39.0 | 140.3 | 14.0 | 0.0 | 0.0 |
| R3 | 11.0 | 136.9 | 11.57 | 340 | N/A | 44.3 | 142.0 | 14.0 | 0.0 | 0.56 |
| R3 | 12.0 | 139.1 | 11.68 | 340 | 4.73 | 48.0 | 140.0 | 14.0 | 0.0 | 0.44 |
| R3 | 13.0 | 141.4 | 12.09 | 342 | 5.69 | 46.0 | 140.8 | 15.0 | 0.0 | 0.5 |
| R3 | 14.0 | 140.7 | 12.87 | 346 | 6.84 | 54.2 | 140.0 | 15.0 | 0.0 | 1.0 |
| R3 | 15.0 | 144.6 | 13.33 | 350 | 7.39 | 0.0 | 0.0 | 0 | 0 | 0 |
| R4 | 0.0 | 118.4 | 10.61 | 307 | N/A | 0 | 0 | 0 | 0 | 0 |
| R4 | 1.0 | 115.5 | 10.35 | 310 | N/A | 0.00 | 0 | 0 | 0 | 0 |
| R4 | 2.0 | 117.1 | 10.32 | 299 | N/A | 0.00 | 0 | 0 | 1 | 0 |
| R4 | 3.0 | 114 | 9.95 | 298 | N/A | 0.00 | 140 | 12 | 0 | 0 |
| R4 | 4.0 | 121.2 | 10.81 | 314 | N/A | 0.00 | 141 | 15 | 1 | 0 |
| R4 | 5.0 | 121.2 | 11.1 | 334 | N/A | 0.00 | 141 | 14 | 1 | 0 |
| R4 | 6.0 | 123.3 | 11.42 | 340 | N/A | 0.00 | 210.55 | 21 | 3 | 0 |
| R4 | 7.0 | 127.5 | 12 | 353 | N/A | 0.00 | 210.4 | 21 | 1 | 0 |
| R4 | 8.0 | 130.2 | 12.31 | 366 | N/A | 12.37 | 209.87 | 22 | 1 | 0 |
| R4 | 9.0 | 133.8 | 12.69 | 371 | N/A | 17.10 | 210.05 | 21 | 0 | 0.51 |
| R4 | 10.0 | 136.7 | 12.91 | 377 | N/A | 27.17 | 209.98 | 22 | 0 | 0 |
| R4 | 11.0 | 139.1 | 13.01 | 375 | N/A | 36.49 | 210.71 | 22 | 0 | 0 |
| R4 | 12.0 | 141.3 | 13.51 | 379 | 4.53 | 45.16 | 209.13 | 22 | 0 | 0.51 |
| R4 | 13.0 | 143.5 | 14.17 | 395 | 5.19 | 31.02 | 209.75 | 22 | 0 | 0.64 |
| R4 | 14.0 | 146.7 | 15.26 | 405 | 6.06 | 31.60 | 209.24 | 22 | 0 | 0 |
| R4 | 15.0 | 148.4 | 16.54 | 424 | 6.66 | 0.00 | 0 | 0 | 0 | 0 |

TABLE 1-24a
Production Data
Study No. 3

| BRX | | VCD | Viability | pH | | | Glutamine | Glutamate | Glucose | Lactate | Ammonium |
|---|---|---|---|---|---|---|---|---|---|---|---|
| ID | Day | (e6 vc/mL) | (%) | offline | pCO2 | pO2 | (g/L) | (g/L) | (g/L) | (g/L) | (g/L) |
| R5 | 0.0 | 0.772 | 97.4 | 7.077 | 44 | 114.8 | 0.04 | 3.16 | 5.77 | 0.68 | 3.03 |
| R5 | 1.0 | 1.875 | 97.8 | 7.046 | 36.9 | 85.6 | 0.07 | 3.29 | 5.29 | 1.1 | 3.44 |
| R5 | 2.0 | 2.435 | 98.25 | 7.009 | 23.7 | 48.3 | 0 | 3.36 | 4.34 | 1.4 | 4.04 |
| R5 | 3.0 | 3.885 | 98.4 | 6.781 | 18.5 | 41 | 0.01 | 3.47 | 3.51 | 1.92 | 4.57 |
| R5 | 4.0 | 5.455 | 98.5 | 6.742 | 21.3 | 49.2 | N/A | 5.16 | 4.28 | 2.2 | 5.03 |
| R5 | 5.0 | 7.6 | 98.1 | 6.758 | 26 | 86.2 | 0 | 6.38 | 4.61 | 2.15 | 4.99 |
| R5 | 6.0 | 9.38 | 97.9 | 6.762 | 36.4 | 62.5 | 0 | 7.3 | 4.43 | 2.2 | 4.56 |
| R5 | 7.0 | 12.55 | 97.75 | 6.759 | 45.3 | 53.8 | 0 | 8.45 | 4.69 | 2.19 | 4.27 |
| R5 | 8.0 | 15.95 | 97.4 | 6.774 | 50 | 35.6 | 0.07 | 8.99 | 4.25 | 2.22 | 4.04 |
| R5 | 9.0 | 18.25 | 97.35 | 6.79 | 80.4 | 29.8 | 0.18 | 8.74 | 3.62 | 2.27 | 3.81 |
| R5 | 10.0 | 19.55 | 96.1 | 6.812 | 73.8 | 27.5 | 0.27 | 7.94 | 3.16 | 2.36 | 3.55 |
| R5 | 11.0 | 22.05 | 95.65 | 6.806 | 78.2 | 24.7 | 0.39 | 6.74 | 2.66 | 2.44 | 3.33 |
| R5 | 12.0 | 22.75 | 93.9 | 6.816 | 82.6 | 24.8 | 0.49 | 5.69 | 2.3 | 2.5 | 3.32 |
| R5 | 13.0 | 23.25 | 92.6 | 6.799 | 86.6 | 4 | 0.57 | 4.84 | 2.71 | 2.63 | 3.47 |
| R5 | 14.0 | 24 | 91.2 | 6.811 | 71.5 | 45.6 | 0.66 | 4.28 | 2.39 | 2.73 | 3.75 |
| R5 | 15.0 | 22.85 | 89 | 6.743 | 72.5 | 35.5 | 0.73 | 4.86 | 3.53 | 2.97 | 4.59 |
| R6 | 0.0 | 0.762 | 97.7 | 7.055 | 48.3 | 124.2 | 0.05 | 3.16 | 5.81 | 0.67 | 3 |
| R6 | 1.0 | 1.325 | 98.1 | 7.001 | 41.5 | 71.8 | 0.06 | 3.18 | 5.3 | 0.99 | 3.39 |
| R6 | 2.0 | 2.135 | 98.05 | 7.006 | 28.5 | 58.6 | 0 | 3.35 | 4.54 | 1.27 | 4.07 |
| R6 | 3.0 | 3.46 | 98.2 | 6.805 | 29 | 47.9 | 0.02 | 3.41 | 3.71 | 1.72 | 4.54 |
| R6 | 4.0 | 5.105 | 98.2 | 6.745 | 31 | 53.3 | 0 | 4.92 | 4.42 | 1.9 | 4.97 |
| R6 | 5.0 | 7.565 | 98.4 | 6.73 | 47 | 48.4 | 0 | 6.24 | 5 | 1.9 | 5.14 |
| R6 | 6.0 | 10.6 | 97.8 | 6.759 | 59.2 | 78.8 | 0.04 | 7.06 | 4.93 | 1.81 | 4.82 |
| R6 | 7.0 | 13.35 | 97.6 | 6.768 | 75.4 | 65.3 | 0.13 | 7.79 | 5.11 | 1.66 | 4.7 |
| R6 | 8.0 | 15.15 | 97.2 | 6.741 | 110.7 | 29.2 | 0.24 | 8.62 | 5.13 | 1.65 | 5.1 |
| R6 | 9.0 | 16.85 | 97.95 | 6.756 | 177.7 | 32.8 | 0.38 | 8.59 | 4.54 | 1.62 | 5.53 |
| R6 | 10.0 | 16.35 | 95.4 | 6.795 | 139.2 | 41 | 0.5 | 8.51 | 3.83 | 1.93 | 5.58 |
| R6 | 11.0 | 16.55 | 94.1 | 6.795 | 136.9 | 42.3 | 0.68 | 8.65 | 3.87 | 2.25 | 5.88 |
| R6 | 12.0 | 17.1 | 92.95 | 6.806 | 139.9 | 63.3 | 0.94 | 8.9 | 4.07 | 2.42 | 6.32 |
| R6 | 13.0 | 18.15 | 90.5 | 6.789 | 145.4 | 44.5 | 1.05 | 9.29 | 4.03 | 2.69 | 7.02 |
| R6 | 14.0 | 18.65 | 88 | 6.805 | 112 | 71.9 | 1 | 9.38 | 3.33 | 3.01 | 7.55 |
| R6 | 15.0 | 17.25 | 86.85 | 6.742 | 99.9 | 55.8 | 1.2 | 9.47 | 3.9 | 3.38 | 8.31 |

TABLE 1-24b
Production Data
Study No. 3

| | | | | | | Glucose | Feed A | Feed B | Base | Antifoam |
|---|---|---|---|---|---|---|---|---|---|---|
| BRX | | Na+ | K+ | Osmolality | Titer | Addition | Addition | Addition | addition | addition |
| ID | Day | (mM) | (mM) | (mOsm/kg) | (g/L) | (mL) | (mL) | (mL) | (mL) | (mL) |
| R5 | 0.0 | 116.9 | 10.39 | 308 | N/A | 0.00 | 0 | 0 | 0 | 0 |
| R5 | 1.0 | 118.1 | 10.46 | 306 | N/A | 0.00 | 0 | 0 | 0 | 0 |
| R5 | 2.0 | 115 | 10.08 | 299 | N/A | 0.00 | 0 | 0 | 1 | 0 |
| R5 | 3.0 | 116.4 | 10.05 | 295 | N/A | 0.00 | 145.76 | 14 | 0 | 0 |
| R5 | 4.0 | 120.7 | 10.76 | 315 | N/A | 0.00 | 140.33 | 14 | 1 | 0 |
| R5 | 5.0 | 121.3 | 11.04 | 329 | N/A | 0.00 | 139.97 | 14 | 2 | 0 |
| R5 | 6.0 | 126.6 | 11.74 | 339 | N/A | 0.00 | 209.93 | 20 | 1 | 0 |
| R5 | 7.0 | 129.5 | 12.24 | 354 | N/A | 0.00 | 210.13 | 20 | 2 | 0 |
| R5 | 8.0 | 133.5 | 12.48 | 370 | N/A | 12.06 | 211.01 | 21 | 1 | 0 |
| R5 | 9.0 | 135.1 | 12.46 | 367 | N/A | 22.29 | 210.52 | 21 | 12 | 0.5 |
| R5 | 10.0 | 139.6 | 12.8 | 372 | N/A | 30.89 | 209.66 | 21 | 2 | 0.39 |
| R5 | 11.0 | 142.1 | 13.13 | 372 | N/A | 41.27 | 211.22 | 20 | 3 | 0.48 |
| R5 | 12.0 | 144.1 | 13.51 | 380 | N/A | 48.41 | 210.69 | 20 | 0 | 0.51 |
| R5 | 13.0 | 147 | 14.33 | 385 | 5.79 | 41.20 | 210.14 | 20 | 3 | 0.51 |
| R5 | 14.0 | 144.2 | 14.92 | 434 | 6.66 | 45.95 | 210.1 | 22 | 0 | 0.54 |
| R5 | 15.0 | 146.6 | 16.08 | 416 | 7.28 | 0.00 | 0 | 0 | 0 | 0 |
| R6 | 0.0 | 117.4 | 10.46 | 311 | N/A | 0 | 0 | 0 | 0 | 0 |
| R6 | 1.0 | 115.7 | 10.27 | 305 | N/A | 0.00 | 0 | 0 | 0 | 0 |
| R6 | 2.0 | 115.6 | 10.2 | 299 | N/A | 0.00 | 0 | 0 | 0 | 0 |
| R6 | 3.0 | 116.5 | 10.06 | 296 | N/A | 0.00 | 140 | 15 | 0 | 0 |
| R6 | 4.0 | 118.9 | 10.66 | 315 | N/A | 0.00 | 140 | 14 | 0 | 0 |
| R6 | 5.0 | 122.5 | 11.25 | 331 | N/A | 0.00 | 142 | 14 | 0 | 0 |
| R6 | 6.0 | 126.3 | 11.68 | 341 | N/A | 0.00 | 210 | 21 | 0 | 0 |
| R6 | 7.0 | 126 | 11.83 | 363 | N/A | 0.00 | 216 | 32 | 0 | 0 |
| R6 | 8.0 | 132.6 | 12.49 | 378 | N/A | 0.00 | 216 | 21 | 0 | 0 |
| R6 | 9.0 | 135.5 | 12.98 | 388 | N/A | 7.00 | 210 | 21 | 0 | 0.6 |
| R6 | 10.0 | 137.8 | 13.29 | 393 | N/A | 19.00 | 210 | 21 | 0 | 0.4 |
| R6 | 11.0 | 142.7 | 14.27 | 404 | N/A | 20.60 | 205 | 25 | 0 | 0.5 |
| R6 | 12.0 | 146.5 | 15.18 | 419 | N/A | 16.00 | 210 | 21 | 0 | 0.5 |
| R6 | 13.0 | 147.7 | 16.06 | 432 | 4.65 | 17.30 | 140 | 13 | 0 | 0.7 |
| R6 | 14.0 | 146.6 | 16.58 | 429 | 5.19 | 30.00 | 140 | 13 | 0 | 0.6 |
| R6 | 15.0 | 147.1 | 17.17 | 455 | 5.35 | 0.00 | 0 | 0 | 0 | 0 |

TABLE 1-25a
Production Data
Study No. 3

| BRX | | VCD | Viability | pH | | | Glutamine | Glutamate | Glucose | Lactate | Ammonium |
|---|---|---|---|---|---|---|---|---|---|---|---|
| ID | Day | (e6 vc/mL) | (%) | offline | pCO2 | pO2 | (g/L) | (g/L) | (g/L) | (g/L) | (g/L) |
| R7 | 0.0 | 0.664 | 97.6 | 6.997 | 50.5 | 101.3 | N/A | 3.11 | 5.7 | 0.53 | 3.1 |
| R7 | 1.0 | 1.58 | 98.8 | 7.02 | 42.5 | 72.4 | N/A | 3.14 | 5.17 | 0.89 | 3.54 |
| R7 | 2.0 | 3.88 | 96.5 | 6.97 | 28 | 54.8 | N/A | 3.24 | 4.48 | 1.21 | 4.06 |
| R7 | 3.0 | 3.7 | 96.7 | 6.805 | 12.3 | 62.2 | N/A | 5.36 | 5.3 | 1.84 | 5.02 |
| R7 | 4.0 | 5.24 | 93.65 | 6.721 | 23 | 61.5 | N/A | 6.78 | 5.77 | 2.1 | 5.86 |
| R7 | 5.0 | 7.64 | 96.9 | 6.698 | 36 | 46.4 | N/A | 8.07 | 6.31 | 2.27 | 6.24 |
| R7 | 6.0 | 9.08 | 97.8 | 6.706 | 48.8 | 55.5 | 0.23 | 9.8 | 6.37 | 2.45 | 6.22 |
| R7 | 7.0 | 11.9 | 96.5 | 6.717 | 60.2 | 51.7 | 0.27 | 11.38 | 6.55 | 2.56 | 6.03 |
| R7 | 8.0 | 14.9 | 97.5 | 6.716 | 67.8 | 41.8 | N/A | 12.36 | 6.37 | 2.67 | 5.95 |
| R7 | 9.0 | 13.97 | 96.27 | 6.717 | 70.6 | 43.1 | N/A | 13.8 | 5.98 | 2.85 | 5.94 |
| R7 | 10.0 | 16.4 | 95.1 | 6.792 | 63.8 | 69.2 | N/A | 12.1 | 4.06 | 2.88 | 5.52 |
| R7 | 11.0 | 16.9 | 94.8 | 6.759 | 69 | 43.3 | 0.97 | 11.51 | 4 | 2.93 | 5.48 |
| R7 | 12.0 | 17.23333333 | 92.3 | 6.757 | 65 | 46.6 | 1.17 | 11.18 | 3.46 | 3.17 | 5.68 |
| R7 | 13.0 | 16.4 | 92.55 | 6.762 | 68.6 | 77.3 | 0.93 | 10.92 | 3.96 | 3.27 | 6.47 |
| R7 | 14.0 | 16.35 | 91.15 | 6.757 | 61.7 | 76.7 | 1.11 | 11.74 | 4.39 | 3.68 | 7.37 |
| R7 | 15.0 | 16.05 | 88.3 | 6.668 | 52.6 | 58.3 | 1.14 | 11.79 | 4.34 | 3.84 | 8.15 |
| R8 | 0.0 | 0.804 | 97.3 | 7.111 | 43.3 | 118.2 | 0.08 | 3.17 | 5.75 | 0.77 | 3.06 |
| R8 | 1.0 | 2.24 | 97.95 | 7.024 | 34.1 | 78.3 | 0.08 | 3.16 | 5.23 | 1.07 | 3.4 |
| R8 | 2.0 | 2.455 | 98.05 | 6.983 | 27.6 | 48.2 | 0.02 | 3.29 | 4.39 | 1.34 | 4.03 |
| R8 | 3.0 | 3.67 | 98.6 | 6.77 | 22.1 | 47.3 | 0.02 | 3.38 | 3.51 | 1.85 | 4.49 |
| R8 | 4.0 | 6.065 | 98.7 | 6.747 | 24 | 53.6 | N/A | 4.78 | 4.05 | 2.04 | 4.82 |
| R8 | 5.0 | 7.85 | 98.25 | 6.75 | 29.1 | 48.3 | N/A | 5.99 | 4.3 | 2.15 | 4.74 |
| R8 | 6.0 | 10.9 | 98.2 | 6.767 | 36.7 | 51.8 | 0.01 | 6.9 | 4.1 | 2.23 | 4.24 |
| R8 | 7.0 | 12.85 | 97.7 | 6.766 | 51.3 | 59.7 | 0.08 | 7.61 | 4.07 | 2.18 | 3.83 |
| R8 | 8.0 | 15.95 | 97.4 | 6.784 | 54.6 | 44.4 | 0.12 | 8.28 | 3.54 | 2.25 | 3.7 |
| R8 | 9.0 | 17.5 | 97.1 | 6.776 | 89.6 | 27 | 0.24 | 7.86 | 3.89 | 2.31 | 3.57 |
| R8 | 10.0 | 18.45 | 96.3 | 6.846 | 94.7 | 62.7 | 0.31 | 7.97 | 4.94 | 2.05 | 3.56 |
| R8 | 11.0 | 20.15 | 95.9 | 6.84 | 110.5 | 37.1 | 0.45 | 7.93 | 4.1 | 2.01 | 3.73 |
| R8 | 12.0 | 19.55 | 95.35 | 6.847 | 117 | 47.6 | 0.57 | 7.99 | 3.52 | 2.15 | 4 |
| R8 | 13.0 | 20.45 | 94.45 | 6.839 | 111.3 | 25.7 | 0.69 | 7.91 | 3.69 | 2.5 | 4.22 |
| R8 | 14.0 | 19.6 | 93 | 6.828 | 88.3 | 59.9 | 0.83 | 7.83 | 3.01 | 2.91 | 4.46 |
| R8 | 15.0 | 18.65 | 92.45 | 6.764 | 97 | 32.8 | 1.09 | 8.43 | 4.36 | 3.14 | 5.15 |

TABLE 1-25b
Production Data
Study No. 3

| | | | | | | Glucose | Feed A | Feed B | Base | Antifoam |
|---|---|---|---|---|---|---|---|---|---|---|
| BRX | | Na+ | K+ | Osmolality | Titer | Addition | Addition | Addition | addition | addition |
| ID | Day | (mM) | (mM) | (mOsm/kg) | (g/L) | (mL) | (mL) | (mL) | (mL) | (mL) |
| R7 | 0.0 | 114.9 | 10.35 | 307 | N/A | 0 | 0 | 0 | 0 | 0 |
| R7 | 1.0 | 117.5 | 10.3 | 308 | N/A | 0.00 | 0 | 0 | 0 | 0 |
| R7 | 2.0 | 115.2 | 10.04 | 304 | N/A | 0.00 | 151 | 15 | 0 | 0 |
| R7 | 3.0 | 122.3 | 11.24 | 326 | N/A | 0.00 | 151 | 15 | 0 | 0 |
| R7 | 4.0 | 122.8 | 11.74 | 354 | N/A | 0.00 | 157 | 18 | 0 | 0 |
| R7 | 5.0 | 126.8 | 12.31 | 364 | N/A | 0.00 | 150 | 15 | 0 | 0 |
| R7 | 6.0 | 130.1 | 12.62 | 376 | N/A | 0.00 | 151 | 15 | 0 | 0 |
| R7 | 7.0 | 133.1 | 13.17 | 401 | N/A | 0.00 | 226 | 22.5 | 0 | 0 |
| R7 | 8.0 | 138 | 13.84 | 410 | N/A | 0.00 | 225.1 | 22.5 | 0 | 0 |
| R7 | 9.0 | 143.6 | 14.57 | 423 | N/A | 0.00 | 225.1 | 22.5 | 0 | 0 |
| R7 | 10.0 | 144.2 | 14.71 | 419 | N/A | 17.70 | 150 | 15 | 0 | 1 |
| R7 | 11.0 | 146 | 15.11 | 426 | N/A | 18.20 | 150 | 15 | 0 | 0.5 |
| R7 | 12.0 | 146.2 | 15.51 | 428 | N/A | 29.00 | 151 | 15 | 0 | 0.5 |
| R7 | 13.0 | 147.5 | 16.23 | 439 | 4.68 | 19.50 | 151.4 | 15 | 0 | 0 |
| R7 | 14.0 | 153.9 | 17.72 | 455 | 5.08 | 12.14 | 150 | 15 | 0 | 0.5 |
| R7 | 15.0 | 151.3 | 18.05 | 472 | 5.38 | 0.00 | 0 | 0 | 0 | 0.5 |
| R8 | 0.0 | 118.5 | 10.53 | 308 | N/A | 0 | 0 | 0 | 0 | 0 |
| R8 | 1.0 | 115.9 | 10.3 | 304 | N/A | 0.00 | 0 | 0 | 0 | 0 |
| R8 | 2.0 | 113.6 | 10.02 | 298 | N/A | 0.00 | 0 | 0 | 0 | 0 |
| R8 | 3.0 | 115.4 | 9.94 | 294 | N/A | 0.00 | 140 | 14 | 0 | 0 |
| R8 | 4.0 | 118.3 | 10.45 | 311 | N/A | 0.00 | 140 | 14 | 0 | 0 |
| R8 | 5.0 | 119.9 | 10.85 | 325 | N/A | 0.00 | 140 | 14 | 0 | 0 |
| R8 | 6.0 | 126.2 | 11.5 | 333 | N/A | 0.00 | 210 | 21 | 0 | 0 |
| R8 | 7.0 | 126.6 | 11.65 | 351 | N/A | 0.00 | 210 | 21 | 0 | 0 |
| R8 | 8.0 | 133.5 | 12.11 | 357 | N/A | 23.10 | 210 | 21 | 0 | 0 |
| R8 | 9.0 | 137 | 12.36 | 368 | N/A | 18.00 | 211 | 21 | 0 | 0.5 |
| R8 | 10.0 | 137 | 12.49 | 384 | N/A | 45.45 | 220 | 21 | 0 | 1 |
| R8 | 11.0 | 139.2 | 12.87 | 391 | N/A | 16.70 | 209 | 24 | 0 | 0.5 |
| R8 | 12.0 | 142.5 | 13.42 | 394 | 4.5 | 26.00 | 210.9 | 21 | 0 | 0.5 |
| R8 | 13.0 | 145.7 | 14.02 | 406 | 5.19 | 24.00 | 210 | 24 | 0 | 0.6 |
| R8 | 14.0 | 147.6 | 14.69 | 406 | 5.61 | 37.00 | 210 | 22 | 0 | 0.5 |
| R8 | 15.0 | 153.2 | 15.5 | 435 | 4.5 | 0.00 | 0 | 0 | 0 | 0 |

TABLE 1-26a
Production Data
Study No. 4

| BRX | | VCD | Viability | pH | | | Glutamine | Glutamate | Glucose | Lactate | Ammonium |
|---|---|---|---|---|---|---|---|---|---|---|---|
| ID | Day | (e6 vc/mL) | (%) | offline | pCO2 | pO2 | (g/L) | (g/L) | (g/L) | (g/L) | (g/L) |
| 200 L | 0.0 | 0.84 | 98.5 | 7.096 | 54.7 | 144.8 | 0 | 3.14 | 5.65 | 0.34 | 2.94 |
| 200 L | 1.0 | 1.47 | 98.2 | 7.034 | 48.9 | 100.9 | 0.02 | 3.14 | 5.16 | 0.63 | 3.19 |
| 200 L | 2.0 | 3.02 | 96.6 | 6.991 | 30.7 | 73.5 | 0.03 | 3.21 | 4.33 | 1.02 | 3.67 |
| 200 L | 3.0 | 4.84 | 97.9 | 6.837 | 11.8 | 92.9 | 0.05 | 3.2 | 3.2 | 1.37 | 4.15 |
| 200 L | 4.0 | 7.99 | 96 | 6.782 | 33 | 57.3 | 0 | 4.61 | 3.54 | 1.47 | 4.3 |
| 200 L | 5.0 | 10.4 | 96.5 | 6.806 | 51.1 | 56.9 | 0.02 | 5.7 | 3.73 | 1.48 | 4.08 |
| 200 L | 6.0 | 13.5 | 97.1 | 6.825 | 73.8 | 36.2 | 0.08 | 6.21 | 3.44 | 1.35 | 3.5 |
| 200 L | 7.0 | 18 | 97.8 | 6.897 | 88.8 | 42.5 | 0.26 | 7.87 | 6.03 | 1.29 | 3.31 |
| 200 L | 8.0 | 21.2 | 96.5 | 6.888 | 83.8 | 37.8 | 0.21 | 6.21 | 3.39 | 1.41 | 2.98 |
| 200 L | 9.0 | 24.6 | 96.9 | 6.899 | 97.8 | 15.8 | 0.13 | 4.99 | 2.97 | 1.32 | 2.87 |
| 200 L | 10.0 | 25.7 | 96.3 | 6.655 | 69.1 | 0 | 0.19 | 4.24 | 3.75 | 2.64 | 3.88 |
| 200 L | 11.0 | 29.5 | 95.95 | 6.812 | 88.7 | 0 | 0.27 | 3.34 | 3.48 | 2.23 | 3.81 |
| 200 L | 12.0 | 27.3 | 94.6 | 6.869 | 87.4 | 22 | 0.33 | 2.58 | 2.58 | 2.04 | 3.23 |
| 200 L | 13.0 | 26.32 | 92.8 | 6.857 | 101.4 | 23.7 | 0.45 | 2.09 | 2.77 | 1.89 | 3.49 |
| 200 L | 14.0 | 25.45 | 91.15 | 6.864 | 85.9 | 42.4 | 0.55 | 2.35 | 2.97 | 2.02 | 3.91 |
| 200 L | 15.0 | 24.7 | 88.65 | 6.768 | 73.8 | 43.3 | 0.6 | 3.01 | 3.12 | 2.38 | 4.4 |
| R9 | 0.0 | 0.855 | 98.3 | 7.148 | 46.1 | 125 | 0 | 3.08 | 5.54 | 0.35 | 2.93 |
| R9 | 1.0 | 1.5 | 97.8 | 7.08 | 30.4 | 64.6 | 0.01 | 3.16 | 5 | 0.74 | 3.22 |
| R9 | 2.0 | 2.86 | 96.3 | 6.978 | 11.2 | 49.6 | 0.04 | 3.26 | 4.02 | 1.22 | 3.79 |
| R9 | 3.0 | 4.51 | 97.4 | 6.762 | 9.7 | 55.1 | 0.04 | 3.29 | 3.07 | 1.54 | 4.32 |
| R9 | 4.0 | 6.43 | 97.5 | 6.769 | 16.9 | 125.2 | 0.02 | 4.92 | 3.78 | 1.64 | 4.54 |
| R9 | 5.0 | 8.13 | 97.6 | 6.752 | 32.3 | 45.2 | 0.08 | 6.31 | 4.26 | 1.75 | 4.52 |
| R9 | 6.0 | 11 | 95.8 | 6.774 | 46.92 | 20.2 | 0.13 | 7.15 | 4.18 | 1.72 | 4.02 |
| R9 | 7.0 | 14.1 | 97.1 | 6.776 | 66.3 | 15.8 | 0.31 | 7.89 | 4.26 | 1.68 | 3.83 |
| R9 | 8.0 | 16.7 | 96.05 | 6.787 | 77.9 | 19.8 | 0.45 | 8.22 | 3.69 | 1.74 | 3.75 |
| R9 | 9.0 | 19.85 | 95.4 | 6.802 | 74.3 | 0 | 0.27 | 8.14 | 3.8 | 1.69 | 3.69 |
| R9 | 10.0 | 23.3 | 95.25 | 6.797 | 70.9 | 0 | 0.42 | 7.74 | 3.77 | 1.89 | 3.59 |
| R9 | 11.0 | 24.65 | 93.7 | 6.81 | 71.6 | 16.3 | 0.38 | 6.89 | 2.71 | 2.02 | 3.52 |
| R9 | 12.0 | 22.3 | 92.1 | 6.782 | 72.3 | 8.6 | 0.44 | 6.02 | 3.07 | 2.14 | 3.53 |
| R9 | 13.0 | 24.2 | 89 | 6.764 | 67 | 0 | 0.51 | 5.62 | 2.96 | 2.29 | 3.91 |
| R9 | 14.0 | 26.75 | 88.75 | 6.712 | 58.4 | 11.1 | 0.6 | 5.88 | 3.73 | 2.58 | 4.74 |
| R9 | 15.0 | 22.5 | 83.1 | 6.598 | 47.1 | 110.8 | 0.59 | 6.56 | 4.18 | 2.94 | 6.01 |
| R10 | 0.0 | 0.82 | 97.6 | 7.07 | 54.4 | 121.3 | 0 | 3.46 | 5.61 | 0.38 | 2.97 |
| R10 | 1.0 | 1.58 | 97.6 | 7.029 | 42.5 | 78.7 | 0 | 3.51 | 5.02 | 0.72 | 3.29 |
| R10 | 2.0 | 2.47 | 97.4 | 6.9701 | 21.1 | 76.5 | 0 | 3.62 | 4.17 | 1.13 | 3.87 |
| R10 | 3.0 | 3.99 | 97.2 | 6.775 | 13.7 | 75.8 | 0 | 3.73 | 3.18 | 1.54 | 4.36 |
| R10 | 4.0 | 6.42 | 95.5 | 6.738 | 22.5 | 94.9 | 0.06 | 5.31 | 4.94 | 1.69 | 4.71 |
| R10 | 5.0 | 7.82 | 97.3 | 6.748 | 34.2 | 77.6 | 0.15 | 5.58 | 4.44 | 1.77 | 4.77 |
| R10 | 6.0 | 10 | 95.5 | 6.732 | 51.3 | 67.1 | 0.4 | 5.79 | 4.36 | 1.76 | 4.3 |
| R10 | 7.0 | 13.35 | 95.4 | 6.755 | 64.7 | 63.9 | 0.71 | 6.46 | 4.57 | 1.8 | 4.25 |
| R10 | 8.0 | 16.2 | 95.7 | 6.756 | 76 | 64.8 | 0 | 8.2 | 4.04 | 1.84 | 4.15 |
| R10 | 9.0 | 18.65 | 95.5 | 6.768 | 79.8 | 50.8 | 0 | 8.25 | 4.08 | 1.78 | 4.2 |
| R10 | 10.0 | 21.3 | 94.05 | 6.754 | 79.5 | 48.8 | 0 | 7.97 | 3.91 | 2.07 | 4.34 |
| R10 | 11.0 | 21.65 | 92.8 | 6.761 | 79.3 | 11.5 | 0 | 7.64 | 2.7 | 2.14 | 4.22 |
| R10 | 12.0 | 22 | 92.25 | 6.759 | 74.7 | 0 | 0.35 | 7.29 | 3.49 | 2.31 | 4.39 |
| R10 | 13.0 | 24 | 90 | 6.732 | 72.6 | 80.6 | 0.99 | 7.08 | 3.56 | 2.47 | 4.95 |
| R10 | 14.0 | 18.95 | 84.5 | 6.685 | 67.5 | 40.9 | 1.37 | 7.42 | 3.97 | 2.74 | 6 |
| R10 | 15.0 | 19.8 | 83.2 | 6.64 | 56.3 | 60.9 | 1.7 | 8.1 | 4.51 | 3.1 | 7.18 |

TABLE 1-26b
Production Data
Study No. 4

| | | | | | | Glucose | Feed A | Feed B | Base | Antifoam |
|---|---|---|---|---|---|---|---|---|---|---|
| BRX | | Na+ | K+ | Osmolality | Titer | Addition | Addition | Addition | addition | addition |
| ID | Day | (mM) | (mM) | (mOsm/kg) | (g/L) | (mL) | (mL) | (mL) | (mL) | (mL) |
| 200 L | 0.0 | 117.7 | 10.39 | 312 | N/A | 0.0 | 0 | 0 | 0.0 | 0.0 |
| 200 L | 1.0 | 115.3 | 10.26 | 306 | N/A | 0.0 | 0 | 0 | 0.0 | 0.0 |
| 200 L | 2.0 | 114.9 | 10.01 | 302 | N/A | 0.0 | 0 | 0 | 0.0 | 0.0 |
| 200 L | 3.0 | 116.3 | 9.75 | 296 | N/A | 0.0 | 2794.0 | 280.0 | 0.0 | 5.1 |
| 200 L | 4.0 | 116.8 | 10.07 | 306 | N/A | 0.0 | 2800.0 | 280.0 | 0.0 | 5.0 |
| 200 L | 5.0 | 122.6 | 10.56 | 319 | N/A | 0.0 | 2820.0 | 280.0 | 0.0 | 5.0 |
| 200 L | 6.0 | 125.2 | 10.76 | 328 | N/A | 0.0 | 4210.0 | 420.0 | 0.0 | 0.0 |
| 200 L | 7.0 | 132.4 | 11.85 | 365 | N/A | 588.0 | 4210.0.0 | 420.0 | 0.0 | 5.2 |
| 200 L | 8.0 | 131.9 | 11.04 | 340 | N/A | 512.0 | 4220.0 | 420.0 | 0.0 | 4.9 |
| 200 L | 9.0 | 135 | 10.97 | 345 | N/A | 996.0 | 5666.0 | 560.0 | 0.0 | 10.2 |
| 200 L | 10.0 | 138 | 11.59 | 364 | N/A | 427.0 | 4200.0 | 420.0 | 0.0 | 10.0 |
| 200 L | 11.0 | 140.7 | 12.03 | 369 | N/A | 533.0 | 4208.0 | 420.0 | 12.60 | 16.18 |
| 200 L | 12.0 | 142.1 | 12.43 | 363 | 5.86 | 872.0 | 4200.0 | 420.0 | 0.0 | 12.0 |
| 200 L | 13.0 | 143.7 | 13.13 | 373 | 6.86 | 826.0 | 4200.0 | 420.0 | 0.0 | 11.2 |
| 200 L | 14.0 | 147.6 | 14.14 | 380 | 7.3 | 773.0 | 4200.0 | 420.0 | 0.0 | 10.3 |
| 200 L | 15.0 | 155.2 | 15.41 | 393 | 8.55 | 0 | 0 | 0 | 0 | 0 |
| R9 | 0.0 | 113.9 | 10.17 | 316 | | 0.0 | 0 | 0 | 0.0 | 0.0 |
| R9 | 1.0 | 116.8 | 10.27 | 314 | | 0.0 | 0 | 0 | 0.0 | 0.0 |
| R9 | 2.0 | 116.1 | 10.11 | 324 | | 0.0 | 0 | 0 | 0.0 | 0.0 |
| R9 | 3.0 | 116.3 | 9.9 | 315 | | 0.0 | 140.0 | 14.0 | 0.0 | 0.0 |
| R9 | 4.0 | 117.8 | 10.37 | 341 | | 0.0 | 140.0 | 14.0 | 0.0 | 0.0 |
| R9 | 5.0 | 123.2 | 11.08 | 352 | | 0.0 | 140.0 | 14.0 | 0.0 | 0.0 |
| R9 | 6.0 | 127.1 | 11.41 | 339 | | 0.0 | 210.0 | 21.0 | 0.0 | 0.0 |
| R9 | 7.0 | 129.9 | 11.95 | 359 | | 0.0 | 210.0 | 21.0 | 0.0 | 0.0 |
| R9 | 8.0 | 134.3 | 12.45 | 373 | | 20.0 | 210.0 | 21.0 | 0.0 | 0.0 |
| R9 | 9.0 | 138.2 | 12.86 | 370 | | 20.4 | 211.1 | 22.0 | 0.0 | 5.5 |
| R9 | 10.0 | 140.6 | 13.37 | 389 | | 22.2 | 210.9 | 21.0 | 0.0 | 0.6 |
| R9 | 11.0 | 144.3 | 13.93 | 384 | | 41.0 | 210.0 | 21.0 | 0.0 | 0.5 |
| R9 | 12.0 | 144.2 | 14.53 | 398 | 5.5 | 35.0 | 210.0 | 21.0 | 0.0 | 1.3 |
| R9 | 13.0 | 144.5 | 15.47 | 402 | 6.38 | 39.4 | 210.0 | 21.0 | 0.0 | 0.6 |
| R9 | 14.0 | 150.2 | 16.92 | 458 | 6.99 | 39.1 | 210.6 | 21.0 | 0.0 | 0.8 |
| R9 | 15.0 | 151.6 | 18.09 | 446 | 7.49 | 0 | 0 | 0 | 0 | 0 |
| R10 | 0.0 | 115.9 | 10.43 | 308 | | 0.0 | 0 | 0 | 0.0 | 0.0 |
| R10 | 1.0 | 114.7 | 10.23 | 305 | | 0.0 | 0 | 0 | 0.0 | 0.0 |
| R10 | 2.0 | 115.8 | 10.13 | 296 | | 0.0 | 0 | 0 | 0.0 | 0.0 |
| R10 | 3.0 | 116.4 | 9.93 | 292 | | 0.0 | 140.0 | 14.0 | 0.0 | 0.0 |
| R10 | 4.0 | 120.1 | 11.26 | 328 | | 0.0 | 140.0 | 14.0 | 0.0 | 0.0 |
| R10 | 5.0 | 124.3 | 11.27 | 331 | | 0.0 | 140.0 | 14.0 | 0.0 | 0.0 |
| R10 | 6.0 | 123.7 | 11.39 | 342 | | 0.0 | 210.0 | 21.0 | 0.0 | 0.0 |
| R10 | 7.0 | 131.2 | 12.3 | 359 | | 0.0 | 210.8 | 21.0 | 0.0 | 0.0 |
| R10 | 8.0 | 132.6 | 12.46 | 370 | | 15.0 | 210.0 | 21.0 | 0.0 | 0.0 |
| R10 | 9.0 | 138 | 13.27 | 374 | | 14.7 | 211.2 | 24.0 | 0.0 | 5.0 |
| R10 | 10.0 | 142.6 | 13.95 | 388 | | 19.1 | 210.2 | 21.0 | 0.0 | 0.6 |
| R10 | 11.0 | 144 | 14.92 | 391 | | 40.0 | 210.0 | 21.0 | 0.0 | 0.5 |
| R10 | 12.0 | 145.9 | 14.95 | 404 | 4.96 | 27.0 | 211.0 | 21.0 | 0.0 | 0.6 |
| R10 | 13.0 | 146.6 | 15.82 | 431 | 5.76 | 27.5 | 211.4 | 21.0 | 0.0 | 0.5 |
| R10 | 14.0 | 152.7 | 17.47 | 439 | 6.19 | 19.5 | 209.5 | 21.0 | 0.0 | 0.8 |
| R10 | 15.0 | 150.8 | 18.59 | 468 | 6.6 | 0 | 0 | 0 | 0 | 0 |

### Example 2: Downstream Process for Antibody Purification Using Cation Exchange Chromatography

Example 2 describes the development of a cation exchange chromatography step for purification process of an anti-GM-CFSRa antibody, mavrilimumab. A high productivity cell culture process was developed as described above. The downstream process are summarized in Table 2-1.

TABLE 2-1
Purification process overview

| Step | |
|---|---|
| 1 | Protein A Chromatography |
| 2 | Low pH Viral Inactivation |
| 3 | Cation Exchange Chromatography - bind-elute mode |
| 4 | Anion Exchange Chromatography - bind-elute mode |
| 5 | Virus Filtration |
| 6 | Ultrafiltration/diafiltration |
| 7 | Formulation, Filtration and Bulk Fill |

ACRONYMS & DEFINITIONS

| Term | Definition |
|---|---|
| AEX | Anion exchange chromatography |
| Brx | Bioreactor |
| CEX | Cation exchange chromatography |
| cGMP | Current good manufacturing practices |
| CHO | Chinese Hamster Ovary Cells |
| CV | Column volume |
| DS | Drug substance |
| FT | Flowthrough |
| g | Gram |
| Hab | Half antibody |
| HCP | Host cell proteins |
| H | Column bed height |
| hr | Hour |
| ID | Column internal diameter |
| IEC | Analytical ion exchange chromatography |
| kDa | Kilodalton |
| L | Liter |
| N/A | Not applicable |
| NR-CE-SDS | Non-reduced capillary electrophoresis |
| | sodium dodecyl sulfate |
| mM | Millimolar |
| min | Minutes |
| mS | Milli-siemens |
| UF/DF | Ultrafiltration/Diafiltration |
| VI | Virus inactivation |
| VIN | Virus inactivation & neutralization |

### Materials

### Harvests

Clarified harvests from 10 L-scale bioreactor runs were used for mavrilimumab downstream process development. Pilot scale capture runs followed by downstream purification through drug substance were performed with harvest from 200 L scale demonstration run 1.

### Column Packing and Qualification

CEX runs were performed using lcm (ID)×20 cm (H), 5 cm (ID)×20 cm (H) and 20 cm (ID)×20 cm (H) columns. The 5 cm×20 cm column was packed in-house. and the remaining two were pre-packed columns obtained from Repligen. Columns were qualified and verified to meet HETP and asymmetry (As) specifications.

### Analytical Methods

A280 values were measured for the purified samples using Solo VPE; protein concentration was then calculated using an extinction coefficient 1.44 mL/mg-cm. Hab, aggregation, and charge profile analyses were performed using methods shown in Table 2-2.

TABLE 2-2
List of analytical methods

| Assay | Method |
|---|---|
| Half antibody | Non-reduced CE-SDS |
| Aggregation | SEC-HPLC |
| Charge species | IEC |

### Results and Discussion

### Establishment Run

Clarified harvest from 2×10 L bioreactors was purified through DS following a purification process as shown in Table 2-3. Hab contents in process intermediates were 8.4-10.6% and Hab contents in DS were 7.6%, and the target Hab level for DS (≤3%). Results indicated that this downstream process was not able to achieve a desired Hab level using the upstream harvest.

TABLE 2-3
Hab results for establishment run 1 process intermediates & DS

| | | | | | | | Target |
|---|---|---|---|---|---|---|---|
| | Protein | | Fractogel | Q | | | Hab level |
| | A | VIN | COO⁻ (M) | Membrane | TFF | | for Phase |
| | Eluate | Product | Eluate | FTW | pool | DS | 3 DS (%) |
| Hab | 10.6 | 9.5 | 9.1 | 9.0 | 8.4 | 7.6 | ≤3 |
| (%) | | | | | | | |

### Development of POROS™ XS Chromatography

POROS™ XS resin was evaluated as an alternative to Fractogel COO⁻ (M) chromatography step to decrease the level of Hab to the target level of ≤3%. Gradient elution and step elution runs were performed to define operational conditions for the POROS™ XS chromatography step. Briefly, results from two salt gradient elution runs indicated better Hab clearance with pH 5.5 elution buffer as compared to pH 6.0. As shown in Table 2-4, POROS™ XS step elution runs were then performed with elution buffers at different pH and salt concentration. Based on eluate Hab % and step yield results, pH 5.4 with 55 mM NaCl was selected as the elution condition for the POROS™ XS chromatography. Table 2-5 summarizes the process parameters for the POROS™ XS chromatography based on the development work performed there.

The proposed loading range for the CEX step is 30 and 60 g/L. The estimated CEX column eluate volume is around 4-5 CVs. The estimated CEX step yield is 75 to 85%.

The POROS™ XS eluate was further purified through DS and confirmed acceptable product quality (Table 2-6).

TABLE 2-4
CEX Development Runs- yield and Hab %

| | Column | | | | | Elution | Eluate | Eluate |
|---|---|---|---|---|---|---|---|---|
| | ID | CV | Load | Load | Elution | NaCl | Yield | Hab |
| | (cm) | (ml) | (Hab %) | (g/L) | pH | (mM) | (%) | (%) |
| CEX-DEV003 | 1.13 | 22 | 9.5 | 60 | 5.5 | 45 | 74.7 | 1.3 |
| CEX-DEV004 | | | | | 5.5 | 90 | 87.5 | 6.9 |
| CEX-DEV005 | | | | | 5.3 | 50 | 53.9 | 0.6 |
| CEX-DEV006 | | | | | 5.7 | 60 | 90.8 | 8.6 |
| CEX-DEV007 | | | | | 5.6 | 60 | 87.5 | 7.0 |
| CEX-DEV008 | | | | | 5.4 | 50 | 65.9 | 1.1 |

TABLE 2-5
Proposed process parameters for the POROS ™ XS chromatography

| | | | Flow | |
|---|---|---|---|---|
| | | | Rate | |
| Step | Buffer/Solution | CV | (cm/h) | Note |
| Pre-run | 0.5M NaOH | 3.0 | 300 | 60 minutes hold |
| Sanitization | | | | |
| RODI | RODI | 3.0 | 300 | |
| Equilibration | 50 mM Sodium Acetate pH 5.0 | 5.0 | 300 | pH 5.0 ± 0.2 |
| | | | | Cond: 2.1-3.0 mS/cm |
| Load | Virus Inactivated Protein A Eluate | Variable | 300 | pH 5.0 ± 0.1 |
| | | | | Cond: ≤4.0 mS/cm |
| Wash | 50 mM Sodium Acetate pH 5.0 | 5.0 | 300 | |
| Elution | 50 mM Acetate, 55 mM NaCl, pH | 10.0 | 300 | pH 5.4 ± 0.1 |
| | 5.4 | | | Cond: 8.5-9.5 mS/cm |
| | | | | Collection Start: |
| | | | | 0.5 AU/cm |
| | | | | Collection End: |
| | | | | 2.0 AU/cm |
| Strip | 50 mM Acetate, 1M NaCl, pH 5.5 | 3.0 | 300 | pH 5.5 ± 0.2 |
| | | | | Cond: FIO |
| Post-run | 0.5M NaOH | 3.0 | 300 | 60 minutes hold |
| Sanitization | | | | |
| Storage | 0.1M NaOH | 3.0 | 300 | Cond: 19-27 mS/cm |

TABLE 2-6
Non-reduced CE-SDS results proving
run process intermediates and DS

| Process Step | Hab (%) | IgG (%) |
|---|---|---|
| Protein A Eluate | 7.2 | 92.0 |
| VIN Product | 7.1 | 92.2 |
| POROS ™ XS Eluate | 1.9 | 97.1 |
| Q Membrane FT/chase | 1.8 | 97.5 |
| TFF pool | 1.9 | 97.0 |
| DS | 1.7 | 97.2 |
| Reference (RSN300111L) | 2.7 | 97.3 |

### Salt Gradient Elution Runs at pH 4.5 and pH 5.0

POROS™ XS runs with salt gradient elution at pH 5.5 and 6.0 resulted in a major elution peak (which contained low Hab %) followed by a minor elution peak enriched in Hab. Additional salt gradient runs were performed at pH 4.5 and 5.0 to evaluate the impacts of lower elution pH on POROS™ XS Hab clearance. UV chromatograms suggests that elution pH 4.5 and 5.0 did not improve the performance of POROS™ XS in terms of Hab clearance as indicated by the poor resolution of the two elution peaks.

### Resins Screening

Additional CEX and mixed mode resins (Table 2-7) were evaluated for Hab clearance and compared to POROS™ XS. Gradient and step elution runs were performed, and selective fractions were tested for Hab. Yield and Hab % results presented below indicated that these resins did not perform better than POROS™ XS in terms of Hab clearance.

TABLE 2-7
List of CEX and mixed-mode resins evaluated

| Resin | Note(s) |
|---|---|
| Capto ™ S ImpAct | CEX resin |
| TOYOPEARL ™ GigaCap CM-650M | CEX resin |
| TOYOPEARL ™ sulfate 650F | CEX resin |
| Capto ™ adhere ImpRes | Mixed mode resin |
| Capto ™ MMC ImpRes | Mixed mode resin |

### Capto™ S ImpAct Gradient Run

Capto™ S ImpAct HiScreen column (0.77 cm (ID)×10 cm (H)) was loaded at 45 g/L, followed by gradient elution as shown in Table 2-8. Only one single elution peak was observed. The elution peak was fractionated into 4 fractions. Fractions 1 and 2 were cloudy before 0.2 μm filtration. Fraction 2, the fraction near the elution peak height, contained 4.3% Hab, which was higher than the target≤3% for Hab. Thereof, Capto™ S ImpAct was not further evaluated.

TABLE 2-8
Process parameters for the Capto ™ S ImpAct gradient elution run

| | | | Flow | |
|---|---|---|---|---|
| | | | Rate | |
| Step | Buffer/Solution | CV | (cm/h) | Note |
| Pre-run | 0.5M NaOH | 3.0 | 150 | 60 minutes hold |
| Sanitization | | | | |
| RODI | RODI | 3.0 | 150 | |
| Equilibration | 50 mM Acetate, pH 5.0 | 5.0 | 150 | |
| Load | Virus Inactivated Protein A Eluate | 2.1 | 150 | pH: 4.92 |
| | | | | Cond: 3.33 mS/cm |
| Wash | 50 mM Acetate, pH 5.4 | 3.0 | 150 | |
| Gradient | Buffer A: 50 mM Acetate, pH 5.4 | 20 | 150 | |
| Elution | Buffer B: 50 mM Acetate, 250 mM NaCl, pH | | | |
| | 5.4 | | | |
| Strip | 20 mM Phosphate, 250 mM NaCl, pH 7.0 | 3.0 | 150 | |
| RODI | RODI | 3.0 | 150 | |

TABLE 2-9
Capto ™ S ImpAct ™ run - yield and non-reduced CE SDS results

| | | | | | Elution | | |
|---|---|---|---|---|---|---|---|
| Load/ | Vol- | Pro- | Pro- | | Cumulative | | |
| Elution | ume | tein | tein | Yield | Yield | Hab | IgG |
| Fractions | (ml) | (mg/ml) | (mg) | (%) | (%) | (%) | (%) |
| Load | 14.0 | 14.96 | 209.4 | | | | |
| F1 | 9.2 | 7.02 | 64.6 | 30.8 | 30.8 | 1.2 | 97.1 |
| F2 | 6.0 | 17.13 | 102.8 | 49.1 | 79.9 | 4.3 | 94.4 |
| F3 | 4.9 | 3.48 | 17.1 | 8.1 | 88.1 | | |
| F4 | 6.5 | 0.72 | 4.7 | 2.2 | 90.3 | | |

### TOYOPEARL™ GigaCap CM-650M Gradient Run

Pre-packed TOYOPEARL™ GigaCap CM-650M column (0.8 cm (ID)×10 cm (H)) was loaded at 44 g/L and a salt gradient run was performed similarly to Capto™ S ImpAct. The elution fractions yields are shown in Table 2-10. Only one single elution peak was observed. Thereof, TOYOPEARL™ GigaCap CM-650M was not further evaluated.

TABLE 2-10
TOYOPEARL ™ GigaCap CM-650M Run - yield results

| | | | | | Elution |
|---|---|---|---|---|---|
| Load/Elution | Volume | Protein | Protein | | Cumulative |
| Fractions | (ml) | (mg/ml) | (mg) | Yield (%) | Yield (%) |
| Load | 14.8 | 14.96 | 221.0 | | |
| F1 | 10.10 | 8.91 | 90.0 | 40.7 | 40.7 |
| F2 | 10.40 | 11.49 | 119.5 | 54.1 | 94.8 |
| F3 | 5.50 | 1.98 | 10.9 | 4.9 | 99.7 |
| F4 | 5.40 | 0.32 | 1.7 | 0.8 | 100.5 |

### TOYOPEARL™ Sulfate 650F Gradient Runs

Five experimental runs (Run 1, 4, 5, 6, 7) were performed on a TOYOPEARL™ sulfate 650F pre-packed column (0.8 cm (ID)×10 cm (H)).

Run 1 is a gradient run performed similarly to Capto™ S ImpAct with a load challenge at 58.4 g/L. Yields (Table 2-11) indicated that 70% of mavrilimumab was lost in the FT/wash fraction. In addition, the FT/wash fraction contained 5.6% Hab (Table 2-11) which is higher than the ≤3% target.

TABLE 2-11
TOYOPEARL ™ sulfate 650F run 1 -
yield and non-reduced CE SDS results

| | | | | | Elution | | |
|---|---|---|---|---|---|---|---|
| Load/ | Vol- | Pro- | Pro- | | Cumulative | | |
| Elution | ume | tein | tein | Yield | Yield | Hab | IgG |
| Fractions | (ml) | (mg/ml) | (mg) | (%) | (%) | (%) | (%) |
| Load | 19.8 | 14.96 | 295.8 | | | | |
| FT/Wash | 20.80 | 9.90 | 205.9 | 69.6 | 69.6 | 5.6 | 93.5 |
| Wash F2 | 6.00 | 0.86 | 5.1 | 1.7 | 71.4 | | |
| F1 | 6.60 | 0.14 | 1.0 | 0.3 | 71.7 | | |
| F2 | 10.00 | 1.01 | 10.1 | 3.4 | 75.1 | | |
| F3 | 53.0 | 0.93 | 49.3 | 16.7 | 91.8 | 26.0 | 74.0 |
| Hab Strip | 15.0 | 0.21 | 3.2 | 1.1 | 92.8 | | |

Subsequential Run 4, was performed with sodium chloride adjusted Protein A eluate to improve product binding to the resin. 5M NaCl was added to Protein A eluate to a final concentration of 100 mM NaCl and then loaded onto the TOYOPEARL™ sulfate 650F pre-packed column at a load challenge of 45 g/L, followed by a gradient elution similar to Capto™ S ImpAct. Yields are provided in Table 2-12.

TABLE 2-12
TOYOPEARL ™ sulfate 650F Run 4 - yield results

| | | | | | Elution |
|---|---|---|---|---|---|
| Load/Elution | Volume | Protein | Protein | | Cumulative |
| Fractions | (ml) | (mg/ml) | (mg) | Yield (%) | Yield (%) |
| Load | 15.4 | 14.67 | 226.2 | | |
| F2 | 10.0 | 4.9 | 48.9 | 21.6 | 21.6 |
| F3 | 9.9 | 8.19 | 81.2 | 35.9 | 57.5 |
| F4 | 15.5 | 1.62 | 25.2 | 11.1 | 68.6 |
| F5 | 16.9 | 0.65 | 11.0 | 4.8 | 73.5 |
| Strip | 15.3 | 2.19 | 33.4 | 14.8 | 88.2 |

Based on Run 4 results, Run 5 was performed with step elution using 50 mM sodium acetate, 125 mM NaCl, pH 5.5 (Run 4 was performed using gradient elution). Eluate was collected into two fractions followed by salt strip steps. Yield values are provided in Table 2-13.

TABLE 2-13
TOYOPEARL ™ sulfate 650F Run 5 - yield results

| | | | | | Elution |
|---|---|---|---|---|---|
| Load/Elution | Volume | Protein | Protein | | Cumulative |
| Fractions | (ml) | (mg/ml) | (mg) | Yield (%) | Yield (%) |
| Load | 15.4 | 14.67 | 226.2 | | |
| Eluate main | 22.3 | 8.33 | 185.8 | 82.1 | 82.1 |
| Fraction | | | | | |
| F2 | 21.2 | 0.45 | 9.5 | 4.2 | 86.3 |
| Hab Strip | 15.3 | 0.53 | 8.1 | 3.6 | 89.9 |
| 1M Salt Strip | 15.1 | 1.37 | 20.7 | 9.1 | 99.1 |

Run 6 was performed with Protein A eluate adjusted to 50 mM NaCl (in Run 4 and Run 5, the Protein A eluate was adjusted to 100 mM NaCl for loading onto the sulfate 650F column). The pre-packed column was loaded at 45 g/L, followed by wash with 50 mM sodium acetate, pH 5.0 and a gradient elution with 50 mM sodium acetate, pH 5.0 (Buffer A) and 50 mM sodium acetate, 500 mM NaCl, pH 5.0 (Buffer B). Yield values are provided in Table 2-14.

TABLE 2-14
TOYOPEARL ™ sulfate 650F Run 6 - Yield Results

| | | | | | Elution |
|---|---|---|---|---|---|
| Load/Elution | Volume | Protein | Protein | | Cumulative |
| Fractions | (ml) | (mg/ml) | (mg) | Yield (%) | Yield (%) |
| Load | 15.2 | 14.91 | 226.2 | | |
| F2 | 5.3 | 3.06 | 16.2 | 7.2 | 7.2 |
| F3 | 6.5 | 7.26 | 46.8 | 20.7 | 27.9 |
| F4 | 8.5 | 1.84 | 15.6 | 6.9 | 34.8 |
| F5 | 5.4 | 1.26 | 6.8 | 3.0 | 37.8 |
| F6 | 35.2 | 1.69 | 59.5 | 26.3 | 64.1 |
| 1M Salt Strip | 35.2 | 1.69 | 59.5 | 26.3 | 90.4 |

Based on Run 6 results, Run 7 was performed with Protein A eluate adjusted to 50 mM NaCl and step elution (Run 6 was performed using gradient elution). The pre-packed column was loaded at 46 g/L followed by wash and step elution using 50 mM sodium acetate, 150 mM NaCl, pH 5.3. Yield and level of Hab values were 43% and 3.9% respectively for the 1^{st} fraction (Table 2-15), which was higher than the POROS™ XS runs. Sulfate 650F was thereof not further evaluated.

TABLE 2-15
TOYOPEARL ™ sulfate 650F Run 7 -
yield and non-reduced CE SDS results

| | | | | | Elution | | |
|---|---|---|---|---|---|---|---|
| Load/ | | Pro- | Pro- | | Cumulative | | |
| Elution | Vol | tein | tein | Yield | Yield | Hab | IgG |
| Fractions | (ml) | (mg/ml) | (mg) | (%) | (%) | (%) | (%) |
| Load | 15.4 | 14.91 | 229.9 | | | | |
| Eluate main | 13.8 | 7.09 | 98.0 | 42.6 | 42.6 | 3.9 | 94.9 |
| Fraction | | | | | | | |
| F2 | 8.0 | 2.26 | 18.1 | 7.9 | 50.5 | | |
| F3 | 19.2 | 0.98 | 18.8 | 8.2 | 58.7 | | |
| Hab Strip | 15.4 | 1.11 | 17.1 | 7.4 | 66.1 | | |
| 1M Salt | 15.2 | 4.77 | 72.5 | 31.5 | 97.7 | | |
| Strip | | | | | | | |

### Capto™ MMC ImpRes Gradient Runs

Three gradient runs were performed with Capto™ MMC ImpRes pre-packed column (0.77 cm (ID)×10 cm (H)) and eluate fractions were tested for Hab. Operational conditions are provided in Tables 2-16, 2-17 and 2-18. Fractions contained low Hab (1.2-1.9%) had cumulative yields around 9.3-26.7%. Given the low yields, Capto™ MMC ImpRes resin was not evaluated further.

TABLE 2-16
Process parameters for the Capto ™ MMC ImpRes Run 8

| | | | Flow | |
|---|---|---|---|---|
| | | | Rate | |
| Step | Buffer/Solution | CV | (cm/h) | Note |
| Pre-run | 0.5M NaOH | 3.0 | 150 | 60 |
| Sanitization | | | | minutes |
| | | | | hold |
| RODI | RODI | 3.0 | 150 | |
| Equilibration | 50 mM Acetate, pH 5.0 | 5.0 | 150 | |
| Load | Virus Inactivated Protein A Eluate | 2.1 | 150 | pH: 5.0 |
| Wash | 50 mM Acetate, pH 5.4 | 3.0 | 150 | |
| Gradient | Buffer A: 50 mM Acetate, pH 5.4 | 25 | 150 | |
| Elution | | | | |
| | Buffer B: 50 mM Acetate, 250 mM NaCl, | | | |
| | pH 5.4 | | | |
| Strip | 20 mM Phosphate, 250 mM NaCl, pH | 3.0 | 150 | |
| | 7.0 | | | |
| RODI | RODI | 3.0 | 150 | |
| Post-run | 0.5M NaOH | 3.0 | 150 | 60 |
| Sanitization | | | | minutes |
| | | | | hold |
| Storage | 0.1M NaOH | 3.0 | 150 | |

TABLE 2-17
Process parameters for the Capto ™ MMC ImpRes Run 9

| | | | Flow | |
|---|---|---|---|---|
| | | | Rate | |
| Step | Buffer/Solution | CV | (cm/h) | Note |
| Pre-run | 0.5M NaOH | 3.0 | 150 | 60 minutes hold |
| Sanitization | | | | |
| RODI | RODI | 3.0 | 150 | |
| Equilibration | 50 mM Acetate, pH 5.0 | 5.0 | 150 | |
| Load | Virus Inactivated Protein A Eluate | 2.1 | 150 | pH: 5.0 |
| Wash | 50 mM Acetate, pH 5.0 | 3.0 | 150 | |
| Gradient | Buffer A: 50 mM Acetate, pH 5.0 | 25 | 150 | |
| Elution | Buffer B: 50 mM Acetate, 500 mM NaCl, | | | |
| | pH 5.0 | | | |
| Strip | 20 mM Phosphate, 250 mM NaCl, pH | 3.0 | 150 | |
| | 7.0 | | | |
| RODI | RODI | 3.0 | 150 | |
| Post-run | 0.5M NaOH | 3.0 | 150 | 60 minutes hold |
| Sanitization | | | | |
| Storage | 0.1M NaOH | 3.0 | 150 | |

TABLE 2-18
Process parameters for the Capto ™ MMC ImpRes Run 13

| | | | Flow | |
|---|---|---|---|---|
| | | | Rate | |
| Step | Buffer/Solution | CV | (cm/h) | Note |
| Pre-run | 0.5M NaOH | 3.0 | 150 | 60 minutes |
| Sanitization | | | | hold |
| RODI | RODI | 3.0 | 150 | |
| Equilibration | 50 mM Acetate, pH 5.0 | 5.0 | 150 | |
| Load | Virus Inactivated Protein A Eluate | 3.1 | 150 | pH: 5.0 |
| Wash | 50 mM Acetate, pH 5.6 | 3.0 | 150 | |
| Gradient | Buffer A: 50 mM Acetate, pH 5.6 | 25 | 150 | |
| Elution | Buffer B: 50 mM Acetate, 150 mM NaCl, | | | |
| | pH 5.6 | | | |
| Strip | 20 mM Phosphate, 250 mM NaCl, pH 7.0 | 3.0 | 150 | |
| RODI | RODI | 3.0 | 150 | |
| Post-run | 0.5M NaOH | 3.0 | 150 | 60 minutes |
| Sanitization | | | | hold |
| Storage | 0.1M NaOH | 3.0 | 150 | |

TABLE 2-19
Capto ™ MMC ImpRes Run 8- yield
and non-reduced CE SDS results

| | | | | | Elution | | |
|---|---|---|---|---|---|---|---|
| Load/ | | | | | Cumulative | | |
| Elution | Vol | Protein | Protein | Yield | Yield | Hab | IgG |
| Fractions | (ml) | (mg/ml) | (mg) | (%) | (%) | (%) | (%) |
| Load | 9.7 | 19.18 | 186.2 | | | | |
| 3 | 11.7 | 0.02 | 0.2 | 0.1 | 0.0 | | |
| 4 | 11.7 | 4.26 | 49.6 | 26.7 | 26.7 | 1.2 | 94.5 |
| 5 | 11.7 | 4.82 | 56.2 | 30.2 | 56.8 | | |
| 6 | 11.7 | 3.02 | 35.2 | 18.9 | 75.7 | 10.0 | 88.5 |
| 7 | 11.7 | 1.24 | 14.4 | 7.8 | 83.5 | 34.2 | 65.8 |
| 8 | 11.7 | 0.41 | 4.8 | 2.6 | 86.0 | | |
| 9 | 11.7 | 0.19 | 2.2 | 1.2 | 87.2 | 55.1 | 44.9 |
| 10 | 11.7 | 0.07 | 0.8 | 0.4 | 87.7 | | |
| Strip | 35.0 | 0.11 | 3.8 | 2.1 | 89.7 | | |

TABLE 2-20
Capto ™ MMC ImpRes Run 9- yield
and non-reduced CE SDS results

| | | | | | Elution | | |
|---|---|---|---|---|---|---|---|
| Load/ | | Pro- | Pro- | | Cumulative | | |
| Elution | Vol | tein | tein | Yield | Yield | Hab | IgG |
| Fractions | (ml) | (mg/ml) | (mg) | (%) | (%) | (%) | (%) |
| Load | 9.7 | 19.18 | 186.2 | N/A | | | |
| 3 | 11.7 | 0.00 | 0.0 | 0.0 | 0.0 | | |
| 4 | 11.7 | 1.49 | 17.4 | 9.3 | 9.3 | 1.9 | 97.1 |
| 5 | 11.7 | 5.24 | 61.0 | 32.8 | 42.1 | | |
| 6 | 11.7 | 4.39 | 51.1 | 27.5 | 69.6 | 3.5 | 93.1 |
| 7 | 11.7 | 2.73 | 31.8 | 17.1 | 86.7 | 16.1 | 83.9 |
| 8 | 11.7 | 1.12 | 13.0 | 7.0 | 93.7 | | |
| 9 | 11.7 | 0.32 | 3.7 | 2.0 | 95.7 | 32.2 | 67.8 |
| 10 | 11.7 | 0.15 | 1.7 | 0.9 | 96.6 | | |
| Strip | 35.0 | 0.23 | 8.0 | 4.3 | 100.9 | | |

TABLE 2-21
Capto ™ MMC ImpRes Run 13 - yield
and non-reduced CE SDS results

| | | | | | Elution | | |
|---|---|---|---|---|---|---|---|
| Load/ | Vol- | Pro- | Pro- | | Cumulative | | |
| Elution | ume | tein | tein | Yield | Yield | Hab | IgG |
| Fractions | (ml) | (mg/ml) | (mg) | (%) | (%) | (%) | (%) |
| Load | 14.6 | 19.18 | 279.45 | 100.0 | | | |
| 1 | 11.7 | 0.67 | 7.81 | 2.8 | 2.8 | | |
| 2 | 11.7 | 3.12 | 36.39 | 13.0 | 15.8 | 1.4 | 94.2 |
| 3 | 11.7 | 5.39 | 62.79 | 22.5 | 38.3 | | |
| 4 | 11.7 | 5.35 | 62.33 | 22.3 | 60.6 | | |
| 5 | 11.7 | 4.31 | 50.21 | 18.0 | 78.6 | 7.5 | 90.4 |
| 6 | 11.7 | 1.97 | 22.95 | 8.2 | 86.8 | | |
| 7 | 11.7 | 0.77 | 8.98 | 3.2 | 90.0 | 34.9 | 61.7 |
| 8 | 11.7 | 0.33 | 3.80 | 1.4 | 91.3 | | |
| 9 | 11.7 | 0.17 | 1.98 | 0.7 | 92.1 | 50.9 | 34.5 |
| 10 | 11.7 | 0.04 | 0.47 | 0.2 | 92.2 | | |
| Strip | 14.2 | 0.15 | 2.13 | 0.8 | 93.0 | | |

### Capto™ Adhere ImpRes Run

Protein A eluate was adjusted to pH 6.5 with 0.5M Tris base and then loaded on to a Capto™ adhere ImpRes HiScreen column (0.77 cm (ID)×10 cm (H)). Table 2-22 lists the chromatography conditions. Mavrilimumab tightly bound to Capto™ adhere ImpRes at pH 6.5 and was eluted from the column at nearly 100% elution Buffer B. In addition, the single elution peak suggests that Hab was not separated from mavrilimumab. Thereof, no further development was performed for Capto™ adhere ImpRes.

TABLE 2-22
Process parameters for the Capto ™ adhere ImpRes chromatography Operation

| | | | Flow | |
|---|---|---|---|---|
| | | | Rate | |
| Step | Buffer/Solution | CV | (cm/h) | Note |
| Pre-run | 0.5M NaOH | 3.0 | 150 | 60 minutes |
| Sanitization | | | | hold |
| RODI | RODI | 3.0 | 150 | |
| Equilibration | 20 mM Sodium Phosphate, pH 7.0 | 5.0 | 150 | |
| Load | Virus Inactivated Protein A Eluate | Variable | 150 | pH: 6.5 |
| | adjusted to pH 6.5 | | | Cond: 4.79 mS/cm |
| Wash | 20 mM Sodium Phosphate, pH 7.0 | 3.0 | 150 | |
| Gradient | Buffer A | 25 | 150 | |
| Elution | 20 mM Sodium Phosphate, pH 7.0 | | | |
| | Buffer B | | | |
| | 20 mM Sodium Citrate, 50 mM | | | |
| | NaCl, pH 4.0 | | | |
| Strip | 0.1M Acetic Acid | 3.0 | 150 | |
| RODI | RODI | 3.0 | 150 | |
| Post-run | 0.5M NaOH | 3.0 | 150 | 60 minutes hold |
| Sanitization | | | | |
| Storage | 0.1 M NaOH | 3.0 | 150 | |

TABLE 2-23
Capto ™ adhere ImpRes run - yields

| | | | | Eluate | |
|---|---|---|---|---|---|
| | | | | Fraction | Eluate |
| Load/Elution | Vol | Protein | Protein | Yield | Cumulative |
| Fractions | (ml) | (mg/ml) | (mg) | (%) | Yield (%) |
| Load | 10.4 | 17.96 | 186.2 | N/A | N/A |
| F6.5 | 1.5 | 0.93 | 1.4 | 0.7 | 0.7 |
| F7 | 11.7 | 8.19 | 95.4 | 51.2 | 52.0 |
| F8 | 11.7 | 4.86 | 56.6 | 30.4 | 82.4 |
| F9 | 11.7 | 1.08 | 12.6 | 6.8 | 89.2 |
| F10 | 11.7 | | | | |
| Strip | | | | | |

### POROS™ XS Chromatography Worst-Case Scenario Runs

The Hab clearance capabilities of POROS™ XS chromatography was evaluated using a high Hab % load and a high pH elution buffer. Results summarized below indicated that POROS™ XS chromatography decreased Hab from 7-10% to 1-2%.

As shown in Table 2-24 (Runs No. 1-4 and 8-15), POROS™ XS chromatography reduced Hab from 4.2-10.3% in loads to 1.9-2.1% in eluates with a 50 mM sodium acetate, 55 mM sodium chloride, pH 5.4 elution buffer. To understand the Hab removal capability of the POROS™ XS resin, the Hab reduction by POROS™ XS chromatography was evaluated using load materials containing high percentage of Hab. POROS™ XS elution fractions enriched with Hab (˜45%) was generated by a salt gradient elution. The Hab-enriched fractions were then spiked back into a 10% Hab Protein A eluate to yield a CEX load containing 21% Hab. POROS™ XS chromatography, at a lower load challenge (39 g/L resin), cleared Hab from 21% in the CEX load to 2.1% in the CEX eluate (Table 2-24, Run No. 5).

To generate additional Hab-enriched materials for further spiking studies, the POROS™ XS column was stripped with an acetate buffer containing 250 mM NaCl after elution.

The high salt stripped Hab pool was spiked into Brx5 clarified harvest. The spiked clarified harvest was purified through Protein A chromatography, low pH viral inactivation, and POROS™ XS chromatography. Load Hab content as well as elution buffer pH were varied to evaluate POROS™ XS chromatography performance under worst process conditions (Table 2-24, Runs No. 16-19). The Hab results indicated that none of these runs were able to clear the Hab level to the target≤3% level, and that the proposed pH 5.4±0.1, 55 mM±5 mM elution condition for the CEX step may not be robust when the CEX column is loaded at the upper end of the proposed loading range of 60 g/L with CEX load containing high amount of Hab. Thereof, additional studies were performed.

TABLE 2-24
Summary of Hab clearances by POROS ™ XS chromatography

| | | | Column | | | | Elution | Eluate | Eluate |
|---|---|---|---|---|---|---|---|---|---|
| Run | | Column Size | Volume | Load | Load | Elution | NaCl | Yield | Hab |
| No. | Run | (ID × H) | (mL) | (Hab) | (g/L) | pH | (mM) | (%) | (%) |
| 1 | Proving run | 5 cm × 19 cm | 373 | 7.1 | 60 | 5.4 | | 83.1 | 1.9 |
| 2 | Establishment Run 2 | 3.2 cm × 20.2 cm | 160 | 4.2 | 60 | 5.5 | 55 | 89.9 | 2.1 |
| 3 | | | | | 45 | | | 87.4 | 1.7 |
| 4 | | | | | 30 | | | 86.6 | 1.3 |
| 5 | Run with Hab | 1.13 cm × 22 cm | 22 | 21.2 | 39 | 5.4 | | 59.2 | 2.1 |
| | spiked load | | | | | | | | |
| 6 | Truncated Run | 0.8 cm × 20 cm | 10 | 8.5 | 60.9 | 5.5 | 55 | 85.0 | 3.0 |
| 7 | | | | 10.4 | 59 | 5.4 | 55 | 72.3 | 2.4 |
| 8 | 10 L Run 1 | 0.8 cm × 20 cm | 10 | 7.4 | 50 | | | 75.8 | 1.4 |
| 9 | 10 L Run 2 | | | 6.2 | 50 | | | 78.0 | 1.1 |
| 10 | 10 L Run 3 | 5 cm × 20.8 cm | 408.4 | 7.2 | 60 | | | 82.3 | 1.0 |
| 11 | 10 L Run 4 | | | 9.7 | 60 | | | 81.2 | 1.7 |
| 12 | 10 L Run 7 | | | 9.9 | 50 | | | 73.0 | 1.4 |
| 13 | | | | | 30 | | | 64.0 | 0.6 |
| 14 | 10 L Run 5 | | | 10.3 | 50 | | | 79.0 | 1.5 |
| 15 | | | | | 28 | | | 76.0 | |
| 16 | Run with Worst | 0.8 cm × 20 cm | 10 | 20.8 | 60 | 5.5 | 60 | 81.8 | 17.7 |
| | Case elution & | | | | | | | | |
| | spiked Hab load | | | | | | | | |
| 17 | Run with Worst | | | 9.6 | 60 | 5.5 | 60 | 87.0 | 6.7 |
| | Case elution buffer | | | | | | | | |
| 18 | Run with Worst | | | 26.1 | 60 | 5.4 | 60 | 67.5 | 8.7 |
| | Case elution & | | | | | | | | |
| | spiked Hab load | | | | | | | | |
| 19 | Run with Worst | | | 26.1 | 60 | 5.5 | 55 | 76.6 | 15.8 |
| | Case elution & | | | | | | | | |
| | spiked Hab load | | | | | | | | |

### Loading Study

Loading study were performed using pre-packed POROS™ XS column (0.8 cm (ID)×20 cm (H)) with CEX load generated from 200 L scale demonstration run 1. Product breakthrough was observed at 102 g/L. Yields and analytical results of the loading study runs are shown in Table 2-25. Higher loading correlates with higher CEX step yield. However, higher loading also correlates with higher Hab % in the CEX eluate and lower IEX main peak %. Balancing step yield and product quality, 30-60 g/L was selected as the loading range for the CEX step.

TABLE 2-25
CEX loading study - IEC & SEC results

| | | | | SEC Results | | | |
|---|---|---|---|---|---|---|---|
| | IEC Results | | | Aggre- | | Frag- | |
| Loading | Acidic | Main | Basic | gation | IgG | ment | |
| (g/L) | (%) | (%) | (%) | (%) | (%) | (%) | Comment |
| 30 | 18.2 | 63.1 | 18.7 | 0.17 | 99.3 | 0.49 | |
| 45 | 17.7 | 60.8 | 21.4 | 0.25 | 99.2 | 0.52 | |
| 60 | 17.2 | 60.7 | 22.1 | 0.33 | 99.2 | 0.52 | |
| 110 | 18.0 | 57.1 | 25.0 | 0.37 | 99.0 | 0.59 | Lower |
| | | | | | | | main peak |

### Lab Scale CEX Runs

Harvest material from 10 L bioreactor runs and 200 L demonstration run 1 were purified through drug substance to assess product quality. POROS™ XS was used as a second chromatography step in these purifications and results are summarized below. With pH 5.4 elution, POROS™ XS chromatography decreased the level of Hab from 7-10% to <2%. POROS™ XS chromatography also does a good job in terms of aggregate reduction and HCP clearance (Tables 2-27 and 2-28). Eluate volume was around 4 to 5 CVs. Yields were 64 to 87%. Decreased loading correlated with decreased Hab in the eluate and lower yield (Table 2-26).

TABLE 2-26
Development CEX Runs - Yield and Hab Results

| | Load | | | Elution | Eluate | Eluate |
|---|---|---|---|---|---|---|
| | (Hab | Load | Elution | NaCl | Yield | Hab |
| Reactor | %) | (g/L) | pH | (mM) | (%) | (%) |
| Truncated Run | 8.5 | 60.9 | 5.5 | 55 | 85.0 | 3.0 |
| | 10.4 | 59 | 5.4 | 55 | 72.3 | 2.4 |
| 10 L Run # 1 | 7.4 | 50 | | | 75.8 | 1.4 |
| 10 L Run # 2 | 6.2 | 50 | | | 78.0 | 1.1 |
| 10 L Run # 3 | 7.2 | 60 | | | 82.3 | 1.0 |
| 10 L Run # 4 | 9.7 | 60 | | | 81.2 | 1.7 |
| 10 L Run # 5 | 8.7 | 50 | | | 79.1 | 1.3 |
| | | 28 | | | 76.0 | 1.0 |
| 10 L Run # 7 | 9.5 | 30 | | | 64.0 | 0.6 |
| | | 50 | | | 73.0 | 1.4 |
| 200 L Demo | 6.9 | 30 | 5.4 | 55 | 75.3 | 1.0 |
| Run 1 | | 45 | | | 80.6 | 1.4 |
| | | 60 | | | 82.0 | 1.8 |
| | | 60 | | | 86.9 | 1.5 |

TABLE 2-27
SEC monomer content in CEX loads and eluates

| Bioreactor Run | Load (%) | Eluate (%) |
|---|---|---|
| Proving Run | 96.8 | 98.5 |
| 10 L Run # 5 | 96.5 | 99.0 |
| 200 L Demo Run 1 | 96.9 | 98.3 |

TABLE 2-28
Residual HCP in CEX loads and eluates

| Bioreactor Run | Load (ppm) | Eluate (ppm) |
|---|---|---|
| Proving Run | 147.9 | 6.7 |
| 10 L Run # 5 | 146.4 | 4.2/2.8 |
| 200 L Demo Run 1 | 359.6 | 7.7 |

### Pilot Scale CEX Run

Pilot scale run was performed using a 20 cm (ID)×20 cm (H) POROS™ XS column at 60 g/L loading with Protein A eluate from 200 L scale demonstration run 1 (Demo run 1). CEX UV chromatogram for the Demo run 1 was similar to lab scale runs. Yield was 86% for Demo run 1, slightly higher than lab scale but was consistent with development runs. Hab was decreased from 6.9% to 1.6% for the Demo run 1. In order to ensure adequate Hab reduction, Demo run 2 targeted an elution pH of 5.3. Compared to pH 5.4 elution, pH 5.3 elution reduced Hab in the CEX elution pool to <1% (RPT-0122). However, pH 5.3 elution resulted in lower step yields (63-65%) and larger elution volumes (6.4-7.0 CV) than pH 5.4 elution (4.9 CV). Based on the Demo runs results and manufacturing capability for pH control, an elution pH of 5.35±0.1 is recommended for 200 L and 2000 L cGMP batches.

CEX Runs with Worst-Case Conditions

POROS™ XS chromatography were performed with worst-case load (high Hab %) and elution conditions (high pH and high salt) conditions. With 13.5% Hab in the load, at 60 g/L load challenge using pH 5.4, 55 mM NaCl elution buffer, the resulting Hab % in the CEX eluate is 3.6%. Given the possibility that the Hab in the CEX load/Protein A eluate from the phase 3 upstream process can be as high as 10-12%, to ensure the resulting Hab level is approximately 3% or lower, the proposed CEX elution conditions for phase 3 process is pH 5.35±0.1, 55±5 mM NaCl with a 30-60 g/L load challenge. The proposed CEX conditions will ensure the final Hab level is approximately 3% or lower even when the Hab level in the Phase 3 clarified harvest is as high as 12-13%.

TABLE 2-29
POROS ™ XS chromatography worst-
case runs - yields and Hab %

| | Load | | | Elution | Eluate | Eluate |
|---|---|---|---|---|---|---|
| Worst-Case | (Hab | Load | Elution | NaCl | Yield | Hab |
| Condition | %) | (g/L) | pH | (mM) | (%) | (%) |
| Load Hab & elution | 20.8 | 60 | 5.5 | 60 | 81.8 | 17.7 |
| Elution | 9.6 | 60 | | | 87.3 | 6.7 |
| Load Hab & elution | 22.2 | 60 | 5.4 | 60 | 67.5 | 7.0 |
| Load Hab & elution | | 60 | 5.5 | 55 | 76.6 | 13.2 |
| Load Hab | 23.4 | 60 | 5.4 | 55 | 71.0 | 13.7 |
| Load Hab | 17.0 | 60 | | | 71.0 | 5.4 |
| Load Hab | 13.5 | 60 | | | 72.2 | 3.6 |
| Load Hab | 13.5 | 45 | | | 70.4 | 2.5 |

### Scale-up Calculations

80×20 cm CEX column is recommended for the proposed Phase 3 2000 L scale manufacturing process. The estimated titer for 2000 L scale bioreactor is 8.5±1.3 g/L. Based on facility fit calculations, the estimated number of CEX cycles at 2000 L bioreactor scale is 3 to 5.

### Conclusion

The main function of the POROS™ XS chromatography is Hab clearance. Results presented in the example demonstrated that the POROS™ XS chromatography step is capable of decreasing the level of Hab from 7-10% to around 1-2%. The results also indicated clearance of HCP and aggregates by POROS™ XS chromatography. Proposed operational conditions for the POROS™ XS chromatography are listed in Table 2-30. The proposed loading range for the CEX column is 30-60 g/L. The estimated yield for the CEX step is 65-85% with an elution volume of 4 to 5 CVs. Analytical data of drug substance and in-process impurity clerance from non-GMP (200 L) and GMP (200 L and 2000 L) production runs, which included the POROS™ XS chromatography process in Table 2-30, are summarized in Tables 4-1 and 4-2.

TABLE 2-30
POROS ™ XS chromatography conditions

| Step | Buffer/Solution | CV | Flow Rate (cm/h) |
|---|---|---|---|
| Pre-run Sanitization | 0.5M NaOH | 3.0 | 80 |
| RODI | RODI | 3.0 | 300 |
| Equilibration | 50 mM Sodium Acetate pH 5.0 | 5.0 | 300 |
| Load | Virus Inactivated Protein A Eluate | Variable | 300 |
| | (pH 5.0 ± 0.1, Conductivity ≤ 4.0 mS/cm) | | |
| Wash | 50 mM Sodium Acetate pH 5.0 | 5.0 | 300 |
| Elution | 50 mM Acetate, 55 mM NaCl, pH 5.35 ± 0.1 | 10.0 | 300 |
| | Collect 0.5 AU/cm-2.0 AU/cm | | |
| Strip | 50 mM Acetate, 1M NaCl, pH 5.5 | 3.0 | 300 |
| Post-run Sanitization | 0.5M NaOH | 3.0 | 80 |
| Storage | 0.1M NaOH | 3.0 | 300 |

### Example 3: Downstream Process for Antibody Purification Using Anion Exchange Chromatography

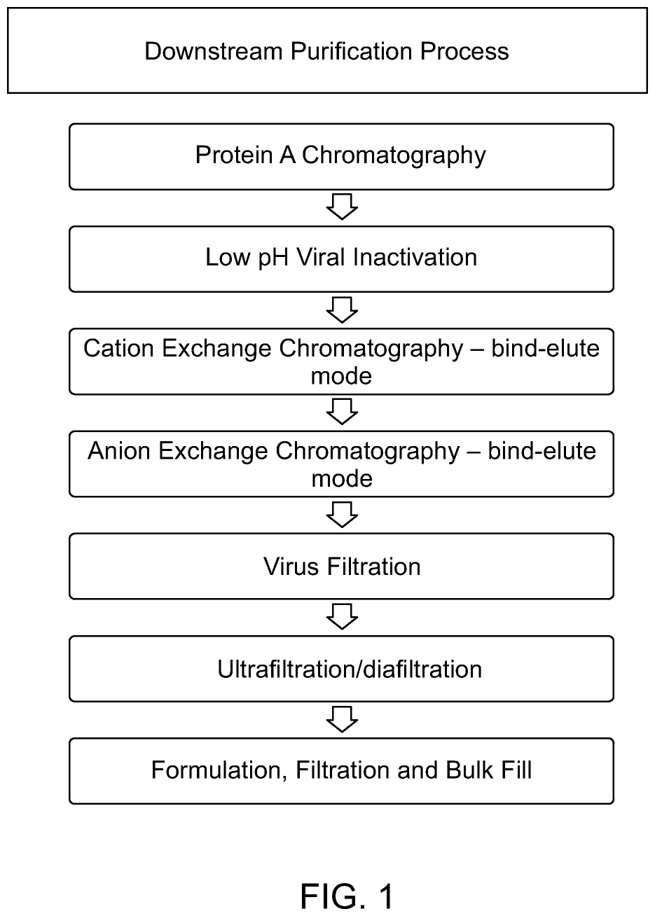

Example 3 describes the development of an anion exchange chromatography step for the purification process of an anti-GM-CFSRa antibody, mavrilimumab. The downstream purification process are summarized in Table 3-1 and FIG. 1.

TABLE 3-1
Purification process overview

| Step | |
|---|---|
| 1 | Protein A Chromatography |
| 2 | Low pH Viral Inactivation |
| 3 | Cation Exchange Chromatography - bind-elute mode |
| 4 | Anion Exchange Chromatography - bind-elute mode |
| 5 | Virus Filtration |
| 6 | Ultrafiltration/diafiltration |
| 7 | Formulation, Filtration and Bulk Fill |

ACRONYMS & DEFINITIONS

| Term | Definition |
|---|---|
| AEX | Anion exchange chromatography |
| Brx | Bioreactor |
| CEX | Cation exchange chromatography |
| CHO | Chinese Hamster Ovary Cells |
| CoA | Certificate of Analysis |
| CV | Column volume |
| DBC | Dynamic Binding Capacity |
| g | Gram |
| HCP | Host Cell Proteins |
| hr | Hour |
| IEC | Ion Exchange Chromatography |
| kDa | Kilodalton |
| L | Liter |
| mM | Millimolar |
| min | Minutes |
| mS | Milli-siemens |
| Ph3 | Phase 3 |
| UF/DF | Ultrafiltration/Diafiltration |
| VI | Virus inactivation |
| VIN | Virus inactivation & neutralization |

### Materials

### Harvests

Harvests collected from 10 L and 200 L bioreactor runs were used to develop AEX column operation for the proposed mavrilimumab downstream process.

### Analytical Methods

A280 values were measured for the purified samples using Solo VPE. Protein concentration was then calculated using extinction coefficient 1.44 mg⁻¹·mL∜cm⁻¹. Size exclusion chromatography, non-reduced CE SDS and ion exchange chromatography analyses were performed.

### Results and Discussion

### Proposed AEX Chromatography Step

Capto™ Q ImpRes bind and elute chromatography was chosen as the third chromatography step in order to reduce acidic species, high molecular weight (HMW) impurities and host cell protein (HCP). The column is typically packed between 16-21 cm and the target bed height may be changed to optimize facility fit. Produce loading residence time is kept consistent at 4 minutes. The column is loaded at 50-60 g/L resin and eluted with high salt buffer. Acidic charge species bind more tightly to the column and are removed in the post-elution high salt strip. The anticipated step yield for the proposed AEX step is 60-84%. The process parameters of the step are described in Table 3-2.

TABLE 3-2
AEX Chromatography Step Description

| | | Volume | Flow Rate | |
|---|---|---|---|---|
| Step | Buffer/Solution | (CVs) | (cm/hr) | Comments |
| Pre-Use | 0.5M NaOH | 3 | 300 for 1 CV, | 30-minute |
| Sanitization | | | then 64 | contact time |
| Charge | 50 mM Sodium Acetate, 1M | 3 | 300 | |
| | Sodium Chloride, pH 5.5 | | | |
| Equilibration | 50 mM Histidine, pH 6.0 | 4 | 300 | |
| Load | AEX Load pH 5.9-6.1 | Variable | Target 4 | 50-60 g/L |
| | | | Minute | resin |
| | | | Contact Time | |
| Wash | 50 mM Histidine, pH 6.0 | 3 | 300 | |
| Elution | 50 mM Histidine, 105 mM Sodium | Variable | 300 | Collect |
| | Chloride, pH 6.0 | | | 0.5 AU/cm- |
| | | | | 2.75 AU/cm |
| Strip | 50 mM Sodium Acetate, 1M NaCl, | 3 | 300 | |
| | pH 5.5 | | | |
| Post-Use | 0.5M NaOH | 3 | 300 for 1 CV, | 30-minute |
| Sanitization | | | then 64 | contact time |
| Storage | 0.1M NaOH | 3 | 300 | |

### 10 L Bioreactor 1-4 Purification and Resin Evaluation

TABLE 3-3
10 L Bioreactor 1-4 Purification Summary

| Goal | Parameters Evaluated |
|---|---|
| Purify harvest material from 10 L reactor 1 | Chromatographic profile |
| and 2 through POROS ™ XS eluate to assess | Yield |
| need for further acidic species modulation | Manufacturability |
| | Analytical icIEF |
| Purify harvest material from 10 L reactor 3 | Chromatographic profile |
| and 4 through DS to assess need for 3^{rd} | Yield |
| polishing step | Manufacturability |
| | Analytical icIEF, IEC |

A subset of samples from bioreactor 1-4 were analyzed by IEC as shown in Table 3-4. Since analytical IEC will be used for release testing, a decision was made to use analytical IEC to evaluate acidic species.

TABLE 3-4
IEC Results for CEX Eluate and DS from Bioreactor 1-4

| Sample Description | Acidic Total (%) | Main Total (%) | Basic Total (%) |
|---|---|---|---|
| Bioreactor 1 CEX Eluate | 19.4 | 63.9 | 16.7 |
| Bioreactor 2 CEX Eluate | 27.4 | 55.4 | 17.2 |
| Bioreactor 3 CEX Eluate | 19.3 | 60.2 | 20.5 |
| Bioreactor 4 CEX Eluate | 17.4 | 60.2 | 22.4 |
| Bioreactor 3 DS | 19.4 | 60.3 | 20.3 |
| Bioreactor 4 DS | 18.5 | 58.4 | 23.2 |

The IEC results from purification runs using harvest from 10 L bioreactor 1-4 indicated that further acidic species reduction was needed. Therefore, additional resins were evaluated for the 3rd polishing column step.

### Resin Evaluation

Three resins were evaluated for their capability to reduce acidic species % while maintaining step yield and manufacturability. These resins were chosen based upon their propensity to bind to negatively charged (acidic) species. The resins evaluated are shown in Table 3-5 and Table 3-6.

TABLE 3-5
Resin Evaluation Summary

| Goal | Parameters Evaluated | Notes and Observations |
|---|---|---|
| Preliminary | Chromatographic profile | All three reins (POROS ™ XQ, Capto ™ Q |
| evaluation | Yield | ImpRes, and Capto ™ adhere ImpRes) were |
| | Manufacturability | moved forward for further evaluation |
| Evaluate resins for | Analytical IEC | IEC results indicated that POROS ™ XQ |
| acidic species | Chromatographic profile | provided most robust reduction of acidic |
| clearance | Yield | species. Capto ™ Q ImpRes and Capto ™ |
| | Manufacturability | adhere ImpRes also provided adequate |
| | | reduction. |

TABLE 3-6
Resin Candidates

| Resin | Resin Type | Ligand | Notes and Observations |
|---|---|---|---|
| POROS ™ XQ | Strong Anion | Quaternary amine | Small scale results indicate similar |
| Capto ™ Q | Exchange | | DBC, POROS ™ XQ has improved |
| ImpRes | | | acidic species clearance relative to |
| | | | Capto ™ Q ImpRes |
| Capto ™ | Multimodal Strong | N-benzyl methyl | Narrow elution pH range, low pH |
| adhere ImpRes | Anion Exchange | ethanolamine | elution resulted in decreased |
| | | | stability |

POROS™ XQ, Capto™ Q ImpRes, and Capto™ adhere ImpRes resins were evaluated across 71 development scale runs for yield, elution profile, and scalability. Selected product pools were analyzed for product quality. The analytical results for these process pools are summarized in Table 3-7. Capto™ adhere ImpRes demonstrated the most robust acidic species clearance but was limited by a narrow elution pH range and thereof poor manufacturability. POROS™ XQ and Capto™ Q ImpRes showed similar manufacturability and yield. POROS™ XQ was chosen as the candidate for further development based upon its greater ability to clear acidic species relative to Capto™ Q ImpRes under the selected conditions.

TABLE 3-7
Analytical IEC Results for Screening Runs

| | | | Load | | | | | | Mock | | | |
|---|---|---|---|---|---|---|---|---|---|---|---|---|
| | Run | Column | Challenge | Loading | Equilibration | Wash 2 | | Fxn | Pool | Acidic | Main | Basic |
| Resin | # | Volume | (g/L resin) | pH | Buffer | Buffer | Elution | #'s | Yield | % | % | % |
| AEX Load Material Sourced from 10 L Bioreactor 3 | | | | | | | | | N/A | 20.2 | 59.6 | 20.1 |
| Capto ™ | 46 | 1 mL | 55 | 6.5 | 50 mM Bis | 50 mM | E1: 50 mM | 19-33 | 52% | 12.9 | 59.3 | 27.8 |
| adhere | | | | | Tris, pH 6.5 | Acetate, | Acetate, | 19-35 | 56% | 13.6 | 58.3 | 28.1 |
| ImpRes | | | | | | pH 5.5 | pH 5.00 | 19-37 | 59% | 12.2 | 60.2 | 27.7 |
| | | | | | | | E2: 50 mM | | | | | |
| | | | | | | | Acetate, | | | | | |
| | | | | | | | pH 4.75 | | | | | |
| | | | | | | | E3: 50 mM | | | | | |
| | | | | | | | Acetate, | | | | | |
| | | | | | | | pH 4.50 | | | | | |
| | 50 | | 55 | | | | E1: 50 mM | 1-8 including | 64% | 14.4 | 60.4 | 25.2 |
| | | | | | | | Acetate, | wash 7% | | | | |
| | | | | | | | pH 4.50 | 9-14 | 26% | 37.3 | 53.4 | 9.3 |
| | | | | | | | E2: 50 mM | | | | | |
| | | | | | | | Acetate, | | | | | |
| | | | | | | | pH 4.25 | | | | | |
| | | | | | | | E3: 50 mM | | | | | |
| | | | | | | | Acetate, | | | | | |
| | | | | | | | pH 4.00 | | | | | |
| | 54 | 5 mL | 55 | | | | 50 mM | 4-5 | 71% | 18.8 | 59.7 | 21.6 |
| | | | | | | | Acetate, | 4-6 | 82% | 19.1 | 60.1 | 20.8 |
| | | | | | | | pH 4.3 | 4-7 | 84% | 20.4 | 58.7 | 21 |
| POROS ™ | 35 | 1 mL | 45 | 7.3 | 50 mM Bis | N/A | 50 mM | 2-6 | 57% | 15.6 | 62.5 | 21.8 |
| XQ | | | | | Tris, pH 7.3 | | Bis Tris, | 2-7 | 60% | 15.9 | 62.2 | 21.8 |
| | | | | | | | 125 mM | | | | | |
| | | | | | | | NaCl, pH | | | | | |
| | | | | | | | 7.3 | | | | | |
| | 47 | | | | | 50 mM Bis | 50 mM | 13-16 | 54% | 14.7 | 61.4 | 24 |
| | | | | | | Tris, pH 7.0 | Bis Tris, | 13-17 | 67% | 16.4 | 61.3 | 22.4 |
| | | | | | | | 0-250 mM | 13-18 | 77% | 17.8 | 61.2 | 21.1 |
| | | | | | | | NaCl | 13-20 | 84% | 21.3 | 58.4 | 20.3 |
| | | | | | | | Gradient, | | | | | |
| | | | | | | | pH 7.0 | | | | | |
| | 42 | | | | | 50 mM Bis | 50 mM | 10-14 | 56% | 15 | 60.3 | 24.7 |
| | | | | | | Tris, pH 6.5 | Bis Tris, | 10-15 | 67% | 16.4 | 60.7 | 22.9 |
| | | | | | | | 0-250 mM | 10-16 | 77% | 20 | 59.7 | 20.3 |
| | | | | | | | NaCl | | | | | |
| | | | | | | | Gradient, | | | | | |
| | | | | | | | pH 6.5 | | | | | |
| | 52 | 5 mL | | | | 50 mM Bis | 50 mM | 8-13 | 66% | 16.3 | 60 | 23.8 |
| | | | | | | Tris, pH 7.0 | Bis Tris, | 8-14 | 77% | 16.2 | 61.6 | 22.2 |
| | | | | | | | 0-250 mM | 8-20 | 94% | 20.5 | 60.7 | 18.9 |
| | | | | | | | NaCl | | | | | |
| | | | | | | | Gradient, | | | | | |
| | | | | | | | pH 7.0 | | | | | |
| Capto ™ | 39 | 1 mL | 55 | 7.3 | 50 mM Bis | 50 mM Bis | 50 mM | 15-19 | 53% | 17.0 | 57.9 | 25.1 |
| Q | | | | | Tris, pH 7.3 | Tris, pH 7.0 | Bis Tris, | 15-20 | 64% | 17.5 | 59.2 | 23.3 |
| ImpRes | | | | | | | 0-250 mM | 15-21 | 73% | 19.7 | 58.0 | 22.3 |
| | | | | | | | NaCl | | | | | |
| | | | | | | | Gradient, | | | | | |
| | | | | | | | pH 7.0 | | | | | |

### POROS™ XQ Development, 10 L Bioreactor 5 and 7 Purification, Demo Run 1

During elution buffer optimization, a decision was made to switch from Bis-Tris buffer to Histidine buffer. This decision was based on the ability to adequately source multi-compendial histidine vs. Bis-Tris. Using the salt gradient runs performed at pH 7.0 as a reference, several step elution conditions were evaluated as summarized in Table 3-9.

TABLE 3-8
POROS ™ XQ Elution Condition Screening Summary

| Goal | Parameters Evaluated | Notes and Observations |
|---|---|---|
| Select step elution conditions | Chromatographic profile | 50 mM Histidine, 125 mM |
| for POROS ™ XQ resin for use | Yield | NaCl, pH 7.0 selected as |
| during 10 L Bioreactor 5 and 7, | Manufacturability | elution buffer |
| and 200 L Demo Run 1 | Analytical IEC | |
| purification | | |

TABLE 3-9
POROS ™ XQ Runs 60-64 Conditions

| | | | Load | | | | |
|---|---|---|---|---|---|---|---|
| | Run | Column | Challenge | Loading | Equilibration | Wash 2 | |
| Resin | # | Volume | (g/L resin) | pH | Buffer | Buffer | Elution |
| POROS ™ XQ | 52 | 5 mL | 45 | 7.3 | 50 mM Bis- | 50 mM Bis- | 50 mM Bis |
| Lot XQ-039 | | | | | Tris, pH 7.3 | Tris, pH 7.0 | Tris, 0-250 mM |
| | | | | | | | NaCl |
| | | | | | | | Gradient, |
| | | | | | | | pH 7.0 |
| | 60 | 1 mL | 50 | 7.3 | 50 mM | 50 mM | 50 mM |
| | | | | | Histidine, | Histidine, | Histidine, |
| | | | | | pH 7.3 | pH 7.0 | 115 mM |
| | | | | | | | NaCl, pH 7.0 |
| | 61 | 1 mL | 50 | 7.3 | 50 mM | 50 mM | 50 mM |
| | | | | | Histidine, | Histidine, | Histidine, |
| | | | | | pH 7.3 | pH 7.0 | 125 mM |
| | | | | | | | NaCl, pH 7.0 |
| | 62 | 1 mL | 50 | 7.3 | 50 mM | 50 mM | 50 mM |
| | | | | | Histidine, | Histidine, | Histidine, |
| | | | | | pH 7.3 | pH 7.0 | 135 mM |
| | | | | | | | NaCl, pH 7.0 |
| | 63 | 1 mL | 50 | 7.0 | 50 mM | N/A | 50 mM |
| | | | | | Histidine, | | Histidine, |
| | | | | | pH 7.0 | | 125 mM |
| | | | | | | | NaCl, pH 7.0 |
| | 64 | 1 mL | 50 | 7.0 | 50 mM | N/A | 50 mM |
| | | | | | Histidine, | | Histidine, |
| | | | | | pH 7.0 | | 135 mM |
| | | | | | | | NaCl, pH 7.0 |

TABLE 3-10
POROS ™ XQ Runs 60-64 Yield Table

| Fractions (CV) | Run 60 | Run 61 | Run 62 | Run 63 | Run 64 |
|---|---|---|---|---|---|
| 1 | 22% | 27% | 33% | 24% | 35% |
| 2 | 45% | 54% | 63% | 52% | 63% |
| 3 | 58% | 67% | 76% | 65%* | 76%* |
| 4 | 65% | 74% | 83% | 73%* | 83%* |
| 5 | 69% | 79% | 86% | 77% | 87% |
| 6 | 72% | 82% | 88% | 79% | 89% |
| 7 | 75% | 84% | 89% | 81% | 90% |

*Submitted for analytical IEC

TABLE 3-11
Analytical IEC Results for POROS ™ XQ
Elution Condition Screening

| Run # | Fractions (CVs) | Yield % | Acidic % | Main % | Basic % |
|---|---|---|---|---|---|
| Load | N/A | N/A | 21.3 | 58.2 | 20.5 |
| Run 63 | 3 | 65% | 17.5 | 59.8 | 22.8 |
| | 4 | 73% | 17.7 | 59.8 | 22.5 |
| Run 64 | 3 | 76% | 18.3 | 60.0 | 21.7 |
| | 4 | 83% | 18.7 | 60.0 | 21.3 |

Based on the yield and acidic species clearance results from these runs, 50 mM Histidine, 125 mM NaCl, pH 7.0 (Run 63 conditions) was chosen as the target elution buffer for this step with a target load pH of 7.0. The analytical IEC results show an acidic species reduction of 3-4%. These parameters allow for balance between yield, acidic species clearance, and manufacturability (elution volume).

### 10 L Bioreactor 5 and 7 Lab-Scale Purification Runs and 200 L Demo Run 1

Bioreactor 5 and 7 were purified using POROS™ XQ as the 3′ polishing step. The analytical IEC data shows that center-point elution conditions (50 mM Histidine, 125 mM Sodium Chloride, pH 7.0) achieve a 3-5% reduction in acidic species with yield of 69-70%. For Bioreactor 5 material, the elution buffer was varied between cycles as shown in Table 3-13 in order to explore product quality (with regards to acidic species) under worst-case process conditions (cycle 2 with bioreactor 5). A decrease in elution buffer pH and an increase in salt concentration resulted in a 2-3% reduction in acidic species across the AEX column (as compared to the 3-5% reduction with elution under target conditions).

TABLE 3-12
10 L Bioreactor 5 and 7, 200 L Demo Run 1 Purification Summary

| Goal | Parameters Evaluated |
|---|---|
| Purify harvest material from 10 L reactor 5 | Chromatographic profile |
| and 7 through to DS to confirm POROS ™ XQ | Yield |
| performance and product quality | Manufacturability |
| | Analytical IEC |
| Utilize POROS ™ XQ as the 3rd polishing step | Chromatographic profile |
| during purification of harvest material | Yield |
| from 200 L Demo Run 1. | Manufacturability |
| | Analytical IEC |

TABLE 3-13
Bioreactor 5 and 7, Demo Run 1 Conditions and Process Performance

| | Cycle 1 | Cycle 2 | Cycle 1 | Cycle 1 |
|---|---|---|---|---|
| Bioreactor | 5 | 5 | 7 | Demo Run 1 |
| Elution Buffer | 50 mM Histidine, 125 mM | 50 mM Histidine, 130 mM | 50 mM Histidine, 125 mM | 50 mM Histidine, 125 mM |
| | Sodium Chloride, pH 7.0 | Sodium Chloride, pH 6.8 | Sodium Chloride, pH 7.0 | Sodium Chloride, pH 7.0 |
| Column ID (cm) | 2.6 | 2.6 | 2.6 | 20.0 |
| BH (cm) | 18.7 | 18.7 | 18.7 | 19.0 |
| Elution CVs | 4.1 | 3.8 | 4.0 | 2.2^{a} |
| Load Challenge (g/L) | 50 | 50 | 50 | 28^{a} |
| Step Yield | 69.2% | 82.3% | 69.7% | 72.6%^{a} |
| % of Load Lost to | 0% | 0% | 0% | 5.7%^{a} |
| Breakthrough | | | | |

^{a}Breakthrough first seen during load at 28 g/L resin. Step yield based upon total amount loaded. Lower elution CVs due to lower load challenge.

TABLE 3-14
Analytical IEC Results for Brx 5, Brx 7, and Demo Run 1

| | Acidic Total | Main Total | Basic Total |
|---|---|---|---|
| Sample Description | (%) | (%) | (%) |
| Brx 5 CEX Eluate Cycle 1 | 15.6 | 60.9 | 23.5 |
| Brx 5 AEX Eluate Cycle 1 | 11.4 | 61.8 | 26.8 |
| Brx 5 AEX Eluate Cycle 2 | 13.4 | 61.3 | 25.3 |
| BRX5 DS | 11.6 | 61.3 | 27.1 |
| Brx 7 AEX Load | 15.8 | 57.9 | 26.4 |
| Brx 7 AEX Eluate | 12.8 | 56.8 | 30.5 |
| Brx 7 DS | 12.1 | 57.4 | 30.5 |
| Demo Run 1 AEX Load | 18.4 | 60.4 | 21.2 |
| Demo Run 1 AEX Eluate | 16.1 | 60.3 | 23.6 |
| Demo Run 1 DS | 15.5 | 62.8 | 21.7 |

Demo Run 1 was performed to confirm the purification process at 200 L scale. During loading of the POROS™ XQ column, breakthrough was observed at 28 g/L resin. The resin lot used was XQ-037. The target load was 45 g/L based upon DBC runs performed at lab-scale (resin lot XQ-042). This indicated variability in mavrilimumab binding capacity between different POROS™ XQ resin lots. To evaluate the binding capacity variability of the POROS™ XQ resin, DBC10 was performed on 5 different POROS™ XQ resin lots at the same conditions as Demo Run 1 as shown in Table 3-15. It should be noted that majority of the development work was performed using high capacity resin lot XQ-042 including purification of Bioreactor 5 and 7.

Based on the DBC10 data in Table 3-14, the upper end of the POROX XQ load range will have to be set at 32 g/L (80% of the lowest DBC10, which is 40 g/L). As a result, using POROS™ XQ resin would significantly challenge manufacturing cadence and potentially require evaluating binding capacity of the resin lot before use in production. Given these associated challenges, a decision was made to evaluate Capto™ Q ImpRes as an alternative to POROS™ XQ as the 3′ chromatography step in the mavrilimumab purification process. Capto™ Q ImpRes has the same ligand as POROS™ XQ (quaternary amine) and had previously shown to clear acidic species.

TABLE 3-15
Summary of Resin Capacity Screening at Lab-Scale

| | | Ionic | BSA | Internal | Column |
|---|---|---|---|---|---|
| Lot | Manufacturing | Capacity | DBC | DBC10 | Dimensions |
| Number | Date | μmol/mL | g/L | g/L | cm × cm |
| XQ-034 | 6 Apr. 2018 | 110.09 | 154.32 | 44.76 | 1.1 × 20 |
| XQ-038 | 10 Aug. 2018 | 110.06 | 158.73 | 41.72 | |
| XQ-037 | 11 Jul. 2018 | 107.93 | 142.73 | 40.36 | |
| XQ-042 | 8 Feb. 2019 | 115.01 | 145.81 | 54.56^{a} | |
| XQ-049 | 18 Nov. 2019 | 111.04 | 147.97 | 40.22 | |

^{a}Load material limited; did not reach breakthrough

### Capto™ Q ImpRes Development, Demo Run 2

### Capto™ Q ImpRes Elution Condition and Loading Capacity Screening

Four Capto™ Q ImpRes resin lots were evaluated for loading capacity at lab-scale using the conditions shown in Table 3-17. pH of 5.9 was used for equilibration buffer and load as this would be the low end of the pH range 5.9-6.1 and worst case condition for binding. The DBC10 for all 4 resin lots screened was comparable. An 80% safety factor was then applied and the high end loading capacity was determined to be 60 g/L resin.

Binding capacity evaluation was also performed at pH 5 and pH 7. As expected, lower load pH resulted in lower load capacity which would not allow column sizing to fit the facility. At pH 7, there is concern that extended stability could be affected. Therefore, loading at pH 6.0+/−0.1 was selected.

TABLE 3-16
Capto ™ Q ImpRes Load Capacity and Elution Condition Screening Summary

| Goal | Parameters Evaluated | Notes and Observations |
|---|---|---|
| Evaluate Capto ™ Q ImpRes | DBC10 | Evaluated 4 different resin lots. High |
| loading capacity using multiple | Facility Fit | end of loading range determined to be |
| resin lots | | 60 g/L |
| Select loading and step elution | Chromatographic profile | 50 mM Histidine, 105 mM NaCl, pH 6.0 |
| conditions for Capto ™ Q | Yield | selected as elution buffer |
| ImpRes resin for use during | Manufacturability | |
| Demo Run 2 | Analytical IEC | |

TABLE 3-17
Capto ™ Q ImpRes Loading Capacity Evaluation

| | | | Load | | | |
|---|---|---|---|---|---|---|
| | EQ | Load | Conductivity | DBC10 | 80% DBC10 | Column |
| Resin Lot | Buffer | pH | (mS/cm) | (g/L resin) | (g/L resin) | Dimensions |
| 10280953 | 50 mM | 5.9 | 9.0 | 73.4 | 58.7 | 0.77 cm × |
| | Histidine, | | | | | 10.0 cm |
| | pH 5.9 | | | | | HiScreen |
| 10274725 | | | | 76.1 | 60.9 | 1.1 cm × |
| 10265853 | | | | 77.0 | 61.6 | 20.0 cm |
| 10281108 | | | | 75.2 | 60.1 | |

TABLE 3-18
Capto ™ Q ImpRes Development Runs and Analytical IEC Data

| Run | | Loading | | Elution | | Acidic | Main | Basic |
|---|---|---|---|---|---|---|---|---|
| # | Run Info | (g/L resin) | Load Info | Conditions | Yield | (%) | (%) | (%) |
| Load Material - Assay IEX | | | | | | 18.9 | 60.3 | 20.8 |
| 4 | 2 Step | 65 | pH 6.9, 9 | 50 mM | 1000 mAU = 62% | 15.8 | 60.3 | 23.9 |
| | Elution | | mS/cm | Histidine | 750 mAU = 67% | 15.9 | 60.8 | 23.4 |
| | | | | 150 mM | 650 mAU = 69% | Not Tested | | |
| | | | | NaCl pH | 550 mAU = 71% | 15.8 | 23.2 | 60.9 |
| | | | | 6.9 | | | | |
| | | | | 50 mM | Entire | Not Tested | | |
| | | | | Histidine | Fraction = 13% | | | |
| | | | | 170 mM | | | | |
| | | | | NaCl pH | | | | |
| | | | | 6.9 | | | | |
| 5 | Single Step | 65 | pH 6.9, 9 | 50 mM | 1000 mAU = 53% | 15.3 | 58.9 | 25.9 |
| | Elution, | | mS/cm | Histidine | 750 mAU = 58% | 15.0 | 60.8 | 24.2 |
| | Collection | | | 140 mM | 650 mAU = 60% | Not Tested | | |
| | Based | | | NaCl pH | 550 mAU = 63% | 15.2 | 60.3 | 24.5 |
| | Upon UV | | | 6.9 | | | | |
| 6 | Single Step | 40 | pH 6.9, 9 | 50 mM | 1000 mAU = 26% | Not Tested | | |
| | Elution, | | mS/cm | Histidine | 750 mAU = 34% | Not Tested | | |
| | Collection | | | 140 mM | 650 mAU = 38% | Not Tested | | |
| | Based | | | NaCl pH | 550 mAU = 42% | 12.9 | 46.2 | 40.9 |
| | Upon UV | | | 6.9 | | | | |
| 8 | Single Step | 58 | pH 6.9, 9 | 50 mM | 1000 mAU = 54% | Not Tested | | |
| | Elution, | | mS/cm | Histidine | 750 mAU = 59% | | | |
| | Collection | | | 140 mM | 650 mAU = 62% | | | |
| | Based | | | NaCl pH | 550 mAU = 65% | | | |
| | Upon UV | | | 6.9 | | | | |
| 9 | Single Step | 50 | pH 6.9, 9 | 50 mM | 1000 mAU = 48% | Not Tested | | |
| | Elution, | | mS/cm | Histidine | 750 mAU = 51% | | | |
| | Collection | | | 140 mM | 650 mAU = 54% | | | |
| | Based | | | NaCl pH | 550 mAU = 56% | | | |
| | Upon UV | | | 6.9 | | | | |
| Load Material - Assay IEX | | | | | | 18.9 | 60.3 | 20.8 |
| 11 | 3 Step | 58 | pH 6.1, 9 | 50 mM | E1 = 46% | Not Tested | | |
| | Elution | | mS/cm | Histidine, | | | | |
| | | | | 70 mM | | | | |
| | | | | NaCl, pH | | | | |
| | | | | 5.9 | | | | |
| | | | | 50 mM | E2 = 73% | 15.0 | 60.5 | 24.5 |
| | | | | Histidine, | | | | |
| | | | | 90 mM | | | | |
| | | | | NaCl, pH | | | | |
| | | | | 5.9 | | | | |
| | | | | 50 mM | E3 = 81% | 15.0 | 61.1 | 23.4 |
| | | | | Histidine, | | | | |
| | | | | 100 mM | | | | |
| | | | | NaCl, pH | | | | |
| | | | | 5.9 | | | | |
| 12 | 3 Step | 58 | pH 6.1, 9 | 50 mM | Load did not | Not Tested | | |
| | Elution | | mS/cm | Histidine, | remain bound | | | |
| | | | | 60 mM | during Wash 2 | | | |
| | | | | NaCl, pH | (pH 5.4) | | | |
| | | | | 5.4 | | | | |
| | | | | 50 mM | | | | |
| | | | | Histidine, | | | | |
| | | | | 80 mM | | | | |
| | | | | NaCl, pH | | | | |
| | | | | 5.4 | | | | |
| | | | | 50 mM | | | | |
| | | | | Histidine, | | | | |
| | | | | 100 mM | | | | |
| | | | | NaCl, pH | | | | |
| | | | | 5.4 | | | | |
| 14 | Single Step | 60 | pH 6.0, 9 | 50 mM | 750 mAU = 58% | Not Tested | | |
| | Elution, | | mS/cm | Histidine, | 650 mAU = 61% | 14.6 | 57.4 | 28.0 |
| | Collection | | | 90 mM | 550 mAU = 65% | 14.4 | 57.9 | 27.7 |
| | Based | | | NaCl, pH | | | | |
| | Upon UV | | | 5.9 | | | | |
| 15 | Single Step | 60 | pH 6.0, 9 | 50 mM | 750 mAU = 70% | | | |
| | Elution, | | mS/cm | Histidine, | 630 mAU = 72% | 15.5 | 58.5 | 26 |
| | Collection | | | 100 mM | 550 mAU = 74% | 15.5 | 59.5 | 24.9 |
| | Based | | | NaCl, pH | | | | |
| | Upon UV | | | 5.9 | | | | |
| 16 | Single Step | 64 | pH 6.5, 9 | 50 mM | 750 mAU = 46% | Not Tested | | |
| | Elution, | | mS/cm | Histidine, | 650 mAU = 49% | | | |
| | Collection | | | 125 mM | 550 mAU = 51% | | | |
| | Based | | | NaCl, pH | | | | |
| | Upon UV | | | 6.4 | | | | |
| 17 | Single Step | 64 | pH 6.5, 9 | 50 mM | 750 mAU = 65% | Not Tested | | |
| | Elution, | | mS/cm | Histidine, | 650 mAU = 67% | 14.5 | 59.5 | 26 |
| | Collection | | | 140 mM | 550 mAU = 69% | 15.6 | 58.8 | 25.6 |
| | Based | | | NaCl, pH | | | | |
| | Upon UV | | | 6.4 | | | | |
| 20 | Single Step | 50 | pH 6.0, 9 | 50 mM | 650 mAU = 57% | Not Tested | | |
| | Elution, | | mS/cm | Histidine, | 550 mAU = 61% | | | |
| | Collection | | | 95 mM | | | | |
| | Based | | | NaCl, pH | | | | |
| | Upon UV | | | 6.0 | | | | |
| 21 | Single Step | 40 | pH 6.0, 9 | 50 mM | 650 mAU = 64% | Not Tested | | |
| | Elution, | | mS/cm | Histidine, | 550 mAU = 67% | | | |
| | Collection | | | 100 mM | | | | |
| | Based | | | NaCl, pH | | | | |
| | Upon UV | | | 5.9 | | | | |
| 22 | Single Step | 40 | pH 6.0, 9 | 50 mM | 550 mAU = 32% | Not Tested | | |
| | Elution, | | mS/cm | Histidine, | | | | |
| | Collection | | | 90 mM | | | | |
| | Based | | | NaCl, pH | | | | |
| | Upon UV | | | 6.1 | | | | |
| Load Material - Assay IEX | | | | | | 17.4 | 63.3 | 19.4 |
| 23 | Single Step | 60 | pH 6.0, 9 | 50 mM | 550 mAU = 84% | 15.2 | 61.9 | 22.9 |
| | Elution, | | mS/cm | Histidine, | | | | |
| | Collection | | | 110 mM | | | | |
| | Based | | | NaCl, pH | | | | |
| | Upon UV | | | 5.9 | | | | |
| 24 | Single Step | 40 | pH 6.0, 9 | 50 mM | 550 mAU = 50% | 12.5 | 59.4 | 28.1 |
| | Elution, | | mS/cm | Histidine, | | | | |
| | Collection | | | 110 mM | | | | |
| | Based | | | NaCl, pH | | | | |
| | Upon UV | | | 6.1 | | | | |
| 25 | Single Step | 60 | pH 6.0, 9 | 50 mM | 550 mAU = 90% | 16.1 | 61.7 | 22.2 |
| | Elution, | | mS/cm | Histidine, | | | | |
| | Collection | | | 110 mM | | | | |
| | Based | | | NaCl, pH | | | | |
| | Upon UV | | | 5.8 | | | | |
| 26 | Single Step | 50 | pH 6.0, 9 | 50 mM | 550 mAU = 60% | 13.7 | 61.4 | 24.9 |
| | Elution, | | mS/cm | Histidine, | | | | |
| | Collection | | | 100 mM | | | | |
| | Based | | | NaCl, pH | | | | |
| | Upon UV | | | 6.1 | | | | |

Using the knowledge gained during POROS™ XQ development, a series of lab-scale runs were performed with the Capto™ Q ImpRes column to determine conditions that would maximize capacity while balancing yield and acidic species reduction. These runs and the associated analytical IEC results are shown in Table 3-17.

Runs 4-9 were performed at a load pH of 6.9 and elution pH of 6.9 with varying elution salt concentrations. Runs 4 and 5 were loaded at 65 g/L resin and demonstrated the ability to clear 3% acidic species while maintaining a yield greater than 63%. Run 6 was loaded at 40 g/L, which resulted in a 42% yield, 6% clearance of acidic species, but a drastic shift in both main and basic species.

Runs 11-22 were performed at a load pH of 6.0, 6.1 and 6.5 while varying the elution pH and salt concentration. The runs eluted with pH 5.9 buffer showed the ability to maintain yield above 65% while achieving acidic species reduction between 4-5%. Loading the column at lower load challenges significantly reduced the yield as demonstrated in Run 22.

Based on results from these runs, single step elution runs 23-26 were performed with the descending UV gate set at 550 mAU (path length 2 mm), using parameters that are at the high and low end of proposed ranges for elution pH, elution salt concentration, and load challenge. As previously observed, loading at 40 g/L (53% of DBC10) in run 24 resulted in low yield (50%). Run 23 represented worst case for product quality (high load challenge, low elution pH, high elution salt concentration) and resulted in 84% yield with 2% reduction in acidic species. Run 26 represented worst case for yield and best case for product quality (lower load challenge, high elution pH, and low elution salt concentration) and resulted in 60% yield with 4% reduction in acidic species.

While the main driver of the addition of a 3′ polishing step was to modulate charged species, the SEC and NR-CE-SDS data in Table 3-19 and Table 3-20 indicate that the AEX column also improves SEC purity while half-antibody levels are maintained over the range of conditions tested.

TABLE 3-19
Run 23-26 SEC Data

| | Total % | % | Total % |
|---|---|---|---|
| Sample Description | Aggregate | Monomer | LMW |
| Capto ™ Q ImpRes Load Material | 0.6 | 98.5 | 0.9 |
| Run 23 Capto ™ Q ImpRes Eluate | 0.2 | 98.9 | 0.9 |
| Run 24 Capto ™ Q ImpRes Eluate | 0.1 | 98.8 | 1.1 |
| Run 25 Capto ™ Q ImpRes Eluate | 0.2 | 98.9 | 0.9 |
| Run 26 Capto ™ Q ImpRes Eluate | 0.1 | 98.9 | 1 |

TABLE 3-20
Run 23-26 NR-CE-SDS Data

| Sample Description | % Other | Hab (%) | igG (%) |
|---|---|---|---|
| Run 23 Capto ™ Q ImpRes Eluate | 1.1 | 1.4 | 96.8 |
| Run 24 Capto ™ Q ImpRes Eluate | 0.9 | 1.3 | 95.3 |
| Run 25 Capto ™ Q ImpRes Eluate | 1.4 | 1.4 | 96.4 |
| Run 26 Capto ™ Q ImpRes Eluate | 1.2 | 1.3 | 95.0 |
| Demo Run 1 AEX Load | 1.7 | 1.5 | 95.1 |

*Load material was sourced from Demo Run 1 for this development work.

Based on the yield and acidic species clearance results from the above runs, 50 mM Histidine, 105 mM NaCl, pH 6.0 was chosen as the target elution buffer for the AEX step with a target load pH of 6.0. The column load challenge was set to 50-60 g/L. The data also indicate that underchallenging the column will result in low yield and a shift in the charge profile. The analytical IEC results show an acidic species reduction of 2-4%. These parameters allow for balance between yield, acidic species clearance, and manufacturability (elution volume).

200 L Demo Run 2 with Capto™ Q ImpRes

200 L Demo Run 2 was performed to provide material for use in enabling studies and to demonstrate process performance at 200 L scale. The operating parameters for the Capto™ Q ImpRes step are summarized in Table 3-22.

TABLE 3-21
200 L Demo Run 2 with Capto ™ Q ImpRes Summary

| Goal | Parameters Evaluated |
|---|---|
| Utilize Capto ™ Q ImpRes as the 3rd | Chromatographic profile |
| polishing step during purification of | Yield |
| harvest material from 200 L Demo | Manufacturability |
| Run 2 | Analytical IEC |

TABLE 3-22
Capto ™ Q ImpRes Operating Parameters for Demo Run 2

| Process Step | Parameter | Unit | Target | Range | Comments |
|---|---|---|---|---|---|
| All Steps | Flow Direction | N/A | Down flow | | N/A |
| Pre-Use Sanitization | Volume | CV | 3 | ≥3 | |
| (0.5M NaOH) | Linear Flow Rate | cm/hr | 300 for 1 CV, then 76 | ≤330 | |
| | Contact Time | Minutes | 30 | 30-60 | |
| Charge (50 mM | Volume | CV | 3 | ≥2.8 | |
| Acetate, 1M Sodium | Linear Flow Rate | cm/hr | 300 | 270-330 | |
| Chloride, pH 5.5) | | | | | |
| Equilibration | Volume | CV | 4 | ≥3.8 | N/A |
| (50 mM Histidine, | Linear Flow Rate | cm/hr | 300 | 270-330 | |
| pH 6.0) | Sample for pH and conductivity to ensure effluent is within buffer range. | | | | |
| | pH | pH | 6.0 | 5.9-6.1 | N/A |
| | Conductivity | mS/cm | TBD | TBD | |
| AEX Load Adjustment | Titrant | CEX Eluate Product | 1.6% | N/A | |
| (0.5M Tris Base) | (0.5M Tris Base) | Weight Fraction | | | |
| | Addition Ratio | (kg/kg) | | | |
| | pH After | pH | 6.0 | 5.9-6.1 | |
| | Adjustment | | | | |
| AEX Load | Cold Storage | ° C. | 2-8 | | In order to mitigate |
| Hold Storage | Condition | | | | potential product |
| | | | | | degradation, hold |
| | | | | | times should be as |
| | | | | | short as possible, |
| | | | | | particularly at |
| | | | | | ambient temperatures. |
| | | Hours | ≤144 | | |
| | Ambient Storage | ° C. | 15-25 | | |
| | Condition | Hours | ≤72 | | |
| Load | Loading | g/L Resin | 50-60 | | Narrow loading range required |
| | | | | | for acceptable step yield |
| | | | | | and product quality |
| | Load Temperature | ° C. | 18 | 15-25 | |
| | Linear Flow Rate | cm/hr | See Comment | | Linear flow rate to be |
| | Residence Time | Minutes | 4 | | determined based on packed |
| | | | | | column bed height to ensure |
| | | | | | 4 minute residence time |
| Wash 1 (50 mM | Volume | CV | 3 | ≥2.8 | |
| Histidine, pH 6.0) | Linear Flow Rate | cm/hr | 300 | 270-330 | |
| Elution (50 mM | Linear Flow Rate | cm/hr | 300 | 270-330 | Ensure skid path length allows |
| Histidine, 105 mM | | | | | for accurate elution collection |
| NaCl, pH 6.0) | Start Collection | AU/cm | 0.5 | N/A | |
| | End Collection | AU/cm | 2.75 | N/A | |
| | at UV280 | | | | |
| | Expected Elution CV | CV | 4.5-5.5 | | |
| Strip (50 mM | Volume | CV | 3 | ≥2.8 | N/A |
| Sodium Acetate, 1M | Linear Flow Rate | cm/hr | 300 | 270-330 | |
| Sodium Chloride, pH 5.5) | | | | | |
| Post-Use Sanitization | Volume | CV | 3 | ≥3 | This step only needs to be |
| (0.5M NaOH) | | | | | performed if no cycles are |
| | | | | | planned within 24 hours. |
| | | | | | Otherwise, proceed to the |
| | | | | | Pre-Use Sanitization Step of |
| | | | | | the next cycle after |
| | | | | | completing Post-Use Strip. |
| | Linear Flow Rate | cm/hr | 300 for 1 CV, then 76 | ≤330 | |
| | Contact Time | minutes | 30 | 30-60 | |
| Storage (0.1M | Volume | CV | 3 | ≥2.8 | Only perform this step if |
| Sodium Hydroxide) | | | | | no cycles are planned within |
| | | | | | 24 hours after Post-Use |
| | | | | | Sanitization. |
| | Linear Flow Rate | cm/hr | 300 | 270-330 | |

| | | | | |
|---|---|---|---|---|
| Filtration Operating | Membrane Type | Description | 0.45/0.2 μm PES | Filter throughput data |
| Conditions | | | | achieved at pilot scale. |
| | | | | Filter sizing study has not |
| | | | | been completed. |
| | Load Capacity | L/m² | <500 | |
| Hold Storage | Cold Storage | ° C. | 2-8 | In order to mitigate |
| | Condition | | | potential product |
| | | | | degradation, hold |
| | | | | times should be as |
| | | | | short as possible, |
| | | | | particularly at |
| | | | | ambient temperatures. |
| | | Hours | ≤144 | |
| | Ambient Storage | ° C. | 15-25 | |
| | Condition | Hours | ≤120 | |

Notes:
Storage limits based on intermediate stability study, RPT-0094. Cumulative hold study will be used to set future manufacturing hold limits, RPT-0119. For current storage limits see RPT-0117.

As previously mentioned, the loading range was set to 50-60 g/L as data indicates that underchallenging the column will result in low yield and a shift in the charge profile. This narrow loading range requires a flexible column bed height that can be used to optimize cycle number in order to maximize the amount of protein purified over the AEX step. In addition, the load linear flow rate is to be determined based upon the packed column bed height to ensure a 4 minute residence time. For Demo Run 2, a 15.4 cm bed height was used and the load flow rate was set to 231 cm/hr to ensure a 4 minute residence time. Two cycles were performed and the results are shown in Table 3-23.

TABLE 3-23
Demo Run 2 AEX Process Performance and Analytical Results

| Step | Parameter | Demo Run 2 | |
|---|---|---|---|
| AEX | Cycle | 1 | 2 |
| | Column Height (cm) | 15.4 | |
| | Column ID (cm) | 20.0 | |
| | Load Concentration (g/L) | 4.9 | |
| | Resin | Capto ™ Q ImpRes | |
| | Resin Challenge (g/L resin) | 60 | 59 |
| | Elution Volume (CV) | 4.9 | 5.2 |
| | Elution Concentration (g/L) | 9.1 | 8.4 |
| | Elution Total Protein (g) | 216 | 212 |
| | HCP AEX Load (ppm) | 2.33 | |
| | HCP AEX Eluate (ppm) | 0.91 | |
| | SEC AEX Load (% agg/monomer/frag) | 0.2/99.3/0.5 | |
| | SEC AEX EL (% agg/monomer/frag) | 0.04/99.4/0.5 | |
| | % Hab AEX Eluate | 0.6 | |
| | IEC AEX Load (main/acidic/basic) | 63.0/19.4/17.6 | |
| | IEC AEX Eluate (main/acidic/basic) | 64.0/16.3/19.7 | |
| | NR CESDS AEX Load (Intact/Peak 1/Hab) | 97.48/1.38/0.63 | |
| | NR CESDS AEX Eluate (Intact/Peak 1/Hab) | 97.62/1.09/0.66 | |
| | Yield | 74% | 75% |

Demo Run 2 process performance and produce quality aligned with small scale process development data. The AEX step provided 3% acidic species reduction, slight reductions in aggregates and HCP, and the step yield was 74-75%. AEX eluate was forward processed through drug substance and demonstrated acceptable product quality.

### Scale-Up and Facility Fit Considerations

A 60 cm×17 cm column is recommended for the proposed 2000 L scale manufacturing process. This assumes an estimated titer of 6.5-8.5 g/L. Based upon the facility fit calculation shown in Table 3-24, the estimated number of AEX cycles at 2000 L scale is 2-5.

TABLE 3-24
Facility Fit Calculations for Phase 3 2000 L Scale Chromatography

| | | Low | | High |
|---|---|---|---|---|
| | | End | Target | End |
| Bioreactor | Titer (g/L) | 6.5 | 7.5 | 8.5 |
| | Packed Cell | 7% | 7% | 7% |
| | Volume (%) | | | |
| | Bioreactor | 2000 | 2000 | 2000 |
| | Volume (L) | | | |
| | Amount (g) | 12090 | 13950 | 15810 |
| Clarification | Yield (%) | 95% | 97% | 100% |
| | Amount (g) | 11486 | 13532 | 15810 |
| Protein A | Column ID (cm) | 60 | 60 | 60 |
| Chromatography | Bed height (cm) | 20 | 20 | 20 |
| (MabSelect Sure | CV (L) | 56.5 | 56.5 | 56.5 |
| LX) | Cycles | 5 | 6 | 7 |
| | Load challenge | 41 | 40 | 40 |
| | (g/L resin) | | | |
| | Yield (%) | 97% | 97% | 97% |
| | Amount (g) | 11141 | 13126 | 15336 |
| Viral | Yield (%) | 97% | 100% | 100% |
| Inactivation | Amount (g) | 10807 | 13126 | 15336 |
| Cation Exchange | Column ID (cm) | 80 | 80 | 80 |
| Chromatography | Bed height (cm) | 20 | 20 | 20 |
| (POROS ™ XS) | CV (L) | 100.5 | 100.5 | 100.5 |
| | Cycles | 2 | 3 | 3 |
| | Load challenge | 55 | 44 | 51 |
| | (g/L resin) | | | |
| | Yield (%) | 55% | 65% | 81% |
| | Amount (g) | 5944 | 8532 | 12422 |
| Anion Exchange | Column ID (cm) | 60 | 60 | 60 |
| Chromatography | Bed height (cm) | 17 | 17 | 17 |
| (Capto ™ Q | CV (L) | 48.1 | 48.1 | 48.1 |
| ImpRes) | Cycles | 2 | 3 | 5 |
| | Load challenge | 62 | 59 | 52 |
| | (g/L resin) | | | |
| | Yield (%) | 60% | 72% | 84% |
| | Amount (g) | 3566 | 6143 | 10434 |

### Conclusion

The AEX development activities focused on providing robust modulation of charged species while maintaining acceptable step yield. The Capto™ Q ImpRes resin demonstrated removal of 2-4% acidic species at both 10 L and 200 L bioreactor scale. The step also contributed to clearance of aggregates and HCP. The proposed loading range for the AEX column is 50-60 g/L. The estimated step yield is 60-84% with an elution volume of 4.5-5.5 CVs. A proposed process is detailed in Table. Analytical data of drug substance and in-process impurity clerance from non-GMP (200 L) and GMP (200 L and 2000 L) production runs, which included the Capto™ Q ImpRes chromatography process in Table 3-25, are summarized in Tables 4-1 and 4-2.

TABLE 3-25
AEX Chromatography Process

| | | Volume | Flow Rate | |
|---|---|---|---|---|
| Step | Buffer/Solution | (CVs) | (cm/hr) | Comments |
| Pre-Use | 0.5M NaOH | 3 | 300 for 1 CV, | 30 minute |
| Sanitization | | | then 64 | contact time |
| Charge | 50 mM Sodium Acetate, 1M | 3 | 300 | |
| | Sodium Chloride, pH 5.5 | | | |
| Equilibration | 50 mM Histidine, pH 6.0 | 4 | 300 | |
| Load | AEX Load pH 5.9-6.1 | Variable | Target 4 Minute | 50-60 g/L |
| | | | Contact Time | resin |
| Wash | 50 mM Histidine, pH 6.0 | 3 | 300 | |
| Elution | 50 mM Histidine, 105 mM | Variable | 300 | Collect |
| | Sodium Chloride, pH 6.0 | | | 0.5 AU/cm- |
| | | | | 2.75 AU/cm |
| Strip | 50 mM Acetate, 1M NaCl, pH 5.5 | 3 | 300 | |
| Post-Use | 0.5M NaOH | 3 | 300 for 1 CV, | 30 minute |
| Sanitization | | | then 64 | contact time |
| Storage | 0.1M NaOH | 3 | 300 | |

### Example 4. 2000 L Scale-Up; Batch Analysis

The upstream and downstream production processes developed in Examples 1, 2 and 3 were scaled-up 10-fold to a 2000 L bioreactor with no impact to product quality, demonstrating that the upstream and downstream are scalable and robust. Analytical data of drug substance and in-process impurity clearance from several non-GMP and GMP production runs, including at 2000 L scale, using the upstream process of Table 1-1 (200 L process, with media feed increase on day 5) and downstream processes of Tables 2-30 and 3-25 are summarized in Tables 4-1, 4-2 and 4-3.

TABLE 4-1
Drug Substance Batch Analysis

| | Process 7 | Process 7 | Process 7 | Process 7 | Process 7 | Process 7a |
|---|---|---|---|---|---|---|
| | 200 L | 200 L | 200 L | 200 L | 200 L | 2000 L |
| | Non-GMP | Non-GMP | GMP | GMP | GMP | GMP |
| Test | Lot 1 | Lot 2 | Lot 1 | Lot 2 | Lot 3 | Lot 1 |
| pH | 5.8 | 5.8 | 5.8 | 5.8 | 5.8 | 5.7 |
| Total protein | 149 | 149 | 160 | 159 | 155 | 153 |
| | mg/mL | mg/mL | mg/mL | mg/mL | mg/mL | mg/ml |
| Ion exchange | Main peak = | Main: | Main: | Main: | Main: | Main: |
| chromatography | 62.8% | 64.4% | 62.5% | 62.1% | 66.9% | 63.6% |
| | Acidic peaks = | Acidic: | Acidic: | Acidic: | Acidic: | Acidic: |
| | 15.5% | 15.7% | 13.5% | 12.6% | 15.2% | 14.0% |
| | Basic peaks = | Basic: | Basic: | Basic: | Basic: | Basic: |
| | 21.7% | 19.9% | 24.0% | 25.3% | 17.9% | 22.4% |
| Reporter gene | 105% | 97% | 98% | 113% | 95% | 96% |
| bioassay | | | | | | |
| Size exclusion | Monomer = | Monomer = | Monomer: | Monomer: | Monomer: | Monomer: |
| chromatography | 98.3% | 99.0% | 98.6% | 98.7% | 98.3% | 98.4% |
| | Aggregate = | Aggregate = | Total | Total | Total | Total |
| | 0.5% | 0.5% | Aggregates: | Aggregates: | Aggregates: | Aggregates: |
| | | | 0.7% | 0.7% | 0.8% | 0.6% |
| | Fragment = | Fragment = | Fragment: | Fragment: | Fragment: | Fragment: |
| | 1.2% | 0.6% | 0.7% | 0.7% | 1.0% | 1.0% |
| Reducing CE- | % Purity | % Purity | % Purity | % Purity | % Purity | % Purity |
| SDS | (HC + LC): | (HC + LC): | (HC + LC): | (HC + EC): | (HC + EC): | (HC + EC): |
| | 98.8% | 99.0% | 97.6% | 97.7% | 97.2% | 97.6% |
| | % Fragments: | % Fragments: | % Fragments: | % Fragments: | % Fragments: | % Fragments: |
| | 0.8% | 0.7% | 1.0% | 1.0% | 1.2% | 0.7% |
| | % Other: | % Other: | % Other: | % Other: | % Other: | % Other: |
| | 0.4% | 0.4% | 1.5% | 1.3% | 1.6% | 1.7% |
| Non-reducing | Major peak = | Major peak = | % Major | % Major | % Major | % Major |
| CE-SDS | 96.2% | 97.3% | Product Peak: | Product Peak: | Product Peak: | Product Peak: |
| | | | 97.3% | 97.7% | 97.3% | 97.2% |
| | ½ antibody = | ½ antibody = | %½ mAb: | %½ mAb: | %½ mAb: | %½ |
| | 1.5% | 0.6% | 0.9% | 0.7% | 0.8% | mAb: <EOQ |
| Host cell DNA | <0.08 | <0.08 | <0.08 | <0.08 | <0.08 | <1 |
| | pg/mg | pg/mg | pg/mg | pg/mg | pg/mg | pg/mg |
| Host cell | <1 | <1 | 0.9 | 2 | 2 | 1 |
| protein | ng/mg | ng/mg | ng/mg | ng/mg | ng/mg | ng/mg |

TABLE 4-2
CEX and AEX Impurity Clearance

| | | Process 7 200 L | | Process 7 200 L | | Process 7 200 L | | Process 7a 2000 L | |
|---|---|---|---|---|---|---|---|---|---|
| Source | Test | GMP Lot 1 | | GMP Lot 2 | | GMP Lot 3 | | GMP Lot 1 | |
| CEX | HCP | 349.30 | ng/mg | 283.79 | ng/mg | 302 | ng/mg | 459.9 | ng/mg |
| Load | SEC | % Monomer: 97.9 | | % Monomer: 98.0 | | % Monomer: 97.7 | | % Monomer: 97.9 | |
| | | % Aggregates: 1.1 | | % Aggregates: 0.9 | | % Aggregates: 1.0 | | % Aggregates: 0.9 | |
| | | % Total Fragments: 1.1 | | % Total Fragments: 1.2 | | % Total Fragments: 1.3 | | % Total Fragments: 1.2 | |
| | NR- | % Main: 94.2 | | % Main: 94.3 | | % Main: 92.9 | | % Main: 92.2 | |
| | CE- | % 1/2 mAb: 3.9 | | % 1/2 mAb: 3.8 | | % 1/2 mAb: 4.7 | | % 1/2 mAb: 3.2 | |
| | SDS | % Other: 1.9 | | % Other: 1.8 | | % Other: 2.4 | | % Other: 1.6 | |
| | IEC | % Main: 57.1 | | % Main: 59.4 | | % Main: 58.4 | | % Main: 58.9 | |
| | | % Total Acidic: 15.3 | | % Total Acidic: 15.0 | | % Total Acidic: 19.9 | | % Total Acidic: 17.0 | |
| | | % Total Basic: 27.6 | | % Total Basic: 25.7 | | % Total Basic: 21.7 | | % Total Basic: 24.1 | |
| | rProA | 0.6 | ng/mg | 0.8 | ng/mg | 0.9 | ng/mg | 28.3 | ng/mg |
| | rDNA | 153.16 | pg/mg | 40.75 | pg/mg | 130.62 | pg/mg | 72 | pg/mg |
| CEX | HCP | 2.76 | ng/mg | 2.26 | ng/mg | 3 | ng/mg | <54.55 | |
| Eluate/ | SEC | % Monomer: 99.0 | | % Monomer: 99.0 | | % Monomer: 98.9 | | % Monomer: 95.7 | |
| AEX | | % Aggregates: 0.3 | | % Aggregates: 0.3 | | % Aggregates: 0.4 | | % Aggregates: 4.1 | |
| Load | | % Total Fragments: 0.6 | | % Total Fragments: 0.7 | | % Total Fragments: 0.8 | | % Total Fragments: 0.1 | |
| | NR- | % Main: 97.7 | | % Main: 97.7 | | % Main: 97.5 | | % Main: 98.4 | |
| | CE- | % 1/2 mAb: 0.6 | | % 1/2 mAb: 0.7 | | % 1/2 mAb: 0.8 | | % 1/2 mAb: 0.5 | |
| | SDS | % Other: 1.7 | | % Other: 1.6 | | % Other: 1.7 | | % Other: 1.1 | |
| | IEC | % Main: 62.2 | | % Main: 64.3 | | % Main: 63.4 | | % Main: 62.3 | |
| | | % Total Acidic: 17.0 | | % Total Acidic: 14.9 | | % Total Acidic: 21.0 | | % Total Acidic: 20.7 | |
| | | % Total Basic: 20.8 | | % Total Basic: 20.8 | | % Total Basic: 15.7 | | % Total Basic: 17.1 | |
| | rProA | 0.3 | ng/mg | 0.5 | ng/mg | 0.4 | ng/mg | 3.95 | ng/mg |
| | rDNA | <1.48 | pg/mg | <1.34 | pg/mg | <1.49 | pg/mg | <1 | pg/mg |
| AEX | HCP | 1.04 | ng/mg | 0.81 | ng/mg | 1 | ng/mg | 1.58 | ng/mg |
| Eluate | SEC | % Monomer: 99.2 | | % Monomer: 99.2 | | % Monomer: 99.0 | | % Monomer: 99.9 | |
| | | % Aggregates: 0.1 | | % Aggregates: 0.0 | | % Aggregates: 0.1 | | % Aggregates: 0.1 | |
| | | % Total Fragments: 0.8 | | % Total Fragments: 0.8 | | % Total Fragments: 0.9 | | % Total Fragments: 0.1 | |
| | NR- | % Main: 97.0 | | % Main: 97.3 | | % Main: 97.0 | | % Main: 98.5 | |
| | CE- | % 1/2 mAb: 0.6 | | % 1/2 mAb: 0.7 | | % 1/2 mAb: 1.0 | | % 1/2 mAb: 0.6 | |
| | SDS | % Total Other: 2.4 | | % Other: 2.0 | | % Other: 2.0 | | % Other: 0.9 | |
| | IEC | % Main: 62.2 | | % Main: 60.3 | | % Main: 65.9 | | % Main: 63.1 | |
| | | % Total Acidic: 14.0 | | % Total Acidic: 14.0 | | % Total Acidic: 17.3 | | % Total Acidic: 16.3 | |
| | | % Total Basic: 23.9 | | % Total Basic: 25.7 | | % Total Basic: 16.7 | | % Total Basic: 20.6 | |
| | rProA | <0.1 | ng/mg | <0.1 | ng/mg | <0.1 | ng/mg | 4.16 | ng/mg |
| | rDNA | <1.35 | pg/mg | <1.52 | pg/mg | <1.34 | pg/mg | <1 | pg/mg |

TABLE 4-3
SEC Results for Process 7a 2000 L GMP Lot 1

| | Mean % | Mean % | Mean % |
|---|---|---|---|
| Sample Description | Monomer | Aggregate | Fragments |
| End of Production | 96.2 | 1.8 | 2.0 |
| Clarified Harvest | 96.7 | 1.5 | 1.8 |
| Protein A (Cycle #1) Eluate | 97.9 | 0.9 | 1.3 |
| Protein A (Cycle #2) Eluate | 97.9 | 0.9 | 1.2 |
| Protein A (Cycle #3) Eluate | 97.8 | 0.9 | 1.3 |
| Protein A (Cycle #4) Eluate | 98.1 | 0.8 | 1.1 |
| Protein A (Cycle #5) Eluate | 98.1 | 0.8 | 1.1 |
| Protein A (Cycle #6) Eluate | 98.0 | 0.8 | 1.2 |
| Protein A Pool | 97.9 | 0.9 | 1.2 |
| Viral Inactivation Pool | 97.9 | 0.9 | 1.2 |
| CEX (Cycle #1) Load | 97.8 | 0.9 | 1.3 |
| CEX (Cycle #1) Eluate | 94.7 | 5.2 | 0.1 |
| CEX (Cycle #2) Eluate | 94.8 | 5.0 | 0.1 |
| CEX (Cycle #3) Eluate | 97.3 | 2.6 | 0.1 |
| CEX Pool | 95.7 | 4.1 | 0.1 |
| AEX (Cycle #1) Eluate | 99.8 | 0.1 | 0.1 |
| AEX (Cycle #2) Eluate | 99.8 | 0.2 | 0.1 |
| AEX (Cycle #3) Eluate | 98.1 | 0.2 | 1.7 |
| AEX (Cycle #4) Eluate | 99.8 | 0.1 | 0.1 |
| AEX Pool | 99.9 | 0.1 | 0.1 |
| Viral Filtration Pool | 99.9 | 0.1 | 0.1 |
| UFDF Load | 96.4 | 0.1 | 0.1 |

Sequences
>NP_006131.2 granulocyte-macrophage
colony-stimulating factor receptor subunit alpha
isoform a precursor [Homo sapiens]
SEQ ID NO: 1
MLLLVTSLLLCELPHPAFLLIPEKSDLRTVAPASSLNVRFDSRTMNLSWDC
QENTTFSKCFLTDKKNRVVEPRLSNNECSCTFREICLHEGVTFEVHVNTSQ
RGFQQKLLYPNSGREGTAAQNFSCFIYNADLMNCTWARGPTAPRDVQYFLY
IRNSKRRREIRCPYYIQDSGTHVGCHLDNLSGLTSRNYFLVNGTSREIGIQ
FFDSLLDTKKIERFNPPSNVTVRCNTTHCLVRWKQPRTYQKLSYLDFQYQL
DVHRKNTQPGTENLLINVSGDLENRYNFPSSEPRAKHSVKIRAADVRILNW
SSWSEAIEFGSDDGNLGSVYIYVLLIVGTLVCGIVLGFLFKRFLRIQRLFP
PVPQIKDKLNDNHEVEDEIIWEEFTPEEGKGYREEVLTVKEIT
HEAVY CHAIN
SEQ ID NO: 2
QVQLVQSGAE VKKPGASVKV SCKVSGYTLT ELSIHWVRQA
PGKGLEWMGG FDPEENEIVY AQRFQGRVTM TEDTSTDTAY
MELSSLRSED TAVYYCAIVG SFSPLTLGLW GQGTMVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPSCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
LIGHT CHAIN
SEQ ID NO: 3
QSVLTQPPSV SGAPGQRVTI SCTGSGSNIG APYDVSWYQQ
LPGTAPKLLI YHNNKRPSGV PDRFSGSKSG TSASLAITGL
QAEDEADYYC ATVEAGLSGS VFGGGTKLTV LGQPKAAPSV
TLFPPSSEEL QANKATLVCL ISDFYPGAVT VAWKADSSPV
KAGVETTTPS KQSNNKYAAS SYLSLTPEQW KSHRSYSCQV
THEGSTVEKT VAPTECS
HEAVY CHAIN VARIABLE REGION
SEQ ID NO: 4
QVQLVQSGAEVKKPGASVKVSCKVSGYTLTELSIHWVRQAPGKGLEWMGGF
DPEENEIVYAQRFQGRVTMTEDTSTDTAYMELSSLRSEDTAVYYCAIVGSF
SPLTLGLWGQGTMVTVSS
LIGHT CHAIN VARIABLE REGION
SEQ ID NO: 5
QSVLTQPPSVSGAPGQRVTISCTGSGSNIGAPYDVSWYQQLPGTAPKLLIY
HNNKRPSGVPDRFSGSKSGTSASLAITGLQAEDEADYYCATVEAGLSGSVF
GGGTKLTVL
HEAVY CHAIN CDR1
SEQ ID NO: 6
ELSIH
HEAVY CHAIN CDR2
SEQ ID NO: 7
GFDPEENEIVYAQRFQG
HEAVY CHAIN CDR3
SEQ ID NO: 8
VGSFSPLTLGL
LIGHT CHAIN CDR1
SEQ ID NO: 9
TGSGSNIGAPYDVS
LIGHT CHAIN CDR2
SEQ ID NO: 10
HNNKRPS
LIGHT CHAIN CDR3
SEQ ID NO: 11
ATVEAGLSGSV

1. A method of producing a preparation comprising a protein of interest having a reduced level of half antibody, the method comprising subjecting a sample comprising the protein of interest and half antibody to a cation exchange chromatography resin or a mixed mode chromatography resin, thereby producing the preparation comprising the protein of interest having a reduced level of half antibody.

2. A method of reducing the level of half antibody in a preparation comprising a protein of interest, the method comprising subjecting a sample comprising the protein of interest and half antibody to a cation exchange chromatography resin or a mixed mode chromatography resin, thereby reducing the level of half antibody in the preparation comprising the protein of interest.

3. The method of claim 1, wherein the protein of interest is an antibody or antigen-binding portion thereof; optionally, wherein the antibody or antigen-binding portion thereof is an anti-GM-CSFRα antibody or antigen-binding portion thereof; optionally, wherein the anti-GM-CSFRα antibody or antigen-binding portion thereof is mavrilimumab.

4. (canceled)

5. (canceled)

6. The method of claim 1, wherein the sample is subject to a cation exchange chromatography resin; optionally,
(a) wherein the cation exchange chromatograph resin comprises a functional group selected from the group consisting of sulfhydryl, sulfonate, sulfate, carboxymethyl, sulfoethyl, sulfopropyl, phosphate and sulfonate;
(b) wherein the cation exchange chromatograph resin is selected from the group consisting of POROS™ XS CEX, Capto™ S ImpAct, TOTOPEARL™ GigaGap CM 650M, and TOYOPEAL™ sulfate 650F; and/or
(c) wherein the cation exchange chromatograph resin runs in bind-elute mode.

7-9. (canceled)

10. The method of claim 1, wherein the sample is subject to a mixed mode chromatography resin; optionally,
(a) wherein the mixed mode chromatography resin comprises a functional group selected from the group consisting of carboxyl, hydroxyl, N-Benzyl-N-methyl ethanol amine, phenylpropylamine and Hexylamine; and/or
(b) wherein the mixed mode chromatography resin is selected from the group consisting of Capt™ MMC ImpRes and Capto™ Adhere ImpRes.

11. (canceled)

12. (canceled)

13. The method of claim 1,
(a) wherein the preparation comprises less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody; optionally, wherein the preparation comprises less than about 2.8% half antibody;
(b) wherein the preparation comprises about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody; optionally, wherein the preparation comprises about 0.6-1.7% half antibody;
(c) the level of half antibody in the preparation is reduced by at least about 90%, about 80%, about 70%, about 60%, about 50%, about 40%, about 30%, about 20% or about 10% as compared to the level of half antibody in the sample; and/or
(d) wherein the level of half antibody is determined by non-reduced CE-SDS (capillary electrophoresis with sodium dodecylsulfate).

14-17. (canceled)

18. The method of claim 1, further comprising collecting an eluate fraction using an elution buffer; optionally,
(a) wherein the eluate fraction comprises less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody;
(b) wherein the eluate fraction comprises about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18%, or about 1-17% half antibody;
(c) wherein the eluate fraction is collected from cation exchange chromatography resin; optionally, wherein the eluate fraction collected from cation exchange chromatography resin comprises about 0.6-18% half antibody;
(d) wherein the eluate fraction is collected from mixed mode chromatography resin; optionally, wherein the eluate fraction collected from mixed mode chromatography resin and comprises about 1-17% half antibody;
(e) wherein the level of half antibody in the eluate fraction is reduced by at least about 90%, about 80%, about 70%, about 60%, about 50%, about 40%, about 30%, about 20% or about 10% as compared to the level of half antibody in the sample;
(f) wherein the elution buffer comprises about 1-500 mM, about 10-250 mM, about 10-150 mM, about 10-100 mM, about 20-90 mM, about 30-80 mM, about 40-70 mM, or about 50-60 mM sodium acetate; optionally, wherein the elution buffer comprises about 40-60 mM sodium acetate;
(g) wherein the elution buffer comprises about 1-500 mM, about 10-250 mM, about 10-150 mM, about 10-100 mM, about 20-90 mM, about 30-80 mM, about 40-70 mM, or about 50-60 mM sodium chloride; optionally, wherein the elution buffer comprises about 40-60 mM sodium chloride;
(h) wherein the elution buffer comprises a pH of about 4-7, about 5-6, about 5-5.5; optionally, wherein the elution buffer comprises a pH of about 5-5.5; and/or
(i) wherein the elution buffer comprises about 50 mM sodium acetate, about 55 mM sodium chloride, and a pH of about 5.35.

19-32. (canceled)

33. The method of claim 1, wherein the protein of interest is loaded onto the cation exchange chromatography resin or the mixed mode chromatography resin at a level of about 10-100 g/L, about 20-90 g/L, about 30-80 g/L, about 40-70 g/L, or about 50-60 g/L; optionally, wherein the protein of interest is loaded onto the cation exchange chromatography resin or the mixed mode chromatography resin at a level of about 30-60 g/L.

34. (canceled)

35. (canceled)

36. A composition comprising an anti-GM-CSFRα antibody, or antigen-binding portion thereof, comprising less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

37. The composition of claim 36,
(a) wherein the composition comprises less than about 2.8% half antibody;
(b) wherein the composition comprises about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody;
(c) wherein the composition comprises about 0.6-1.7% half antibody;
(d) wherein the level of half antibody is determined by non-reduced CE-SDS (capillary electrophoresis with sodium dodecylsulfate); and/or
(e) wherein the anti-GM-CSFRα antibody or antigen-binding portion thereof is mavrilimumab.

38. (canceled)

39. (canceled)

40. A composition comprising an anti-GM-CSFRα antibody, or antigen-binding portion thereof, wherein the composition comprises an eluate fraction collected from a cation exchange chromatography resin or a mixed mode chromatography resin, and wherein the eluate fraction comprises less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2.8%, about 2%, about 1% or about 0.5% half antibody.

41. The composition of claim 40,
(a) wherein the eluate fraction comprises about 0.1-20%, about 0.1-10%, about 0.1-9%, about 0.1-8%, about 0.1-7%, about 0.1-6%, about 0.1-5%, about 0.1-4%, about 0.1%-3%, about 0.1%-2.8%, about 0.5%-2.5%, about 0.5%-1.5%, about 0.6-1.7%, about 0.6-18% or about 1-17% half antibody;
(b) wherein the eluate fraction is collected from a cation exchange resin and comprises about 0.6-18% half antibody;
(c) wherein the eluate fraction is collected from a mixed mode resin and comprises about 1-17% half antibody;
(d) wherein the level of half antibody is determined by non-reduced CE-SDS (capillary electrophoresis with sodium dodecylsulfate); and/or
(e) wherein the anti-GM-CSFRα antibody or antigen-binding portion thereof is mavrilimumab.

42. (canceled)

43. (canceled)

44. A composition comprising an anti-GM-CSFRα antibody, or antigen-binding portion thereof, wherein the composition comprises a flow through and/or a wash fraction collected from a cation exchange chromatography resin, and wherein the flow through and/or wash fraction comprises less than about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2% or about 1% half antibody.

45. The composition of claim 44,
(a) wherein the flow through and/or wash fraction comprises less than about 6% half antibody;
(b) wherein the level of half antibody is determined by non-reduced CE-SDS (capillary electrophoresis with sodium dodecylsulfate); and/or
(c) wherein the anti-GM-CSFRα antibody or antigen-binding portion thereof is mavrilimumab.

46. (canceled)

47. (canceled)

48. A pharmaceutical composition comprising the composition of claim 36, and a pharmaceutically acceptable carrier.

49-250. (canceled)

251. A pharmaceutical composition comprising the composition of claim 40, and a pharmaceutically acceptable carrier.

252. A pharmaceutical composition comprising the composition of claim 44, and a pharmaceutically acceptable carrier.
